(19) United States
(12) Patent Application Publication
(10) Pub. No.: US 2021/0020846 A1
(43) Pub. Date: Jan. 21, 2021

(54) ORGANIC ELECTROLUMINESCENT ELEMENT AND ELECTRONIC DEVICE

(71) Applicant: IDEMITSU KOSAN CO.,LTD., Chiyoda-ku (JP)

(72) Inventors: Kei YOSHIZAKI, Sodegaura-shi, (JP); Toshinari OGIWARA, Sodegaura-shi (JP); Yuichiro KAWAMURA, Sodegaura-shi (JP); Keiichi YASUKAWA, Sodegaura-shi (JP); Kei YOSHIDA, Sodegaura-shi (JP)

(73) Assignee: IDEMITSU KOSAN CO.,LTD., Chiyoda-ku (JP)

(21) Appl. No.: 16/981,945

(22) PCT Filed: Mar. 18, 2019
(86) PCT No.: PCT/JP2019/011192

§ 371 Date: Sep. 17, 2020

(30) Foreign Application Priority Data
Mar. 19, 2018 (JP) 2018-051804

(51) Int. Cl. H01L 51/00; C09K 11/06

(52) U.S. Cl. CPC H01L 51/0067; H01L 51/0072; H01L 51/5012; H01L 2251/5384; C09K 11/06

(57) ABSTRACT

An organic electroluminescence device includes an anode, a first organic layer, and a cathode in this order. The first organic layer contains a first compound, a second compound, and a third compound. The first compound is a compound represented by a formula (1). The second compound is a delayed fluorescent compound. The third compound is a compound represented by a formula (3). In the formula (1), X is a nitrogen atom or a carbon atom bonded to Y, Y being a hydrogen atom or a substituent. In the formula (3), n is 1 to 4, X_{B} is a group represented by a formula (3A). In the formula (3A), Ar₁ and Ar₂ are each independently a monovalent or polyvalent aromatic hydrocarbon group, and Cz is a group represented by a formula (3B-1) and the like.

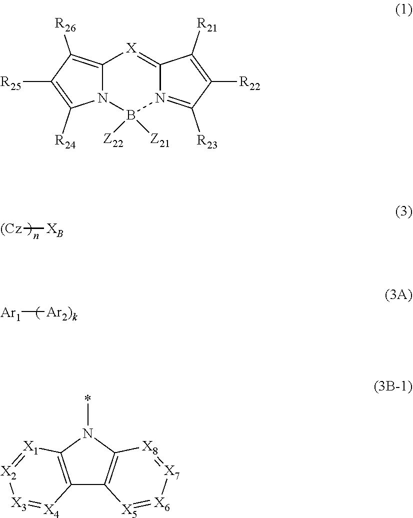

### TECHNICAL FIELD

The present invention relates to an organic electroluminescence device and an electronic device.

### BACKGROUND ART

When a voltage is applied to an organic electroluminescence device (hereinafter, occasionally referred to as “organic EL device”), holes and electrons are injected into an emitting layer from an anode and a cathode, respectively. The injected electrons and holes are recombined in the emitting layer to form excitons. At this time, according to the electron spin statistics theory, singlet excitons and triplet excitons are generated at a ratio of 25%:75%.

A fluorescent organic EL device with emission caused by singlet excitons, which has been applied to a full-color display for a mobile phone, TV and the like, is inferred to exhibit an internal quantum efficiency of 25% at a maximum. Accordingly, studies has been made to improve a performance of the organic EL device.

Moreover, it is expected to further efficiently emit the organic EL device using triplet excitons in addition to singlet excitons. In view of the above, a highly efficient fluorescent organic EL device with thermally activated delayed fluorescence (sometimes referred to as “delayed fluorescence, hereinafter) has been proposed and studied.

For instance, a TADF (Thermally Activated Delayed Fluorescence) mechanism has been studied. The TADF mechanism uses a phenomenon where inverse intersystem crossing from triplet excitons to singlet excitons thermally occurs when a material having a small energy difference (ΔST) between singlet energy level and triplet energy level is used. As for thermally activated delayed fluorescence, refer to, for instance, ADACHI, Chihaya, ed. (Apr. 1, 2012), “Yuki Hando-tai no Debaisu Bussei (Device Physics of Organic Semiconductors)”, Kodansha, pp. 261-268. Organic EL devices with such a TADF mechanism are disclosed in, for instance, Patent Literature 2. Further, Patent Literature 1 discloses a compound having a structure similar to a compound disclosed in Patent Literature 2.

### CITATION LIST

### Patent Literature(S)

Patent Literature 1: JP 2016-6033 A

Patent Literature 2: International Publication No. WO2016/056559

### SUMMARY OF THE INVENTION

### Problems to be Solved by the Invention

However, further improvement in performance is demanded in the field of organic EL devices.

An object of the invention is to provide an organic electroluminescence device capable of achieving highly efficient emission and an electronic device including the organic electroluminescence device.

### Means for Solving the Problems

According to an aspect of the invention, an organic electroluminescence device is provided that includes: an anode;

a cathode; and

a first organic layer interposed between the anode and the cathode, in which

the first organic layer contains a first compound, a second compound, and a third compound,

the first compound is a compound represented by a formula (1) below,

the second compound is a delayed fluorescent compound, and

the third compound is a compound represented by a formula (3) below.

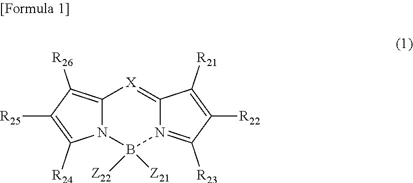

In the formula (1): X is a nitrogen atom, or a carbon atom bonded to Y;

Y is a hydrogen atom or a substituent;

R₂₁ to R₂₆ are each independently a hydrogen atom or a substituent, or at least one of a pair of R₂₁ and R₂₂, a pair of R₂₂ and R₂₃, a pair of R₂₄ and R₂₅, or a pair of R₂₅ and R₂₆ are mutually bonded to form a ring;

Y and R₂₁ to R₂₆ as the substituents are each independently selected from the group consisting of a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted aryl group having 6 to 30 ring carbon atoms, a substituted or unsubstituted alkoxy group having 1 to 30 carbon atoms, a substituted or unsubstituted alkoxy halide group having 1 to 30 carbon atoms, a substituted or unsubstituted alkylthio group having 6 to 30 carbon atoms, a substituted or unsubstituted aryloxy group having 6 to 30 ring carbon atoms, a substituted or unsubstituted arylthio group having 6 to 30 ring carbon atoms, a substituted or unsubstituted alkenyl group having 2 to 30 carbon atoms, a substituted or unsubstituted aralkyl group having 7 to 30 carbon atoms, a substituted or unsubstituted heteroaryl group having 5 to 30 ring atoms, a halogen atom, a carboxy group, a substituted or unsubstituted ester group, a substituted or unsubstituted carbamoyl group, a substituted or unsubstituted amino group, a nitro group, a cyano group, a substituted or unsubstituted silyl group, and a substituted or unsubstituted siloxanyl group;

Z₂₁ and Z₂₂ are each independently a substituent, or are mutually bonded to form a ring; and

Z₂₁ and Z₂₂ as the substituent are each independently selected from the group consisting of a halogen atom, a substituted or unsubstituted alkyl group having 1 to 30 carbon atoms, a substituted or unsubstituted alkyl halide group having 1 to 30 carbon atoms, a substituted or unsubstituted aryl group having 6 to 30 ring carbon atoms, a substituted or unsubstituted alkoxy group having 1 to 30 carbon atoms, a substituted or unsubstituted alkoxy halide group having 1 to 30 carbon atoms, and a substituted or unsubstituted aryloxy group having 6 to 30 ring carbon atoms.

[Formula 2]

(Cz)ₙ-X_{B} (3)

In the formula (3): n is 1, 2, 3 or 4;

when n is 2, 3, or 4, a plurality of Cz are mutually the same or different;

X_{B} is a group represented by a formula (3A) below; and

Cz is a group represented by a formula (3B-1) or a formula (3B-2) below.

[Formula 3]

Ar₁—(Ar₂)ₖ (3A)

In the formula (3A): Ar₁ and Ar₂ are each independently a substituted or unsubstituted monovalent or polyvalent aromatic hydrocarbon group having 6 to 18 ring carbon atoms;

k is 0, 1, or 2;

when k is 2, a plurality of Ar₂ are mutually the same or different;

Ar₁ is optionally substituted by a substituent D1, the substituent D1 being each independently selected from the group consisting of a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted aryl group having 6 to 30 ring carbon atoms, a substituted or unsubstituted aralkyl group having 7 to 30 carbon atoms, a substituted or unsubstituted alkoxy group having 1 to 30 carbon atoms, a substituted or unsubstituted alkoxy halide group having 1 to 30 carbon atoms, a substituted or unsubstituted aryloxy group having 6 to 30 ring carbon atoms, a substituted or unsubstituted silyl group, a halogen atom, a cyano group, and a nitro group;

Ar₂ is optionally substituted by a substituent D2, the substituent D2 representing the same as the substituent D1;

when n is 1, Cz is bonded to Ar₁ or Ar₂; and

when n is 2, 3 or 4, a plurality of Cz are each independently bonded to Ar₁ or Ar₂.

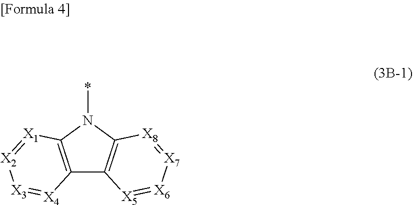

In the formula (3B-1): X₁ to X₈ each independently represent a nitrogen atom or CR_{A}, R_{A} being a hydrogen atom or a substituent, at least one pair of adjacent ones of R_{A} being optionally mutually bonded to form a ring;

R_{A} as the substituent is each independently selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted silyl group, a substituted or unsubstituted alkoxy group having 1 to 30 carbon atoms, a substituted or unsubstituted alkoxy halide group having 1 to 30 carbon atoms, a substituted or unsubstituted aryloxy group having 6 to 30 carbon atoms, a substituted or unsubstituted alkylthio group having 1 to 30 carbon atoms, a substituted or unsubstituted arylthio group having 6 to 30 ring carbon atoms, a substituted or unsubstituted aralkyl group having 7 to 30 carbon atoms, a substituted or unsubstituted alkenyl group having 2 to 30 carbon atoms, a halogen atom, a substituted or unsubstituted alkynyl group having 2 to 30 carbon atoms, a cyano group, a hydroxy group; a nitro group, and a carboxy group;

the substituted or unsubstituted heteroaryl group having 5 to 30 ring atoms for R_{A} is each independently a carbazolyl group or an azacarbazolyl group;

a plurality of R_{A} are mutually the same or different; and

the nitrogen atom in the formula (3B-1) is bonded to one of Ar₁ or Ar₂ in the formula (3A).

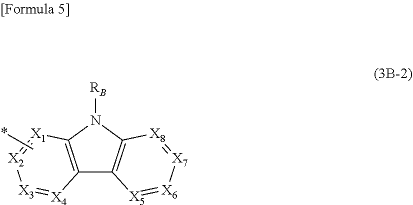

In the formula (3B-2), X₁ to X₄ are each a carbon atom bonded to one of Ar₁ and Ar₂ in the formula (3A), a nitrogen atom, or CR_{C}, R_{C} being a hydrogen atom or a substituent, at least one pair of adjacent ones of R_{C} being optionally mutually bonded to form a ring, at least one of X₁ to X₄ being a carbon atom bonded to one of Ar₁ and Ar₂ in the formula (3A);

X₅ to X₈ are each a nitrogen atom or CR_{D}, R_{D} being a hydrogen atom or a substituent, at least one pair of adjacent ones of R_{D} being optionally mutually bonded to form a ring;

R_{B}, R_{C} and R_{D} each independently represent the same as R_{A} in the formula (3B-1), a plurality of R_{C} being mutually the same or different, a plurality of R_{D} being mutually the same or different;

a substituent E in the third compound meant by “substituted or unsubstituted” is a substitutent selected from the group consisting of a substituted or unsubstituted aryl group having 6 to 30 ring carbon atoms, a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted silyl group, a substituted or unsubstituted alkoxy group having 1 to 30 carbon atoms, a substituted or unsubstituted alkoxy halide group having 1 to 30 carbon atoms, a substituted or unsubstituted aryloxy group having 6 to 30 carbon atoms, a substituted or unsubstituted alkylthio group having 1 to 30 carbon atoms, a substituted or unsubstituted arylthio group having 6 to 30 ring carbon atoms, a substituted or unsubstituted aralkyl group having 7 to 30 carbon atoms, a substituted or unsubstituted alkenyl group having 2 to 30 carbon atoms, a halogen atom, a substituted or unsubstituted alkynyl group having 2 to 30 carbon atoms, a cyano group, a hydroxy group; a nitro group, and a carboxy group;

the substituent E optionally includes a substituent F, the substituent F being selected from the group consisting of an unsubstituted aryl group having 6 to 30 ring carbon atoms, an unsubstituted alkyl group having 1 to 30 carbon atoms, an unsubstituted alkyl halide group having 1 to 30 carbon atoms, an unsubstituted cycloalkyl group having 3 to 30 ring carbon atoms, an unsubstituted silyl group, an unsubstituted alkoxy group having 1 to 30 carbon atoms, an unsubstituted halogenated alkoxy group having 1 to 30 carbon atoms, an unsubstituted aryloxy group having 6 to 30 carbon atoms, an unsubstituted alkylthio group having 1 to 30 carbon atoms, an unsubstituted arylthio group having 6 to 30 ring carbon atoms, an unsubstituted aralkyl group having 7 to 30 carbon atoms, an unsubstituted alkenyl group having 2 to 30 carbon atoms, a halogen atom, an unsubstituted alkynyl group having 2 to 30 carbon atoms, a cyano group, a hydroxy group; a nitro group, and a carboxy group, the substituent F not being further substituted.

According to another aspect of the invention, an electronic device provided with the organic electroluminescence device according to the above aspect of the invention is provided.

According to the above aspects of the invention, an organic electroluminescence device capable of achieving highly efficient emission and an electronic device including the organic electroluminescence device can be provided.

### BRIEF EXPLANATION OF DRAWING(S)

FIG. 1 schematically illustrates an overall arrangement of an organic electroluminescence device according to an exemplary embodiment.

FIG. 2 schematically shows a device of measuring transient PL.

FIG. 3 shows an example of a decay curve of the transient PL.

FIG. 4 is an illustration showing a relationship between an energy level and energy transfer for first, second and third compounds in a first organic layer (emitting layer).

### DESCRIPTION OF EMBODIMENT(S)

### First Exemplary Embodiment

Arrangement(s) of an organic EL device according to a first exemplary embodiment of the invention will be described below.

The organic EL device includes an anode, a cathode, and an at least one organic layer between the anode and the cathode. The organic layer includes at least one layer formed of an organic compound(s). Alternatively, the organic layer includes a plurality of laminated layers formed of an organic compound(s). The organic layer may further contain an inorganic compound. At least one of the organic layer(s) of the organic EL device of the present exemplary embodiment is an emitting layer. Accordingly, the organic layer may, for instance, be provided by a single emitting layer, or include a layer(s) usable for an organic EL device. Examples of the layer(s) usable for the organic EL device, which are not specifically limited, include at least one layer selected from the group consisting of a hole injecting layer, a hole transporting layer, an electron injecting layer, an electron transporting layer, and a blocking layer.

The organic EL device of the present exemplary embodiment includes a first organic layer interposed between the cathode and the anode. The first organic layer is preferably the emitting layer.

In the organic EL device of the present exemplary embodiment, the first organic layer is the emitting layer.

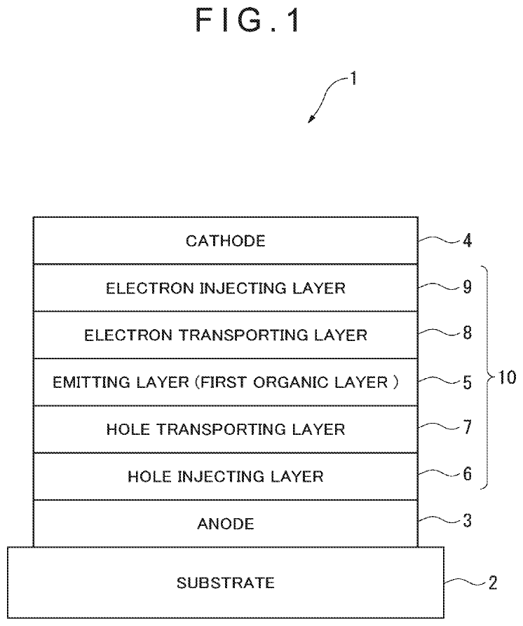

FIG. 1 schematically shows an exemplary arrangement of the organic EL device of the present exemplary embodiment.

The organic EL device 1 includes a light-transmissive substrate 2, an anode 3, a cathode 4, and an organic layer 10 provided between the anode 3 and the cathode 4. The organic layer 10 includes a hole injecting layer 6, a hole transporting layer 7, the emitting layer 5 (the first organic layer), an electron transporting layer 8, and an electron injecting layer 9, which are sequentially laminated on the anode 3.

The emitting layer 5 of the organic EL device 1 in the present exemplary embodiment contains first, second, and third compounds.

The first compound is a compound represented by the above formula (1). The second compound is a delayed fluorescent compound. The third compound is a compound represented by the above formula (3).

The emitting layer 5 may contain a metal complex.

The emitting layer 5 preferably contains no phosphorescent metal complex. It is also preferable that the emitting layer 5 does not contain a metal complex.

It is preferable that the first compound is a dopant material (sometimes referred to as a guest material, emitter, or emitting material) and the second compound is preferably a host material (sometimes referred to as a matrix material).

The second compound and the third compound are each preferably a host material. One of the second compound and the third compound may be sometimes referred to as a first host material, and the other may be sometimes referred to as a second host material.

The third compound is also preferably a material (third component) for dispersing the dopant material in the emitting layer.

The inventors have found that highly efficient emission can be achieved when the first organic layer contains the first compound (i.e. the compound represented by the formula (1)), the delayed fluorescent second compound, and the third compound (i.e. the compound represented by the formula (3)).

The inventors have selected the third compound in a form of a compound having a specific skeleton (sometimes referred to as a “specific Cz-aryl skeleton” hereinafter), in which a carbazolyl group and an aromatic hydrocarbon group are bonded. In the present exemplary embodiment, it is speculated that the presence of the compound having the specific Cz-aryl skeleton in the first organic layer (emitting layer in the present exemplary embodiment) restrains the amount of holes supplied to the first organic layer to improve a carrier balance factor in the first organic layer, thereby enhancing the efficiency of the device.

Especially, due to the presence in the first organic layer of the compound having the specific Cz-aryl skeleton (the third compound), which tends to show a large value of an ionization potential (Ip), it is believed that the amount of holes supplied to the first organic layer is eminently reduced.

Accordingly, an organic EL device that provides highly efficient emission is achieved by the present exemplary embodiment.

Arrangement(s) of an organic EL device according to the present exemplary embodiment will be detailed below. The codes will be omitted in the description below.

### First Organic Layer

The first organic layer (emitting layer in the present exemplary embodiment) contains the first compound, the second compound, and the third compound.

### First Compound

The first compound is a compound represented by a formula (1) below.

The first compound is preferably a fluorescent compound.

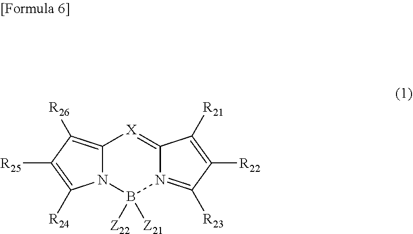

In the formula (1): X is a nitrogen atom, or a carbon atom bonded to Y;

Y is a hydrogen atom or a substituent;

R₂₁ to R₂₆ are each independently a hydrogen atom or a substituent, or at least one of a pair of R₂₁ and R₂₂, a pair of R₂₂ and R₂₃, a pair of R₂₄ and R₂₅, or a pair of R₂₅ and R₂₆ are mutually bonded to form a ring;

Y and R₂₁ to R₂₆ as the substituents are each independently selected from the group consisting of a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted aryl group having 6 to 30 ring carbon atoms, a substituted or unsubstituted alkoxy group having 1 to 30 carbon atoms, a substituted or unsubstituted alkoxy halide group having 1 to 30 carbon atoms, a substituted or unsubstituted alkylthio group having 6 to 30 carbon atoms, a substituted or unsubstituted aryloxy group having 6 to 30 ring carbon atoms, a substituted or unsubstituted arylthio group having 6 to 30 ring carbon atoms, a substituted or unsubstituted alkenyl group having 2 to 30 carbon atoms, a substituted or unsubstituted aralkyl group having 7 to 30 carbon atoms, a substituted or unsubstituted heteroaryl group having 5 to 30 ring atoms, a halogen atom, a carboxy group, a substituted or unsubstituted ester group, a substituted or unsubstituted carbamoyl group, a substituted or unsubstituted amino group, a nitro group, a cyano group, a substituted or unsubstituted silyl group, and a substituted or unsubstituted siloxanyl group;

Z₂₁ and Z₂₂ are each independently a substituent, or are mutually bonded to form a ring; and

Z₂₁ and Z₂₂ as the substituent are each independently selected from the group consisting of a halogen atom, a substituted or unsubstituted alkyl group having 1 to 30 carbon atoms, a substituted or unsubstituted alkyl halide group having 1 to 30 carbon atoms, a substituted or unsubstituted aryl group having 6 to 30 ring carbon atoms, a substituted or unsubstituted alkoxy group having 1 to 30 carbon atoms, a substituted or unsubstituted alkoxy halide group having 1 to 30 carbon atoms, and a substituted or unsubstituted aryloxy group having 6 to 30 ring carbon atoms.

In the formula (1), when, for instance, the pair of R₂₅ and R₂₆ are mutually bonded to form a ring, the first compound is represented by a formula (11) below.

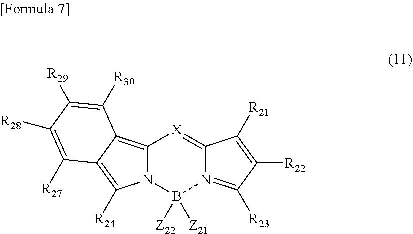

In the formula (11), X, Y, R₂₁ to R₂₄, Z₂₁, and Z₂₂ represent the same as X, Y, R₂₁ to R₂₄, Z₂₁, and Z₂₂ in the formula (1), respectively. R₂₇ to R₃₀ are each independently a hydrogen atom or a substituent, the substituent for R₂₇ to R₃₀ being the same as the substituent for R₂₁ to R₂₄.

In the formula (1), when Z₂₁ and Z₂₂ are mutually bonded to form a ring, the first compound is represented by, for instance, a formula (1A) or a formula (1B) below. However, the structure of the first compound is not limited to the structures below.

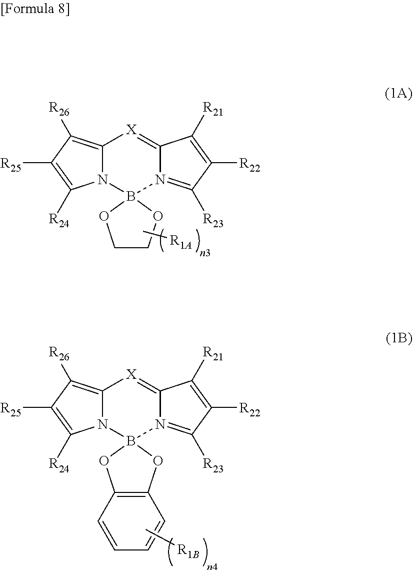

In the formula (1A), X, Y and R₂₁ to R₂₆ are the same as X, Y, and R₂₁ to R₂₆ in the formula (1), respectively. R_{1A} are each independently a hydrogen atom or a substituent, R_{1A} as a substituent being the same as the substituent for R₂₁ to R₂₆. n3 is 4.

In the formula (1B), X, Y and R₂₁ to R₂₆ represent the same as X, Y, and R₂₁ to R₂₆ in the formula (1), respectively. R_{1B} are each independently a hydrogen atom or a substituent, the substituent for R_{1B} being selected from the same group for the substituent for R₂₁ to R₂₆. n4 is 4.

At least one of Z₂₁ and Z₂₂ (preferably Z₂₁ and Z₂₂) is preferably a group selected from the group consisting of a substituted or unsubstituted alkyl group having 1 to 30 carbon atoms, a substituted or unsubstituted alkyl halide group having 1 to 30 carbon atoms, a substituted or unsubstituted aryl group having 6 to 30 ring carbon atoms, a substituted or unsubstituted alkoxy group having 1 to 30 carbon atoms, a substituted or unsubstituted alkoxy halide group having 1 to 30 carbon atoms, and a substituted or unsubstituted aryloxy group having 6 to 30 ring carbon atoms.

It is more preferable that at least one of Z₂₁ and Z₂₂ is a group selected from the group consisting of a fluorine-atom-substituted alkoxy group having 1 to 30 carbon atoms, fluorine-atom-substituted aryloxy group having 6 to 30 ring carbon atoms, and a fluoroalkyl-group-substituted aryloxy group having 6 to 30 ring carbon atoms.

Further preferably, at least one of Z₂₁ or Z₂₂ is a fluorine-substituted alkoxy group having 1 to 30 carbon atoms. Furthermore preferably, both of Z₂₁ and Z₂₂ are fluorine-substituted alkoxy groups having 1 to 30 carbon atoms.

It is also preferable that both of Z₂₁ and Z₂₂ are the same with each other.

Meanwhile, it is also preferable that at least one of Z₂₁ and Z₂₂ is a fluorine atom. More preferably, Z₂₁ and Z₂₂ are fluorine atoms.

At least one of Z₂₁ and Z₂₂ is also preferably a group represented by a formula (1a) below.

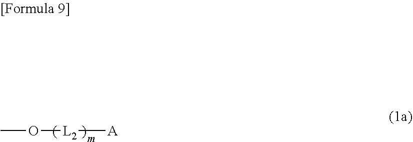

In the formula (1a), A is a substituted or unsubstituted alkyl group having 1 to 6 carbon atoms, a substituted or unsubstituted alkyl halide group having 1 to 6 carbon atoms, or a substituted or unsubstituted aryl group having 6 to 12 ring carbon atoms, L₂ is a substituted or unsubstituted alkylene group having 1 to 6 carbon atoms, or a substituted or unsubstituted arylene group having 6 to 12 ring carbon atoms, m is 0, 1, 2, 3, 4, 5, 6, or 7, and, when m is 2, 3, 4, 5, 6, or 7, a plurality of L₂ are mutually the same or different. m is preferably 0, 1 or 2. When m is 0, A is directly bonded to O (oxygen atom).

In the formula (1), when Z₂₁ and Z₂₂ are groups represented by the formula (1a), the first compound is a compound represented by a formula (10) below.

It is also preferable that the first compound is a compound represented by a formula (10) below.

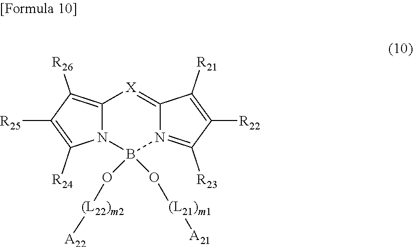

In the formula (10), X, Y when X is a carbon atom bonded to Y, and R₂₁ to R₂₆ represent the same as X, Y, and R₂₁ to R₂₆ in the formula (1). A₂₁ and A₂₂ are selected from the same group for A in the formula (1a), A₂₁ and A₂₂ being mutually the same or different. L₂₁ and L₂₂ are the same as L₂ in the formula (1a), L₂₁ and L₂₂ being mutually the same or different. m1 and m2 are each independently 0, 1, 2, 3, 4, 5, 6 or 7, preferably 0, 1 or 2. When m1 is 2, 3, 4, 5, 6 or 7, a plurality of L₂₁ are mutually the same or different. When m2 is 2, 3, 4, 5, 6 or 7, a plurality of L₂₂ are mutually the same or different. When m1 is 0, A₂₁ is directly bonded to O (oxygen atom). When m2 is 0, A₂₂ is directly bonded to O (oxygen atom).

At least one of A and L₂ in the formula (1a) is preferably substituted by a halogen atom, more preferably by a fluorine atom.

A in the formula (1a) is more preferably a perfluoroalkyl group having 1 to 6 carbon atoms, or a perfluoroaryl group having 6 to 12 ring carbon atoms, further preferably a perfluoroalkyl group having 1 to 6 carbon atoms.

L₂ in the formula (1a) is more preferably a perfluoroalkylene group having 1 to 6 carbon atoms, or a perfluoroarylene group having 6 to 12 ring carbon atoms, further preferably a perfluoroalkylene group having 1 to 6 carbon atoms.

Specifically, it is also preferable that the first compound is the compound represented by a formula (10a) below.

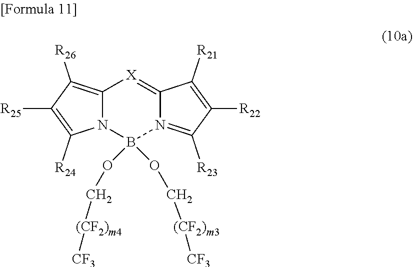

In the formula (10a): X represents the same as X in the formula (1), Y when X is a carbon atom bonded to Y represents the same as Y in the formula (1);

R₂₁ to R₂₆ each independently represent the same as R₂₁ to R₂₆ in the formula (1);

m3 is in a range from 0 to 4;

m4 is in a range from 0 to 4; and

m3 and m4 are mutually the same or different.

In the formulae (1), (11), (10) and (10a), more preferably:

X is a carbon atom bonded to Y; Y is a hydrogen atom or a substituent; and

Y as a substituent is preferably a substituent selected from the group consisting of a substituted or unsubstituted alkyl group having 1 to 30 carbon atoms, a substituted or unsubstituted alkyl halide group having 1 to 30 carbon atoms, and a substituted or unsubstituted aryl group having 6 to 30 ring carbon atoms, more preferably a substituted or unsubstituted aryl group having 6 to 30 ring carbon atoms.

In the formulae (1), (11), (10) and (10a), more preferably:

X is a carbon atom bonded to Y; Y is a hydrogen atom or a substituent;

Y as a substituent is a substituted or unsubstituted aryl group having 6 to 30 ring carbon atoms; and when Y as a substituent is an aryl group having 6 to 30 ring carbon atoms having a substituent, the substituent is a substituted or unsubstituted alkyl group having 1 to 30 carbon atoms, a substituted or unsubstituted alkyl halide group having 1 to 30 carbon atoms, a substituted or unsubstituted alkoxy group having 1 to 30 carbon atoms, a substituted or unsubstituted alkoxy halide group having 1 to 30 carbon atoms, or an aryl group having 6 to 30 ring carbon atoms and substituted by an alkyl group having 1 to 30 carbon atoms.

Though Z₂₁ and Z₂₂ of the first compound may be mutually bonded to form a ring, Z₂₁ and Z₂₂ are preferably not bonded to form no ring.

It is preferable that at least one of R₂₁, R₂₃, R₂₄, and R₂₆ in the formulae (1), (10), and (10a) is a substituted or unsubstituted alkyl group having 1 to 30 carbon atoms or a substituted or unsubstituted alkyl halide group having 1 to 30 carbon atoms.

It is preferable that R₂₁, R₂₃, R₂₄, and R₂₆ in the formulae (1), (10), and (10a) are each a substituted or unsubstituted alkyl group having 1 to 30 carbon atoms or a substituted or unsubstituted alkyl halide group having 1 to 30 carbon atoms. In this case, R₂₂ and R₂₅ are preferably hydrogen atoms.

It is preferable that at least one of R₂₁, R₂₃, R₂₄, and R₂₆ in the formulae (1), (10), and (10a) is a substituted or unsubstituted aryl group having 6 to 30 ring carbon atoms.

It is more preferable that R₂₁, R₂₃, R₂₄, and R₂₆ in the formulae (1), (10), and (10a) are each a substituted or unsubstituted aryl group having 6 to 30 ring carbon atoms. In this case, R₂₂ and R₂₅ are preferably hydrogen atoms.

In the formulae (1), (10) and (10a), more preferably, R₂₁, R₂₃, R₂₄ and R₂₆ are each independently a substituted or unsubstituted alkyl group having 1 to 30 (preferably 1 to 6) carbon atoms, a substituted or unsubstituted alkyl halide group having 1 to 30 (preferably 1 to 6) carbon atoms, or an aryl group having 6 to 30 (preferably 6 to 12) ring carbon atoms substituted by an alkyl group having 1 to 30 carbon atoms, and R₂₂ and R₂₅ are hydrogen atoms.

It is preferable that at least one of R₂₁, R₂₃, and R₂₄ in the formula (11) is a substituted or unsubstituted alkyl group having 1 to 30 carbon atoms or a substituted or unsubstituted alkyl halide group having 1 to 30 carbon atoms.

It is more preferable that R₂₁, R₂₃, and R₂₄ in the formula (11) are each a substituted or unsubstituted alkyl group having 1 to 30 carbon atoms or a substituted or unsubstituted alkyl halide group having 1 to 30 carbon atoms. In this case, R₂₂ is preferably a hydrogen atom.

It is preferable that at least one of R₂₁, R₂₃, and R₂₄ in the formula (11) is a substituted or unsubstituted aryl group having 6 to 30 ring carbon atoms.

It is more preferable that R₂₁, R₂₃, and R₂₄ in the formula (11) are each a substituted or unsubstituted aryl group having 6 to 30 ring carbon atoms. In this case, R₂₂ is preferably a hydrogen atom.

In the formula (11), more preferably, R₂₁, R₂₃, and R₂₄ are each independently a substituted or unsubstituted alkyl group having 1 to 30 (preferably 1 to 6) carbon atoms, a substituted or unsubstituted alkyl halide group having 1 to 30 (preferably 1 to 6) carbon atoms, or an aryl group having 6 to 30 (preferably 6 to 12) ring carbon atoms substituted by an alkyl group having 1 to 30 carbon atoms, and R₂₂ is a hydrogen atom.

In the first compound, examples of the fluorine-atom-substituted alkoxy group include: 2,2,2-trifluoroethoxy group, 2,2-difluoroethoxy group, 2,2,3,3,3-pentafluoro-1-propoxy group, 2,2,3,3-tetrafluoro-1-propoxy group, 1,1,1,3,3,3-hexafluoro-2-propoxy group, 2,2,3,3,4,4,4-heptafluoro-1-butyloxy group, 2,2,3,3,4,4-hexafluoro-1-butyloxy group, nonafluoro-tert-butyloxy group, 2,2,3,3,4,4,5,5,5-nonafluoropentanoxy group, 2,2,3,3,4,4,5,5,6,6,6-undecafluorohexanoxy group, 2,3-bis(trifluoromethyl)-2,3-butanedioxy group, 1,1,2,2-tetra(trifluoromethyl)ethyleneglykoxy group, 4,4,5,5,6,6,6-heptafluorohexane-1,2-dioxy group, and 4,4,5,5,6,6,7,7,8,8,9,9,9-tridecafluorononane-1,2-dioxy group.

In the first compound, examples of the fluorine-atom substituted aryloxy group, or the fluoroalkyl-group-substituted aryloxy group include pentafluorophenoxy group, 3,4,5-trifluorophenoxy group, 4-trifluoromethylphenoxy group, 3,5-bistrifluoromethylphenoxy group, 3-fluoro-4-trifluoromethylphenoxy group, 2,3,5,6-tetrafluoro-4-trifluoromethylphenoxy group, 4-fluorocatecholato group, 4-trifluoromethylcatecholato group, and 3,5-bis-trifluoromethylcatecholato group.

The substituent meant by “substituted or unsubstituted” in the first compound is preferably a substituent selected from the group consisting of an aryl group having 6 to 30 ring carbon atoms, heteroaryl group having 5 to 30 ring atoms, alkyl group having 1 to 30 carbon atoms, alkyl halide group having 1 to 30 carbon atoms, cycloalkyl group having 3 to 30 ring carbon atoms, cyano group, amino group, substituted amino group, halogen atom, alkoxy group having 1 to 30 carbon atoms, aryloxy group having 6 to 30 carbon atoms, arylthio group having 6 to 30 ring carbon atoms, aralkyl group having 7 to 30 carbon atoms, substituted phosphoryl group, substituted silyl group, nitro group, carboxy group, alkenyl group having 2 to 30 carbon atoms, alkynyl group having 2 to 30 carbon atoms, alkylthio group having 1 to 30 carbon atoms, alkylsilyl group having 3 to 30 carbon atoms, arylsilyl group having 6 to 30 ring carbon atoms, and hydroxy group.

The substituent meant by “substituted or unsubstituted” for the first compound is more preferably a substituent selected from the group consisting of a substituted or unsubstituted aryl group having 6 to 30 ring carbon atoms, substituted or unsubstituted heteroaryl group having 5 to 30 ring atoms, alkyl group having 1 to 30 carbon atoms, alkyl halide group having 1 to 30 carbon atoms, and cycloalkyl group having 3 to 30 ring carbon atoms.

The substituent meant by “substituted or unsubstituted” for the first compound is further preferably a substituent selected from the group consisting of a substituted or unsubstituted aryl group having 6 to 12 ring carbon atoms, substituted or unsubstituted heteroaryl group having 5 to 12 ring atoms, alkyl group having 1 to 6 carbon atoms, alkyl halide group having 1 to 6 carbon atoms, and cycloalkyl group having 3 to 12 ring carbon atoms.

The first compound is preferably a fluorescent compound.

In this case, the first compound preferably emits light having a main peak wavelength ranging from 400 nm to 700 nm.

The use of the fluorescent first compound in the emitting layer in combination with the second compound (delayed fluorescent compound) and the third compound enhances the likelihood of highly efficient emission.

Herein, the main peak wavelength means a peak wavelength of an fluorescence spectrum exhibiting a maximum luminous intensity among fluorescence spectra measured in a toluene solution in which a measurement target compound is dissolved at a concentration ranging from 10⁻⁶ mol/l to 10⁻⁵ mol/l. A spectrophotofluorometer (F-7000 manufactured by Hitachi High-Tech Science Corporation) is used as a measurement device.

The first compound preferably exhibits a red or green light emission.

Herein, the red light emission refers to light emission whose main peak wavelength of fluorescence spectrum is in a range from 600 nm to 660 nm.

When the first compound is a red fluorescent compound, the main peak wavelength of the first compound is preferably in a range from 600 nm to 660 nm, more preferably in a range from 600 nm to 640 nm, further preferably in a range from 610 nm to 630 nm.

Herein, the green light emission refers to light emission whose main peak wavelength of fluorescence spectrum is in a range from 500 nm to 560 nm.

When the first compound is a green fluorescent compound, the main peak wavelength of the first compound is preferably in a range from 500 nm to 560 nm, more preferably in a range from 500 nm to 540 nm, further preferably in a range from 510 nm to 530 nm.

An example of the organic EL device according to the present exemplary embodiment includes an anode, a cathode, and a first organic layer between the anode and the cathode, the first organic layer containing the first, second, and third compounds, the first compound being represented by the formula (1) and having its main peak wavelength in a range from 600 nm to 660 nm, the second compound being a delayed fluorescent compound, the third compound being represented by the formula (3).

Another example of the organic EL device according to the present exemplary embodiment includes an anode, a cathode, and a first organic layer between the anode and the cathode, the first organic layer containing the first, second, and third compounds, the first compound being represented by the formula (1) and having its main peak wavelength in a range from 500 nm to 560 nm, the second compound being a delayed fluorescent compound, the third compound being represented by the formula (3).

It should be noted that the invention is not limited to the examples of the organic EL device mentioned above.

### Method of Preparing First Compound

The first compound can be prepared by any known method.

Specific examples of the first compound of the present exemplary embodiment are shown below. It should be noted that the first compound according to the invention is not limited to these specific examples. It should also be noted that coordinate bond between a boron atom and a nitrogen atom in a pyrromethene skeleton herein can be illustrated in a variety of manners (e.g. in a dotted line, in an arrow, or omitted). The coordinate bond is illustrated in a dotted line or omitted herein.

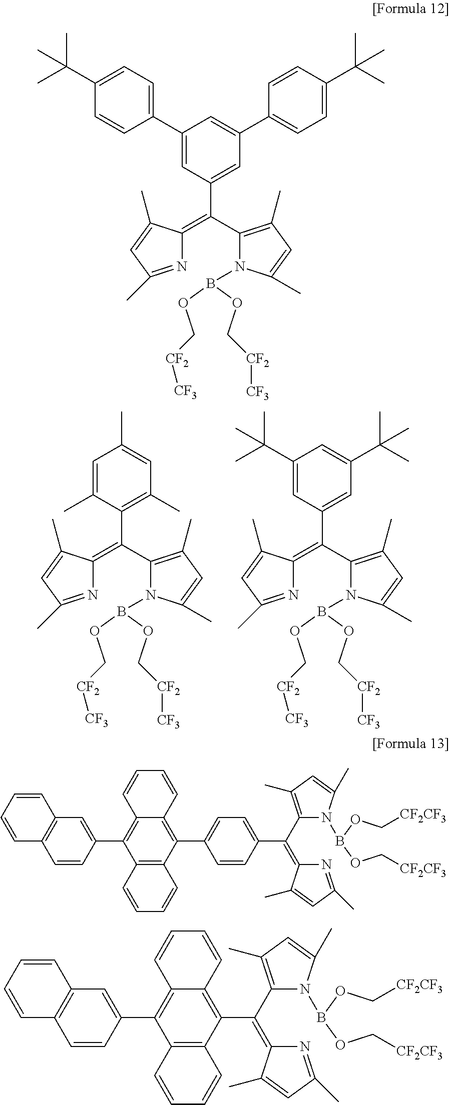

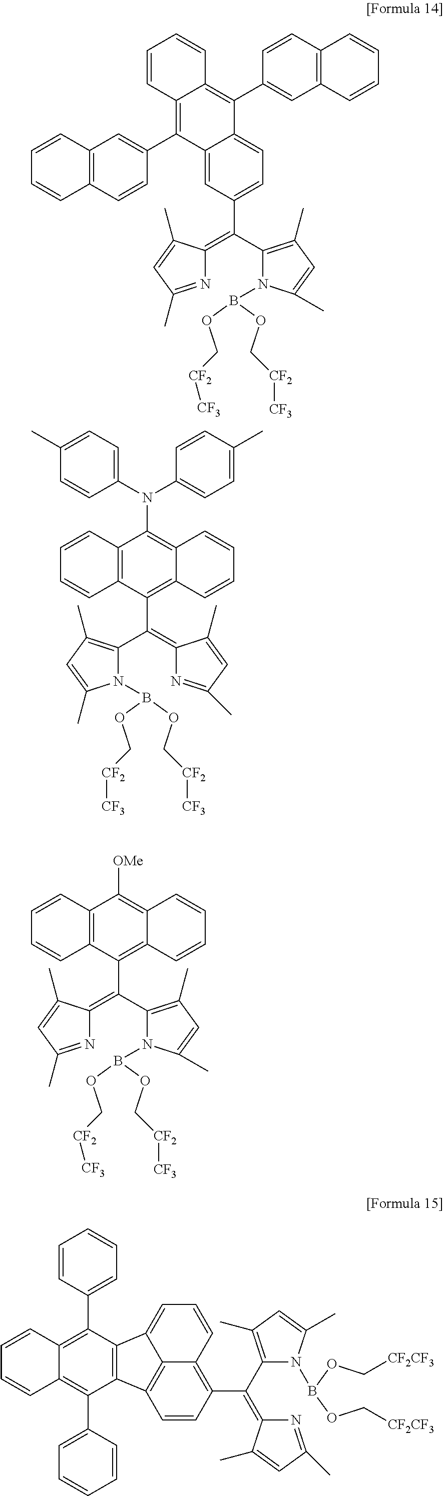

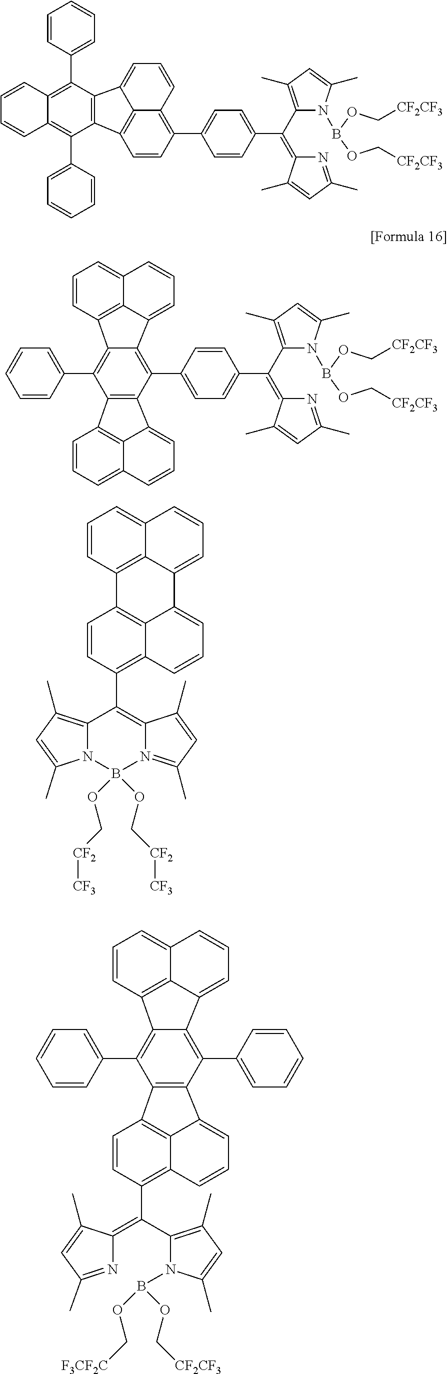

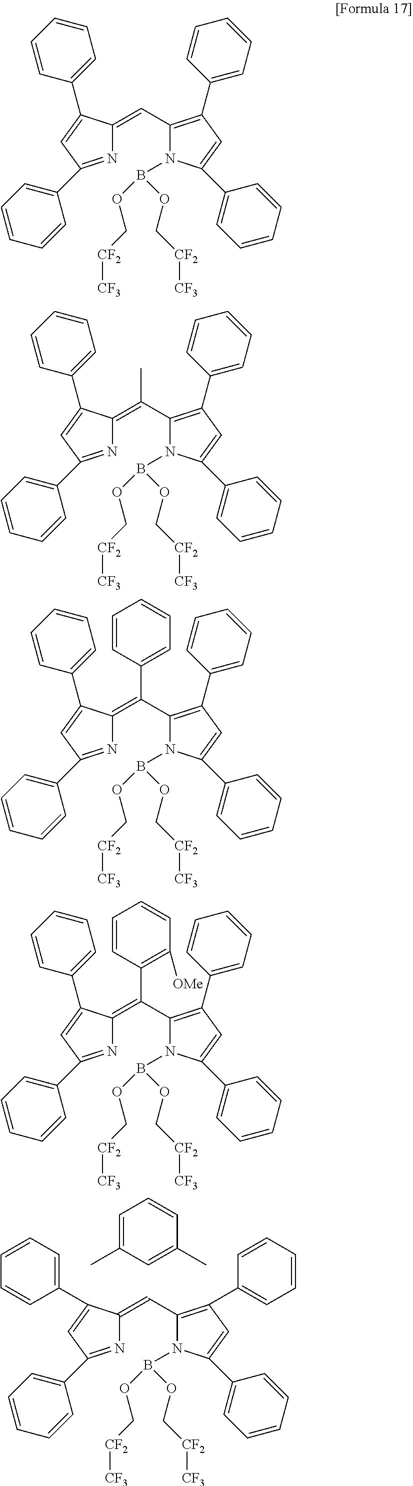

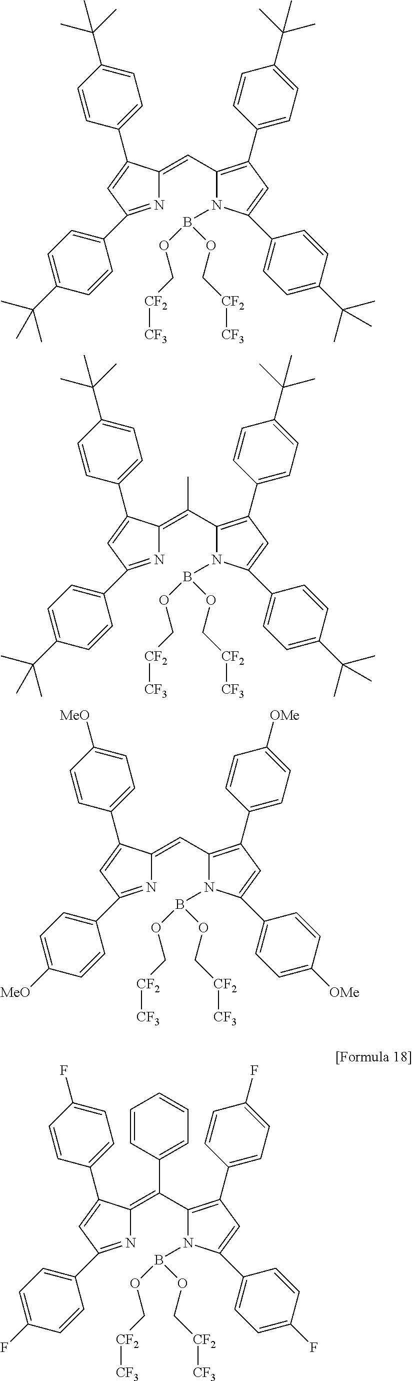

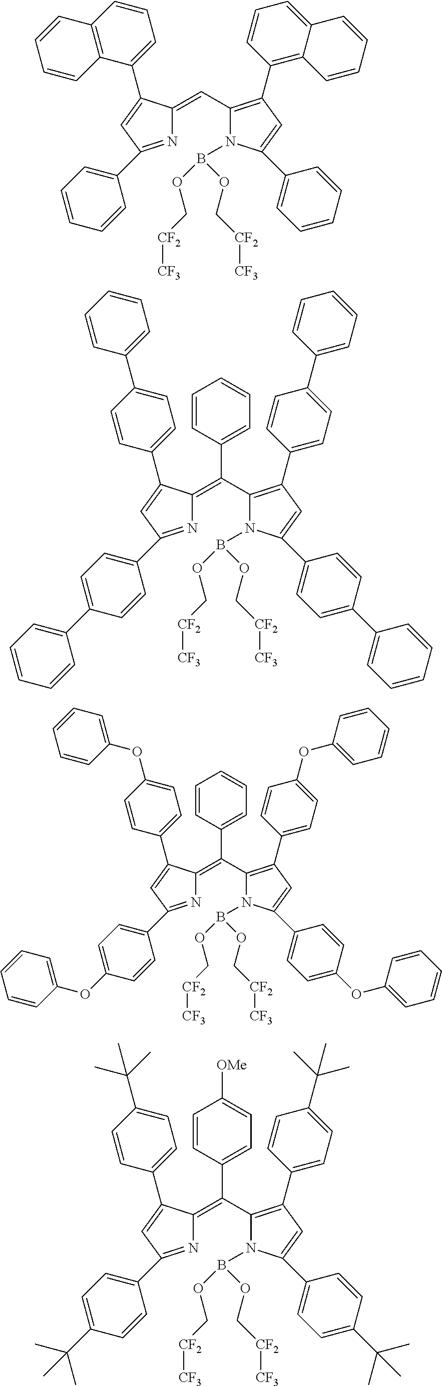

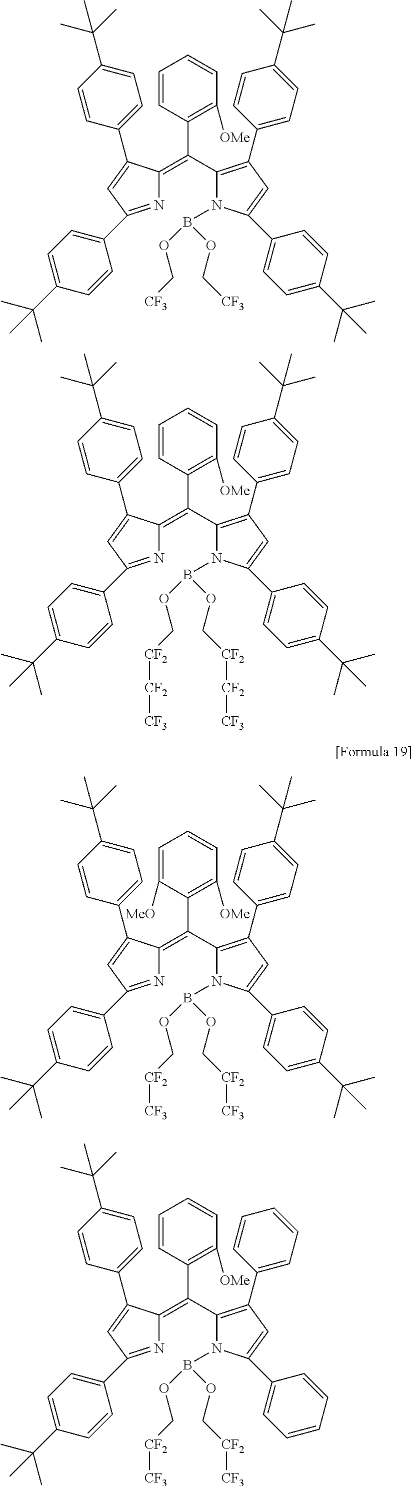

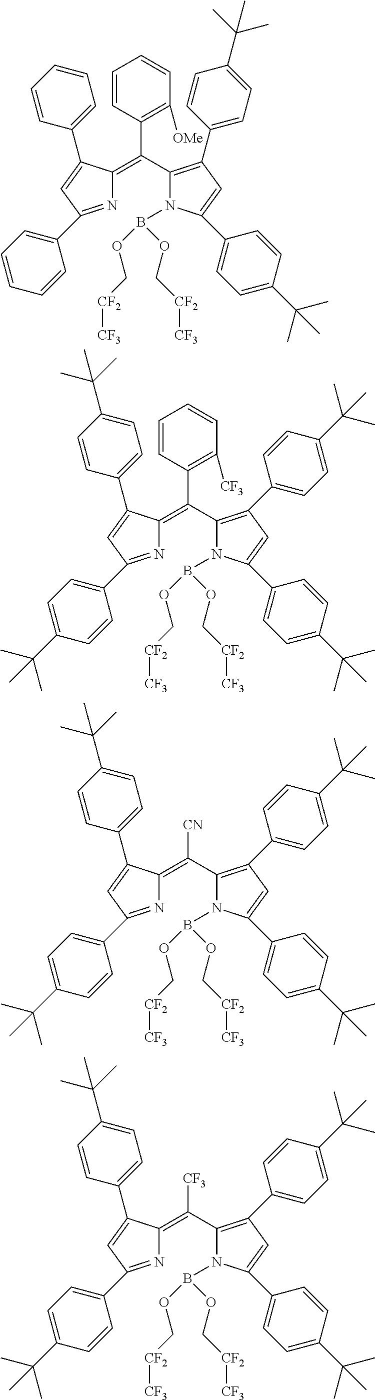

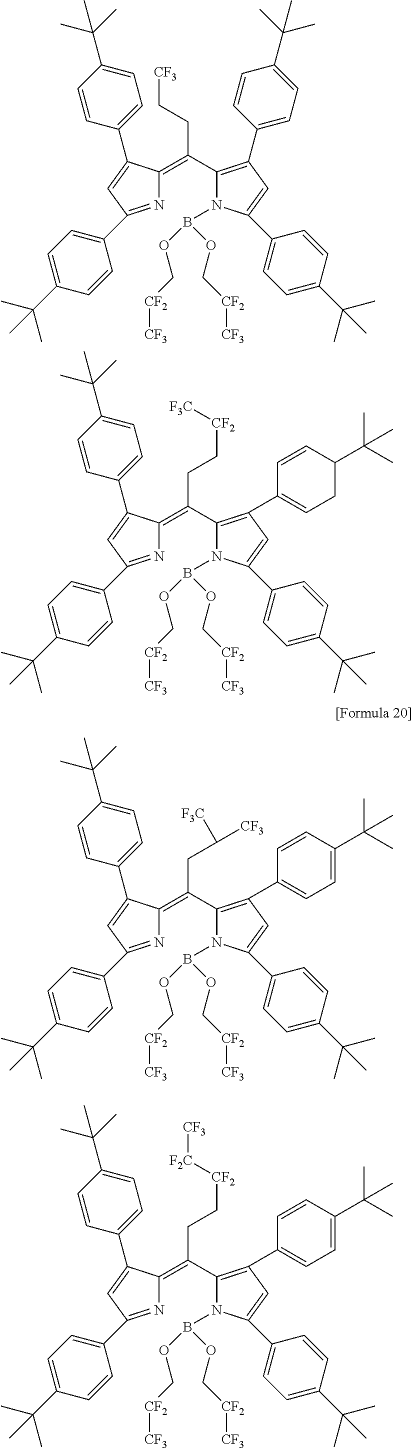

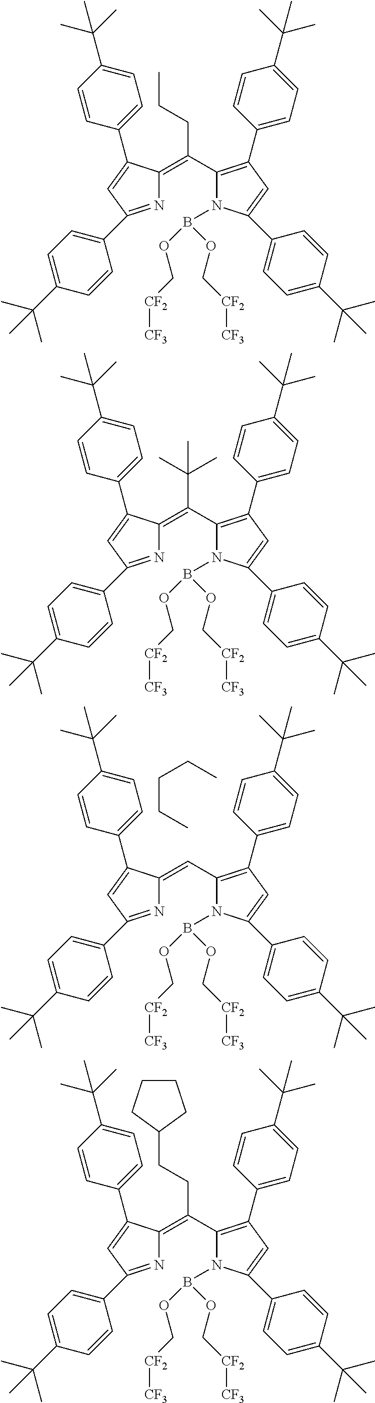

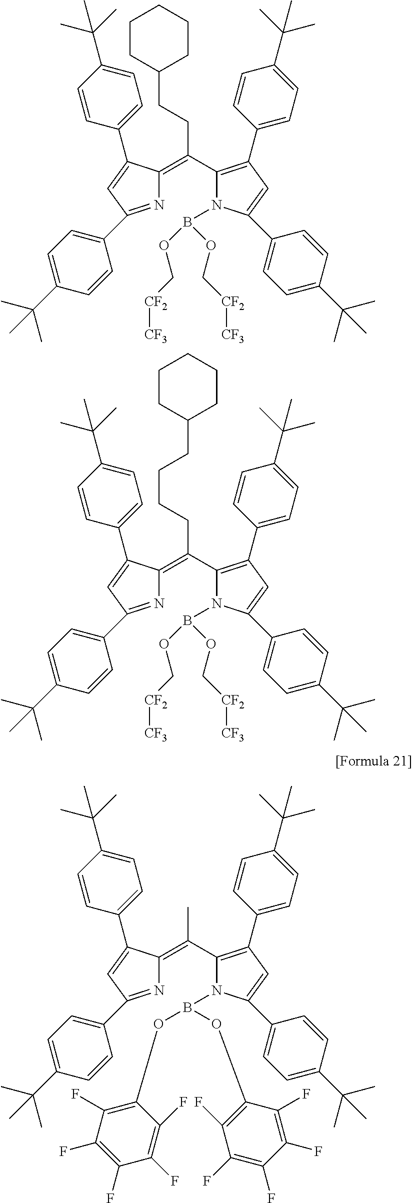

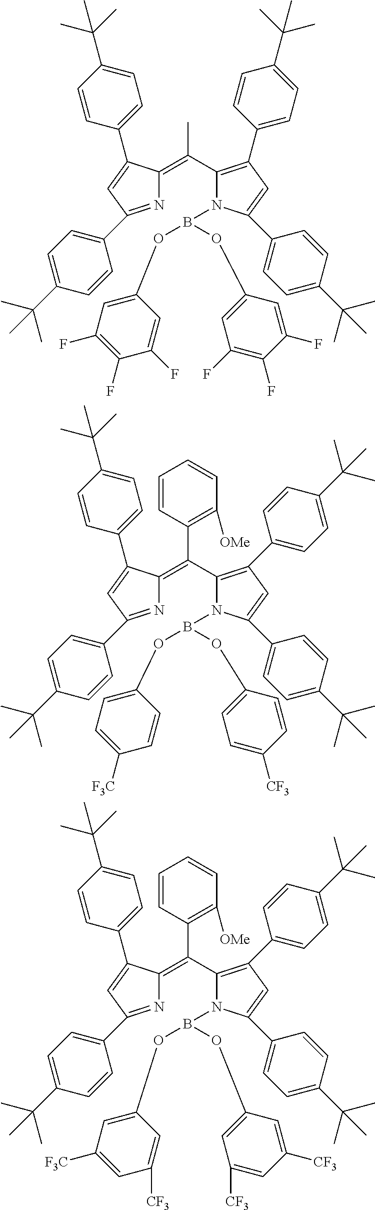

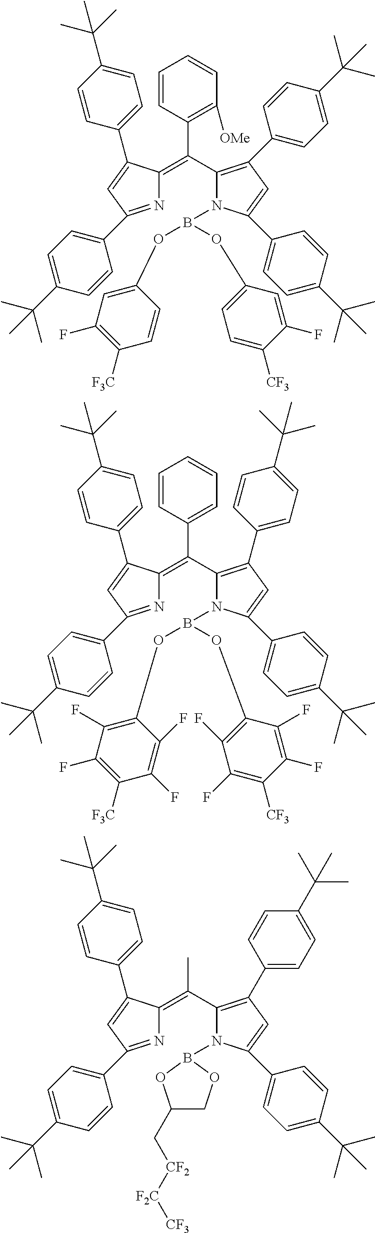

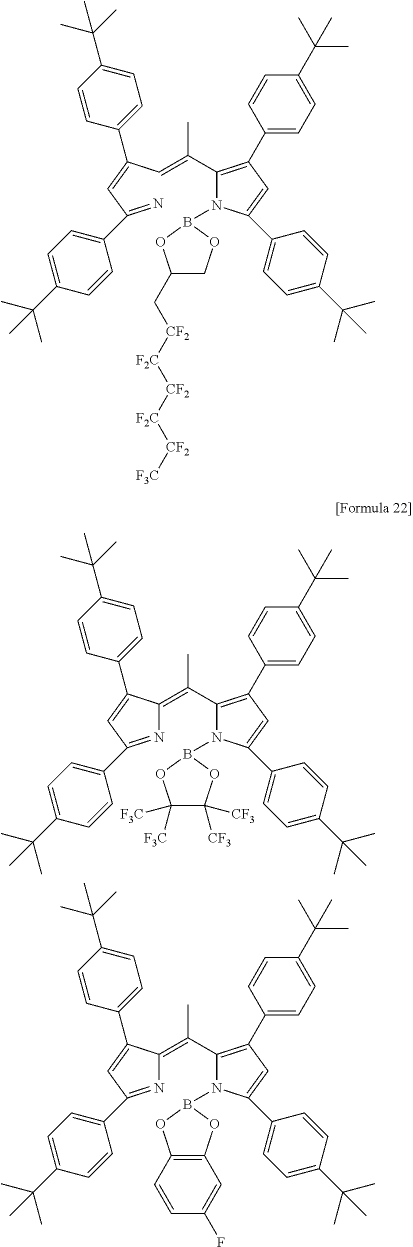

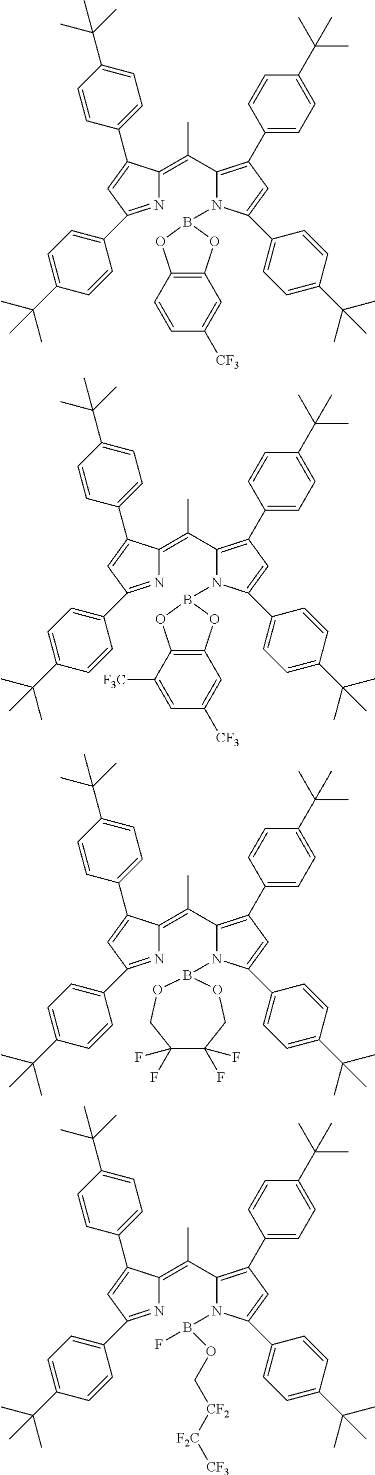

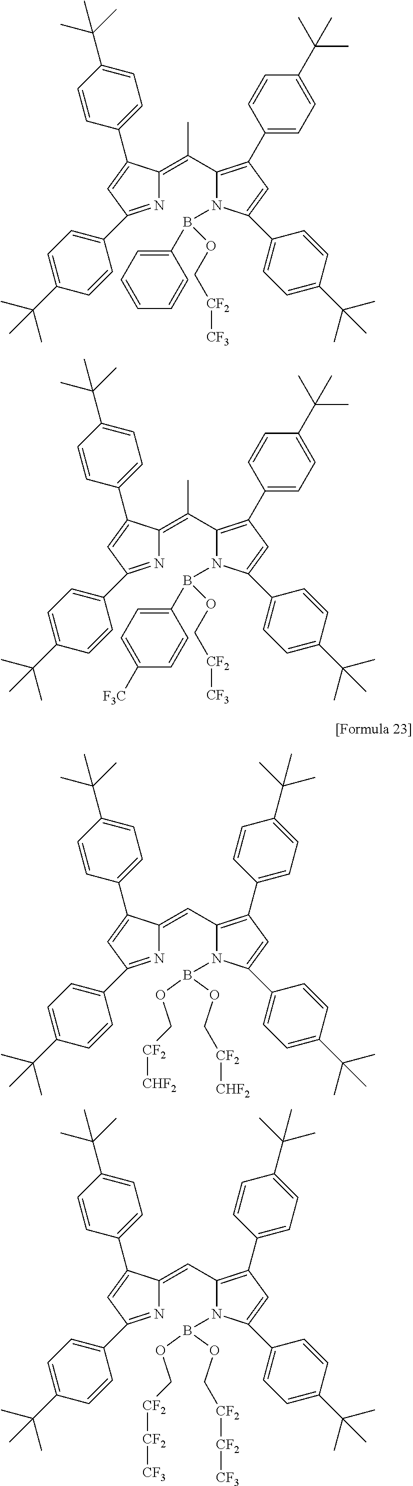

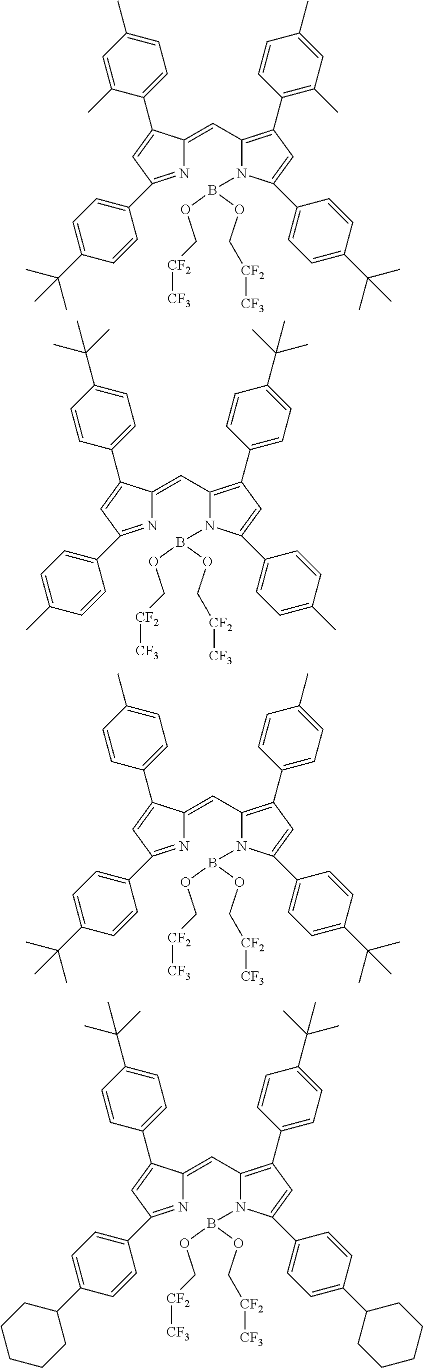

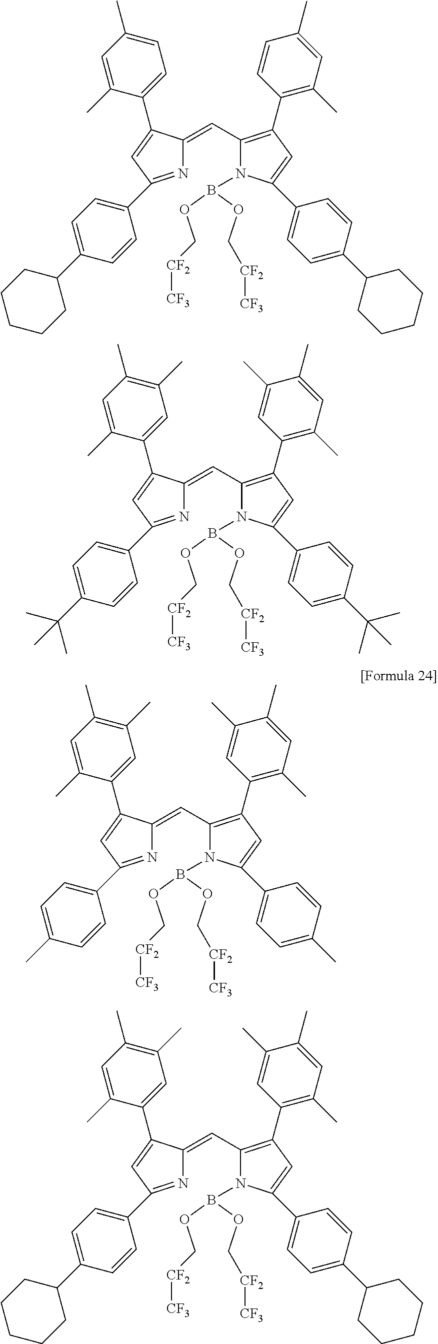

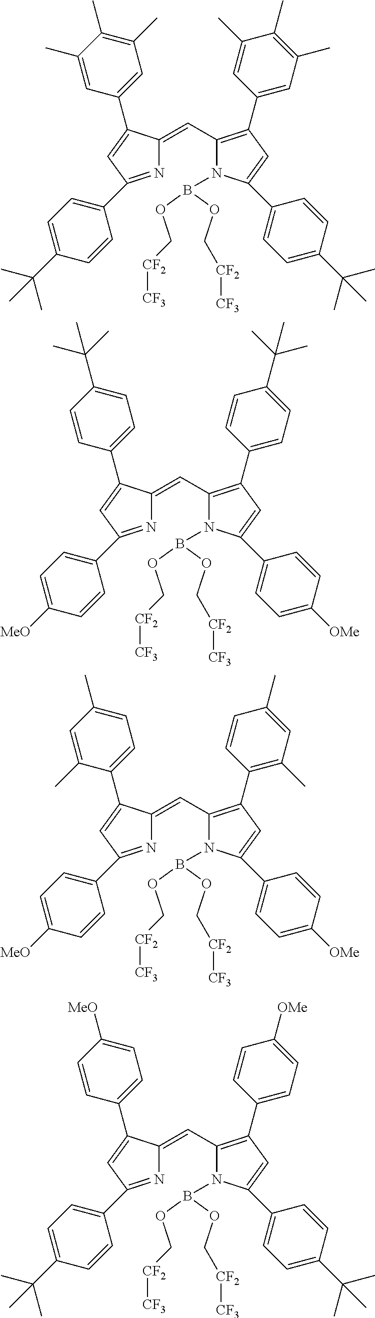

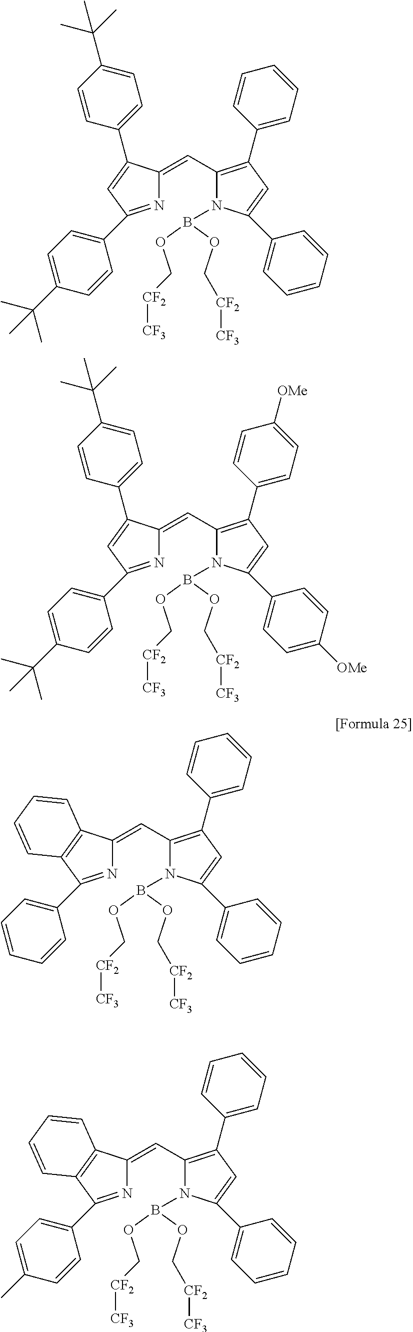

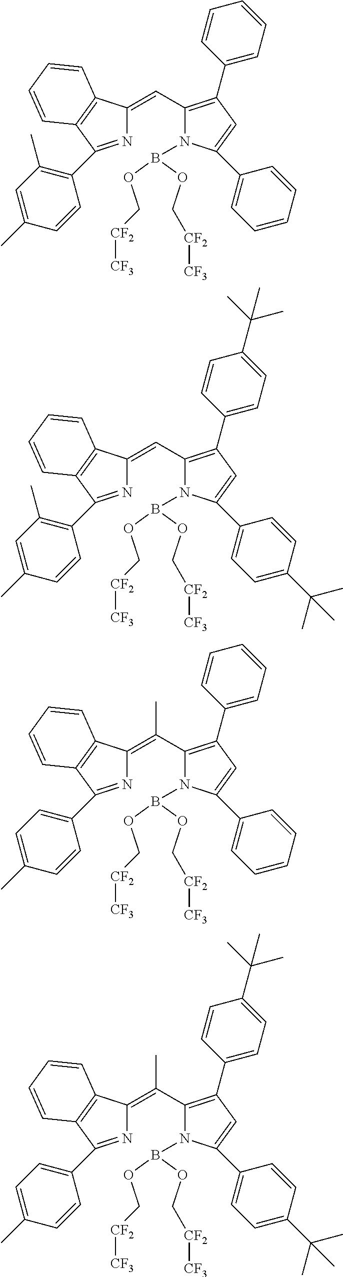

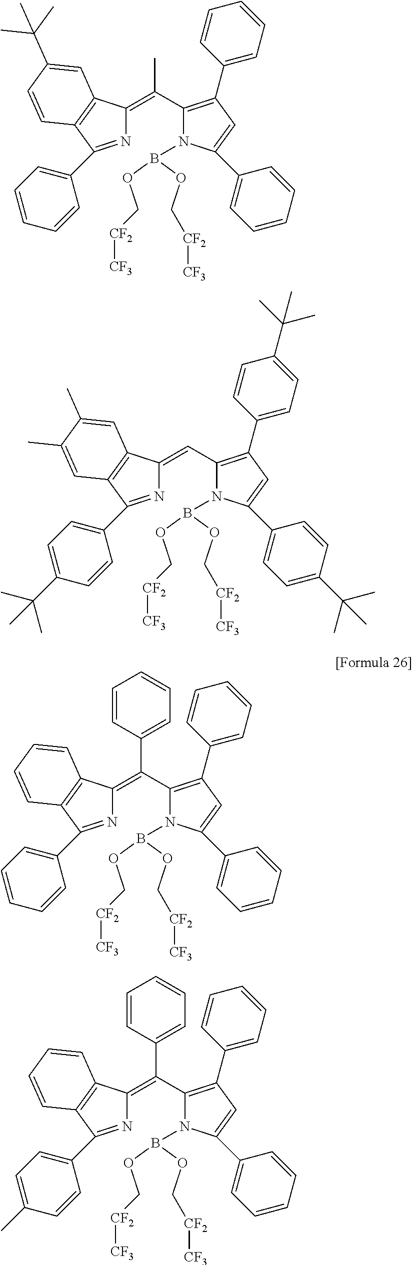

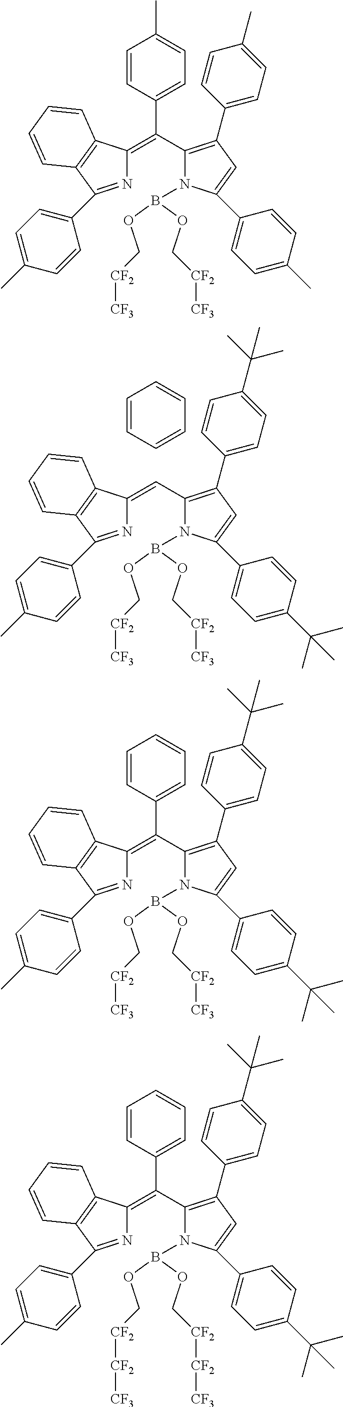

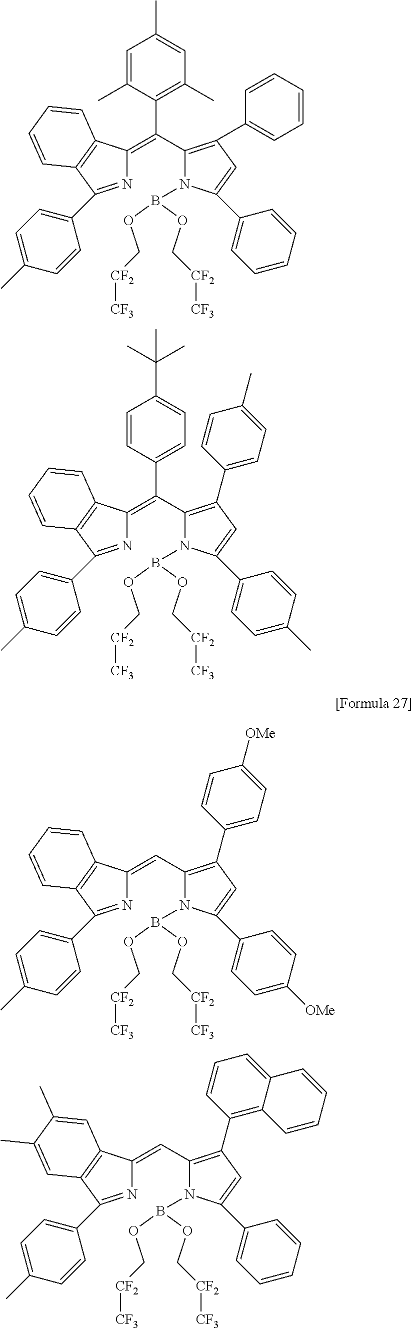

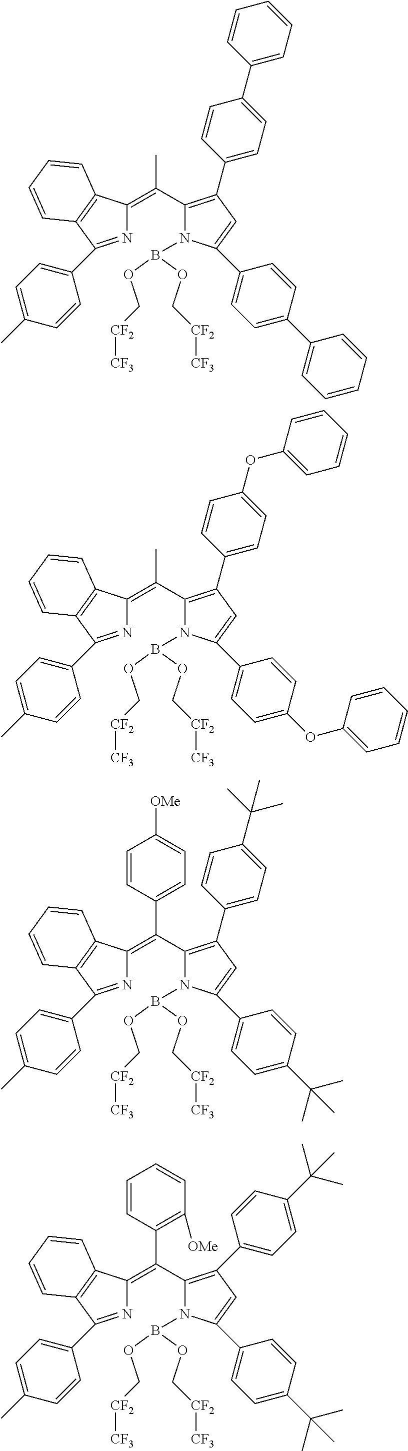

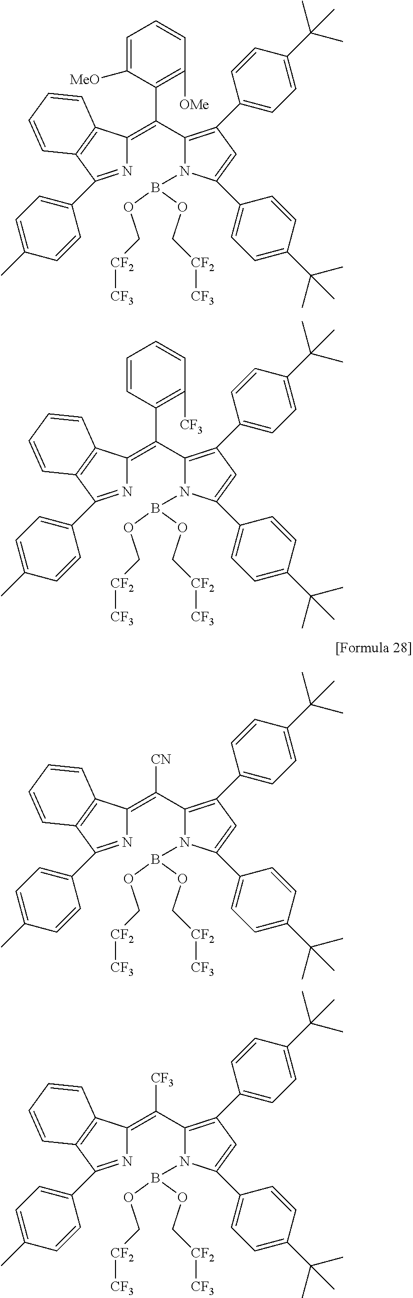

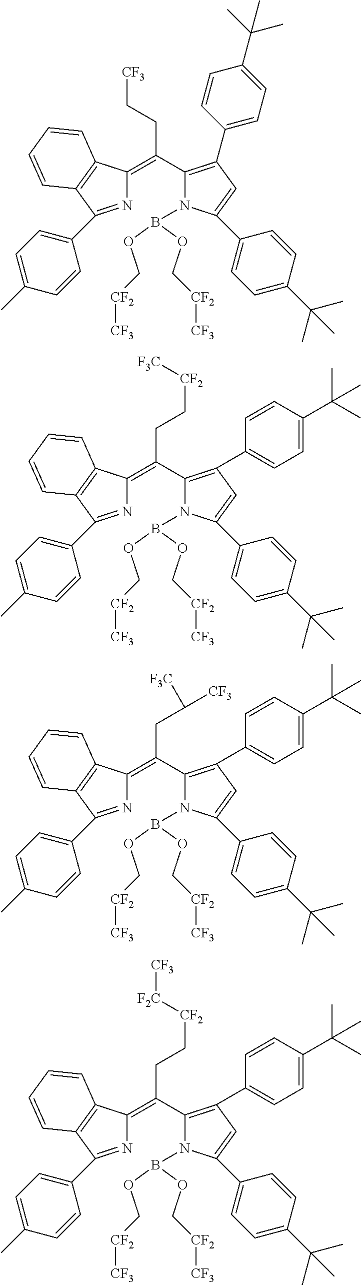

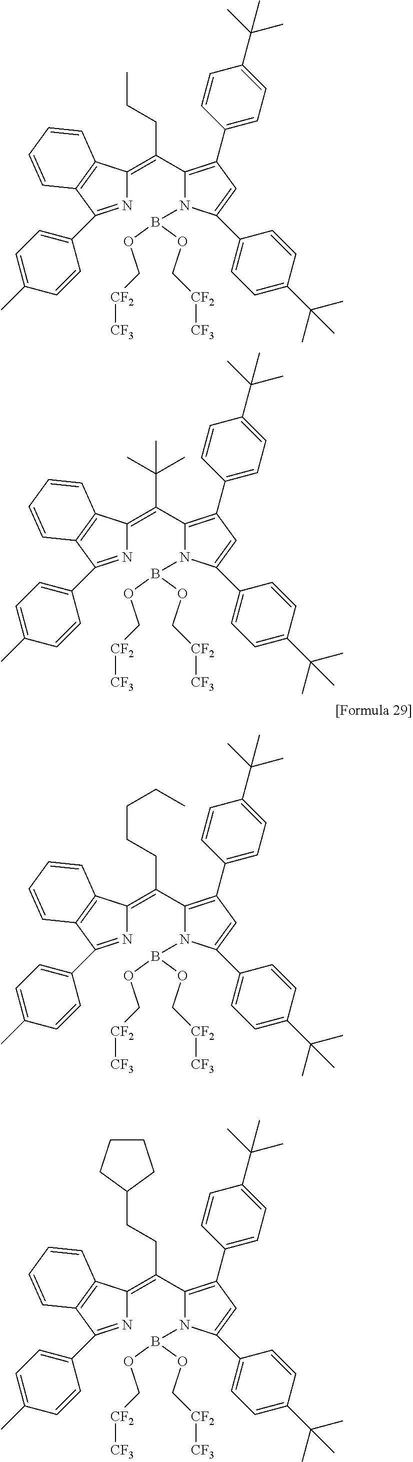

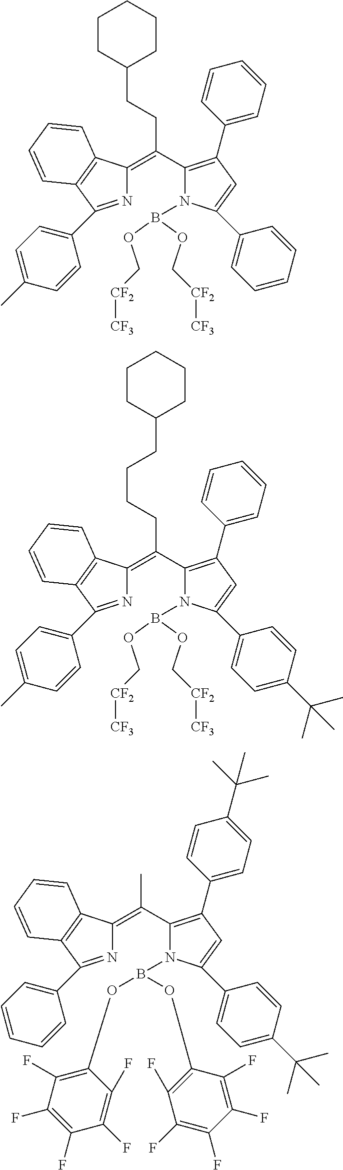

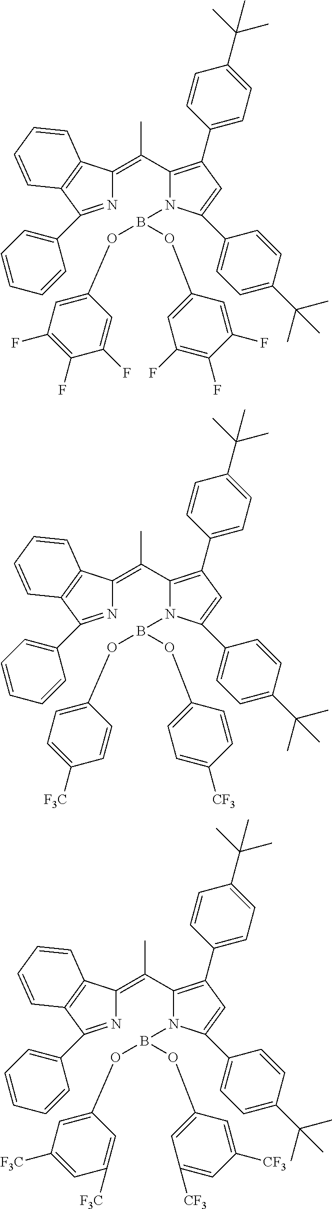

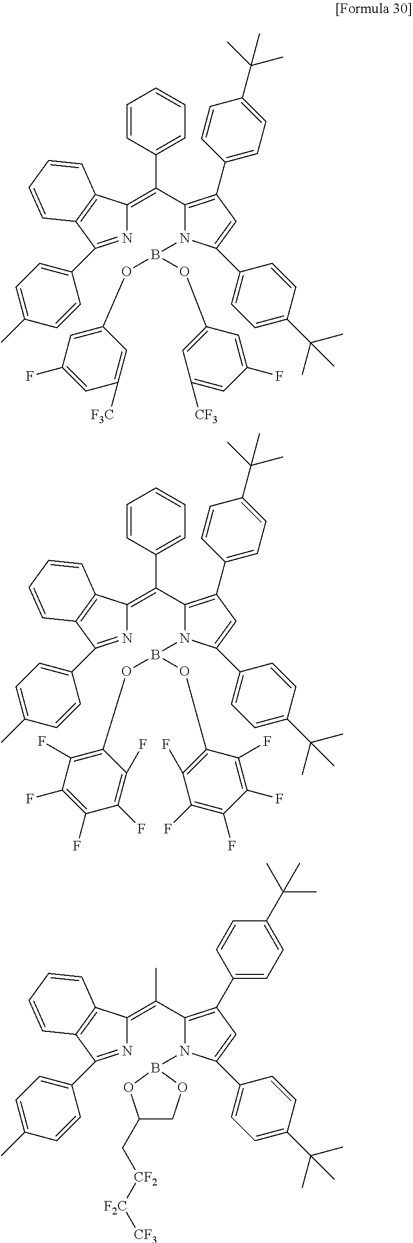

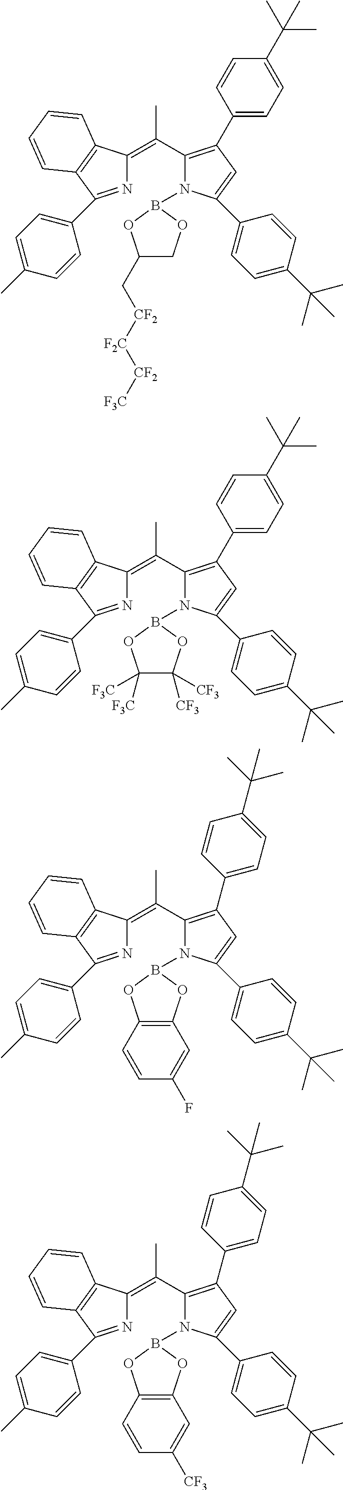

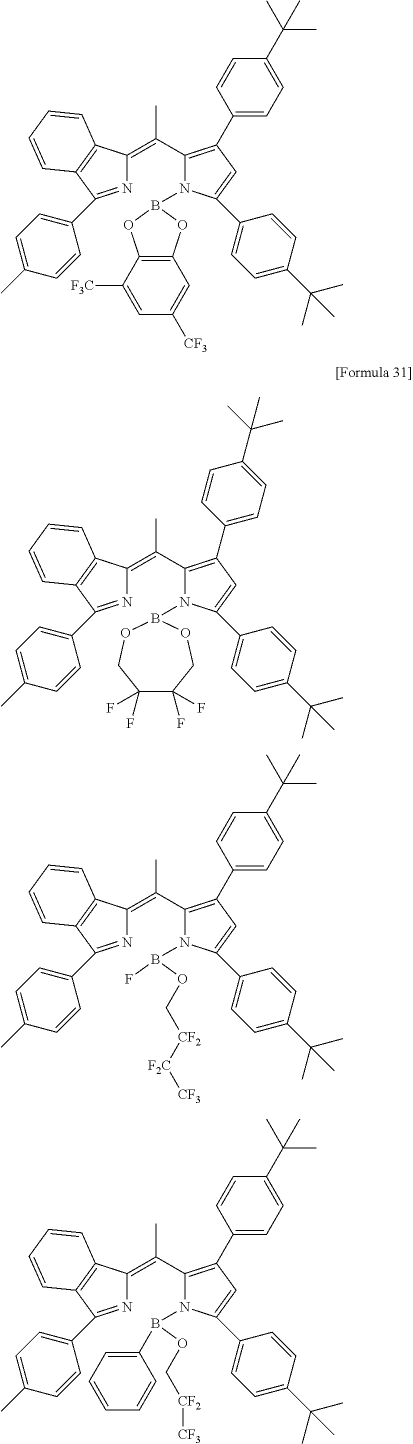

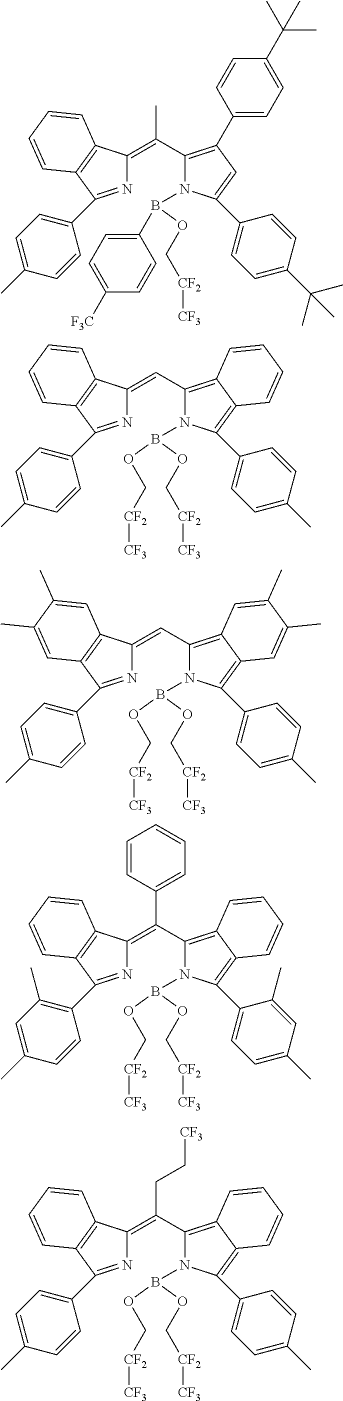

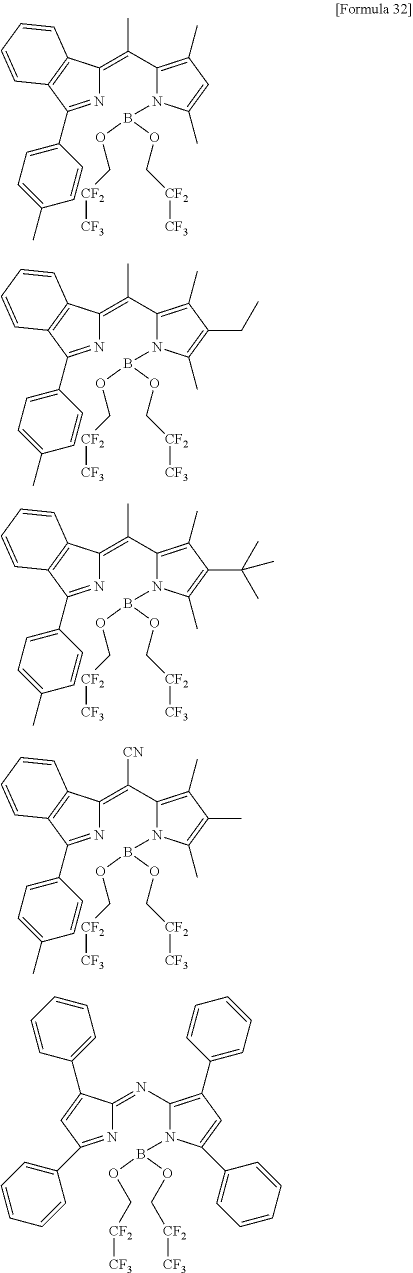

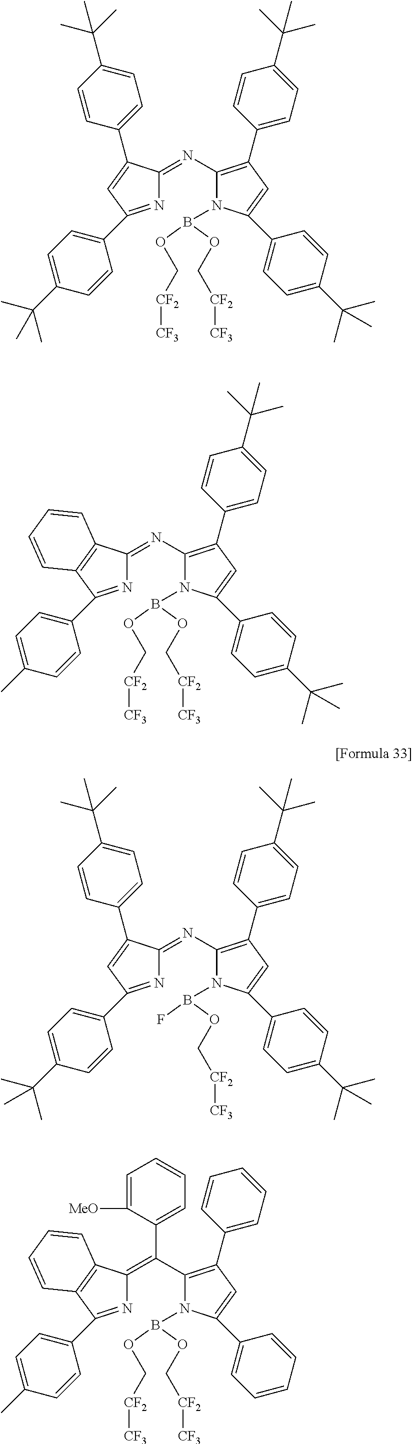

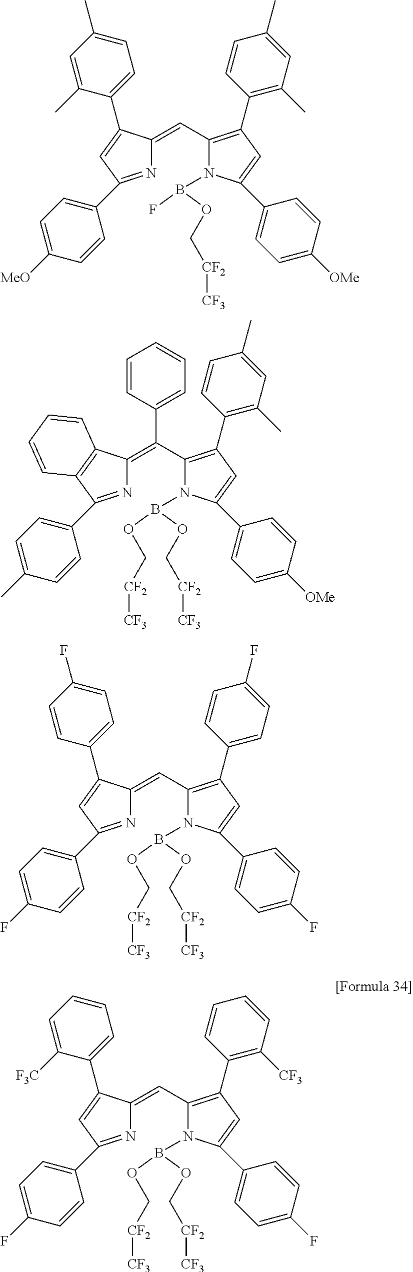 [SMILES: COc1ccc(C2=N/C(=C\c3c(-c4ccc(C)cc4C)cc(-c4ccc(OC)cc4)n3B(F)OCC(F)(F)C(F)(F)F)C(c3ccc(C)cc3C)=C2)cc1 ; COc1ccc(-c2cc(-c3ccc(C)cc3C)c(/C(=C3\N=C(c4ccc(C)cc4)c4ccccc43)c3ccccc3)n2B(OCC(F)(F)C(F)(F)F)OCC(F)(F)C(F)(F)F)cc1 ; Fc1ccc(C2=CC(c3ccc(F)cc3)=N/C2=C\c2c(-c3ccc(F)cc3)cc(-c3ccc(F)cc3)n2B(OCC(F)(F)C(F)(F)F)OCC(F)(F)C(F)(F)F)cc1 ; Fc1ccc(C2=N/C(=C\c3c(-c4ccccc4C(F)(F)F)cc(-c4ccc(F)cc4)n3B(OCC(F)(F)C(F)(F)F)OCC(F)(F)C(F)(F)F)C(c3ccccc3C(F)(F)F)=C2)cc1]

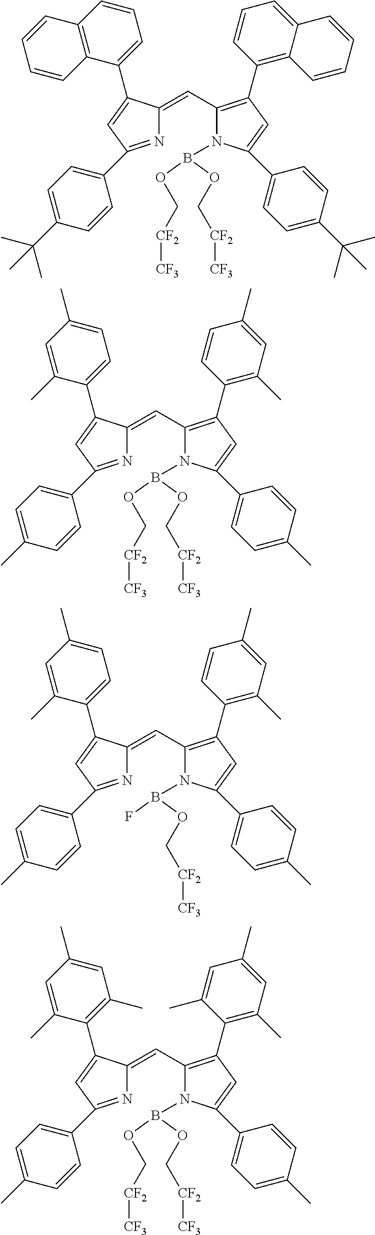

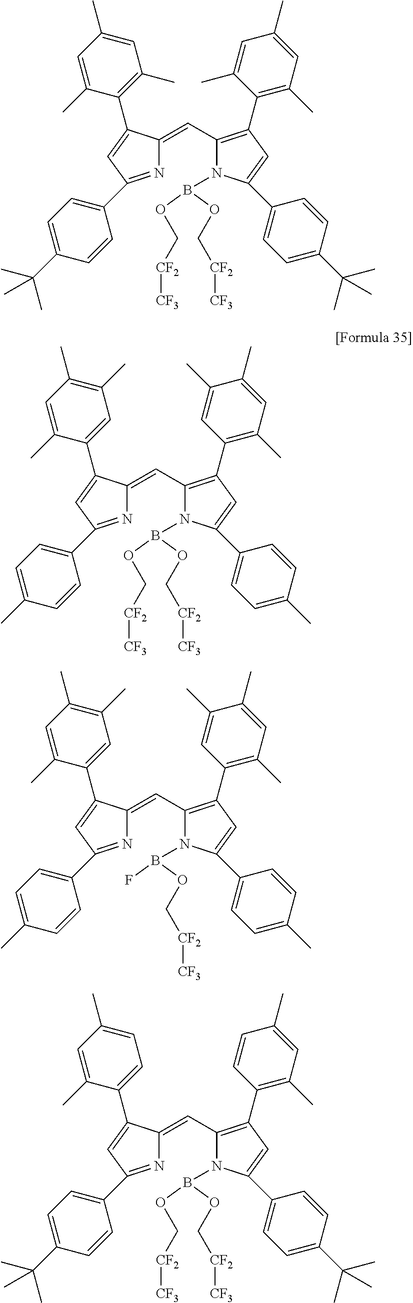

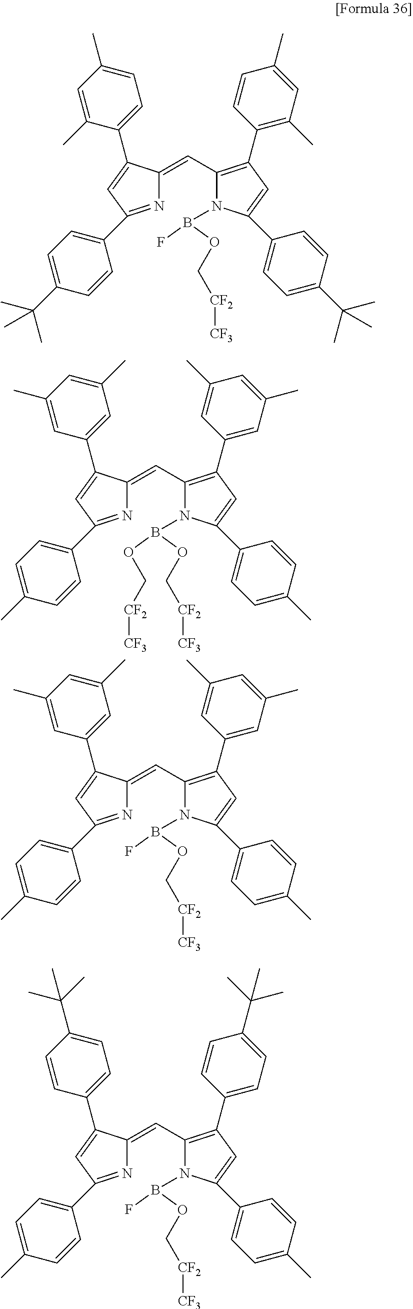

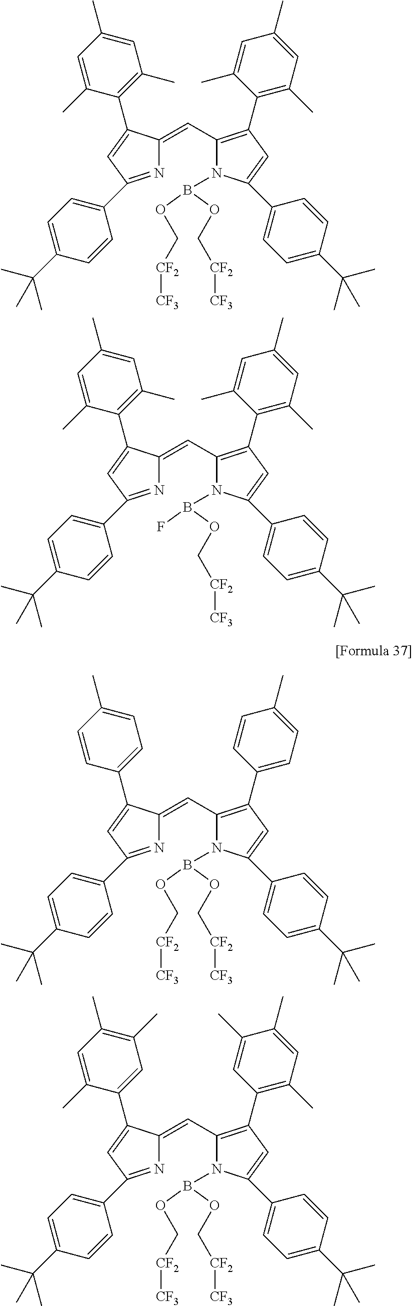

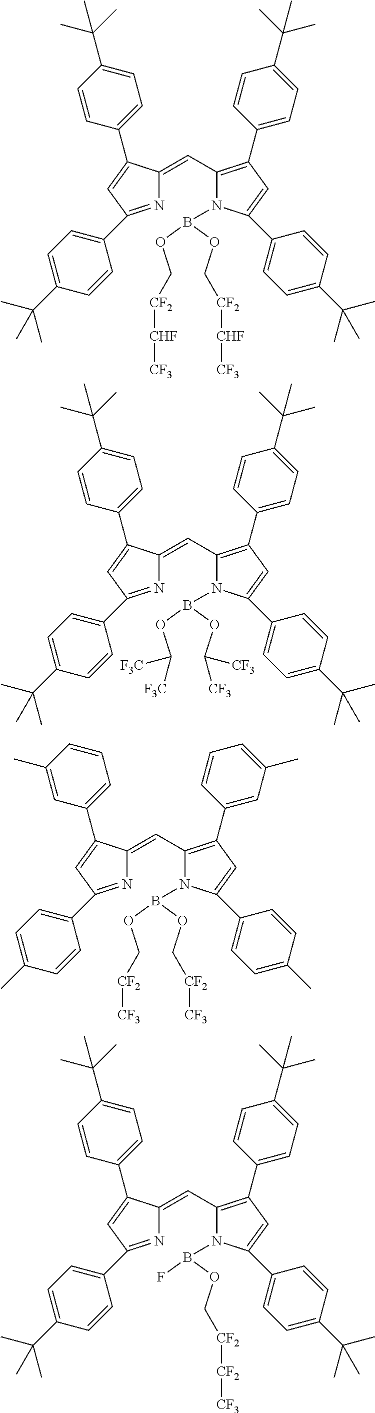

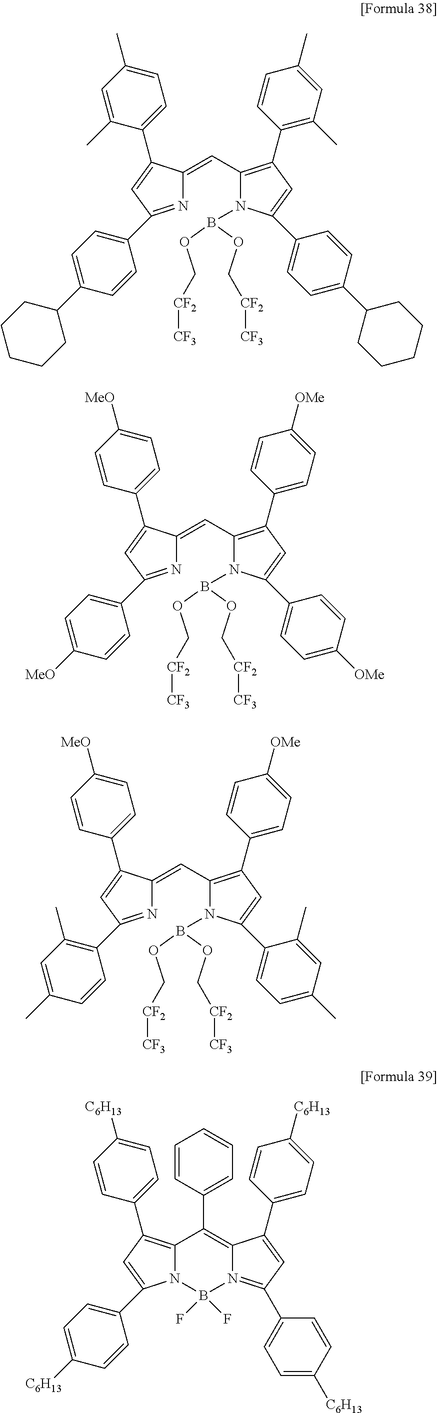

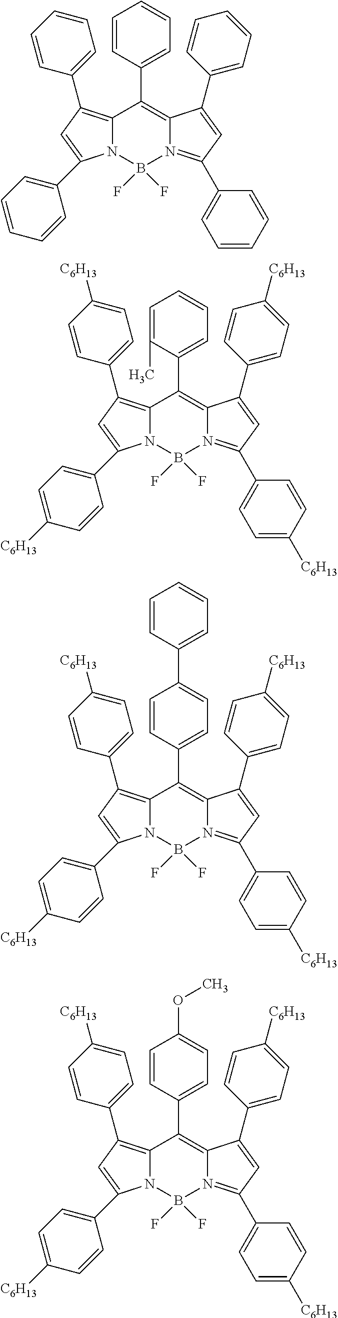

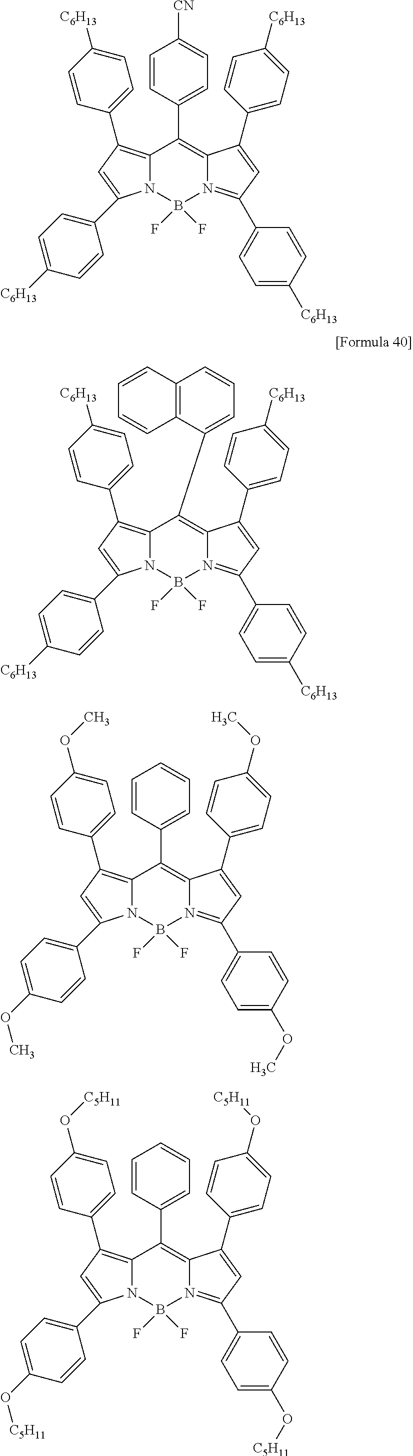

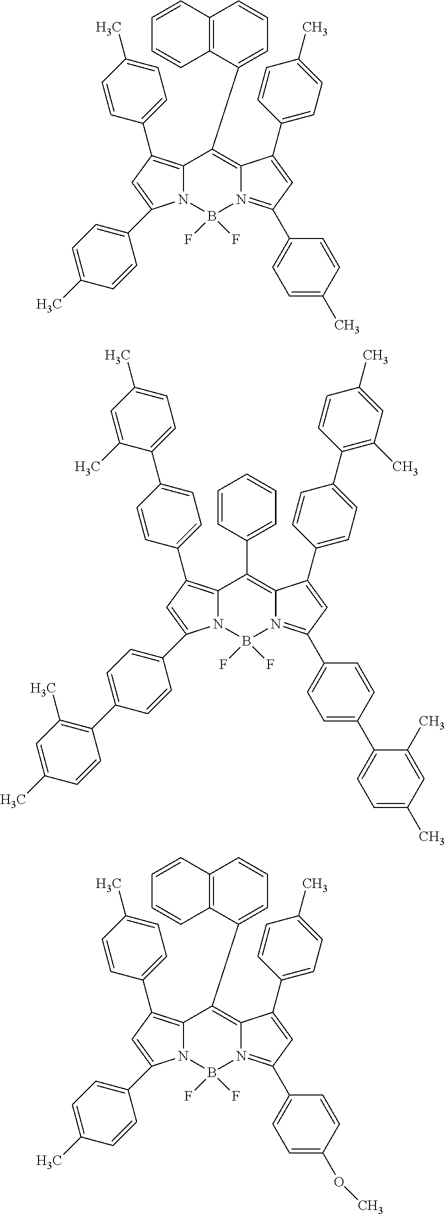

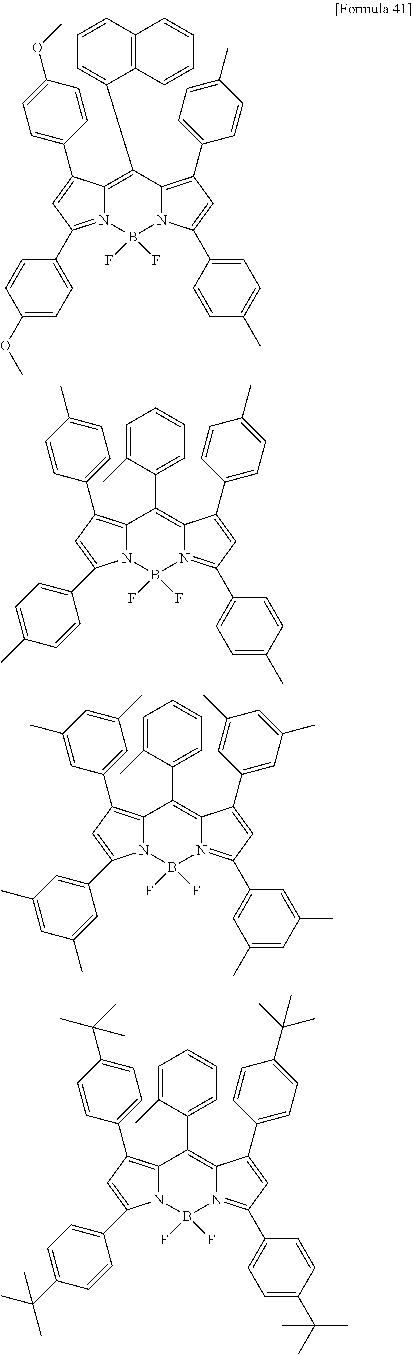

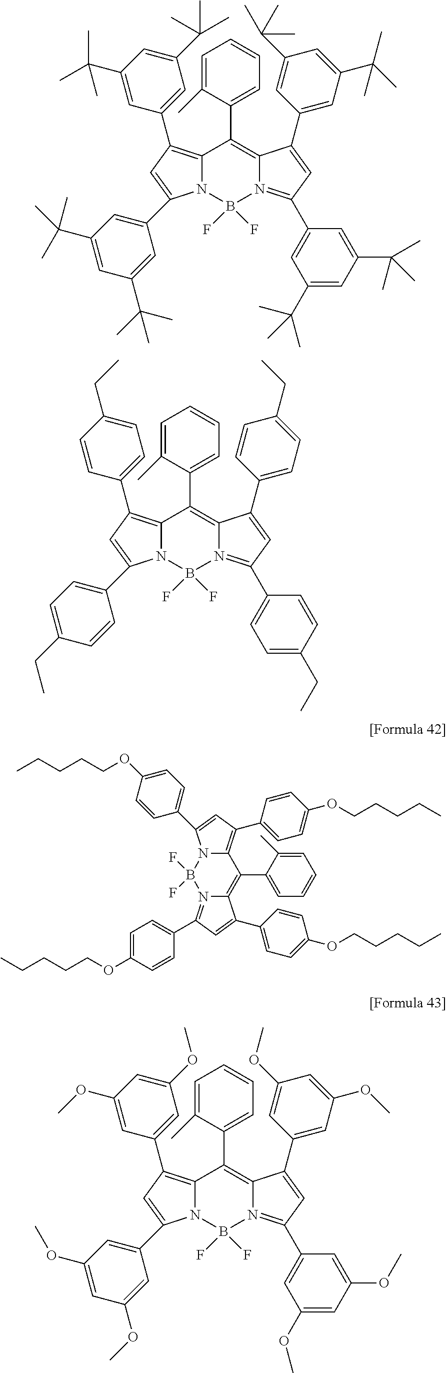

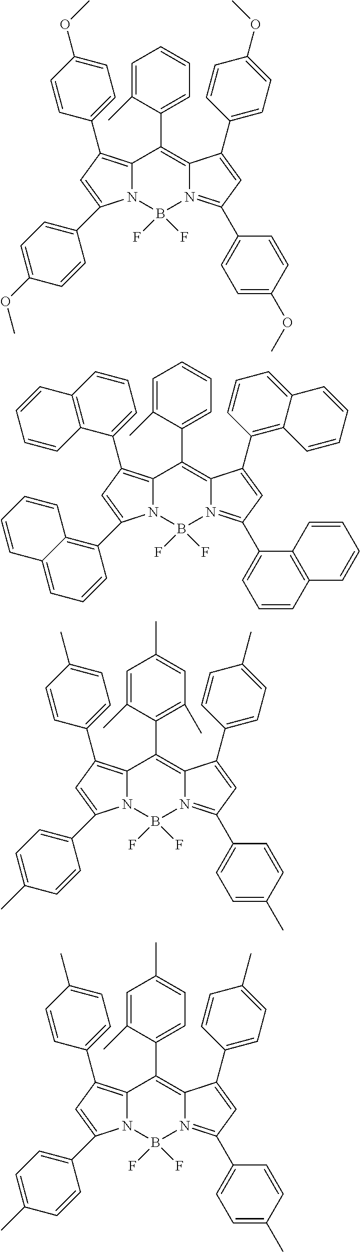

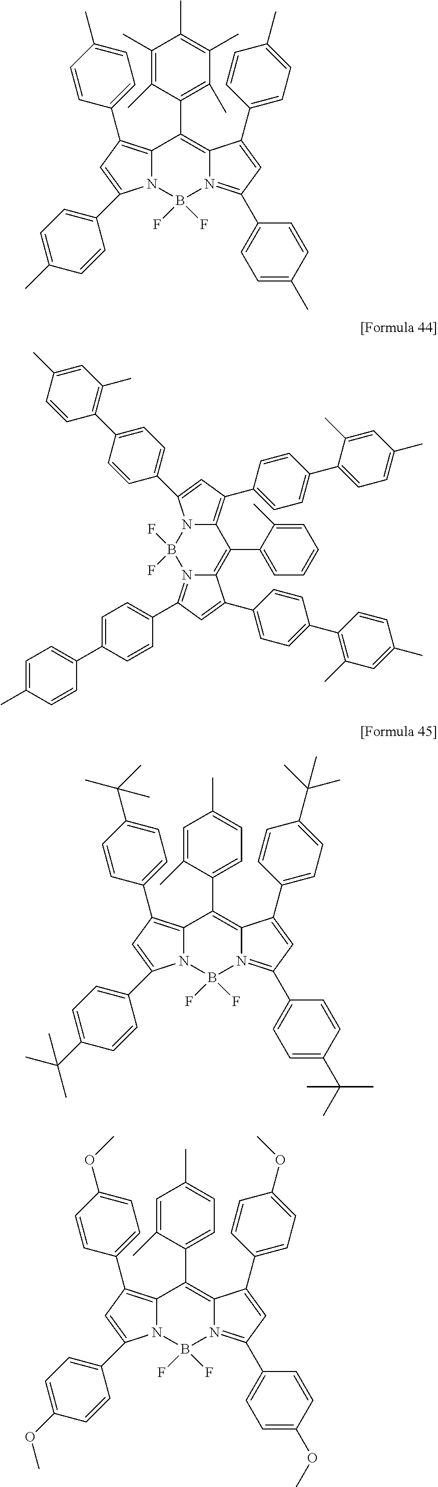

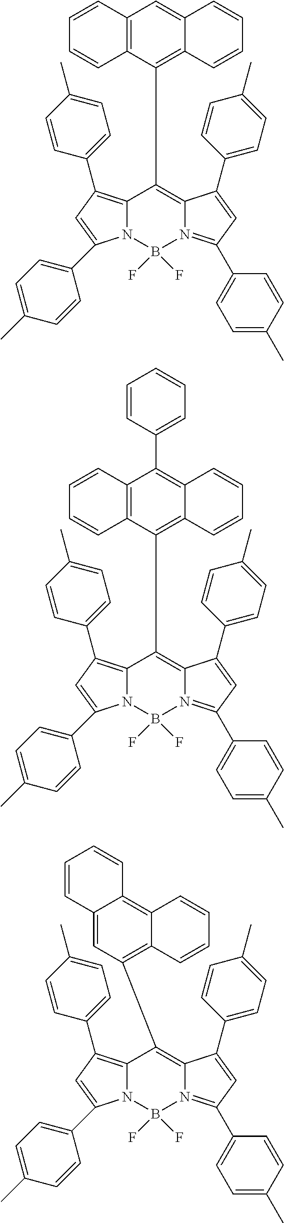

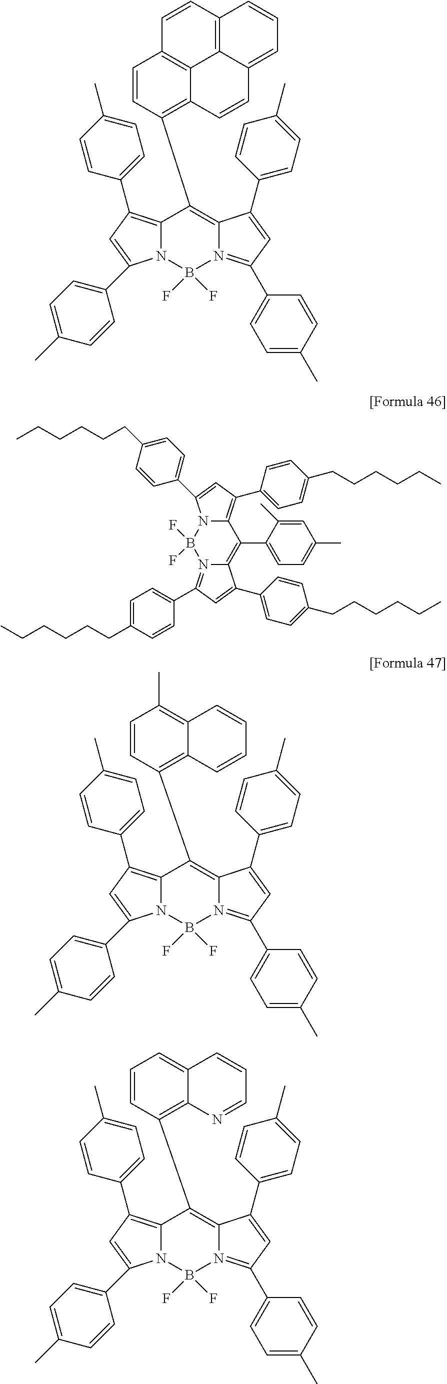

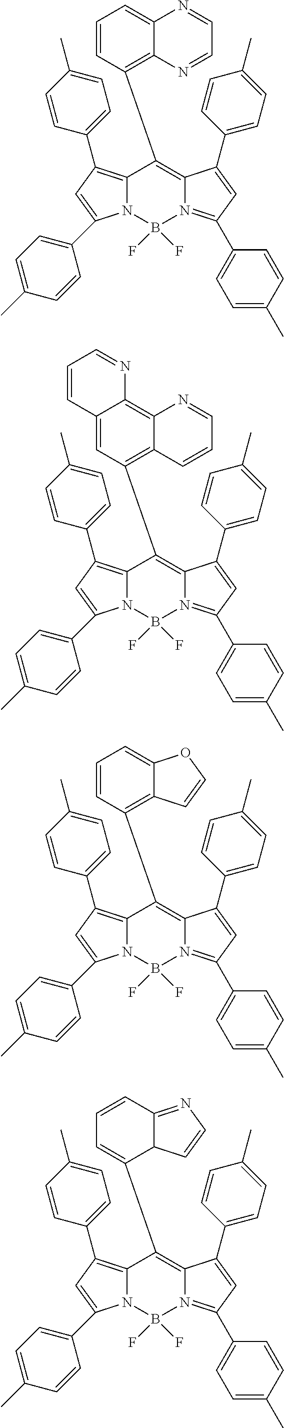

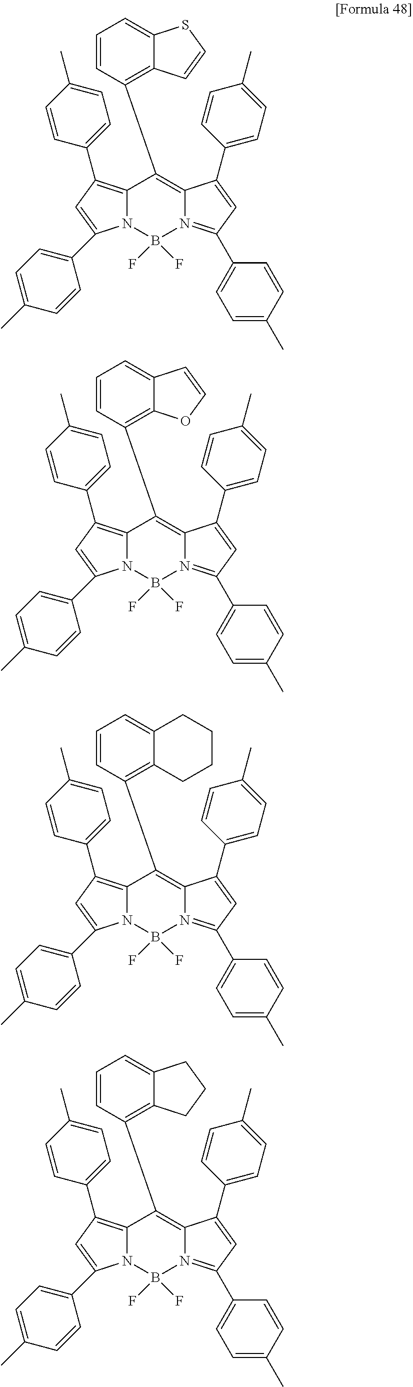

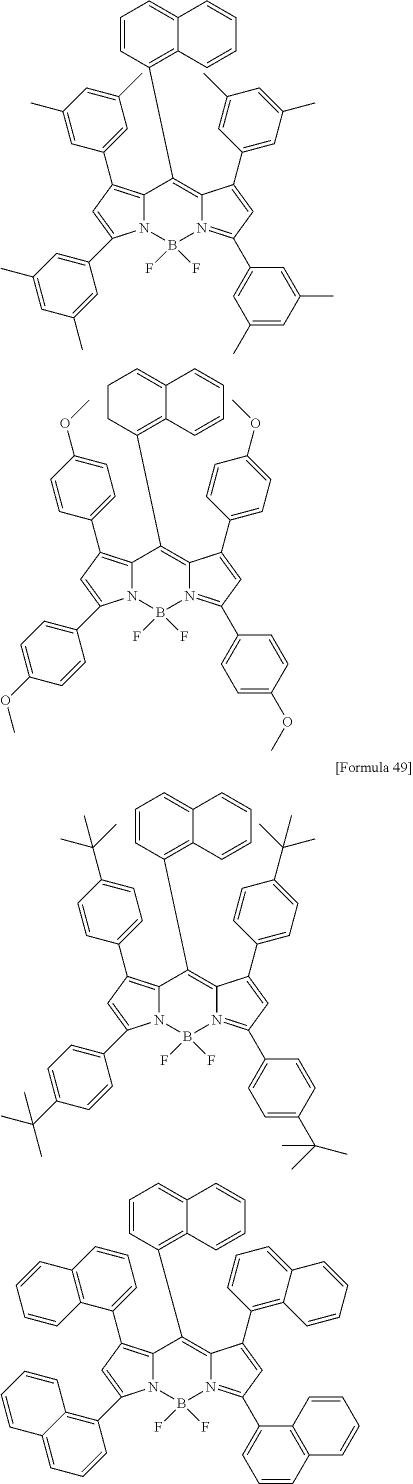

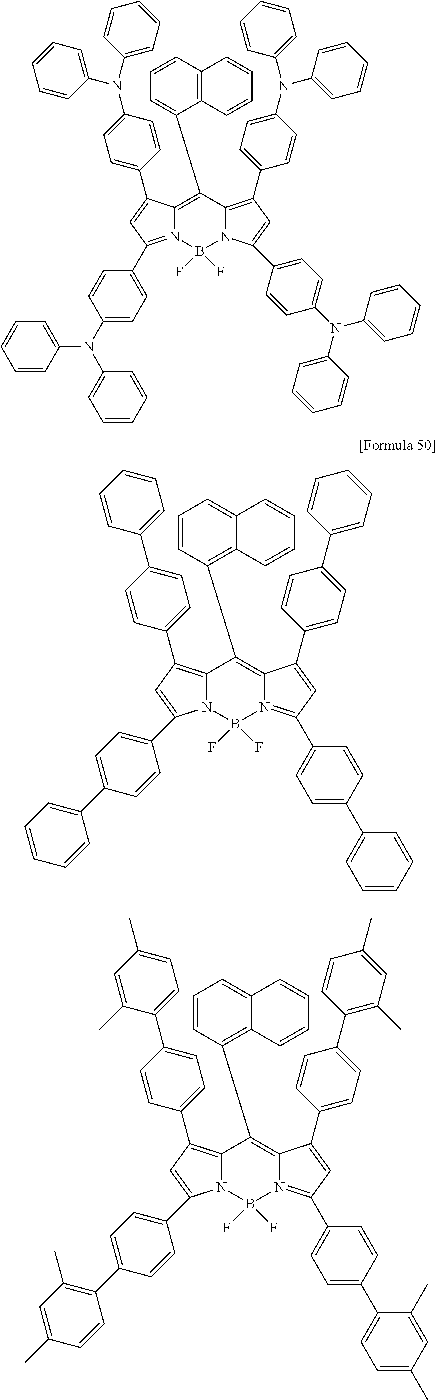

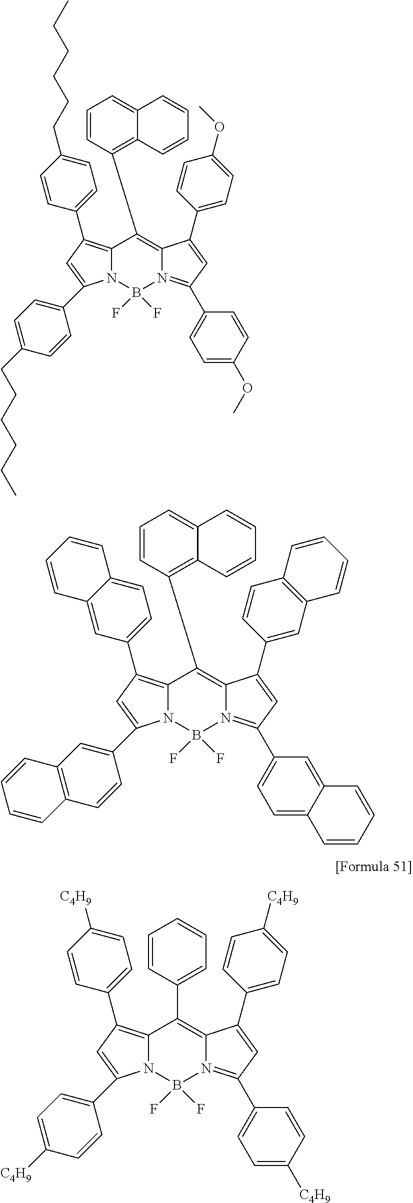

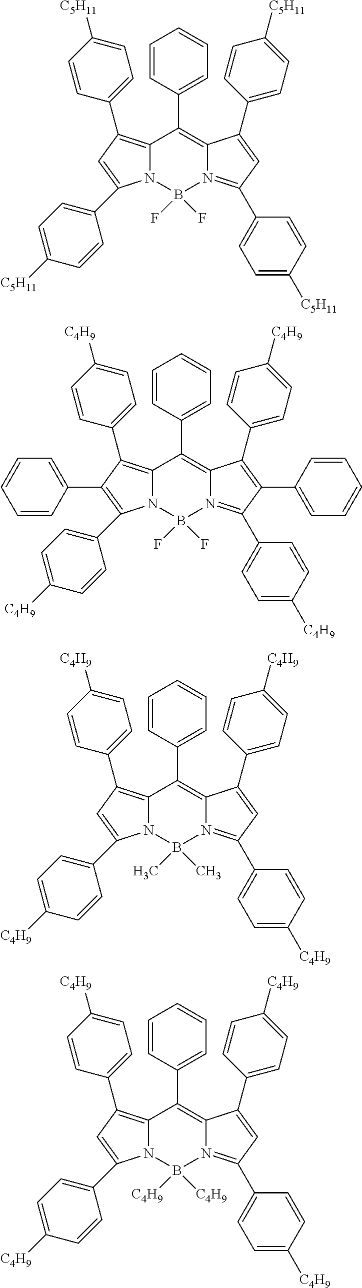

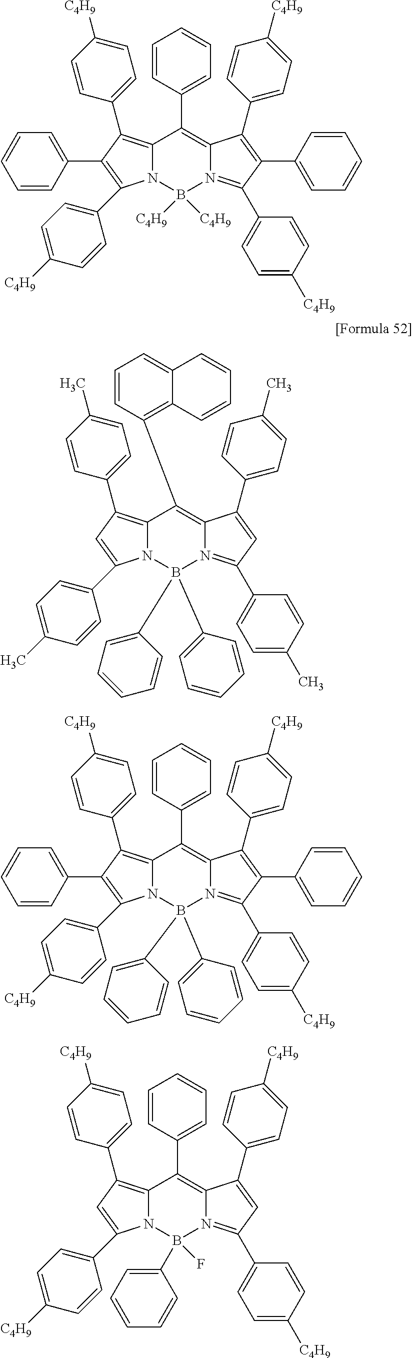

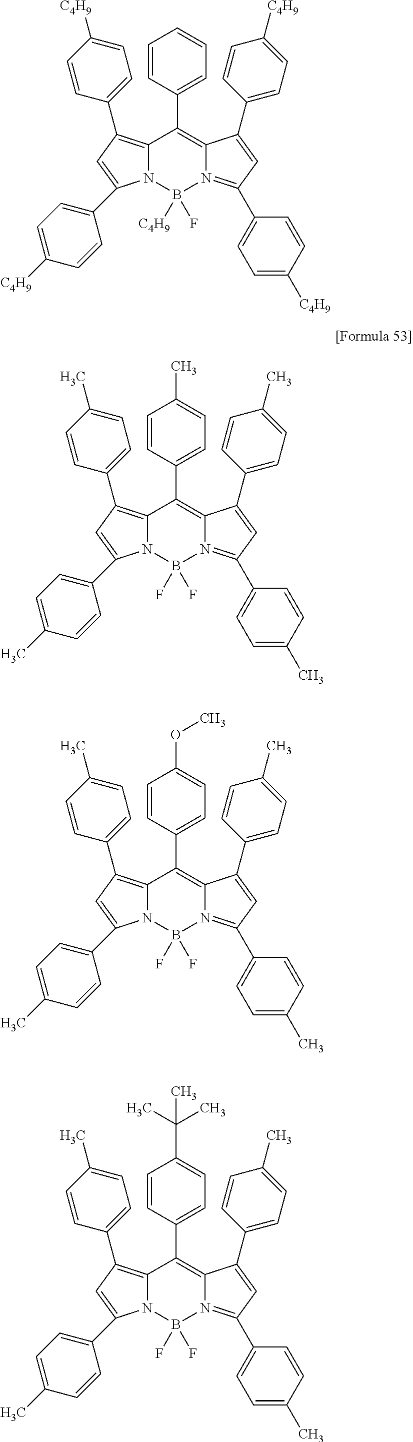

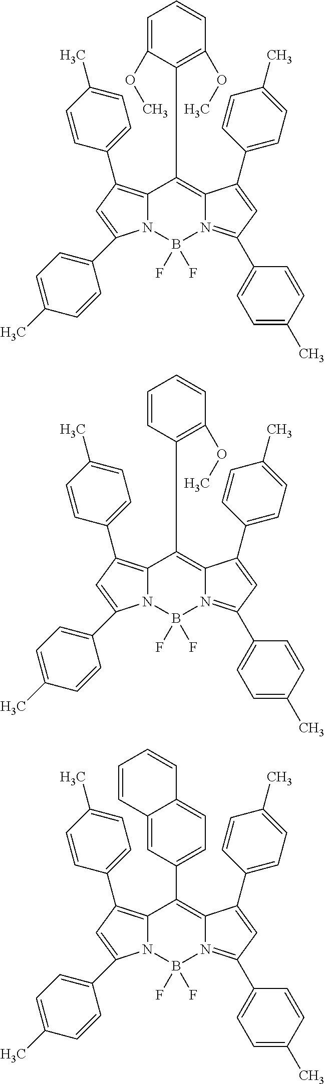

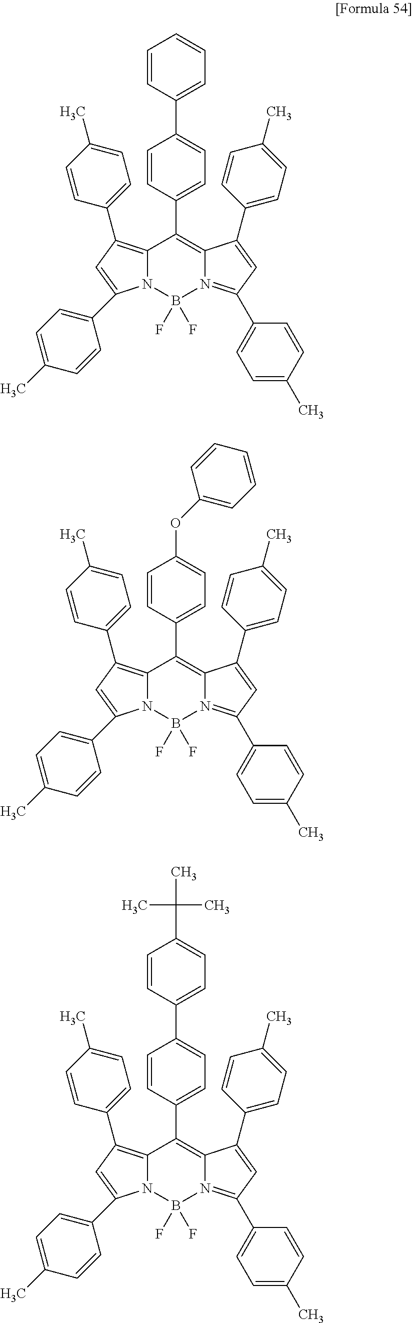

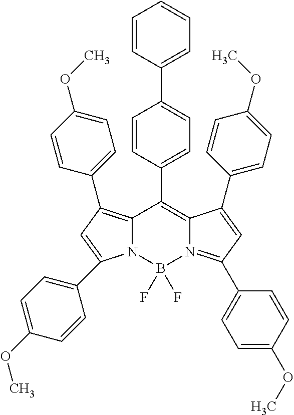

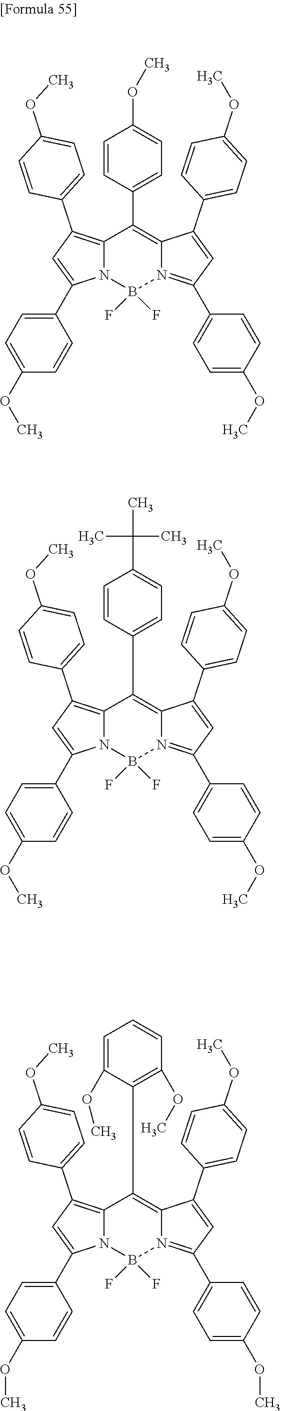

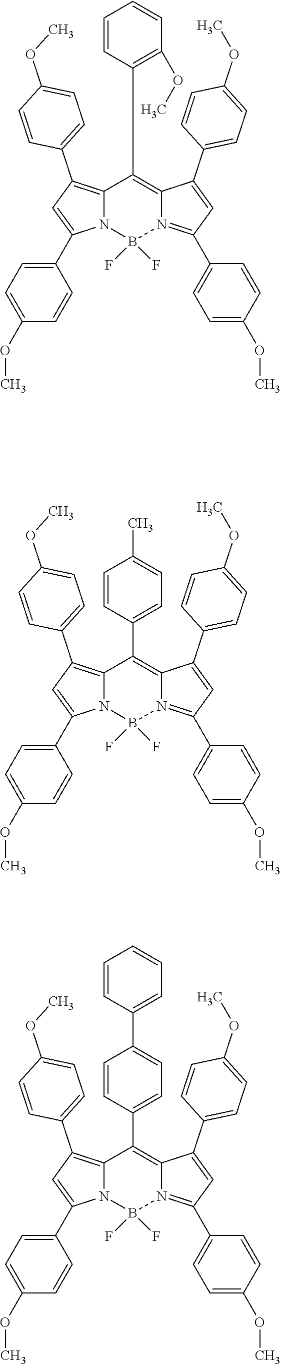

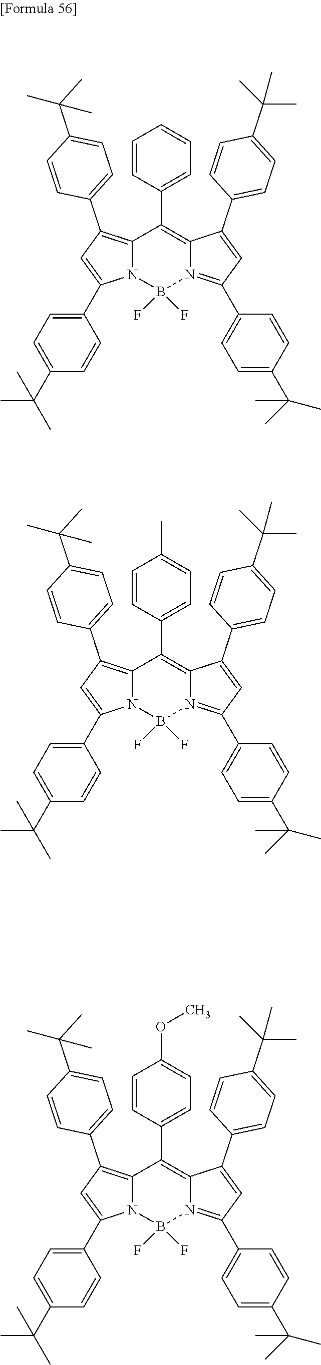

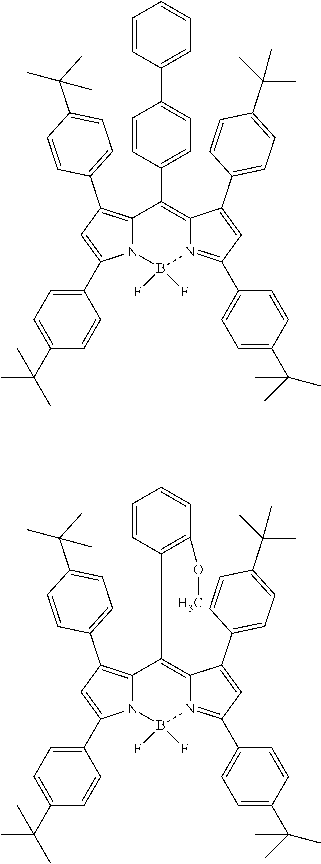

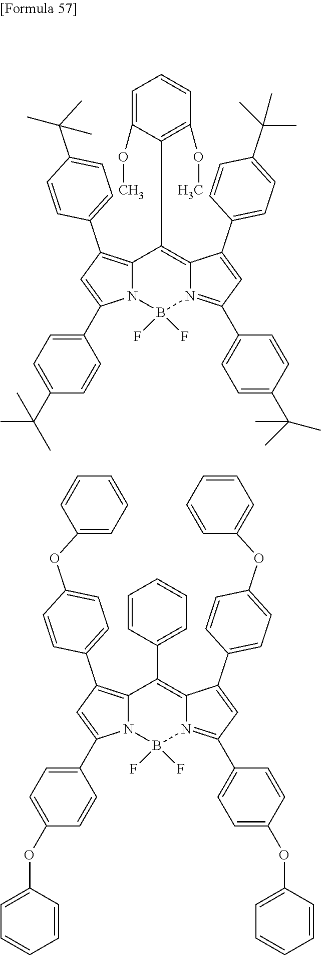

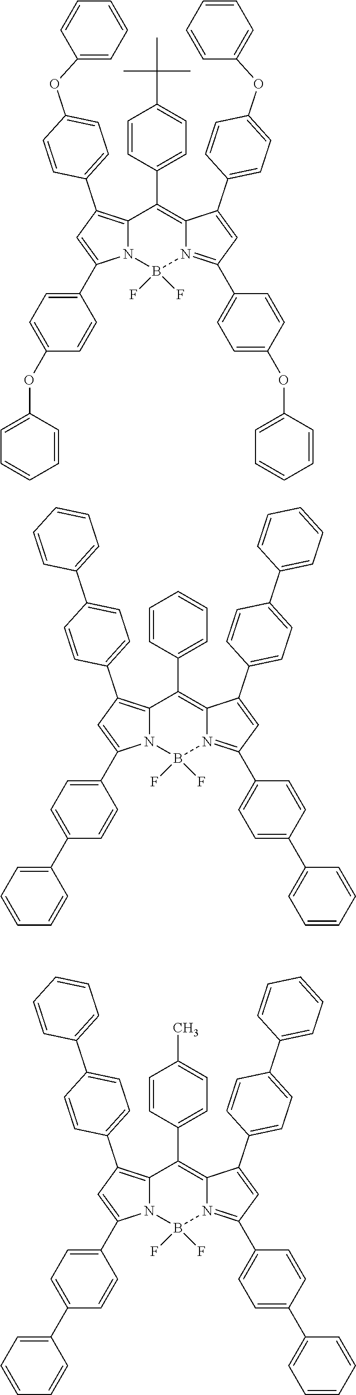

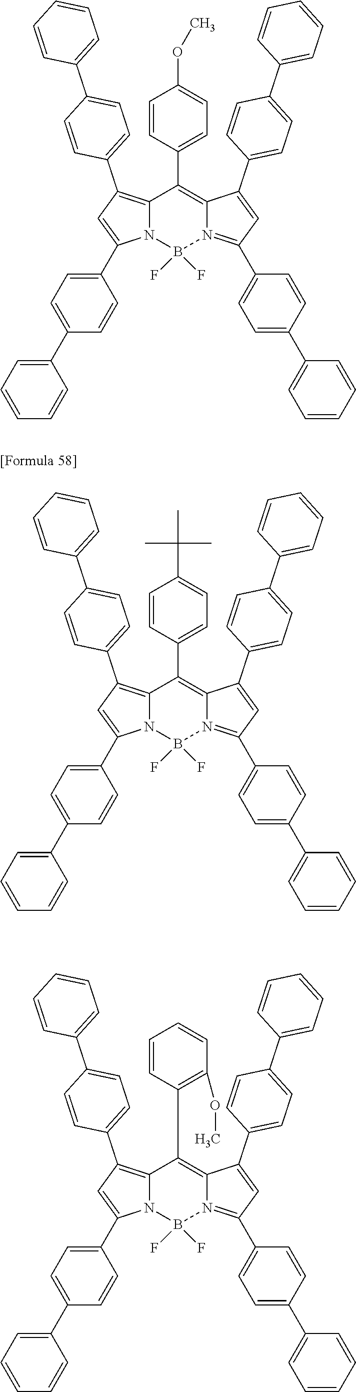

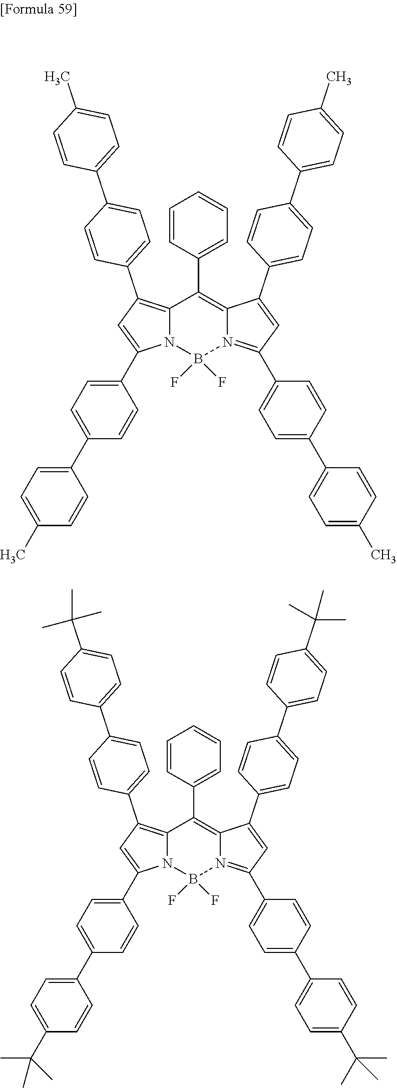

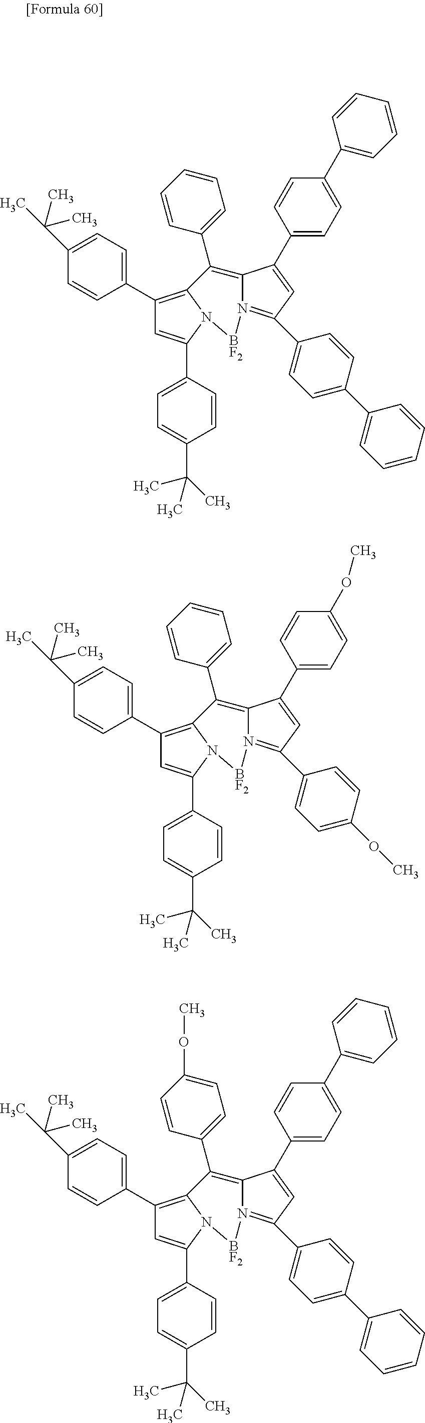

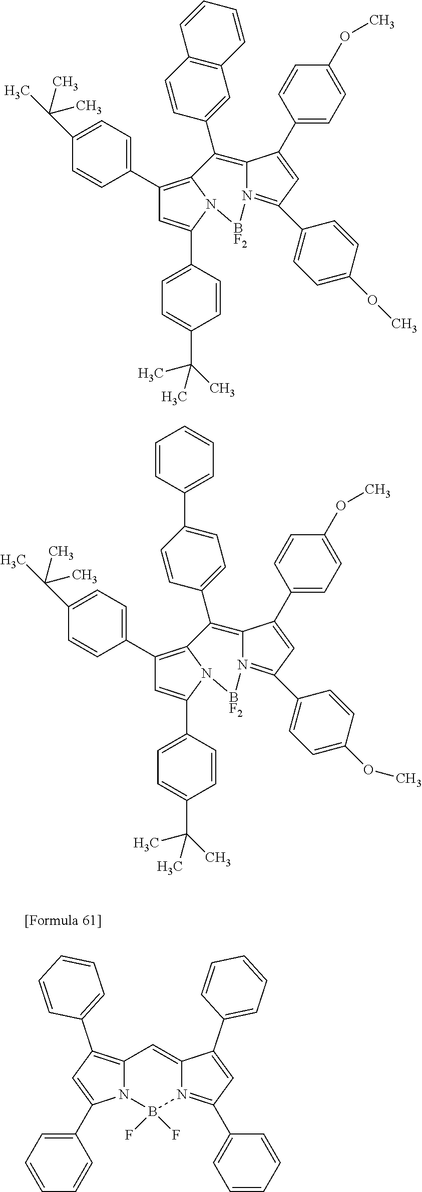

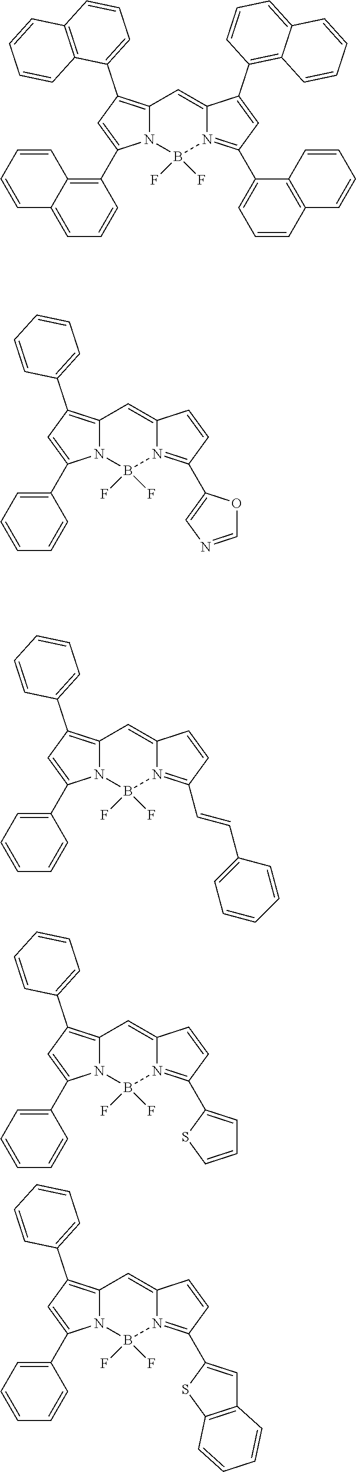

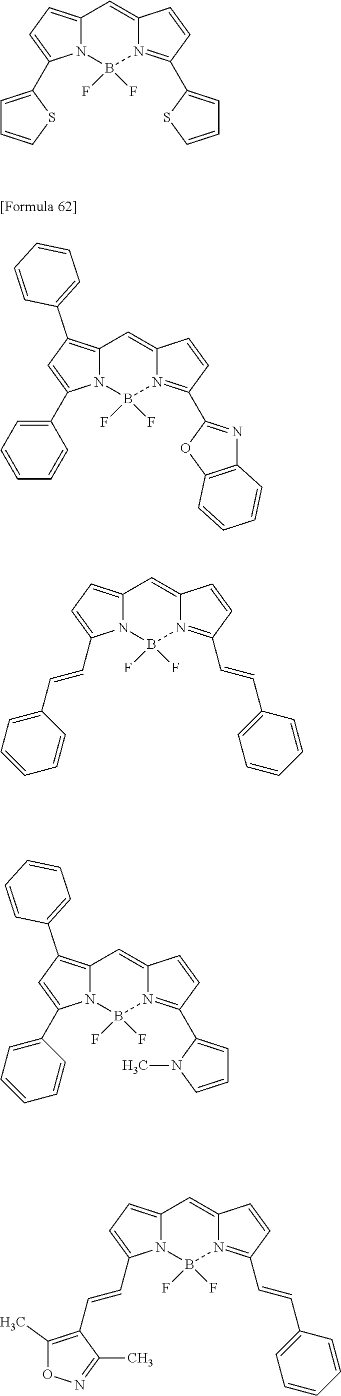

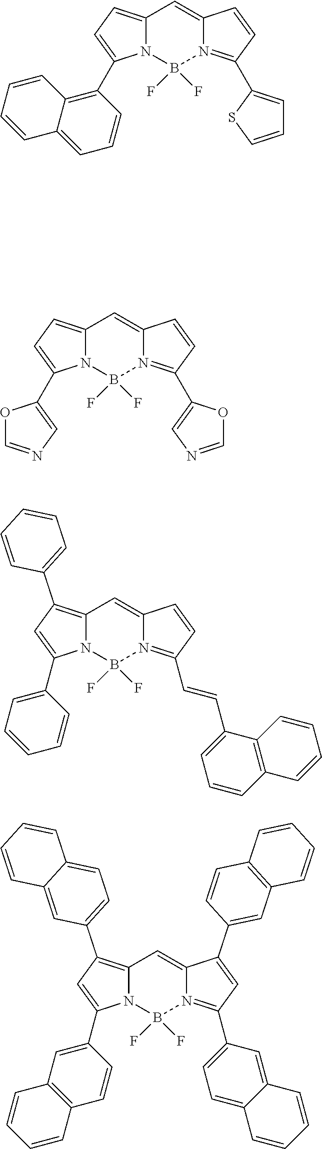

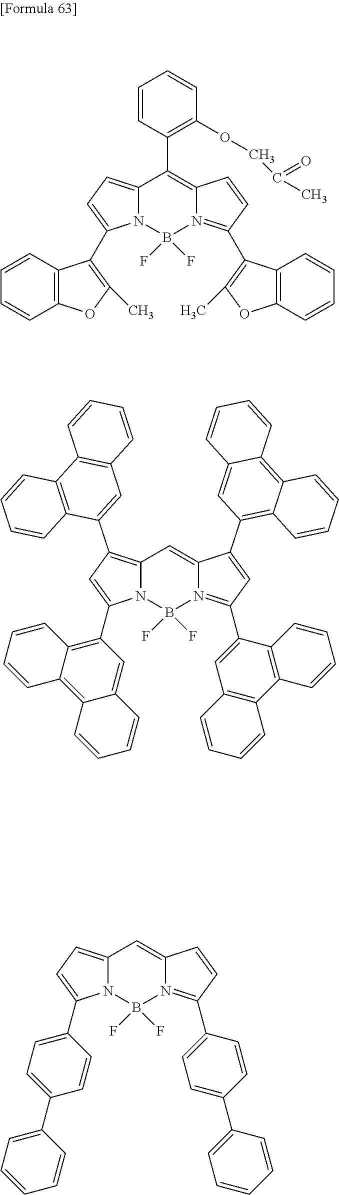

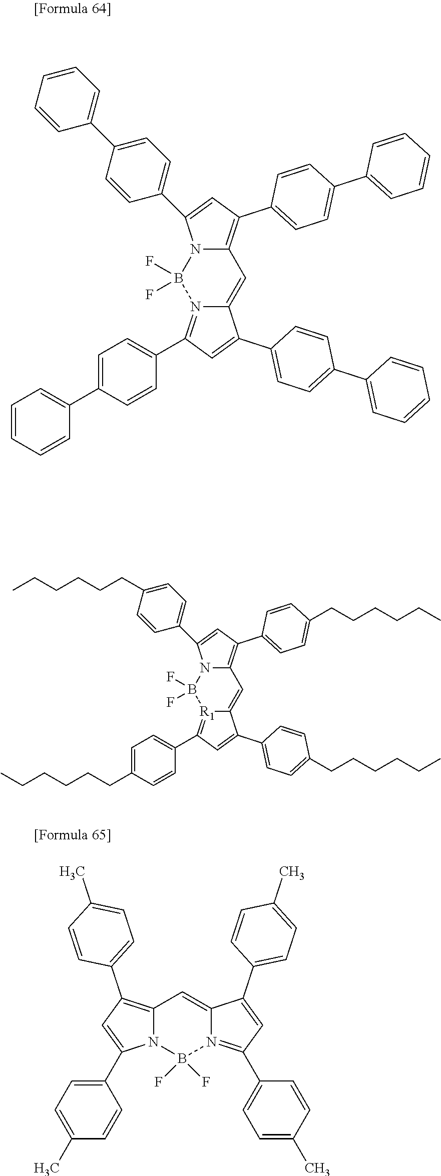

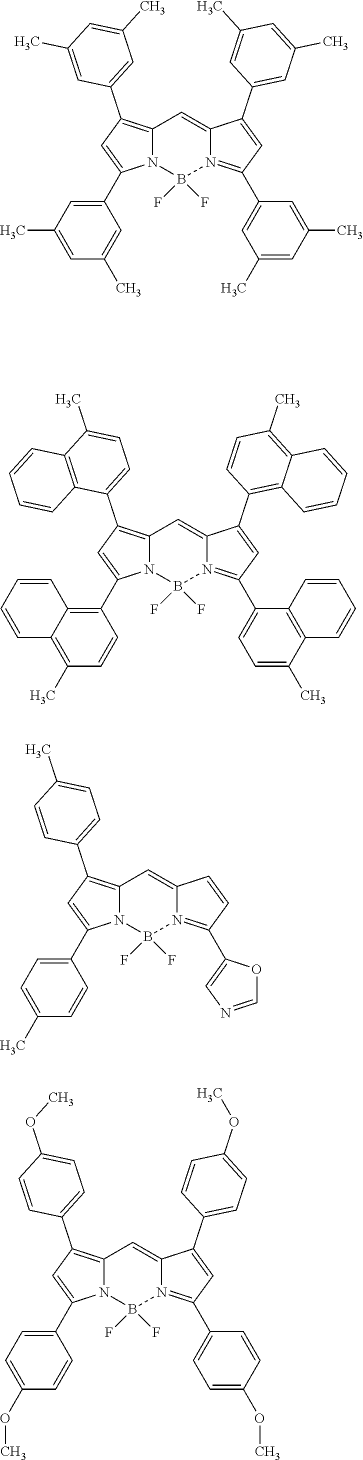

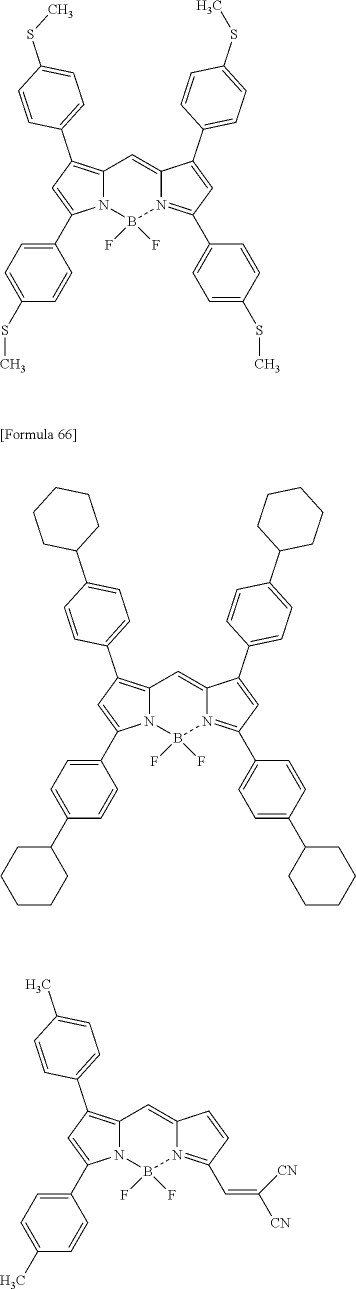

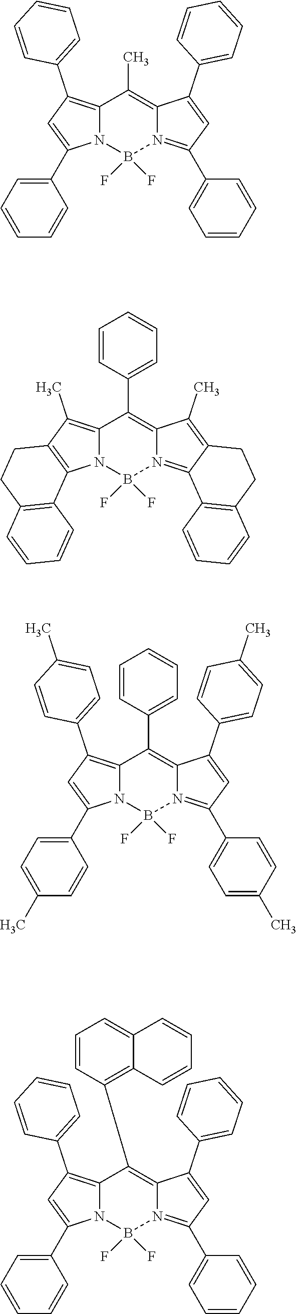

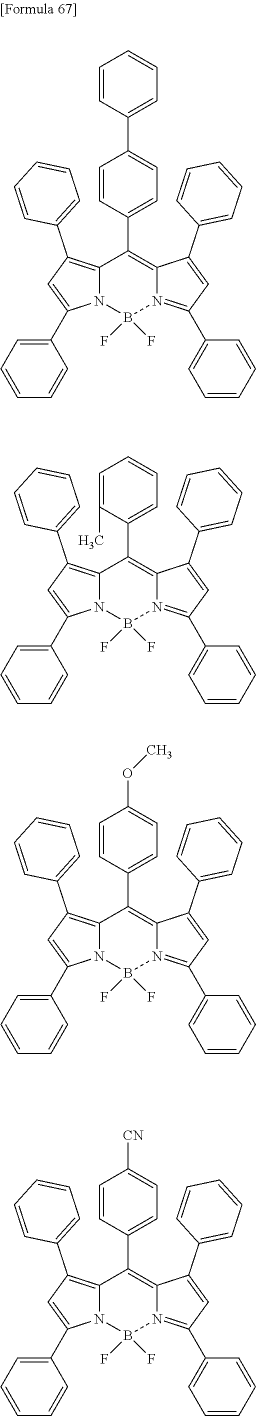

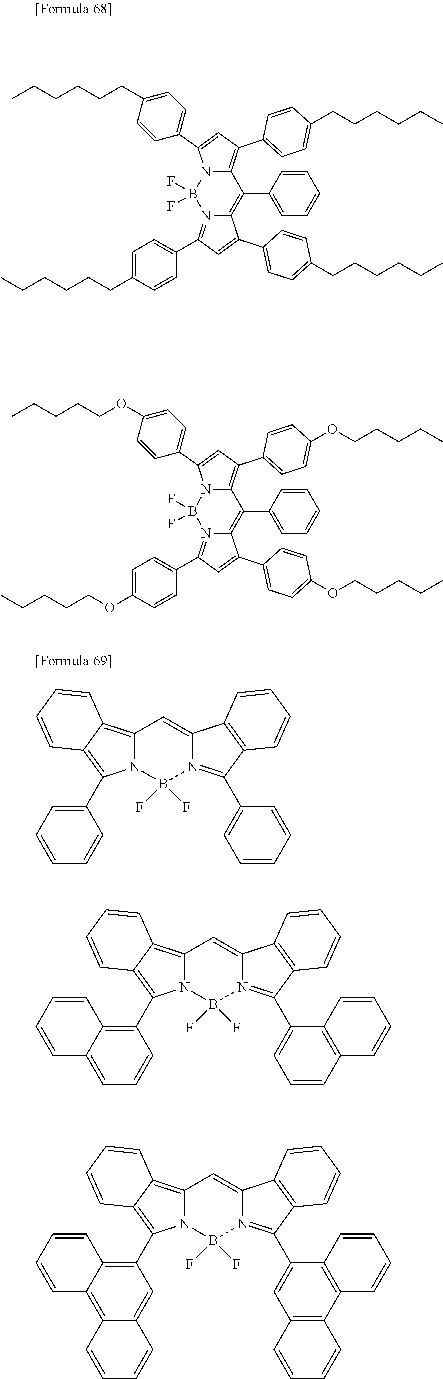

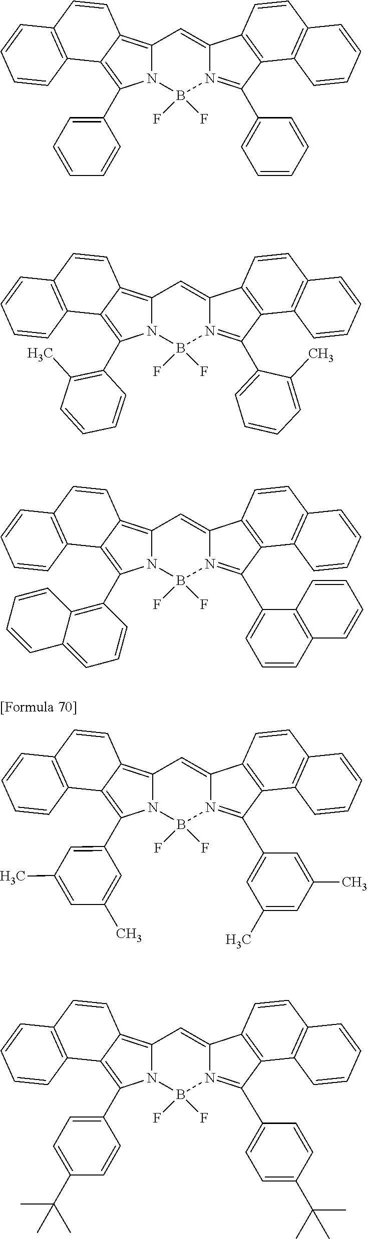

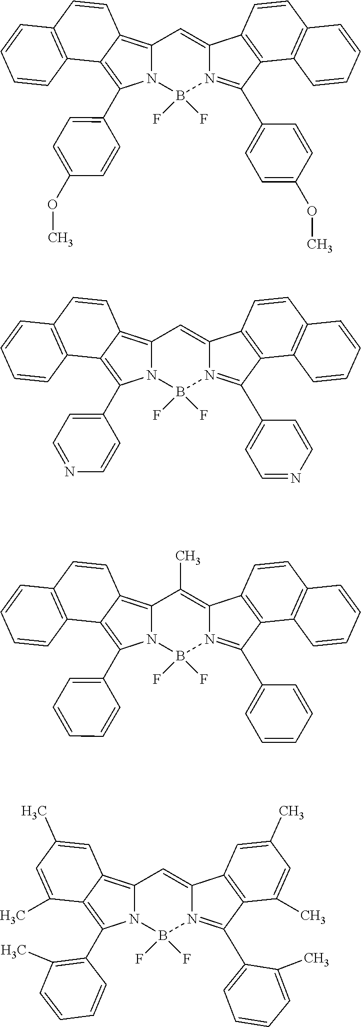

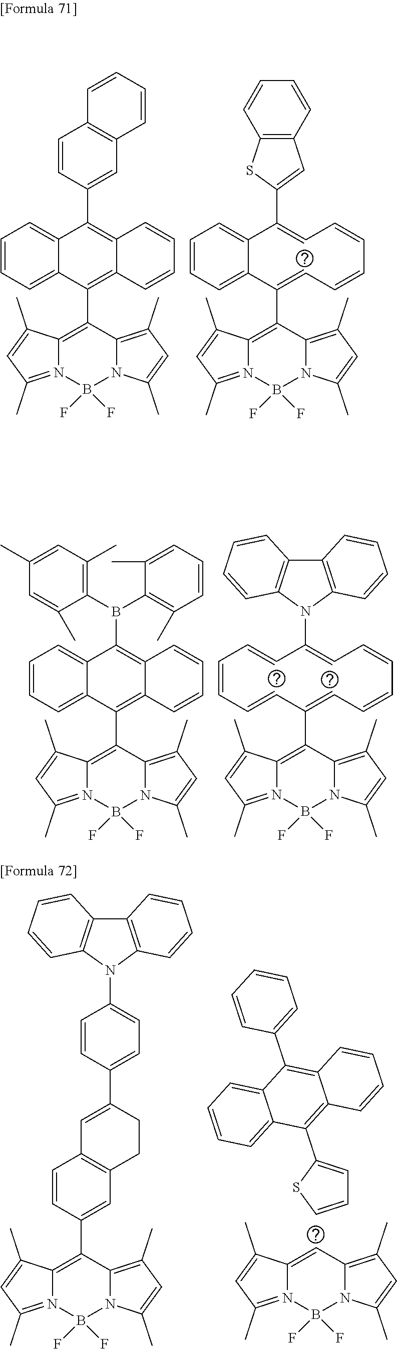

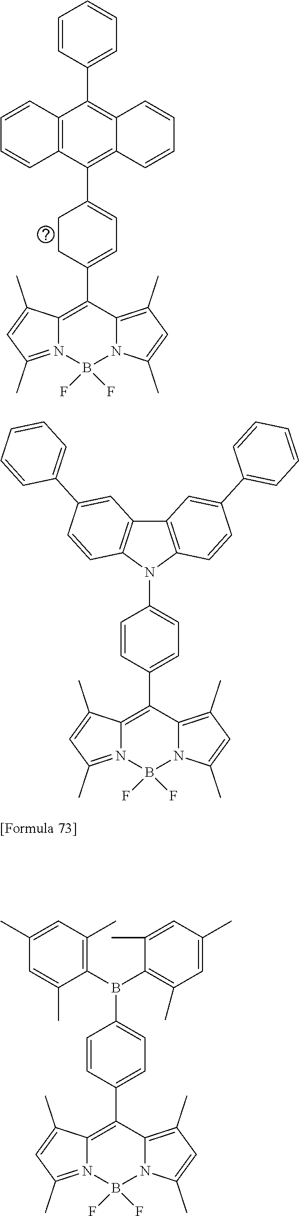

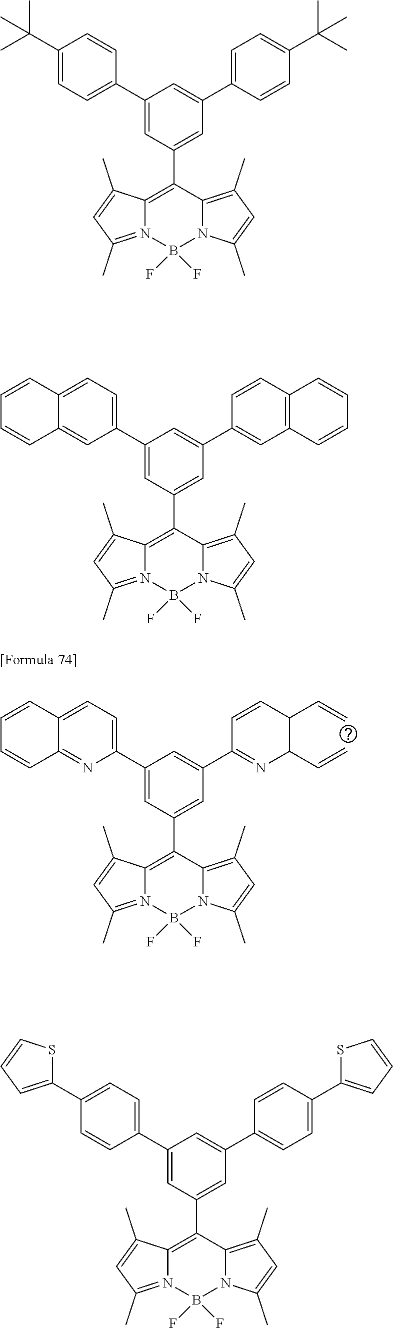

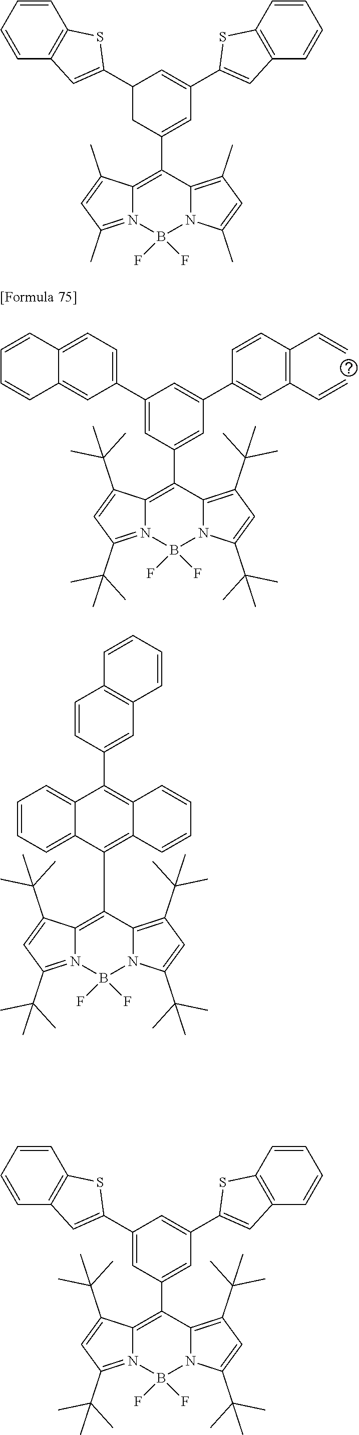

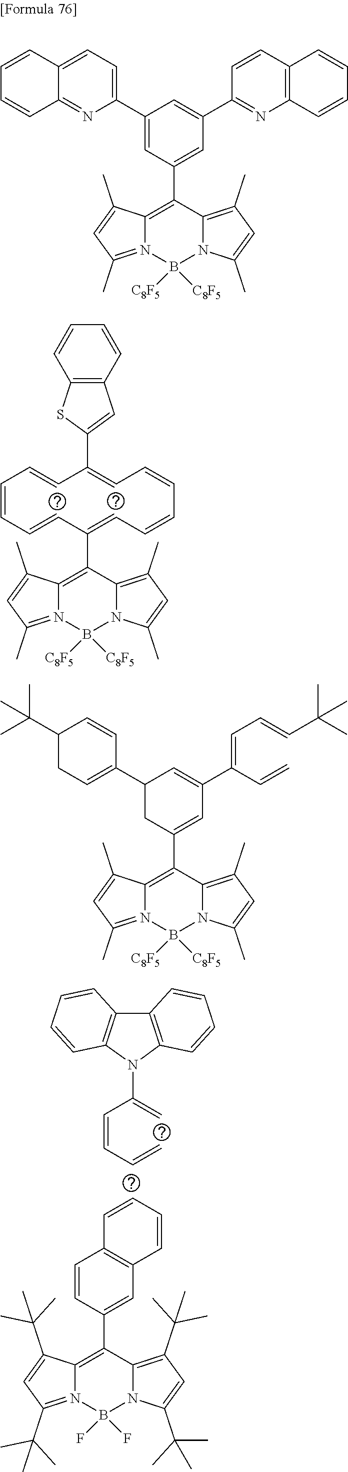

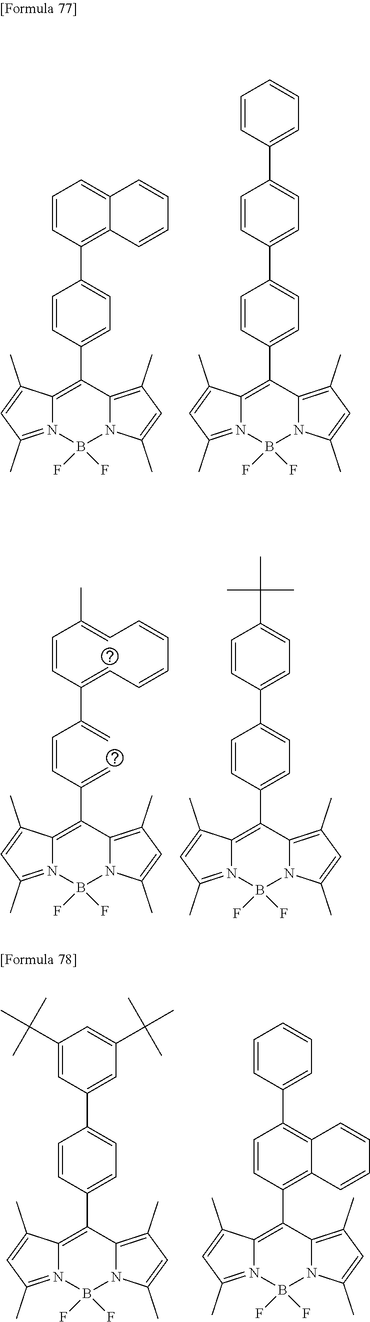

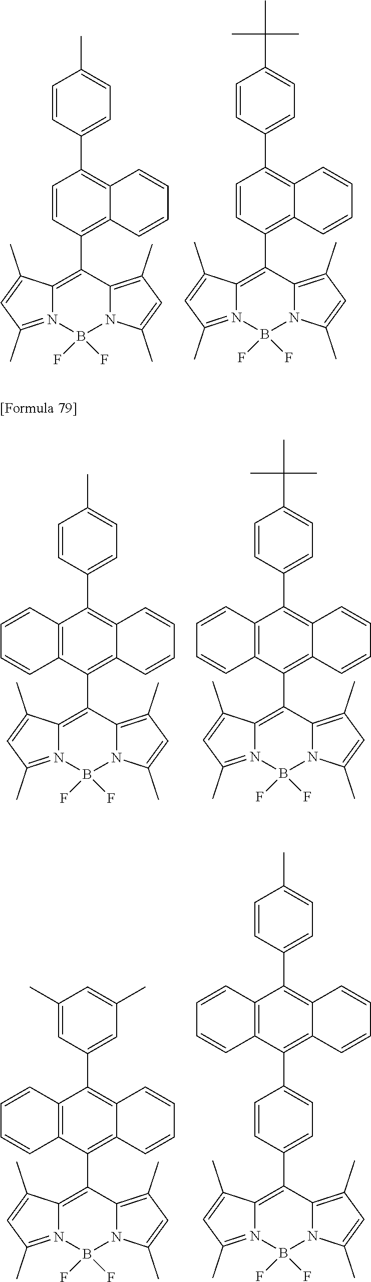

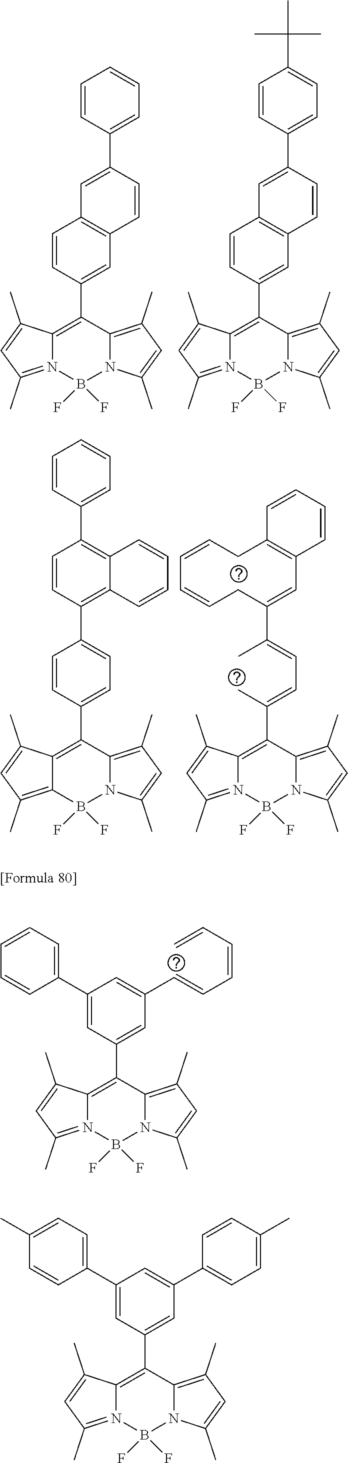

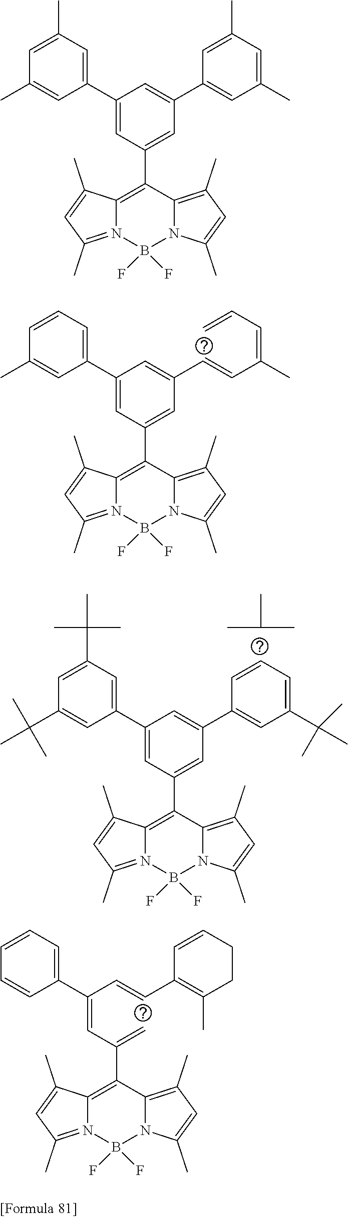

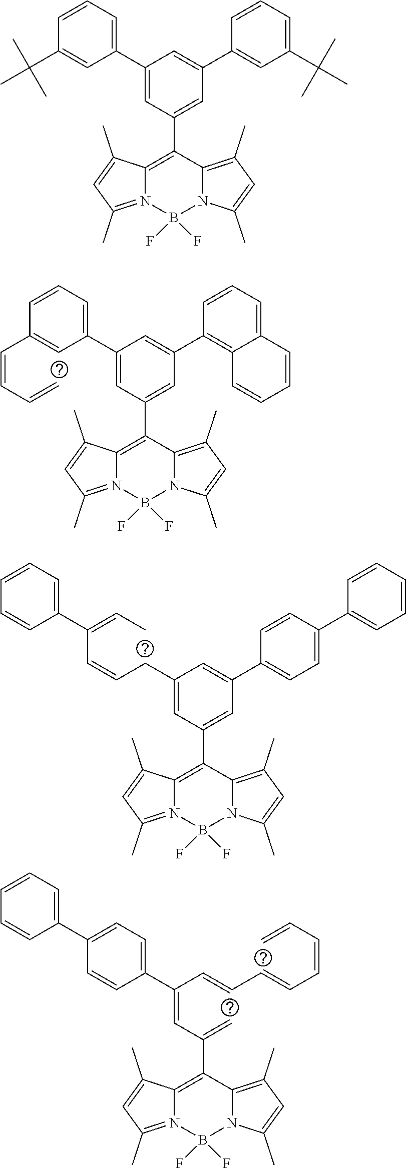

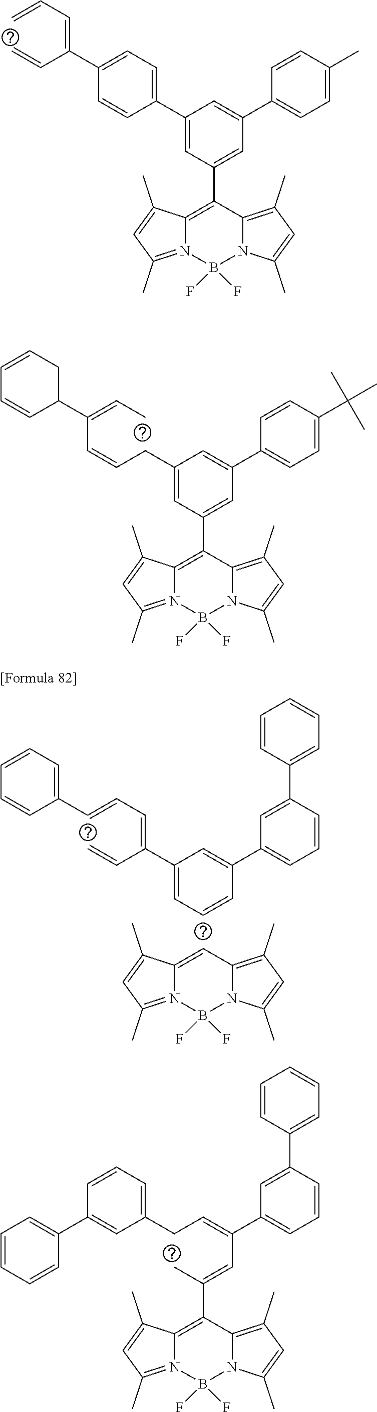

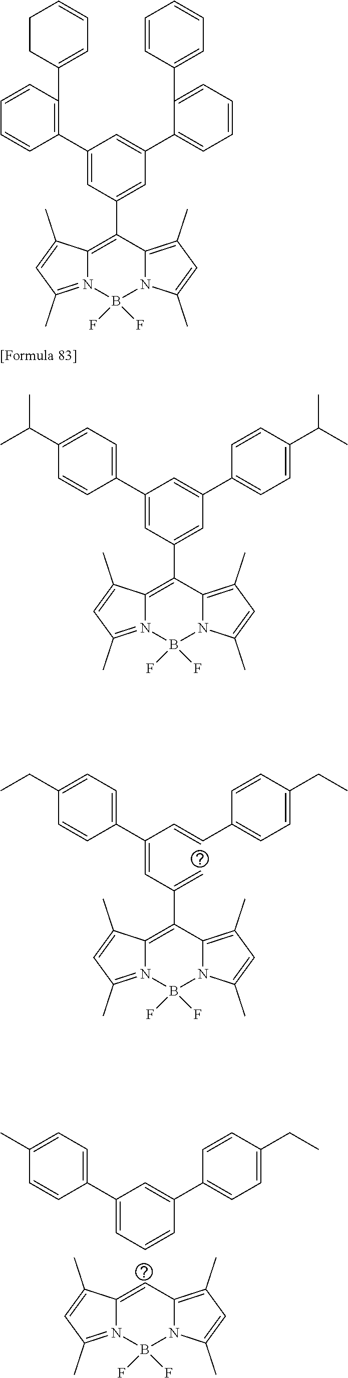

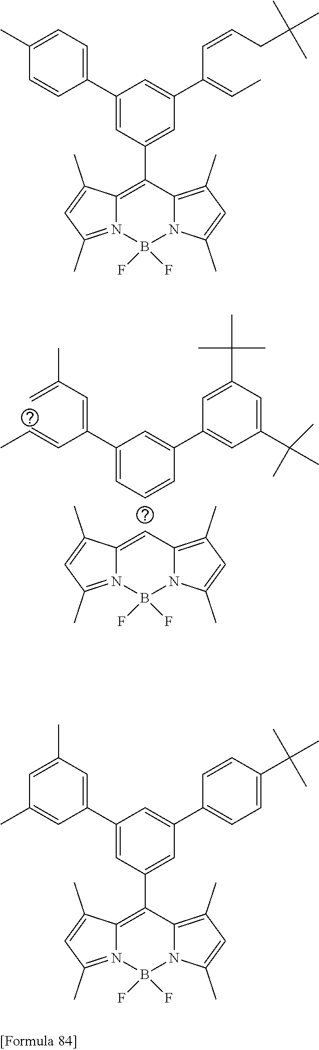

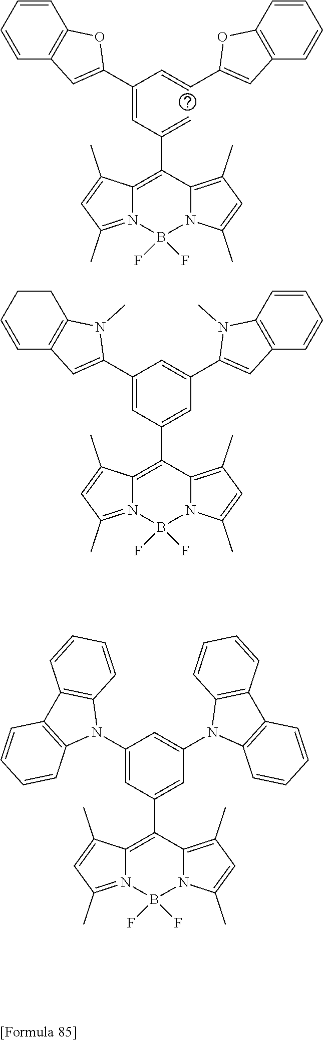

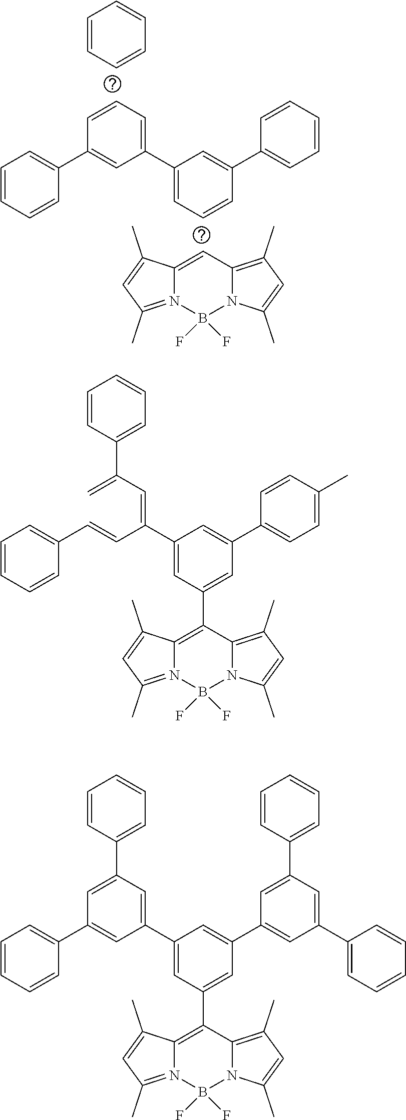

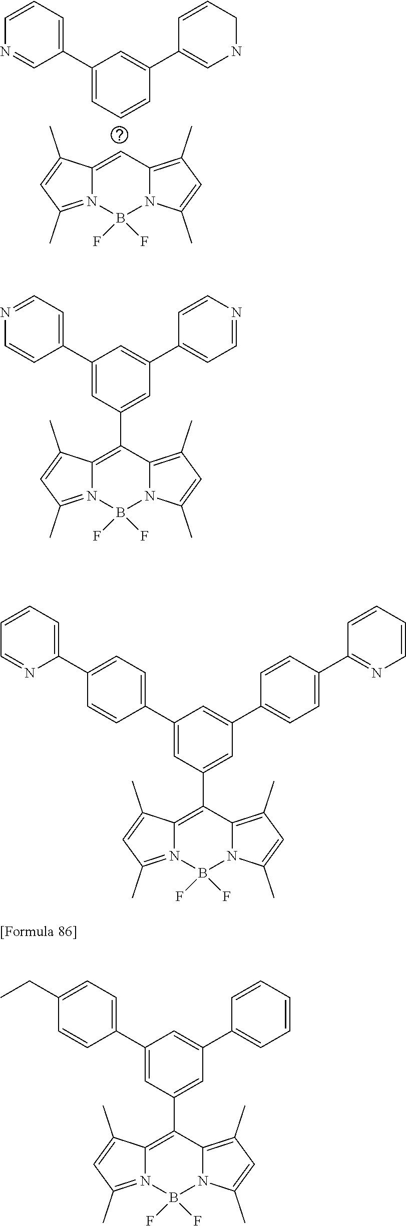

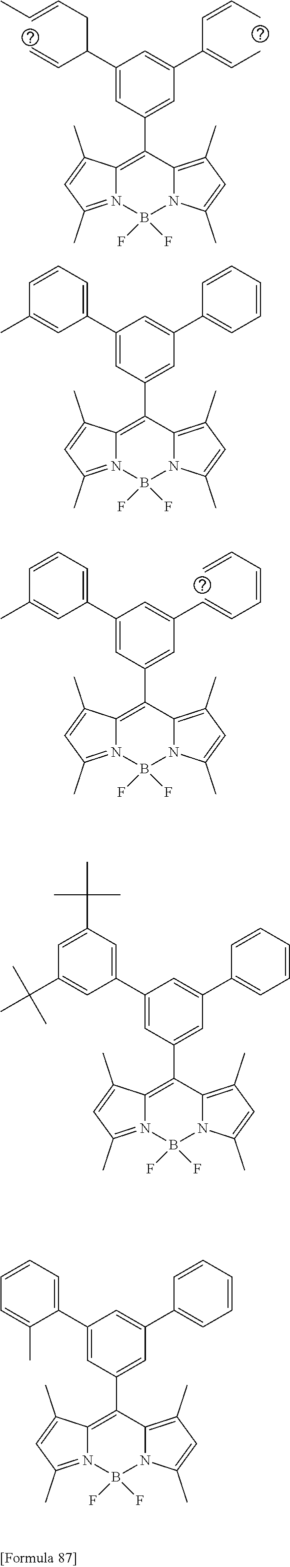

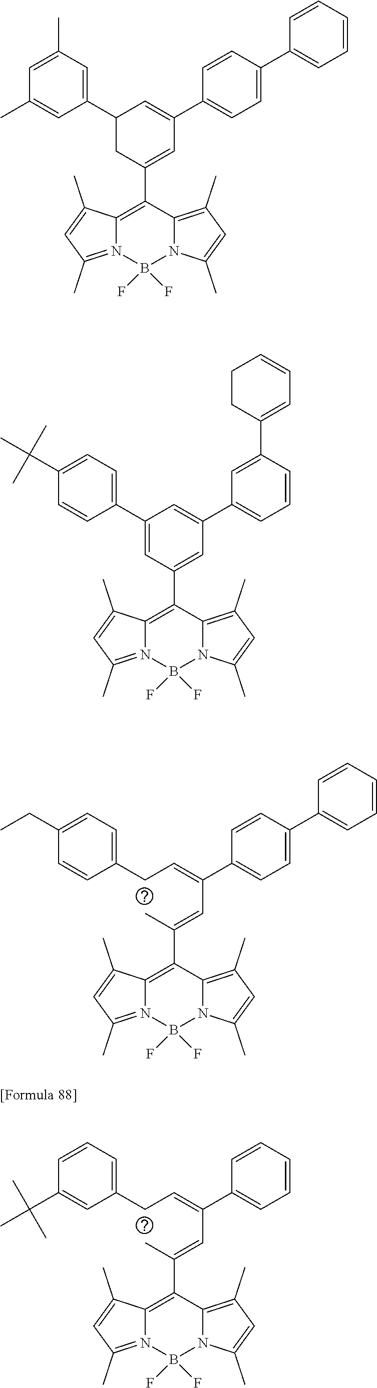

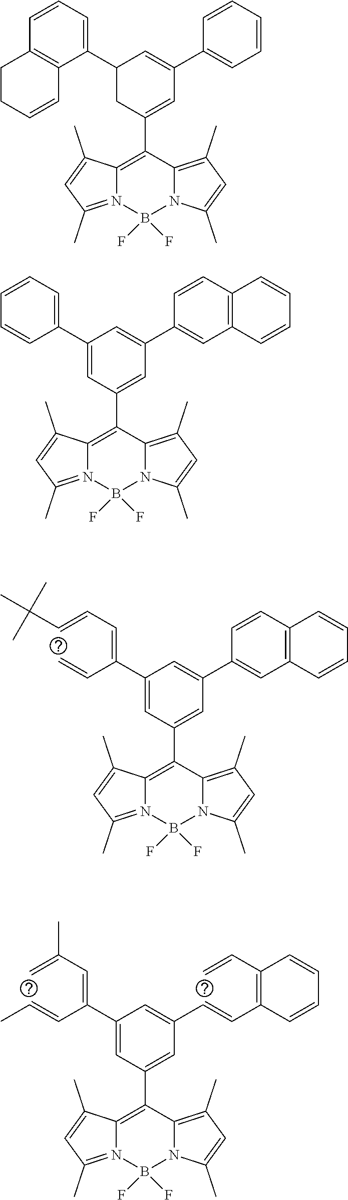

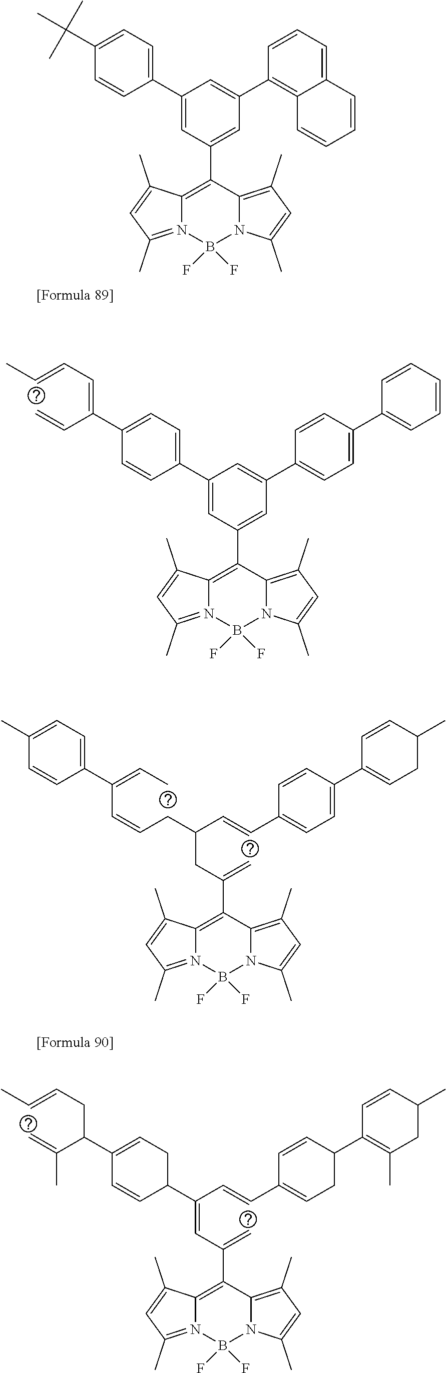

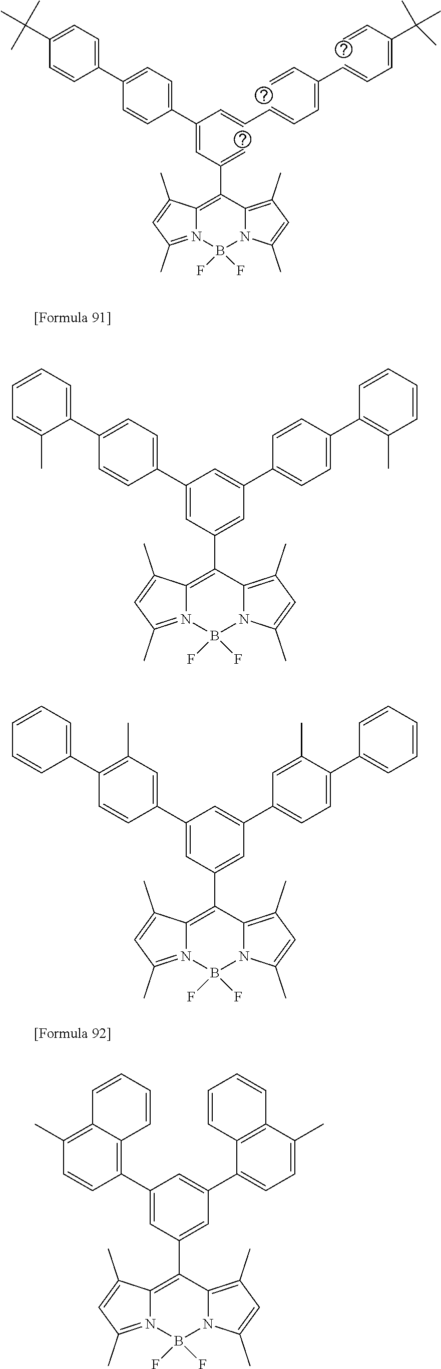

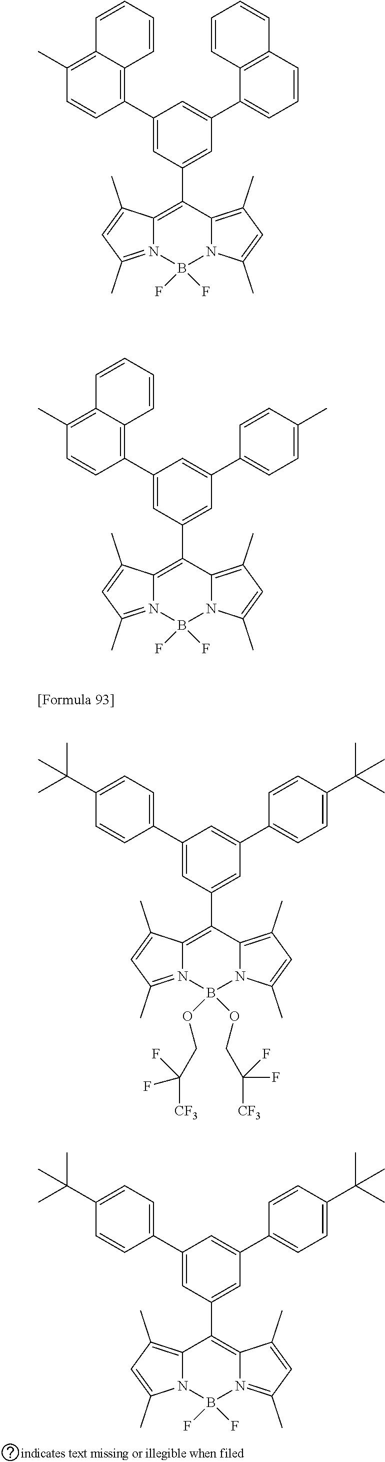

### Second Compound

The second compound is a delayed fluorescent compound.

The second compound in the present exemplary embodiment is not a phosphorescent metal complex. Preferably, the second compound in the present exemplary embodiment is not a metal complex.

In the present exemplary embodiment, the second compound is exemplified by a compound represented by a formula (2) below.

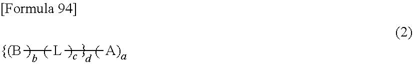

In the formula (2):

A is an acceptor (electron accepting) moiety having a moiety selected from formulae (a-1) to (a-7) below, a plurality of A being mutually the same or different when a plurality of A are present, the plurality of A being optionally mutually bonded to form a saturated or unsaturated ring;

B is a donor (electron dibatubg) moiety having a moiety selected from formulae (b-1) to (b-6) below, a plurality of B being mutually the same or different when a plurality of B are present, the plurality of B being optionally mutually bonded to form a saturated or unsaturated ring;

a, b, and d are each independently 1, 2, 3, 4, or 5;

c is 0, 1, 2, 3, 4 or 5;

when c is 0, A and B are bonded via a single bond or a spiro bond;

when c is 1, 2, 3, 4 or 5, L is a linking group selected from the group consisting of a substituted or unsubstituted aromatic hydrocarbon group having 6 to 30 ring carbon atoms, and a substituted or unsubstituted heterocyclic group having 5 to 30 ring atoms; and when a plurality of L are present, the plurality of L are mutually the same or different and are optionally mutually bonded to form a saturated or unsaturated ring.

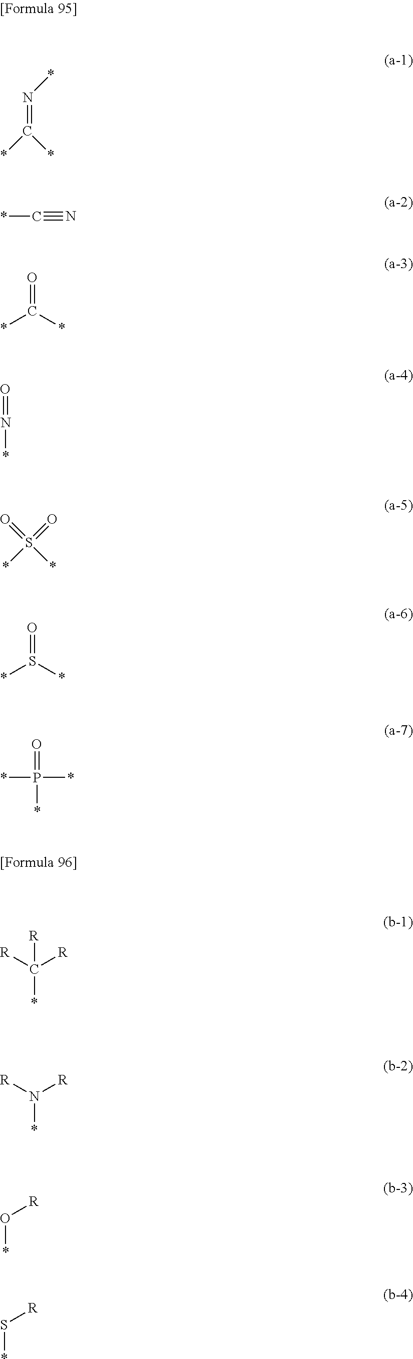

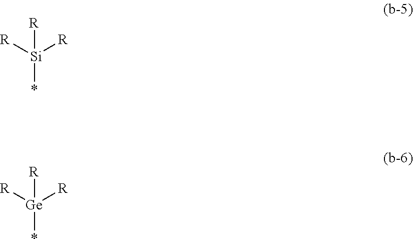

In the formulae (b-1) to (b-6): R each independently represent a hydrogen atom or a substituent, the substituent for R being selected from the group consisting of a substituted or unsubstituted aromatic hydrocarbon group having 6 to 30 ring carbon atoms, a substituted or unsubstituted heterocyclic group having 5 to 30 ring atoms, and a substituted or unsubstituted alkyl group having 1 to 30 carbon atoms; and when a plurality of R are present, the plurality of R are mutually the same or different and are optionally mutually bonded to form a saturated or unsaturated ring.

Examples of the bonding form of the compound represented by the formula (2) are shown in Table 1 below.

TABLE 1

| No. | a | b | c | d | Bonding form |
|---|---|---|---|---|---|
| (1A) | 1 | 1 | 0 | 1 | B—A |
| (1B) | 1 | 1 | 1 | 1 | B—L—A |
| (1C) | 2 | 1 | 0 | 1 | 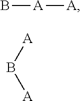 |
| (1D) | 1 | 2 | 0 | 1 | 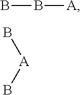 |
| (1E) | 2 | 1 | 1 | 1 | 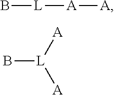 |
| (1F) | 1 | 2 | 1 | 1 | 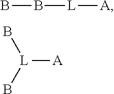 |
| (1G) | 1 | 1 | 2 | 1 | B—L—L—A |
| (1H) | 1 | 1 | 1 | 2 | 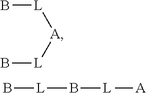 |

In the present exemplary embodiment, the second compound preferably has a moiety represented by a formula (200) below and a moiety represented by a formula (2Y) below in one molecule.

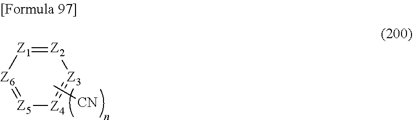

In the formula (200), CN is a cyano group; and n is an integer of 1 or more, n being preferably an integer in a range from 1 to 5, more preferably 2 or 4.

Z₁ to Z₆ are each independently a nitrogen atom, a carbon atom bonded to CN, or a carbon atom bonded to another atom in the molecule of the second compound. For instance, when Z₁ is a carbon atom bonded to CN, at least one of remaining five atoms (i.e. Z₂ to Z₆) is a carbon atom bonded to another atom in the molecule of the second compound. The another atom may be an atom of a moiety represented by the formula (2Y) below or an atom of a substituent or a linking group for bonding the moiety.

The second compound of the present exemplary embodiment may have a moiety in a form of a six-membered ring formed by Z₁ to Z₆, or, alternatively, have a moiety in a form of a fused ring formed by fusing a further ring to the six-membered ring.

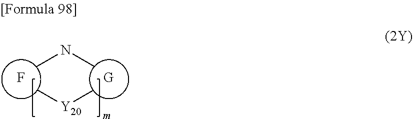

In the formula (2Y): F and G each independently represent a cyclic structure; m is 0 or 1; and when m is 1, Y₂₀ represents a single bond, an oxygen atom, a sulfur atom, a selenium atom, a carbon atom, a silicon atom, or a germanium atom.

When m is 0 in the formula (2Y), the formula (2Y) is represented by a formula (20Y) below.

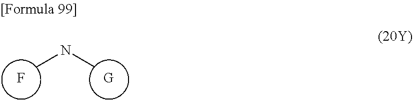

The cyclic structures F and G in the formula (20Y) represent the same as the cyclic structures F and G in the formula (2Y).

When m is 1 in the formula (2Y), the formula (2Y) is represented by any one of formulae (22) to (28) below.

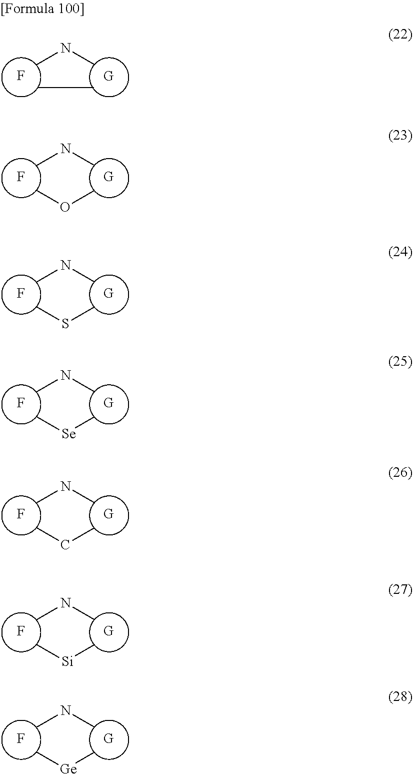

The cyclic structures F and G in the formulae (22) to (28) represent the same as the cyclic structures F and G in the formula (2Y).

In the present exemplary embodiment, the cyclic structures F and G are each preferably a five-membered ring or a six-membered ring, the five-membered or six-membered ring preferably being an unsaturated ring, more preferably an unsaturated six-membered ring.

In the present exemplary embodiment, the second compound is preferably a compound represented by a formula (20) below.

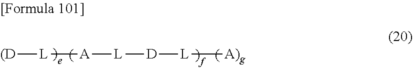

In the formula (20), A is represented by the formula (200), where CN represents a cyano group, n is an integer of 1 or more, Z₁ to Z₆ each independently represent a nitrogen atom, a carbon atom bonded to CN, a carbon atom bonded to R, a carbon atom bonded to L, or a carbon atom bonded to D, at least one of Z₁ to Z₆ being the carbon atom bonded to CN and at least one of Z₁ to Z₆ being the carbon atom bonded to L or D; and

R is each independently a hydrogen atom or a substituent, the substituent for R being selected from the group consisting of a halogen atom, a substituted or unsubstituted aryl group having 6 to 30 ring carbon atoms, a substituted or unsubstituted aromatic heterocyclic group having 5 to 30 ring atoms, a substituted or unsubstituted alkyl group having 1 to 30 carbon atoms, a substituted or unsubstituted alkylsilyl group having 3 to 30 carbon atoms, a substituted or unsubstituted arylsilyl group having 6 to 60 ring carbon atoms, a substituted or unsubstituted alkoxy group having 1 to 30 carbon atoms, a substituted or unsubstituted aryloxy group having 6 to 30 ring carbon atoms, a substituted or unsubstituted alkylamino group having 2 to 30 carbon atoms, a substituted or unsubstituted arylamino group having 6 to 60 ring carbon atoms, a substituted or unsubstituted alkylthio group having 1 to 30 carbon atoms, and a substituted or unsubstituted arylthio group having 6 to 30 ring carbon atoms.

In the formula (20), D is represented by the formula (2Y), where the cyclic structures F and G are unsubstituted or substituted, m is 0 or 1, and, when m is 1, Y₂₀ represents a single bond, an oxygen atom, a sulfur atom, a selenium atom, a carbonyl group, CR₂₁R₂₂, SiR₂₃R₂₄ or GeR₂₅R₂₆, R₂₁ to R₂₆ representing the same group for R. When m is 1 in the formula (2Y), the formula (2Y) is represented by any one of the formulae (22) to (25) and formulae (21Y) to (24Y) below.

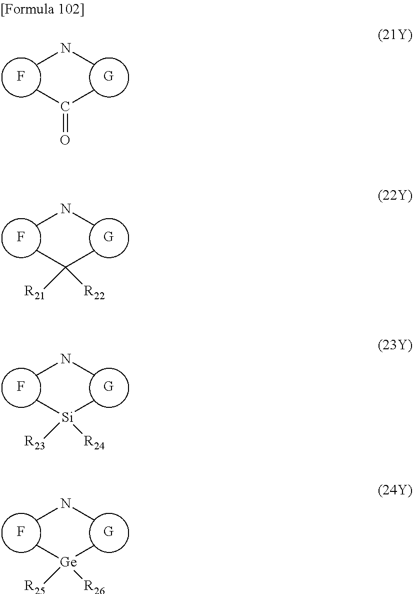

In the formula (20): (i) when L is present between A and D,

L is a single bond, a substituted or unsubstituted aromatic hydrocarbon group having 6 to 14 ring carbon atoms, a substituted or unsubstituted aromatic heterocyclic group having 5 to 14 ring atoms, CR₈₁R₈₂, NR₈₃, O, S, SiR₈₄R₈₅, CR₈₆R₈₇-CR₈₈R₈₉, CR₉₀═CR₉₁, a substituted or unsubstituted alicyclic hydrocarbon group, or a substituted or unsubstituted aliphathic heterocyclic group; and

R₈₁ to R₉₁ each independently represent the same as R.

In the formula (20), (ii) when L s at a terminal end of a molecule of the second compound, L represents the same as R.

In the formula (20): f is an integer of 1 or more; e and g are each independently an integer of 0 or more; a plurality of A are optionally mutually the same or different; a plurality of D are optionally mutually the same or different; and a plurality of L are optionally mutually the same or different.

The formula (20) is represented by, for instance, formulae (201) to (220) below.

TABLE 2

| Formula | e, f, g in the | |
|---|---|---|
| No. | Formula (20) | Formula |
| (201) | e = 0, f = 1, g = 0 | 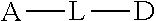 |
| (202) | e = 0, f = 1, g = 0 | 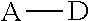 |
| (203) | e = 0, f = 1, g = 1 | 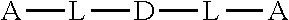 |
| (204) | e = 0, f = 1, g = 1 | 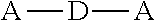 |
| (205) | e = 1, f = 1, g = 0 | 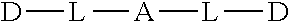 |
| (206) | e = 1, f = 1, g = 0 | 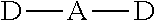 |

TABLE 3

| Formula | e, f, g in the | |
|---|---|---|
| No. | Formula (20) | Formula |
| (207) | e = 1, f = 1, g = 1 | 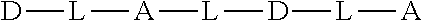 |
| (208) | e = 1, f = 1, g = 1 | 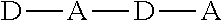 |
| (209) | e = 1, f = 2, g = 0 | 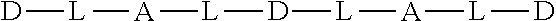 |
| (210) | e = 1, f = 2, g = 0 | 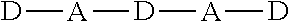 |
| (211) | e = 0, f = 2, g = 1 | 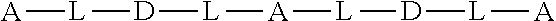 |
| (212) | e = 0, f = 2, g = 1 | 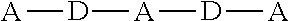 |

TABLE 4

| Formula | e, f, g in the | |
|---|---|---|
| No. | Formula (20) | Formula |
| (213) | e = 2, f = 1, g = 0 | 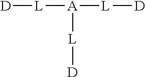 |
| (214) | e = 2, f = 1, g = 0 | 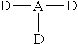 |
| (215) | e = 3, f = 1, g = 0 | 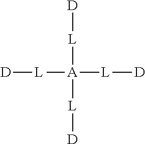 |
| (216) | e = 3, f = 1, g = 0 | 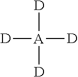 |

TABLE 5

| Formula | e, f, g in the | |
|---|---|---|
| No. | Formula (20) | Formula |
| (217) | e = 0, f = 1, g = 2 | 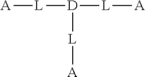 |
| (218) | e = 0, f = 1, g = 2 | 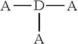 |
| (219) | e = 0, f = 1, g = 3 | 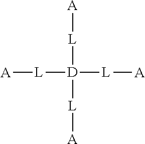 |
| (220) | e = 0, f = 1, g = 3 | 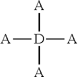 |

Further, in the repeating unit in parentheses suffixed by a repeating number fin the formula (20), D may be bonded to A via L, or A may be, bonded to D via L. For instance, the repeating unit may be branched as shown in formulae (221) to (228) below,

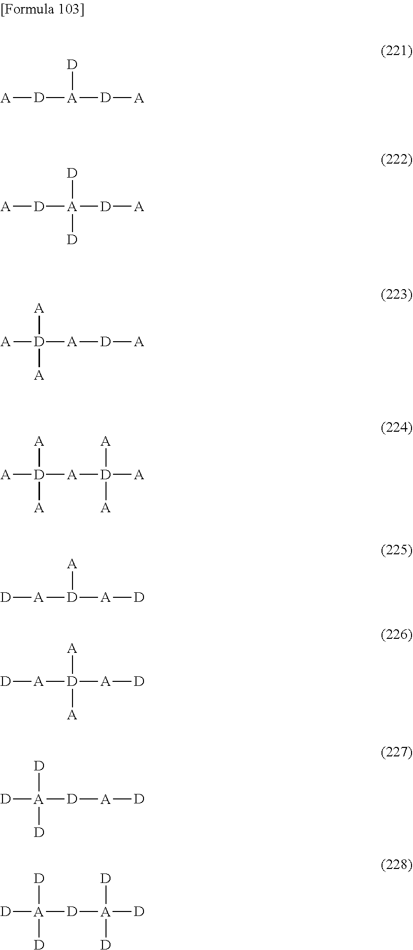

The second compound in the present exemplary embodiment is not limited to the compounds represented by the formulae (201) to (228). It should be noted that omission of L in the formulae (201) to (228) means that L is a single bond between A and D or L is a hydrogen atom at a terminal end of the molecule of the second compound.

In order to keep ΔST of the molecule at a small level, it is preferable that L is not a fused aromatic ring in terms of molecule design. However, a fused aromatic ring may be used as long as thermally activated delayed fluorescence can be obtained. Further, since the molecule has to be designed so that A and D are accurately disposed in a single molecule, the second compound of the present exemplary embodiment is preferably a low molecular material. Accordingly, it is preferable that the molecular weight of the second compound of the present exemplary embodiment is 5000 or less, more preferably 3000 or less. The second compound in the present exemplary embodiment preferably includes the moiety represented by the formula (200) and the moiety represented by the formula (2Y).

The organic EL device containing the second compound emits light through a thermally activated delayed fluorescence mechanism.

In the present exemplary embodiment, the formula (2Y) is preferably represented by at least one of a formula (2a) and a formula (2x) below.

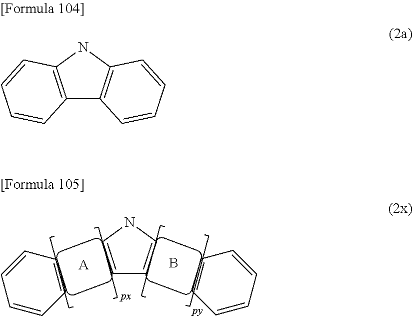

In the formula (2x), A and B each independently represent a cyclic structure represented by a formula (2c) below or a cyclic structure represented by a formula (2d) below, the cyclic structures A and B being fused to adjacent cyclic structure(s) at any position(s). px and py are each independently an integer ranging from 0 to 4, px and py representing the number of the cyclic structure A and the number of the cyclic structure B, respectively. When px is an integer ranging from 2 to 4, a plurality of the cyclic structures A may be mutually the same or different. When py is an integer ranging from 2 to 4, a plurality of the cyclic structures B may be mutually the same or different. Accordingly, when, for instance, px is 2, the cyclic structures A may be two cyclic structures each represented by a formula (2c), two cyclic structures each represented by a formula (2d), or a combination of one cyclic structure represented by the formula (2c) and one cyclic structure represented by the formula (2d).

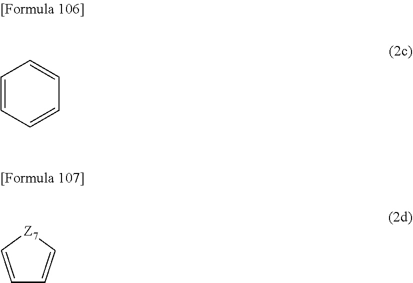

In the formula (2d), Z₇ represents a carbon atom, a nitrogen atom, a sulfur atom, or an oxygen atom.

When px is 0 and the number of py is c in the formula (2x), the formula (2x) is represented by a formula (2b) below.

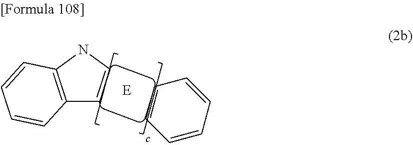

In the formula (2b), c is an integer ranging from 1 to 4. When c is an integer ranging from 2 to 4, a plurality of the cyclic structures E may be mutually the same or different. In the formula (2b), E represents the cyclic structure represented by the formula (2c) or the cyclic structure represented by the formula (2d), the cyclic structure E being fused to adjacent cyclic structure(s) at any position(s). Accordingly, when, for instance, c is 2, the two cyclic structures E may be two cyclic structures each represented by the formula (2c), two cyclic structures each represented by the formula (2d), or a combination of one cyclic structure represented by the formula (2c) and one cyclic structure represented by the formula (2d).

The presence of the moieties represented by the formula (200) and the formula (2Y) in a single molecule allows a design capable of effectively reducing ΔST.

The second compound in the present exemplary embodiment preferably includes a moiety represented by a formula (2e) below in a molecule.

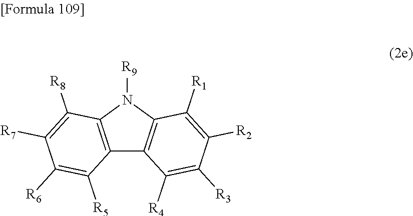

R₁ to R₉ in the formula (2e) each independently represent a hydrogen atom, a substituent, or a single bond to another atom in the molecule of the second compound.

The substituent for R₁ to R₉ is a substituent selected the group consisting of a halogen atom, a substituted or unsubstituted aryl group having 6 to 30 ring carbon atoms, a substituted or unsubstituted aromatic heterocyclic group having 5 to 30 ring atoms, a substituted or unsubstituted alkyl group having 1 to 30 carbon atoms, a substituted or unsubstituted alkylsilyl group having 3 to 30 carbon atoms, a substituted or unsubstituted arylsilyl group having 6 to 60 ring carbon atoms, a substituted or unsubstituted alkoxy group having 1 to 30 carbon atoms, a substituted or unsubstituted aryloxy group having 6 to 30 ring carbon atoms, a substituted or unsubstituted alkylamino group having 2 to 30 carbon atoms, a substituted or unsubstituted arylamino group having 6 to 60 ring carbon atoms, a substituted or unsubstituted alkylthio group having 1 to 30 carbon atoms, and a substituted or unsubstituted arylthio group having 6 to 30 ring carbon atoms. It should be noted that at least one of R₁ to R₉ is a single bond to another atom in the molecule of the second compound.

In the formula (2e), at least one pair of the substituents selected from R₁ to R₉ are optionally bonded to form a cyclic structure. When the cyclic structure is formed, among substituents R₁ to R₉ bonded to the carbon atoms of the six-membered ring or the nitrogen atom of the five-membered ring in the formula (2e), a pair of substituents selected from R₁ to R₈ each bonded to adjacent carbon atoms of the six-membered ring and R₉ of the five-membered ring form the cyclic structure. Specifically, at least one of pairs of substituents consisting of R₁ and R₂, R₂ and R₃, R₃ and R₄, R₄ and R₅, R₅ and R₆, R₆ and R₇, R₇ and R₈, R₈ and R₉, and R₁ and R₉ forms a mutual bonding to form a cyclic structure.

In the present exemplary embodiment, the cyclic structure formed by the bonding of the substituents is preferably a fused ring. For instance, the cyclic structure formable in the formula (2e) is a fused six-membered cyclic structure.

The second compound in the present exemplary embodiment preferably includes a moiety represented by a formula (2y) below in a molecule.

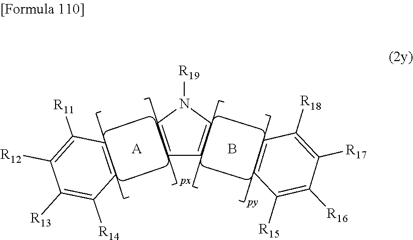

In the formula (2y), R₁₁ to R₁₉ each independently represent the same as R₁ to R₉ of the formula (2e). It should be noted that at least one of R₁₁ to R₁₉ is a single bond to another atom in the molecule of the second compound. In the formula (2y), at least one pair of the substituents selected from R₁₁ to R₁₉ are optionally bonded to form a cyclic structure. In the formula (2y), A and B each independently represent a cyclic structure represented by a formula (2g) below or a cyclic structure represented by a formula (2h) below, the cyclic structures A and B being fused to adjacent cyclic structure(s) at any position(s). px represents the number of the cyclic structure A and is an integer ranging from 0 to 4. When px is an integer ranging from 2 to 4, a plurality of the cyclic structures A may be mutually the same or different. When py is an integer ranging from 2 to 4, a plurality of the cyclic structures B may be mutually the same or different. py represents the number of the cyclic structure B and is an integer ranging from 0 to 4. Accordingly, when, for instance, px is 2, the two cyclic structures A may be two cyclic structures each represented by the formula (2g) below, two cyclic structures each represented by the formula (2h) below, or a combination of one cyclic structure represented by the formula (2g) and one cyclic structure represented by the formula (2h).

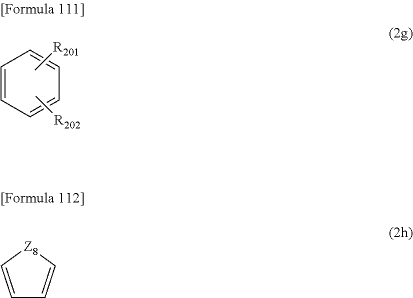

In the formula (2g), R₂₀₁ and R₂₀₂ each independently are selected from the same for the above R₁ to R₉, R₂₀₁ and R₂₀₂ being optionally mutually bonded to form a cyclic structure. R₂₀₁ and R₂₀₂ are each bonded to corresponding one of carbon atoms of the six-membered ring of the formula (2g).

In the formula (2h), Z₈ represents CR₂₀₃R₂₀₄, NR₂₀₅, a sulfur atom, or an oxygen atom, R₂₀₂ to R₂₀₅ each independently selected from the same group for the substituent for R₁ to R₉.

In the formula (2y), at least one pair of the substituents selected from R₁₁ to R₁₉ and R₂₀₁ to R₂₀₅ are optionally bonded to form a cyclic structure.

When px is 0 and the number of py is c in the formula (2y), the formula (2y) is represented by a formula (2f) below.

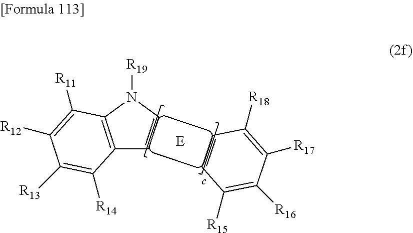

In the formula (2f), R₁₁ to R₁₉ each independently represent the same as R₁ to R₉ of the formula (2e). It should be noted that at least one of R₁₁ to R₁₉ is a single bond to another atom in the molecule of the second compound. In the formula (2f), at least one pair of the substituents selected from R₁₁ to R₁₉ are optionally bonded to form a cyclic structure. In the formula (2f), E represents the cyclic structure represented by the formula (2g) or the cyclic structure represented by the formula (2h), the cyclic structure E being fused to adjacent cyclic structure(s) at any position(s). c represents the number of the cyclic structure E and is an integer ranging from 1 to 4. When c is an integer ranging from 2 to 4, a plurality of the cyclic structures E may be mutually the same or different. Accordingly, when, for instance, c is 2, the two cyclic structures E may be two cyclic structures each represented by the formula (2g) below, two cyclic structures each represented by the formula (2h) below, or a combination of one cyclic structure represented by the formula (2g) and one cyclic structure represented by the formula (2h).

The second compound of the present exemplary embodiment is preferably represented by a formula (2A) below.

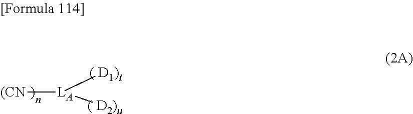

In the formula (2A), n is an integer of 1 or more, t is an integer of 1 or more and u is an integer of 0 or more. L_{A} is a substituted or unsubstituted aromatic hydrocarbon ring having 6 to 30 ring carbon atoms, or a substituted or unsubstituted aromatic heterocyclic ring having 6 to 30 ring atoms. CN is a cyano group. D₁ and D₂ are each independently represented by the formula (2Y), where the cyclic structures F and G are unsubstituted or substituted, m is 0 or 1, and, when m is 1, Y₂₀ represents a single bond, an oxygen atom, a sulfur atom, a selenium atom, a carbonyl group, CR₂₁R₂₂, SiR₂₃R₂₄ or GeR₂₅R₂₆, R₂₁ to R₂₆ being selected from the same group for R. When m is 1 in the formula (2Y), the formula (2Y) is represented by any one of the formulae (22) to (25) and the formulae (21Y) to (24Y). D₁ and D₂ may be the same or different. When t is 2 or more, a plurality of D₁ may be mutually the same or different. When u is 2 or more, a plurality of D₂ may be mutually the same or different.

In the present exemplary embodiment, it is preferable that L_{A} is a substituted or unsubstituted aromatic hydrocarbon ring having 6 to 14 ring carbon atoms. Examples of the aromatic hydrocarbon ring having 6 to 14 ring carbon atoms include benzene, naphthalene, fluorene, and phenanthrene. L_{A} is further preferably an aromatic hydrocarbon ring having 6 to 10 ring carbon atoms.

Examples of the aromatic heterocyclic ring having 6 to 30 ring atoms for L_{A} include pyridine, pyrimidine, pyrazine, quinoline, quinazoline, phenanthroline, benzofuran, and dibenzofuran.

In the present exemplary embodiment, D₁ or D₂ is optionally bonded to a first carbon atom of the aromatic hydrocarbon ring represented by L_{A} in the formula (2A), and CN is optionally bonded to a second carbon atom adjacent to the first carbon atom. For instance, in the second compound according to the present exemplary embodiment, D may be bonded to a first carbon atom C₁ and a cyano group may be bonded to a second carbon atom C₂ adjacent to the first carbon atom C₁ as in a moiety represented by a formula (2B) below. D in the formula (2B) below is the same as the above D₁ or D₂. Wavy lines in the formula (2B) below each represent a bonding position to another structure or atom.

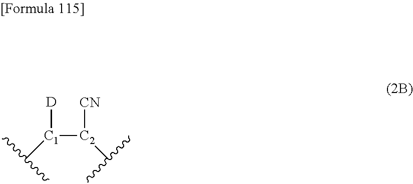

D₁ or D₂ having the structure represented by the formula (2a) or (2b) and a cyano group, which are adjacently bonded to the aromatic hydrocarbon ring represented by L_{A}, reduces the value of ΔST of the compound.

In the present exemplary embodiment, t is preferably an integer of 2 or more. When two or more D₁ are bonded to the aromatic hydrocarbon ring represented by L_{A}, the structures of the plurality of D₁ may be the same or different.

The second compound of the present exemplary embodiment is preferably represented by a formula (21) below.

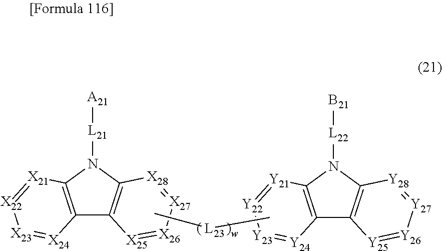

In the formula (21), A₂₁ and B₂₁ each independently represent a substituted or unsubstituted aromatic hydrocarbon group having 6 to 30 ring carbon atoms, or a substituted or unsubstituted aromatic heterocyclic group having 5 to 30 ring atoms.

X₂₁ to X₂₈ and Y₂₁ to Y₂₈ each independently represent a nitrogen atom, a carbon atom bonded to R^{D}, or a carbon atom bonded to L₂₃, where at least one of X₂₅ to X₂₈ is a carbon atom bonded to L₂₃, and at least one of Y₂₁ to Y₂₄ is a carbon atom bonded to L₂₃.

R^{D} each independently represent a hydrogen atom or a substituent. The substituent for R^{D} is selected from the group consisting of a halogen atom, a substituted or unsubstituted aromatic hydrocarbon group having 6 to 30 ring carbon atoms, a substituted or unsubstituted aromatic heterocyclic group having 5 to 30 ring atoms, a substituted or unsubstituted alkyl group having 1 to 30 carbon atoms and a substituted or unsubstituted silyl group.

L₂₁ and L₂₂ are each independently a single bond or a linking group, the linking group for L₂₁ and L₂₂ being a substituted or unsubstituted aromatic hydrocarbon group having 6 to 30 ring carbon atoms, a substituted or unsubstituted heterocyclic group having 5 to 30 ring atoms, a multiple linking group provided by bonding two to four groups selected from the aromatic hydrocarbon group, a multiple linking group provided by bonding two to four groups selected from the heterocyclic group, or a multiple linking group provided by bonding two to four groups selected from the aromatic hydrocarbon group and the heterocyclic group.

L₂₃ is a substituted or unsubstituted monocyclic hydrocarbon group having 6 or less ring carbon atoms, or a substituted or unsubstituted monocyclic heterocyclic group having 6 or less ring atoms.

w represents an integer ranging from 0 to 3. When w is 0, at least one of X₂₅ to X₂₈ and at least one of Y₂₁ to Y₂₄ are directly bonded.

It should be noted that the monocyclic hydrocarbon group is not a fused ring but is a group derived from a single hydrocarbon ring (alicyclic hydrocarbon or aromatic hydrocarbon) and the monocyclic heterocyclic group is a group derived from a single heterocycle.

It should be noted that the formula (21) satisfies at least one of conditions (i) and (ii) below.

(i) At least one of A₂₁ and B₂₁ is a cyano-group-substituted aromatic hydrocarbon group having 6 to 30 ring carbon atoms, or a cyano-group-substituted aromatic heterocyclic group having 6 to 30 ring atoms.
(ii) At least one of X₂₁ to X₂₄ and Y₂₅ to Y₂₈ is a carbon atom bonded to R^{D}, and at least one of R^{D} is a cyano-group-substituted aromatic hydrocarbon group having 6 to 30 ring carbon atoms, or a cyano-group-substituted aromatic heterocyclic group having 6 to 30 ring atoms.

When a plurality of R^{D} are present, the plurality of R^{D} may be the same or different.

In the formula (21), when the aromatic hydrocarbon group having 6 to 30 ring carbon atoms or the aromatic heterocyclic group having 6 to 30 ring atoms represented by A₂₁ and B₂₁ has a substituent, the substituent is preferably one or more group selected from the group consisting of a cyano group, a halogen atom, an alkyl group having 1 to 20 carbon atoms, a cycloalkyl group having 3 to 20 carbon atoms, an alkoxy group having 1 to 20 carbon atoms, a haloalkyl group having 1 to 20 carbon atoms, a haloalkoxy group having 1 to 20 carbon atoms, an alkylsilyl group having 1 to 10 carbon atoms, an aryl group having 6 to 30 ring carbon atoms, an aryloxy group having 6 to 30 ring carbon atoms, an aralkyl group having 6 to 30 carbon atoms, and a heterocyclic group having 5 to 30 ring atoms. When A₂₁ and B₂₁ have a plurality of substituents, the substituents may be the same or different.

It is preferable that the formula (21) satisfies the above condition (i) and does not satisfy the above condition (ii).

Alternatively, it is preferable that the formula (21) satisfies the above condition (ii) and does not satisfy the above condition (i).

Further alternatively, it is preferable that the formula (21) satisfies the above conditions (i) and (ii).

In the formula (21), at least one of A₂₁ and B₂₁ is preferably a phenyl group substituted by a cyano group, a naphthyl group substituted by a cyano group, a phenanthryl group substituted by a cyano group, a dibenzofuranyl group substituted by a cyano group, a dibenzothiophenyl group substituted by a cyano group, a biphenyl group substituted by a cyano group, a terphenyl group substituted by a cyano group, a 9,9-diphenylfluorenyl group substituted by a cyano group, a 9,9′-spirobi[9H-fluorene]-2-yl group substituted by a cyano group, a 9,9-dimethylfluorenyl group substituted by a cyano group, or a triphenylenyl group substituted by a cyano group.

In the formula (21), it is preferable that at least one of X₂₁ to X₂₄ and Y₂₅ to Y₂₈ is CR^{D}, and at least one of R^{D} in X₂₁ to X₂₄ and Y₂₅ to Y₂₈ is a phenyl group substituted by a cyano group, a naphthyl group substituted by a cyano group, a phenanthryl group substituted by a cyano group, a dibenzofuranyl group substituted by a cyano group, a dibenzothiophenyl group substituted by a cyano group, a biphenyl group substituted by a cyano group, a terphenyl group substituted by a cyano group, a 9,9-diphenylfluorenyl group substituted by a cyano group, a 9,9′-spirobi[9H-fluorene]-2-yl group substituted by a cyano group, a 9,9-dimethylfluorenyl group substituted by a cyano group, or a triphenylenyl group substituted by a cyano group.

In the formula (21), it is preferable that X₂₆ and Y₂₃ are bonded via L₂₃, or directly bonded.

In the formula (21), it is also preferable that X₂₆ and Y₂₂ are bonded via L₂₃, or directly bonded.

In the formula (21), it is also preferable that X₂₇ and Y₂₃ are bonded via L₂₃, or directly bonded.

In the formula (21), w is preferably 0.

Alternatively, in the formula (21), w is preferably 1.

In the formula (21), L₂₁ and L₂₂ are each preferably a single bond or a substituted or unsubstituted aromatic hydrocarbon group having 6 to 30 ring carbon atoms.

Specific examples of the second compound are shown below. It should be noted that the second compound according to the invention is not limited to these specific examples.

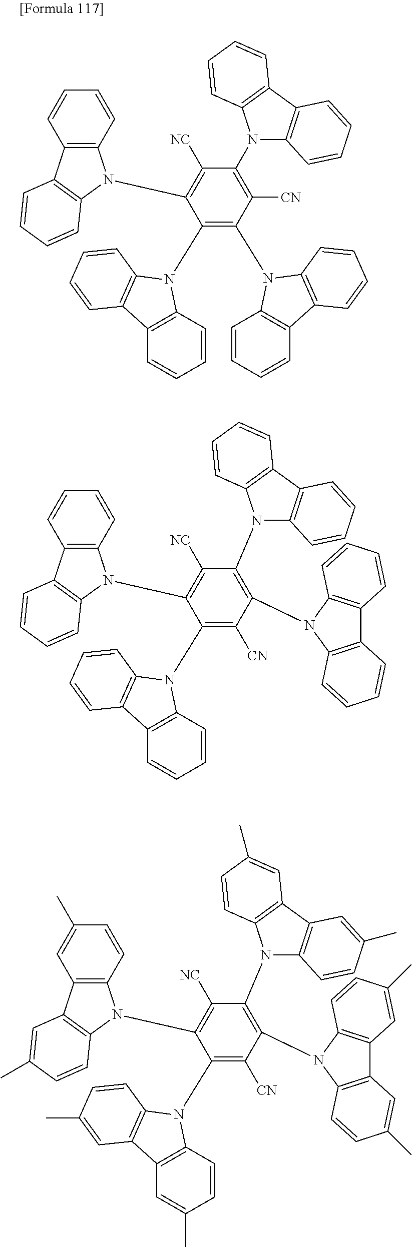

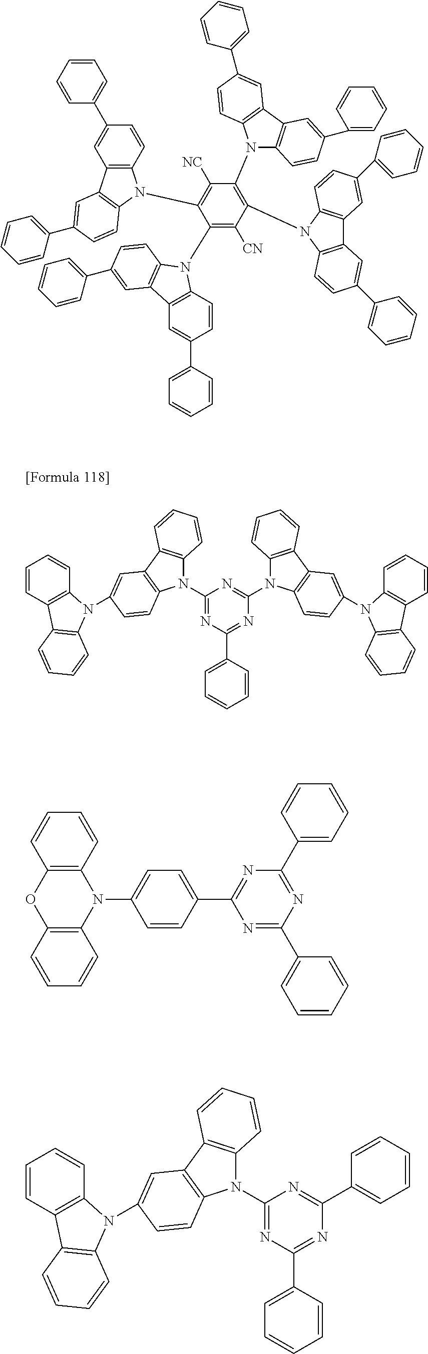 [SMILES: N#Cc1c(-n2c3ccc(-c4ccccc4)cc3c3cc(-c4ccccc4)ccc32)c(-n2c3ccc(-c4ccccc4)cc3c3cc(-c4ccccc4)ccc32)c(C#N)c(-n2c3ccc(-c4ccccc4)cc3c3cc(-c4ccccc4)ccc32)c1-n1c2ccc(-c3ccccc3)cc2c2cc(-c3ccccc3)ccc21 ; c1ccc(-c2nc(-n3c4ccccc4c4cc(-n5c6ccccc6c6ccccc65)ccc43)nc(-n3c4ccccc4c4cc(-n5c6ccccc6c6ccccc65)ccc43)n2)cc1 ; c1ccc(-c2nc(-c3ccccc3)nc(-c3ccc(N4c5ccccc5Oc5ccccc54)cc3)n2)cc1 ; c1ccc(-c2nc(-c3ccccc3)nc(-n3c4ccccc4c4cc(-n5c6ccccc6c6ccccc65)ccc43)n2)cc1]

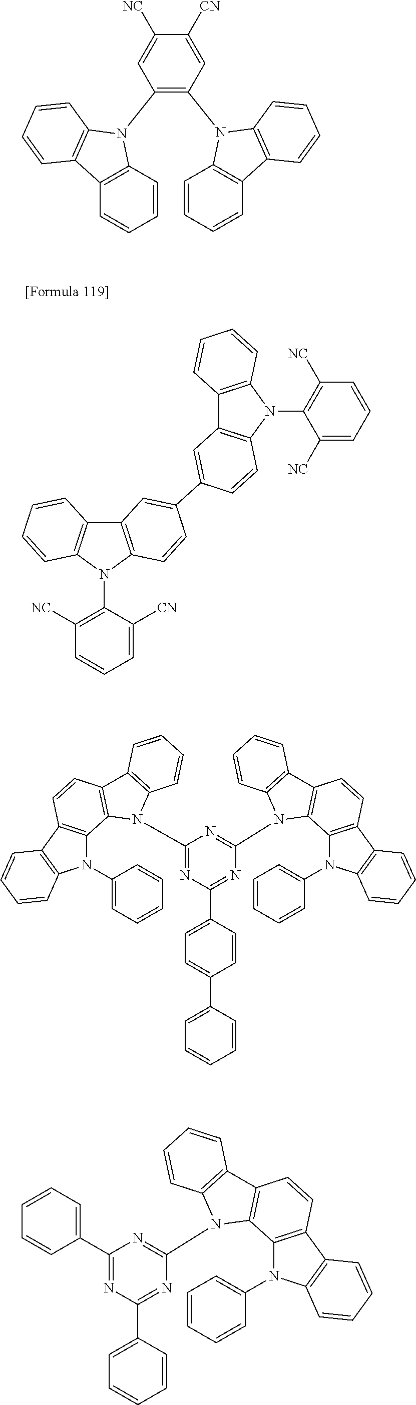 [SMILES: N#Cc1cc(-n2c3ccccc3c3ccccc32)c(-n2c3ccccc3c3ccccc32)cc1C#N ; N#Cc1cccc(C#N)c1-n1c2ccccc2c2cc(-c3ccc4c(c3)c3ccccc3n4-c3c(C#N)cccc3C#N)ccc21 ; c1ccc(-c2ccc(-c3nc(-n4c5ccccc5c5ccc6c7ccccc7n(-c7ccccc7)c6c54)nc(-n4c5ccccc5c5ccc6c7ccccc7n(-c7ccccc7)c6c54)n3)cc2)cc1 ; c1ccc(-c2nc(-c3ccccc3)nc(-n3c4ccccc4c4ccc5c6ccccc6n(-c6ccccc6)c5c43)n2)cc1]

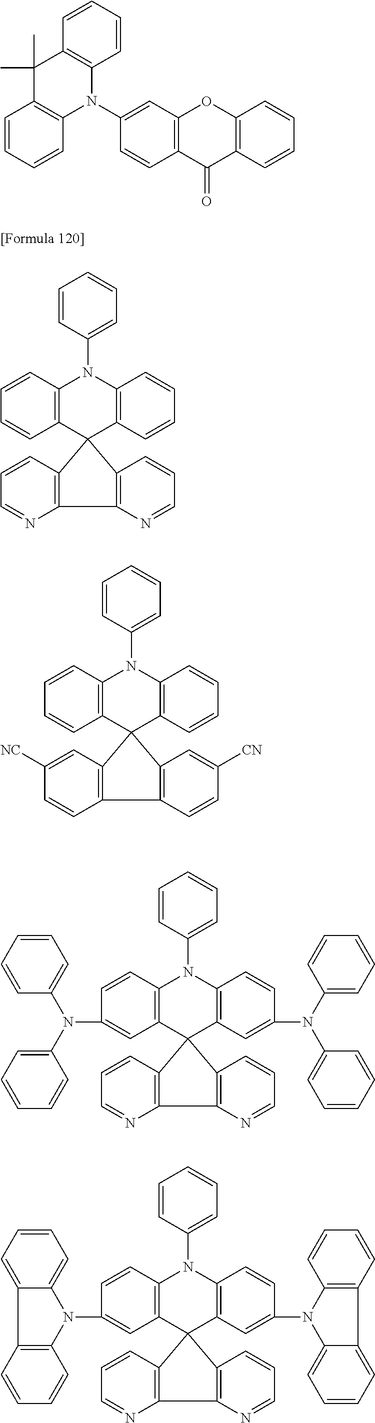

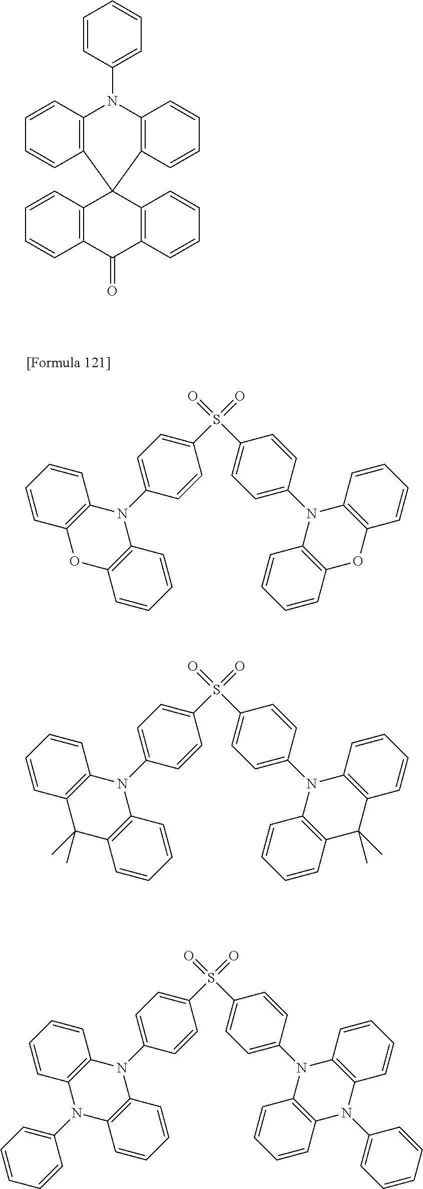 [SMILES: O=C1c2ccccc2C2(c3ccccc31)c1ccccc1N(c1ccccc1)c1ccccc12 ; O=S(=O)(c1ccc(N2c3ccccc3Oc3ccccc32)cc1)c1ccc(N2c3ccccc3Oc3ccccc32)cc1 ; CC1(C)c2ccccc2N(c2ccc(S(=O)(=O)c3ccc(N4c5ccccc5C(C)(C)c5ccccc54)cc3)cc2)c2ccccc21 ; O=S(=O)(c1ccc(N2c3ccccc3N(c3ccccc3)c3ccccc32)cc1)c1ccc(N2c3ccccc3N(c3ccccc3)c3ccccc32)cc1]

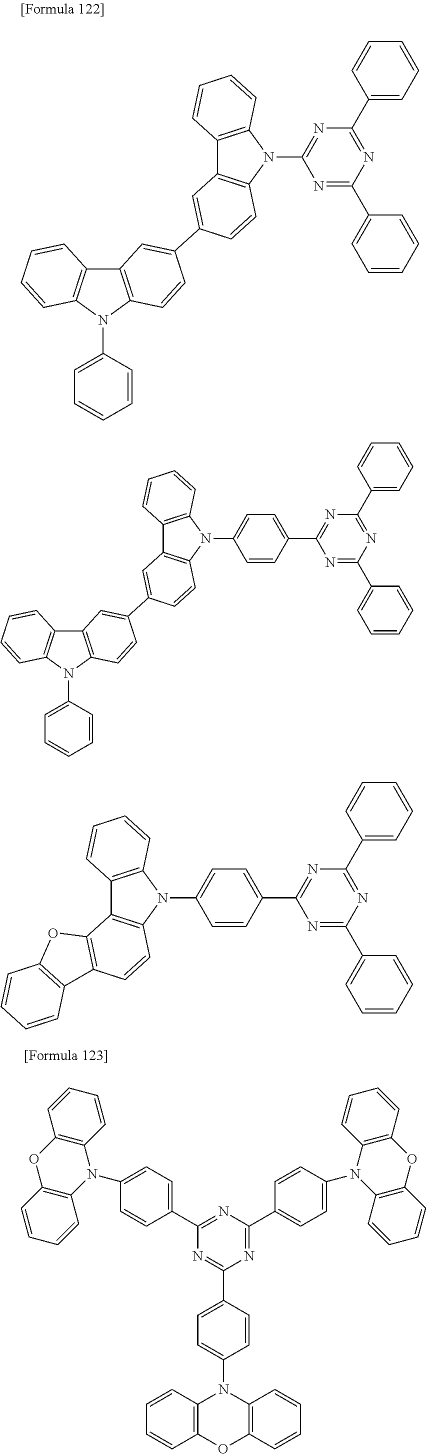 [SMILES: c1ccc(-c2nc(-c3ccccc3)nc(-n3c4ccccc4c4cc(-c5ccc6c(c5)c5ccccc5n6-c5ccccc5)ccc43)n2)cc1 ; c1ccc(-c2nc(-c3ccccc3)nc(-c3ccc(-n4c5ccccc5c5cc(-c6ccc7c(c6)c6ccccc6n7-c6ccccc6)ccc54)cc3)n2)cc1 ; c1ccc(-c2nc(-c3ccccc3)nc(-c3ccc(-n4c5ccccc5c5c6oc7ccccc7c6ccc54)cc3)n2)cc1 ; c1ccc2c(c1)Oc1ccccc1N2c1ccc(-c2nc(-c3ccc(N4c5ccccc5Oc5ccccc54)cc3)nc(-c3ccc(N4c5ccccc5Oc5ccccc54)cc3)n2)cc1]

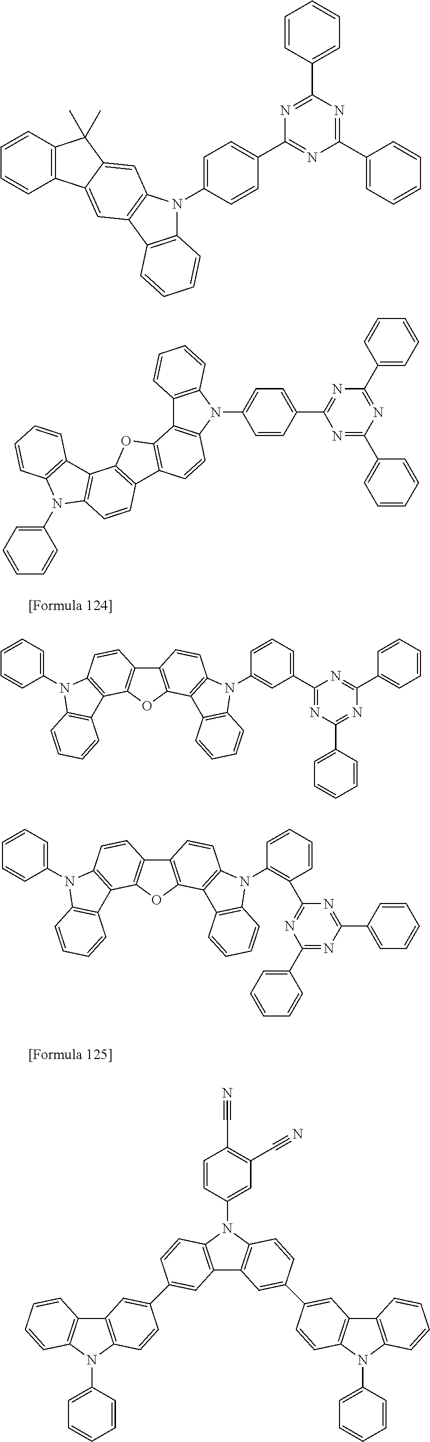 [SMILES: CC1(C)c2ccccc2-c2cc3c4ccccc4n(-c4ccc(-c5nc(-c6ccccc6)nc(-c6ccccc6)n5)cc4)c3cc21 ; c1ccc(-c2nc(-c3ccccc3)nc(-c3ccc(-n4c5ccccc5c5c6oc7c(ccc8c7c7ccccc7n8-c7ccccc7)c6ccc54)cc3)n2)cc1 ; c1ccc(-c2nc(-c3ccccc3)nc(-c3cccc(-n4c5ccccc5c5c6oc7c(ccc8c7c7ccccc7n8-c7ccccc7)c6ccc54)c3)n2)cc1 ; c1ccc(-c2nc(-c3ccccc3)nc(-c3ccccc3-n3c4ccccc4c4c5oc6c(ccc7c6c6ccccc6n7-c6ccccc6)c5ccc43)n2)cc1 ; N#Cc1ccc(-n2c3ccc(-c4ccc5c(c4)c4ccccc4n5-c4ccccc4)cc3c3cc(-c4ccc5c(c4)c4ccccc4n5-c4ccccc4)ccc32)cc1C#N]

### Preparation Method of Second Compound

The second compound can be prepared by methods disclosed in, for instance, International Publication Nos. WO2013/180241, WO2014/092083, WO2014/104346 and the like.

### Delayed Fluorescence

Delayed fluorescence (thermally activated delayed fluorescence) is described in “Yuki Hando-tai no Debaisu Bussei (Device Physics of Organic Semiconductor)” edited by Chihaya Adachi, published by Kodansha Company Ltd, pages 261 to 268. This document describes that, when an energy gap ΔE₁₃ between a singlet state and a triplet state of a fluorescent material can be decreased, in spite of a typical low transition probability, inverse energy transfer from the triplet state to the singlet state occurs at a high efficiency to express TADF (Thermally Activated Delayed Fluorescence). Further, a generating mechanism of delayed fluorescence is described in FIG. 10.38 in this document. The second compound in the present exemplary embodiment is a compound emitting thermally activated delayed fluorescence to be generated by such a mechanism.

Delayed fluorescence can be observed by measuring transient PL (Photo Luminescence).

Behavior of delayed fluorescence can also be analyzed based on the decay curve obtained by measuring the transient PL. The transient PL measurement is a method for measuring reduction behavior (transitional property) of PL emission obtained after irradiating a sample with pulse laser to excite the sample and stopping irradiation by the pulse laser. PL emission in TADF materials is classified into a light emission component from a singlet exciton generated by the first PL excitation and a light emission component from a singlet exciton generated via a triplet exciton. Lifetime of the singlet excitons initially generated in the PL excitation is very short at a nano-second order. Accordingly, the emission from the singlet excitons is rapidly reduced after pulse laser radiation.

In contrast, since delayed fluorescence provides emission from singlet excitons generated through long-life triplet excitons, emission is gradually reduced. As described above, there is a large temporal difference between the light emission from the singlet exciton generated by the first PL excitation and the light emission from the singlet exciton generated via the triplet exciton. Accordingly, a luminous intensity derived from delayed fluorescence is obtainable.

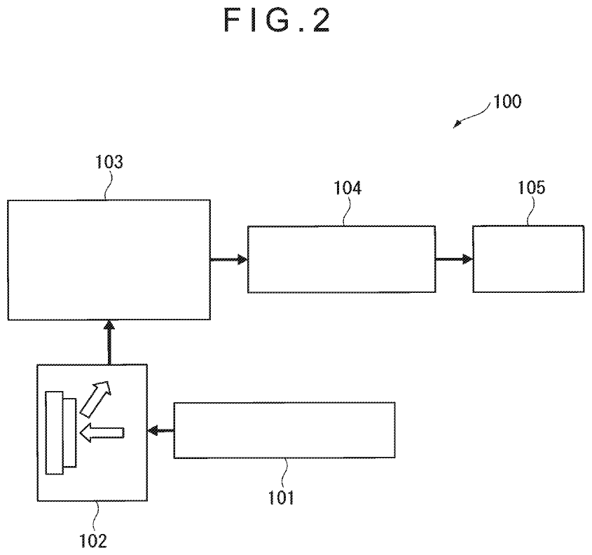

FIG. 2 is a schematic illustration of an exemplary device for measuring the transient PL.

A transient PL measuring device 100 in the present exemplary embodiment includes: a pulse laser 101 capable of radiating a light having a predetermined wavelength; a sample chamber 102 configured to house a measurement sample; a spectrometer 103 configured to divide a light radiated from the measurement sample; a streak camera 104 configured to provide a two-dimensional image; and a personal computer 105 configured to import and analyze the two-dimensional image. A device usable for the measurement of the transient PL is not limited to the device described in the present exemplary embodiment.

The sample housed in the sample chamber 102 is obtained by forming a thin film, in which a matrix material is doped with a doping material at a concentration of 12 mass %, on the quartz substrate.

The thin film sample housed in the sample chamber 102 is irradiated with the pulse laser from the pulse laser 101 to excite the doping material. Emission is extracted at 90 degrees angle relative to a radiation direction of the excited light. The extracted emission is dispersed with the spectrometer 103 to form a two-dimensional image in the streak camera 104. As a result, the two-dimensional image expressed in coordinates whose ordinate axis and abscissa axis indicate time and and a wavelength, respectively, in which a luminous point indicates a luminous intensity, can be obtained. If the two-dimensional image is cut out along a predetermined time axis, emission spectrum expressed in coordinates whose ordinate axis and abscissa axis indicate a luminous intensity and the wavelength, respectively, can be obtained. If the two-dimensional image is cut out along a wavelength axis, a decay curve (transient PL) expressed in coordinates whose ordinate axis and abscissa axis indicate a logarithm of the luminous intensity and time, respectively, can be obtained.

For instance, a thin film sample A was prepared as described above from a reference compound H1 as the matrix material and a reference compound D1 as the doping material and was measured in terms of the transient PL.

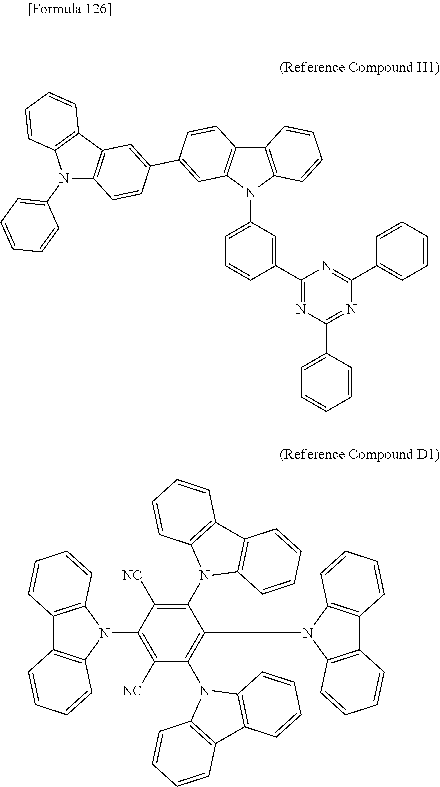 [SMILES: c1ccc(-c2nc(-c3ccccc3)nc(-c3cccc(-n4c5ccccc5c5ccc(-c6ccc7c(c6)c6ccccc6n7-c6ccccc6)cc54)c3)n2)cc1 ; N#Cc1c(-n2c3ccccc3c3ccccc32)c(C#N)c(-n2c3ccccc3c3ccccc32)c(-n2c3ccccc3c3ccccc32)c1-n1c2ccccc2c2ccccc21]

Herein, the decay curve was analyzed using the above-described thin film sample A and a thin film sample B. The thin film sample B was manufactured in the same manner as described above from a reference compound H2 as the matrix material and the reference compound D1 as the doping material.

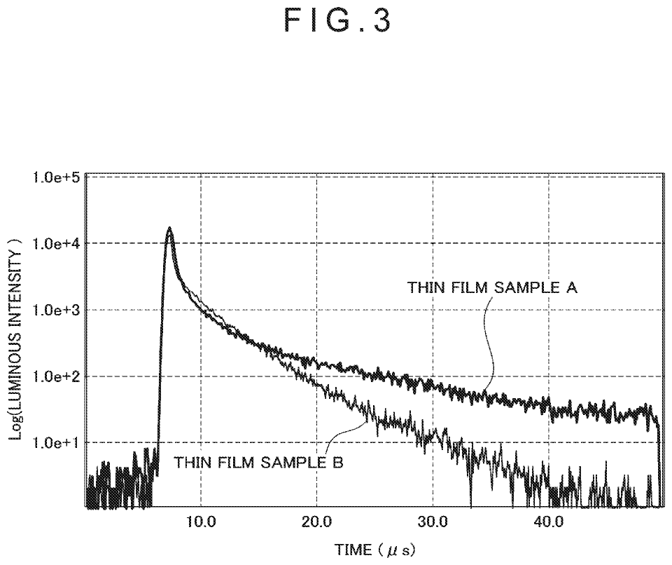

FIG. 3 shows a decay curve obtained from the measured transitional PL of the thin film sample A and the thin film sample B.

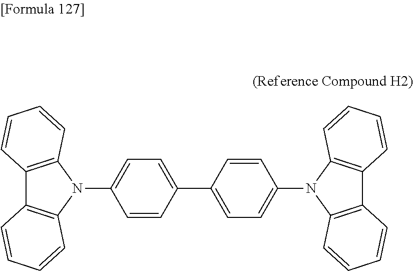 [SMILES: c1ccc2c(c1)c1ccccc1n2-c1ccc(-c2ccc(-n3c4ccccc4c4ccccc43)cc2)cc1]

An emission decay curve expressed in coordinates whose ordinate axis and abscissa axis indicate a luminous intensity and time, respectively, can be obtained by measuring the transient PL as described above. Based on the emission decay curve, a fluorescence intensity ratio between fluorescence emitted from a singlet state generated by photo-excitation and delayed fluorescence emitted from a singlet state generated by inverse energy transfer via a triplet state can be estimated. In a delayed fluorescent material, a ratio of the intensity of the slowly decaying delayed fluorescence to the intensity of the promptly decaying fluorescence is relatively large.

In the present exemplary embodiment, an amount of the delayed fluorescence can be calculated using the device of FIG. 2. In the first compound after excited with pulse light (light emitted from the pulse laser) having a wavelength to be absorbed in the second compound, Prompt Emission that is immediately observed in the excited state and Delay Emission that is not observed immediately after the excitation but is later observed are present. In the present exemplary embodiment, an amount of Delay Emission is preferably 5% or more with respect to an amount of Prompt Emission.

The amount of Prompt Emission and the amount of Delay Emission can be obtained according to the same method as a method described in “Nature 492, 234-238, 2012.” The amount of Prompt emission and the amount of Delay emission may be calculated using a device different from one described in the above Reference Literature.

Moreover, a sample usable for measuring delayed fluorescence is obtained, for instance, by co-depositing the first compound and a compound TH-2 on a quartz substrate at a ratio of the first compound of 12 mass % to form a 100-nm-thick thin film.

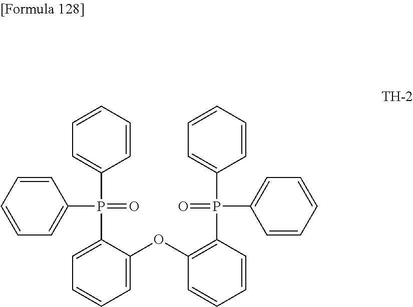 [SMILES: O=P(c1ccccc1)(c1ccccc1)c1ccccc1Oc1ccccc1P(=O)(c1ccccc1)c1ccccc1]

### TADF Mechanism

In the organic EL device of the present exemplary embodiment, the second compound is preferably a compound having a small ΔST(Mat1) so that inverse intersystem crossing from the triplet energy level of the second compound to the singlet energy level thereof is easily caused by a heat energy given from the outside. An energy state conversion mechanism to perform spin exchange from the triplet state of electrically excited excitons within the organic EL device to the singlet state by inverse intersystem crossing is referred to as the TADF Mechanism.

### Relationship Between First Compound and Second Compound in Emitting Layer

In the present exemplary embodiment, the singlet energy S₁(Mat1) of the first compound and the singlet energy S₁(Mat2) of the second compound preferably satisfy a relationship of a numerical formula (Numerical Formula 1) below.

S₁(Mat2)>S₁(Mat1) (Numerical Formula 1).

An energy gap T_{77K}(Mat2) at 77 [K] of the second compound is preferably larger than an energy gap T_{77K}(Mat1) at 77 [K] of the first compound.

### Relationship Between Triplet Energy and Energy Gap at 77[K]

Description will be made on a relationship between a triplet energy and an energy gap at 77[K]. In the present exemplary embodiment, the energy gap at 77 [K] is different from a typical triplet energy in some aspects.

The triplet energy is measured as follows. Firstly, a solution in which a compound (measurement target) is dissolved in an appropriate solvent is encapsulated in a quartz glass tube to prepare a sample. A phosphorescent spectrum (ordinate axis: phosphorescent luminous intensity, abscissa axis: wavelength) of each of the samples was measured at a low temperature (77[K]). A tangent was drawn to the rise of the phosphorescent spectrum on the short-wavelength side. Triplet energy was calculated according to a predetermined conversion equation based on a wavelength value at an intersection of the tangent and the abscissa axis.

Herein, the delayed fluorescent compound used in the present exemplary embodiment is preferably a compound having a small ΔST. When ΔST is small, intersystem crossing and inverse intersystem crossing are likely to occur even at a low temperature (77[K]), so that the singlet state and the triplet state coexist. As a result, the spectrum to be measured in the same manner as the above includes emission from both the singlet state and the triplet state. Although it is difficult to distinguish the emission from the singlet state from the emission from the triplet state, the value of the triplet energy is basically considered dominant.

Accordingly, in the present exemplary embodiment, a value measured in the following manner, which is measured by the same method as a typical triplet energy T but is different from the triplet energy T in a strict meaning, is referred to as an energy gap T_{77K} in order to differentiate the measured energy from the typical triplet energy. The compound to be measured is dissolved in EPA (diethylether:isopentane:ethanol=5:5:2 in volume ratio) at a concentration of 10 μmol/L, and the resulting solution is set in a quartz cell to provide a measurement sample. A phosphorescent spectrum (ordinate axis: phosphorescent luminous intensity, abscissa axis: wavelength) of the sample is measured at a low temperature (77[K]). A tangent is drawn to the rise of the phosphorescent spectrum on the short-wavelength side. An energy amount is calculated as the energy gap T_{77K} at 77[K] according to a conversion equation (F1) below based on a wavelength value λ_{edge} (nm) at an intersection of the tangent and the abscissa axis.

T_{77K} [eV]=1239.85/λ_{edge} Conversion Equation (F1):

The tangent to the rise of the phosphorescence spectrum at the short-wavelength side is drawn as follows. While moving on a curve of the phosphorescence spectrum from the short-wavelength side to the maximum spectral value closest to the short-wavelength side among the maximum spectral values, a tangent is checked at each point on the curve toward the long-wavelength side of the phosphorescence spectrum. A gradient of the tangent increases along the rise of the curve (i.e., a value of the ordinate axis increases). A tangent drawn at a point of the maximum gradient (i.e., a tangent at an inflection point) is defined as the tangent to the rise of the phosphorescence spectrum close to the short-wavelength side.

The maximum with peak intensity being 15% or less of the maximum peak intensity of the spectrum is not included in the above-mentioned maximum closest to the short-wavelength side. The tangent drawn at a point of the maximum spectral value being closest to the short-wavelength side and having the maximum gradient is defined as a tangent to the rise of the phosphorescence spectrum close to the short-wavelength side.

For phosphorescence measurement, a spectrophotofluorometer body F-4500 (manufactured by Hitachi High-Technologies Corporation) is usable. Any device for phosphorescence measurement is usable. A combination of a cooling unit, a low temperature container, an excitation light source and a light-receiving unit may be used for phosphorescence measurement.

### Singlet Energy S₁

A method of measuring a singlet energy S₁ with use of a solution (occasionally referred to as a solution method) is exemplified by a method below.

A 10 μmol/L toluene solution of a measurement target compound is prepared and put in a quartz cell. An absorption spectrum (ordinate axis: absorption intensity, abscissa axis: wavelength) of the this sample is measured at a normal temperature (300K). A tangent is drawn to the fall of the absorption spectrum close to the long-wavelength side, and a wavelength value λedge (nm) at an intersection of the tangent and the abscissa axis is obtained. The wavelength value λedge (nm) is substituted in a conversion equation (F2) below to calculate a singlet energy.

S₁ [eV]=1239.85/λedge Conversion Equation (F2):

Any device for measuring absorption spectrum is usable. For instance, a spectrophotometer (U3310 manufactured by Hitachi, Ltd.) is usable.

The tangent to the fall of the absorption spectrum close to the long-wavelength side is drawn as follows. While moving on a curve of the absorption spectrum from the maximum spectral value closest to the long-wavelength side in a long-wavelength direction, a tangent at each point on the curve is checked. A gradient of the tangent is decreased and increased in a repeated manner as the curve falls (i.e., a value of the ordinate axis is decreased). A tangent drawn at a point of the minimum gradient closest to the long-wavelength side (except when absorbance is 0.1 or less) is defined as the tangent to the fall of the absorption spectrum close to the long-wavelength side.

The maximum absorbance of 0.2 or less is not included in the above-mentioned maximum absorbance close to the long-wavelength side.

In the present exemplary embodiment, a difference (S₁−T_{77K}) between the singlet energy S₁ and the energy gap T_{77K} at 77[K] is defined as ΔST.

In the present exemplary embodiment, a difference ΔST(Mat2) between the singlet energy S₁(Mat2) of the second compound and the energy gap T_{77K}(Mat2) at 77[K] of the second compound is preferably less than 0.3 eV, more preferably less than 0.2 eV, further preferably less than 0.1 eV. In other words, ΔST(Mat2) preferably satisfies a relationship of at least one of the following numerical formulae (Formulae 10 to 12).

ΔST(Mat2)=S₁(Mat2)−T_{77K}(Mat2)<0.3 eV (Numerical Formula 10)

ΔST(Mat2)=S₁(Mat2)−T_{77K}(Mat2)<0.2 eV (Numerical Formula 11)

ΔST(Mat2)=S₁(Mat2)−T_{77K}(Mat2)<0.1 eV (Numerical Formula 12)

### Third Compound

The third compound is a compound represented by a formula (3) below.

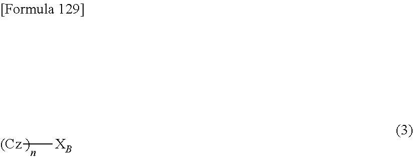

In the formula (3): n is 1, 2, 3 or 4;

when n is 2, 3, or 4, a plurality of Cz are mutually the same or different;

X_{B} is a group represented by a formula (3A) below; and

Cz is a group represented by a formula (3B-1) or a formula (3B-2) below.

[Formula 130]

Ar₁—(Ar₂)ₖ (3A)

In the formula (3A): Ar₁ and Ar₂ are each independently selected from the group consisting of a substituted or unsubstituted monovalent or polyvalent aromatic hydrocarbon group having 6 to 18 ring carbon atoms;

k is 0, 1, or 2;

when k is 2, a plurality of Ar₂ are mutually the same or different;

Ar₁ is optionally substituted by a substituent D1, the substituent D1 being each independently selected from the group consisting of a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted aryl group having 6 to 30 ring carbon atoms, a substituted or unsubstituted aralkyl group having 7 to 30 carbon atoms, a substituted or unsubstituted alkoxy group having 1 to 30 carbon atoms, a substituted or unsubstituted alkoxy halide group having 1 to 30 carbon atoms, a substituted or unsubstituted aryloxy group having 6 to 30 ring carbon atoms, a substituted or unsubstituted silyl group, a halogen atom, a cyano group, and a nitro group;

Ar₂ is optionally substituted by a substituent D2, the substituent D2 representing the same as the substituent D1;

when n is 1, Cz is bonded to Ar₁ or Ar₂; and

when n is 2, 3 or 4, a plurality of Cz are each independently bonded to Ar₁ or Ar₂.

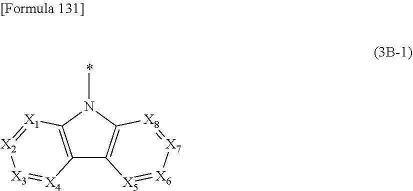

In the formula (3B-1): X₁ to X₈ each independently represent a nitrogen atom or CR_{A}, R_{A} being a hydrogen atom or a substituent, at least one pair of adjacent ones of R_{A} being optionally mutually bonded to form a ring;

R_{A} as the substituent is each independently selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted silyl group, a substituted or unsubstituted alkoxy group having 1 to 30 carbon atoms, a substituted or unsubstituted alkoxy halide group having 1 to 30 carbon atoms, a substituted or unsubstituted aryloxy group having 6 to 30 carbon atoms, a substituted or unsubstituted alkylthio group having 1 to 30 carbon atoms, a substituted or unsubstituted arylthio group having 6 to 30 ring carbon atoms, a substituted or unsubstituted aralkyl group having 7 to 30 carbon atoms, a substituted or unsubstituted alkenyl group having 2 to 30 carbon atoms, a halogen atom, a substituted or unsubstituted alkynyl group having 2 to 30 carbon atoms, a cyano group, a hydroxy group; a nitro group, and a carboxy group;

the substituted or unsubstituted heteroaryl group having 5 to 30 ring atoms for R_{A} is each independently a carbazolyl group or an azacarbazolyl group;

a plurality of R_{A} are mutually the same or different; and

the nitrogen atom in the formula (3B-1) is bonded to one of Ar₁ or Ar₂ in the formula (3A).

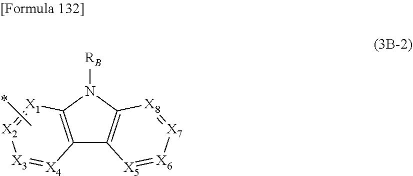

In the formula (3B-2): X₁ to X₄ are each a carbon atom bonded to one of Ar₁ and Ar₂ in the formula (3A), a nitrogen atom, or CR_{C}, R_{C} being a hydrogen atom or a substituent, at least one pair of adjacent ones of R_{C} being optionally mutually bonded to form a ring, at least one of X₁ to X₄ being a carbon atom bonded to one of Ar₁ and Ar₂ in the formula (3A);

X₅ to X₈ are each a nitrogen atom or CR_{D}, R_{D} being a hydrogen atom or a substituent, at least one pair of adjacent ones of R_{D} being optionally mutually bonded to forma ring;

R_{B}, R_{C} and R_{D} each independently represent the same as R_{A} in the formula (3B-1), a plurality of R_{C} being mutually the same or different, a plurality of R_{D} being mutually the same or different;

the substituent E in the third compound meant by “substituted or unsubstituted” is a substitutent selected from the group consisting of a substituted or unsubstituted aryl group having 6 to 30 ring carbon atoms, a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted silyl group, a substituted or unsubstituted alkoxy group having 1 to 30 carbon atoms, a substituted or unsubstituted alkoxy halide group having 1 to 30 carbon atoms, a substituted or unsubstituted aryloxy group having 6 to 30 carbon atoms, a substituted or unsubstituted alkylthio group having 1 to 30 carbon atoms, a substituted or unsubstituted arylthio group having 6 to 30 ring carbon atoms, a substituted or unsubstituted aralkyl group having 7 to 30 carbon atoms, a substituted or unsubstituted alkenyl group having 2 to 30 carbon atoms, a halogen atom, a substituted or unsubstituted alkynyl group having 2 to 30 carbon atoms, a cyano group, a hydroxy group; a nitro group, and a carboxy group; and

the substituent E optionally includes a substituent F, the substituent F being selected from the group consisting of an unsubstituted aryl group having 6 to 30 ring carbon atoms, an unsubstituted alkyl group having 1 to 30 carbon atoms, an unsubstituted alkyl halide group having 1 to 30 carbon atoms, an unsubstituted cycloalkyl group having 3 to 30 ring carbon atoms, an unsubstituted silyl group, an unsubstituted alkoxy group having 1 to 30 carbon atoms, an unsubstituted halogenated alkoxy group having 1 to 30 carbon atoms, an unsubstituted aryloxy group having 6 to 30 carbon atoms, an unsubstituted alkylthio group having 1 to 30 carbon atoms, an unsubstituted arylthio group having 6 to 30 ring carbon atoms, an unsubstituted aralkyl group having 7 to 30 carbon atoms, an unsubstituted alkenyl group having 2 to 30 carbon atoms, a halogen atom, an unsubstituted alkynyl group having 2 to 30 carbon atoms, a cyano group, a hydroxy group; a nitro group, and a carboxy group, the substituent F not being further substituted.

In the formula (3), Cz bonded to X_{B} may be bonded to any one of Ar₁ or Ar₂ in the formula (3A) representing X_{B}.

The bonding form of the compound represented by the formulae (3) and (3A) is determined depending on the value of n and k. Examples of the bonding form include the forms below.

The bonding form when n in the formula (3) is 1 and k in the formula (3A) is 0

[Formula 133]

Cz-Ar₁ (3-1)

The bonding forms when n in the formula (3) is 1 and k in the formula (3A) is 1

[Formula 134]

Ar₁—Ar₂-Cz (3-2)

Cz-Ar₁—Ar₂ (3-3)

The bonding forms when n in the formula (3) is 1 and k in the formula (3A) is 2

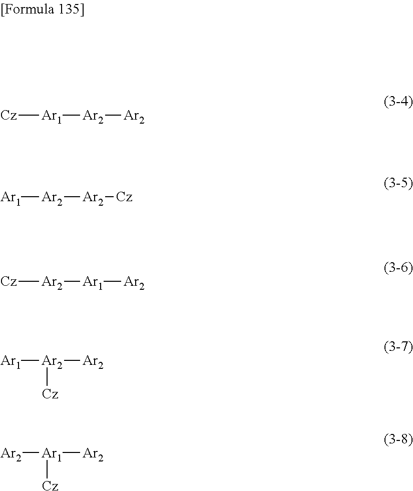

The bonding form when n in the formula (3) is 2 and k in the formula (3A) is 0

[Formula 136]

Cz-Ar₁-Cz (3-9)

The bonding forms when n in the formula (3) is 2 and k in the formula (3A) is 1

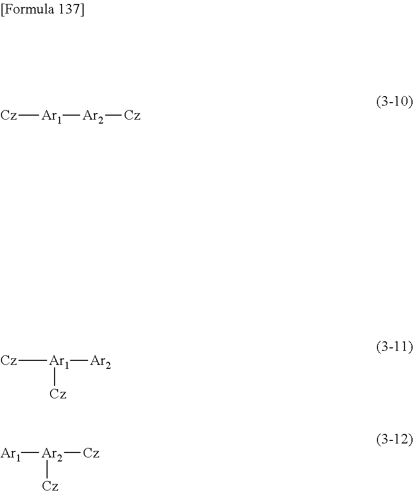

The bonding forms when n in the formula (3) is 2 and k in the formula (3A) is 2

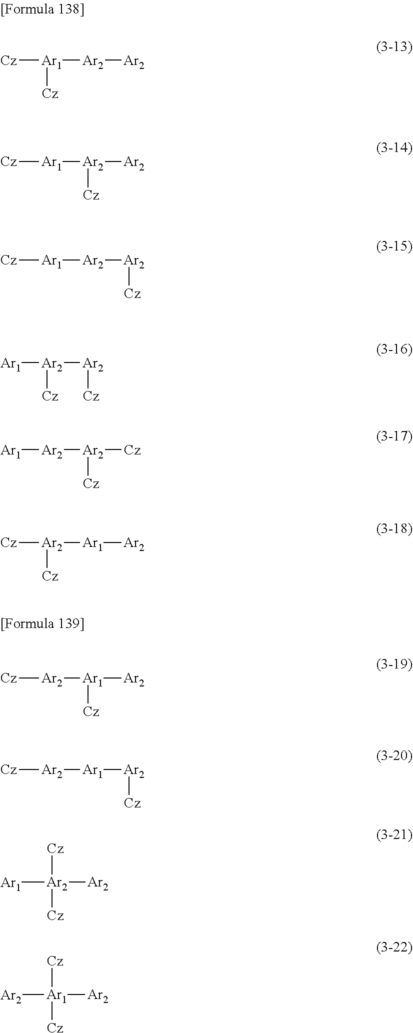

The bonding form when n in the formula (3) is 3 and k in the formula (3A) is 0

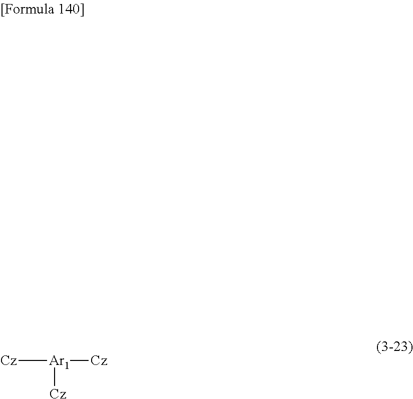

The bonding forms when n in the formula (3) is 3 and k in the formula (3A) is 1

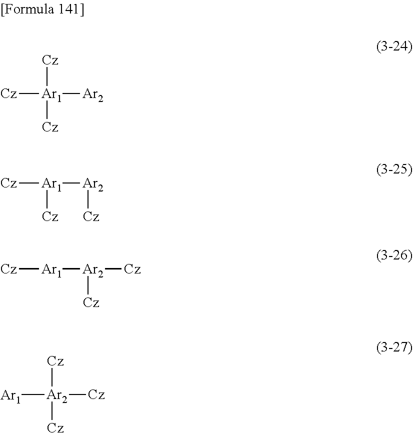

The bonding forms when n in the formula (3) is 3 and k in the formula (3A) is 2

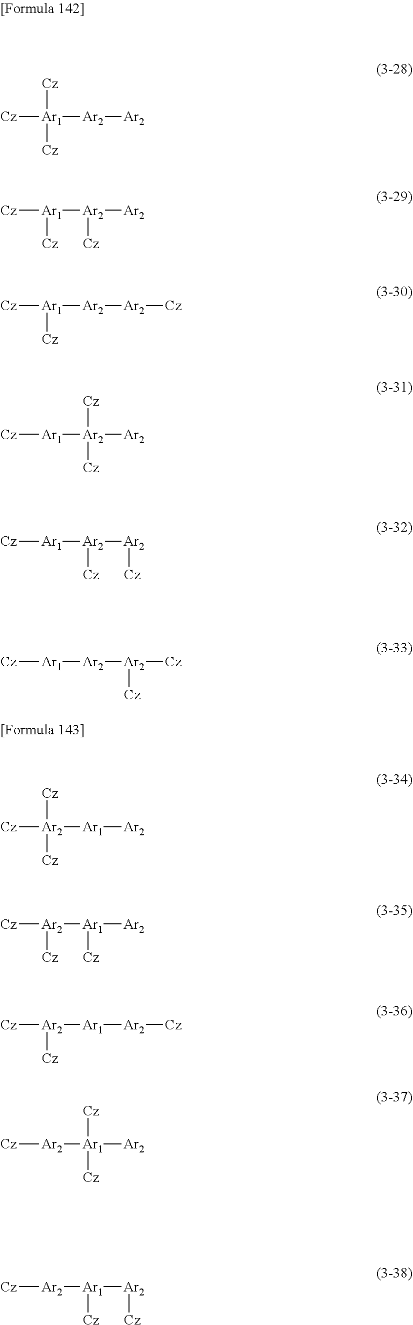

The bonding form when n in the formula (3) is 4 and k in the formula (3A) is 0

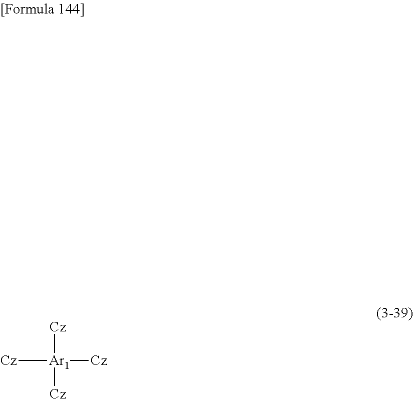

The bonding forms when n in the formula (3) is 4 and k in the formula (3A) is 1

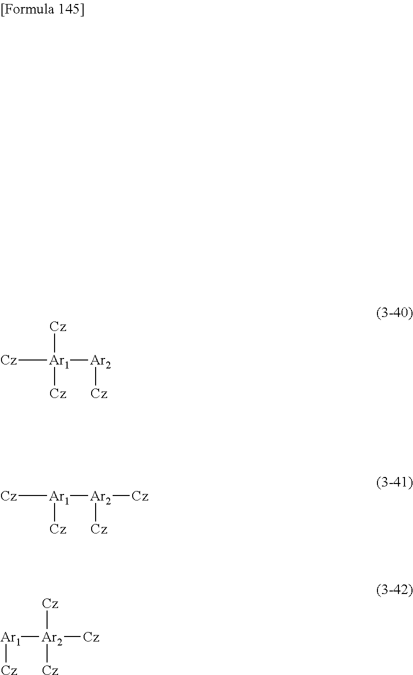

The bonding forms when n in the formula (3) is 4 and k in the formula (3A) is 2

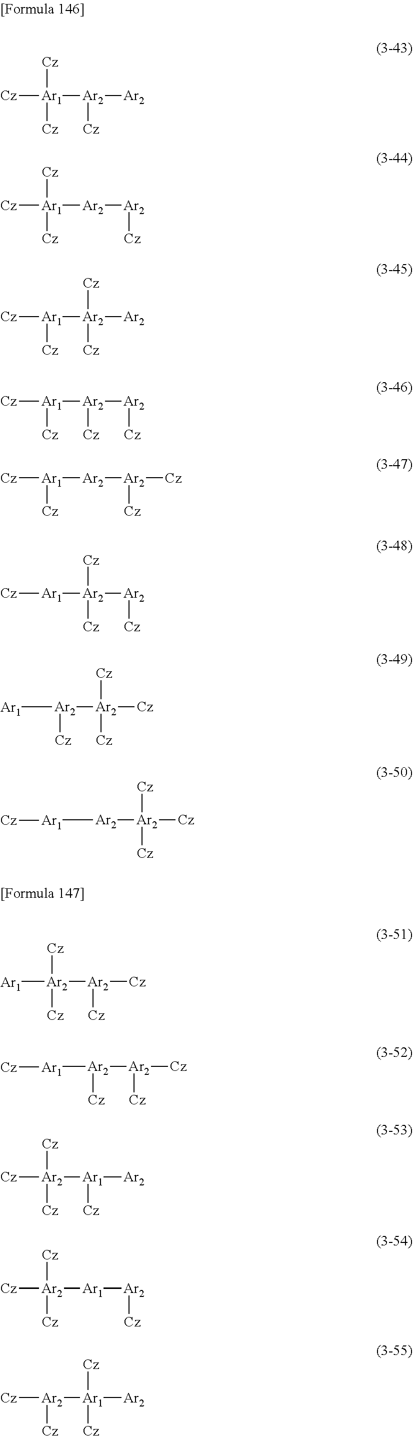

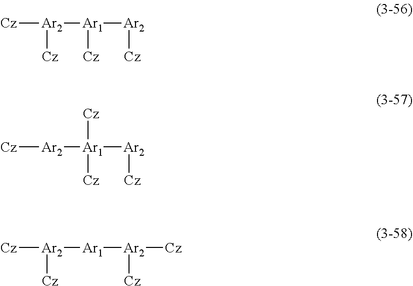

In the above formulae (3-1) to (3-58), when a plurality of Cz are present, the plurality of Cz are mutually the same or different and, when a plurality of Ar₂ are present, the plurality of Ar₂ are mutually the same or different.

Examples of the third compound having the above bonding form are shown below. In these compounds, it is preferable that C_{z1} to C_{z4} are each independently any one group selected from the groups shown in Tables 6 to 13 below. When a plurality of C_{z1} to C_{z4} are present in one molecule, C_{z1} to C_{z4} are mutually the same or different. * represents a bonding position.

In the third compound shown below: the group corresponding to Ar₁ in the formula (3A) optionally includes the substituent D1; the group corresponding Ar₂ in the formula (3A) optionally includes the substituent D2; and the group corresponding R_{A} in the formula (3B-1) optionally includes at least one of the substituents listed as the substituents for R_{A}. The substituent D1, the substituent D2, and the substituent(s) for R_{A} are optionally each independently substituted by the substituent E, the substituent E being optionally substituted by the substituent F.

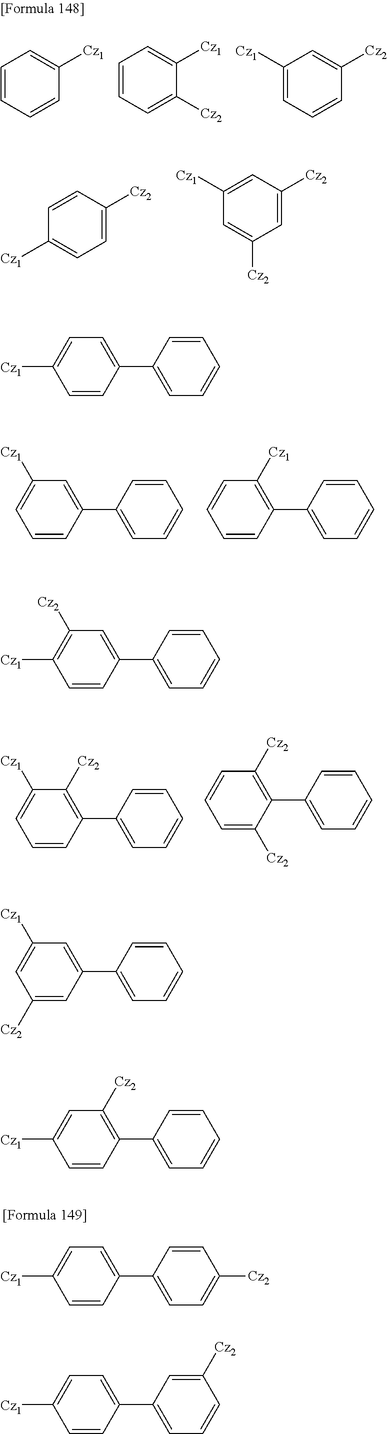

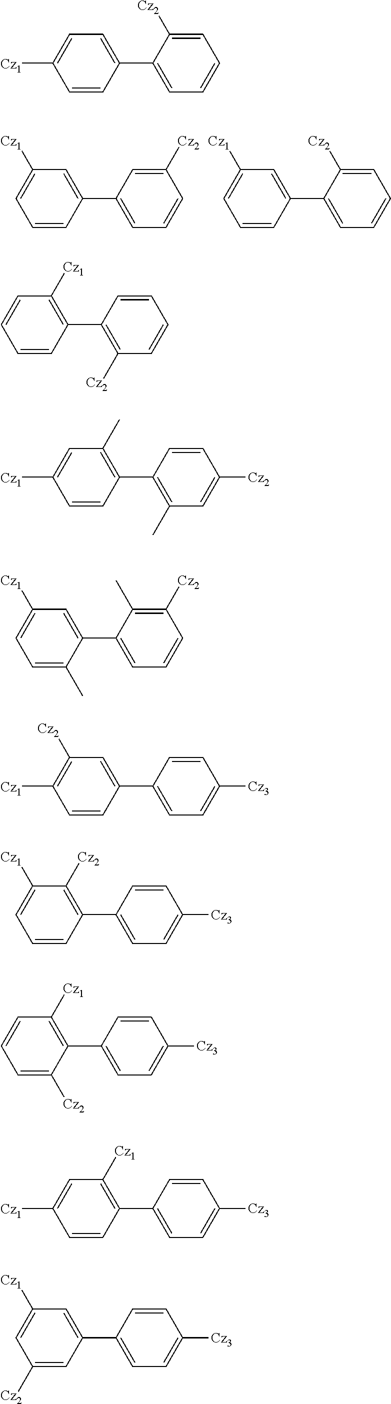

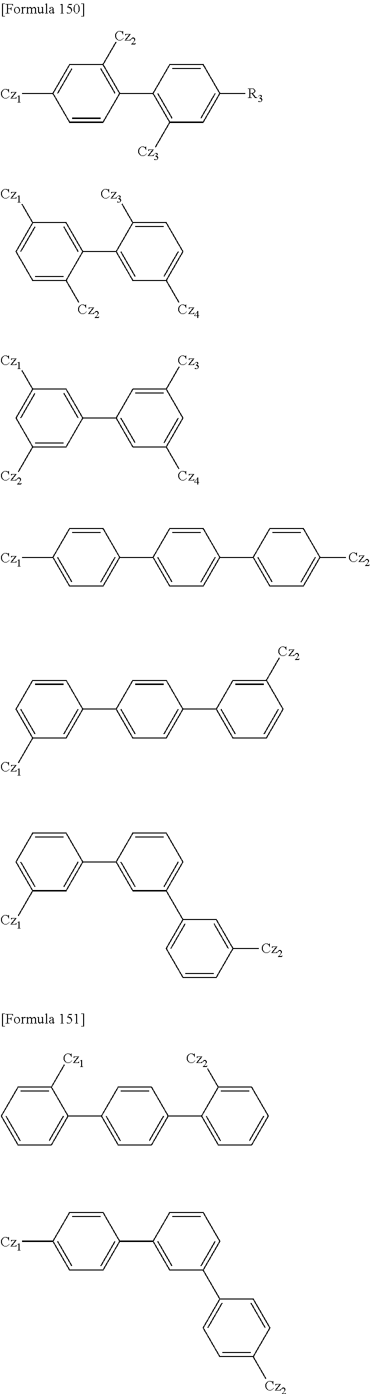

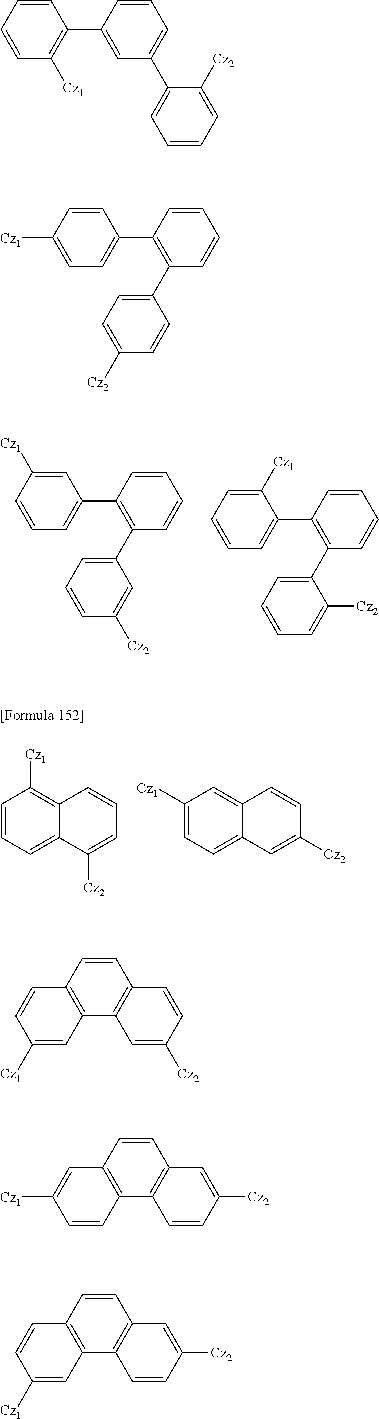

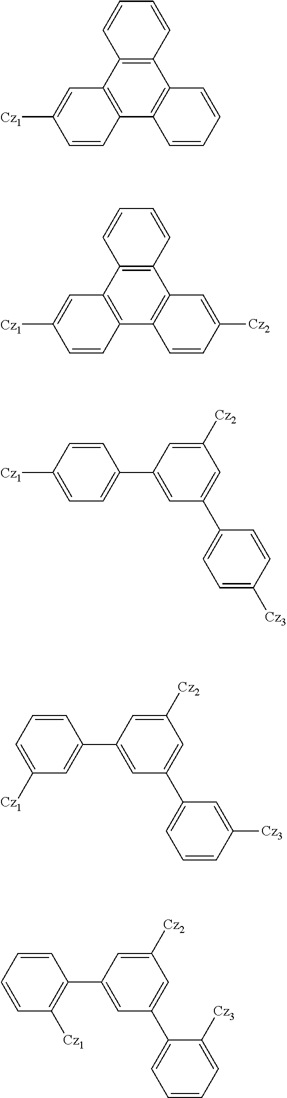

TABLE 6

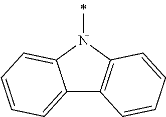

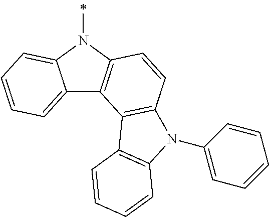

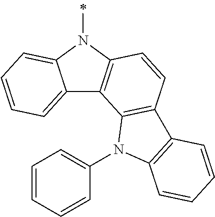

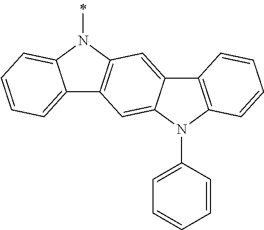

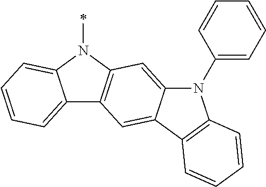

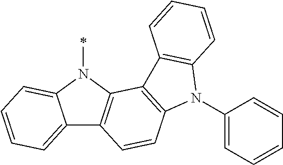

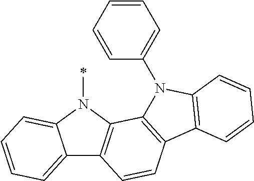

TABLE 7

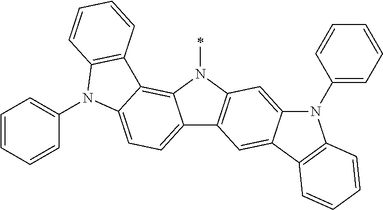

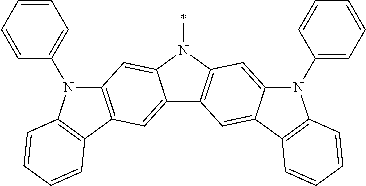

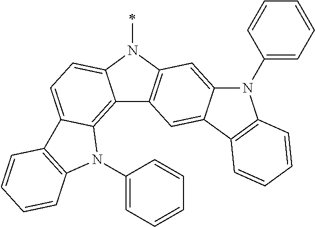

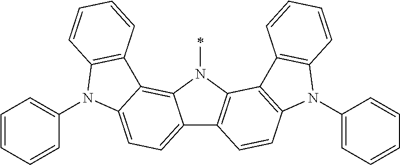

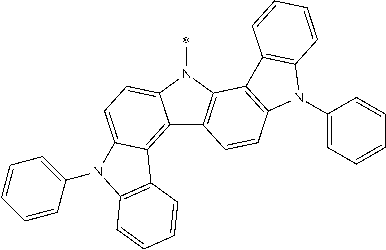

TABLE 8

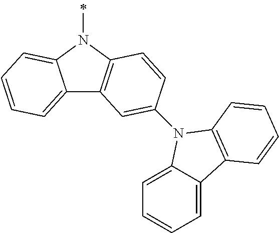

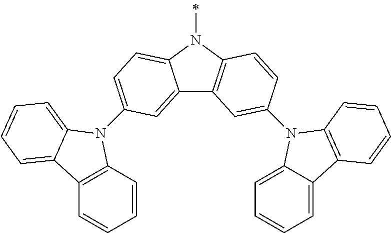

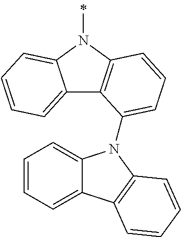

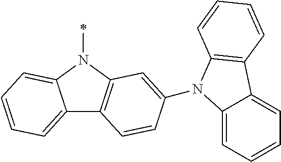

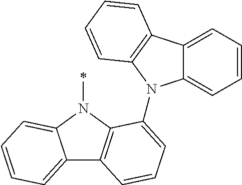

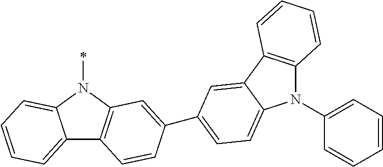

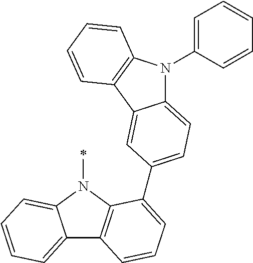

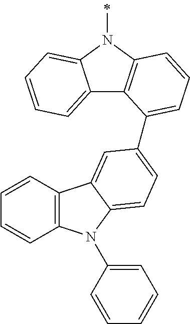

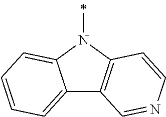

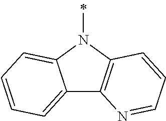

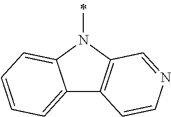

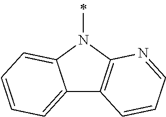

TABLE 9

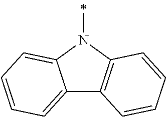

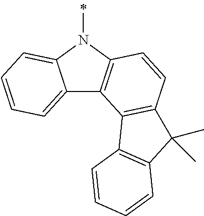

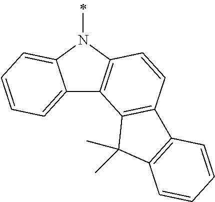

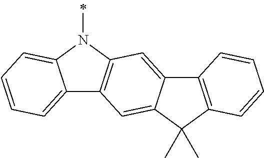

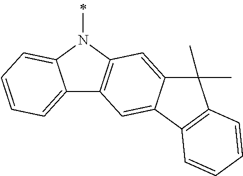

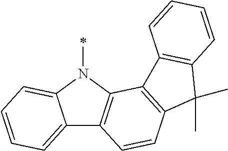

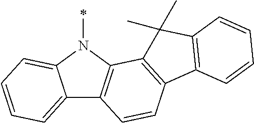

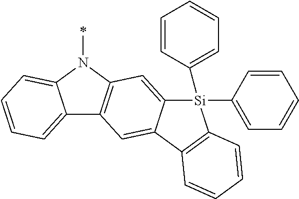

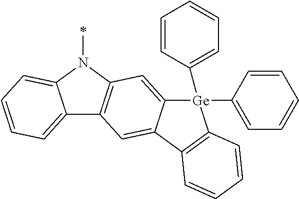

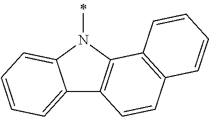

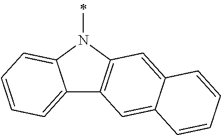

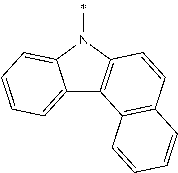

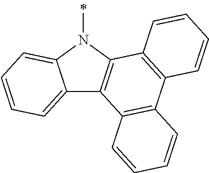

TABLE 10

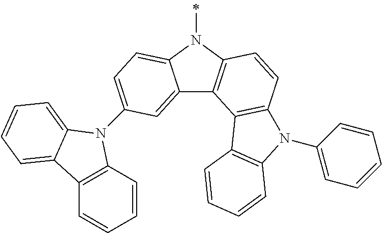

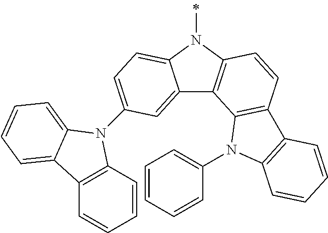

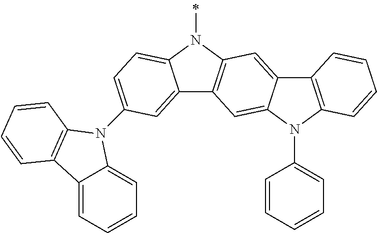

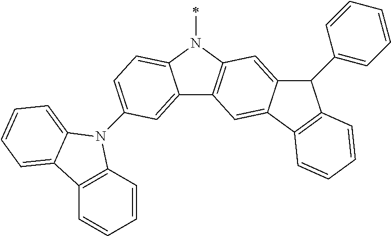

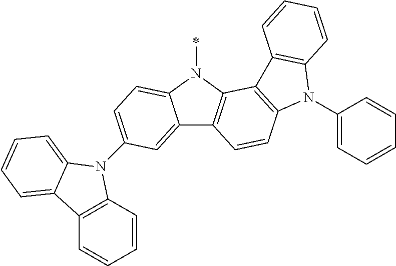

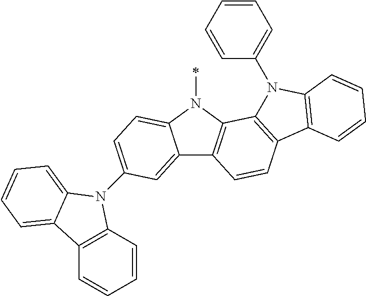

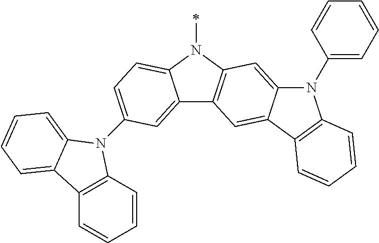

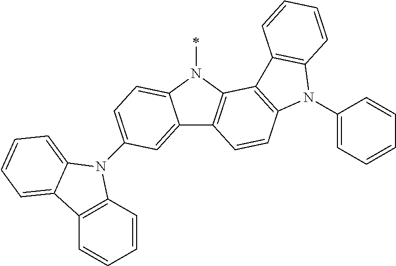

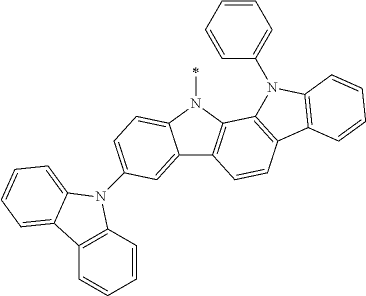

TABLE 11

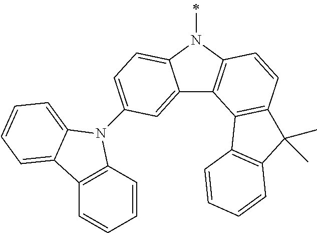

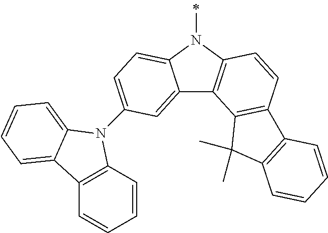

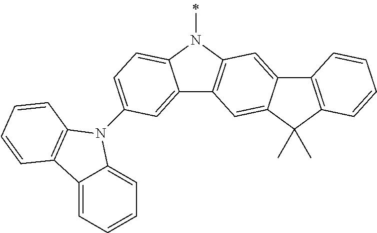

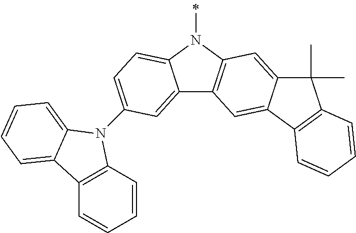

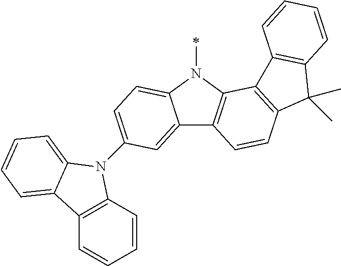

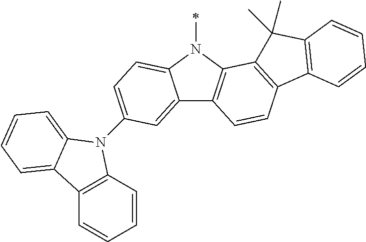

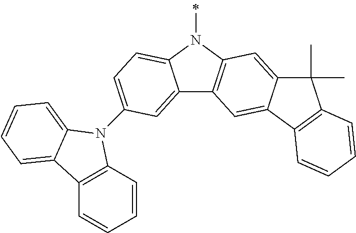

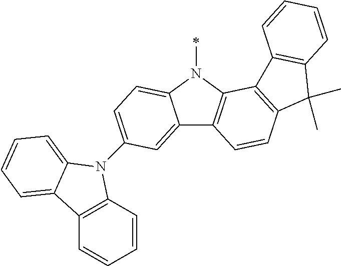

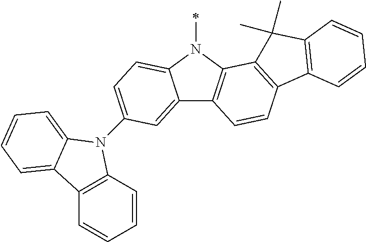

TABLE 12

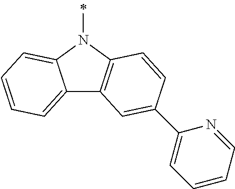

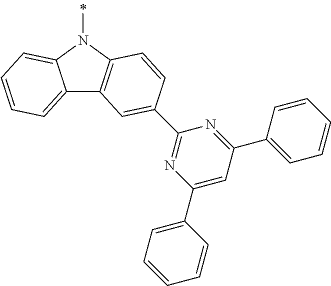

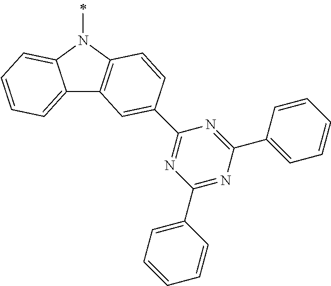

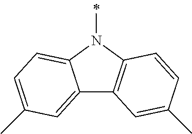

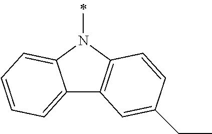

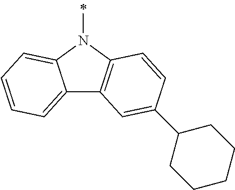

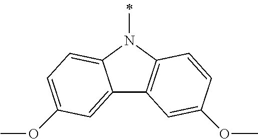

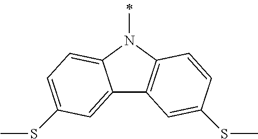

TABLE 13

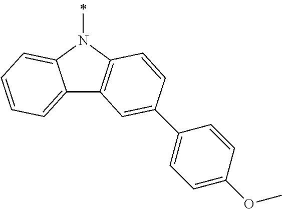

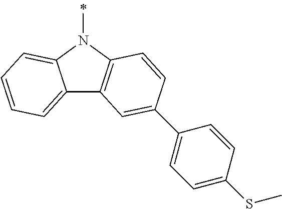

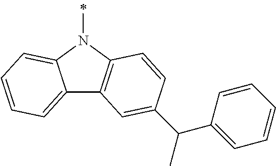

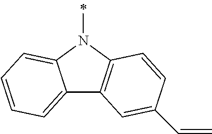

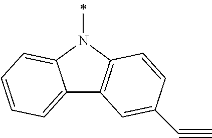

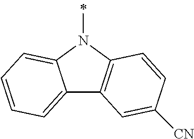

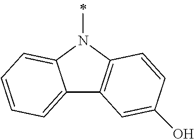

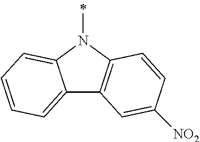

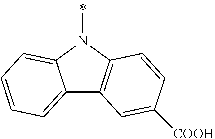

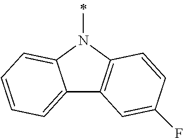

Preferable embodiment(s) of the third compound will be described below.

Cz in the formula (3) is preferably the group represented by the formula (3B-1).

n in the formula (3) is preferably 1 or 2.

Ar₁ and Ar₂ in the formula (3A) are preferably each independently a monovalent or polyvalent residue derived from any one of benzene, biphenyl, terphenyl, naphthalene, anthracene, benzanthracene, phenanthrene, benzophenanthrene, phenalene, picene, pentacene, pyrene, chrysene, benzochrysene, fluoranthene, and triphenylene, and more preferably a monovalent or polyvalent residue derived from any one of benzene, biphenyl, terphenyl, naphthalene, phenanthrene, and triphenylene.

In the formula (3A), Ar₁ in a form of a divalent group and Ar₂ in a form of a divalent group are each independently at least one group selected from the group consisting of an o-phenylene group, m-phenylene group, p-phenylene group, 4,4′-biphenylene group, 4,3′-biphenylene group, 4,2′-biphenylene group, 3,3′-biphenylene group, 2,2′-biphenylene group, 3,2′-biphenylene group, 4,4″-p-terphenylene group, 4,3″-p-terphenylene group, 3,3″-p-terphenylene group, 2,2′″-p-terphenylene group, 4,2″-p-terphenylene group, 4,2′-p-terphenylene group, 4,3′-p-terphenylene group, 4,4″-m-terphenylene group, 4,3″-m-terphenylene group, 4,2″-m-terphenylene group, 4,3′-m-terphenylene group, 3,3′-m-terphenylene group, 2,2′-m-terphenylene group, 4,4″-o-terphenylene group, 3,3″-o-terphenylene group, 2,2′″-o-terphenylene group, 1,4-naphthylene group, 2,6-naphthylene group, 1,5-naphthylene group, 2,7-naphthylene group, 2,6-phenanthrylene group, 3,6-phenanthrylene group, 2,7-phenanthrylene group, fluoranthene-3,8-diyl group, and 2,7-triphenylene group, more preferably at least one group selected from the group consisting of groups represented by formulae (3a-1) to (3a-27) below.

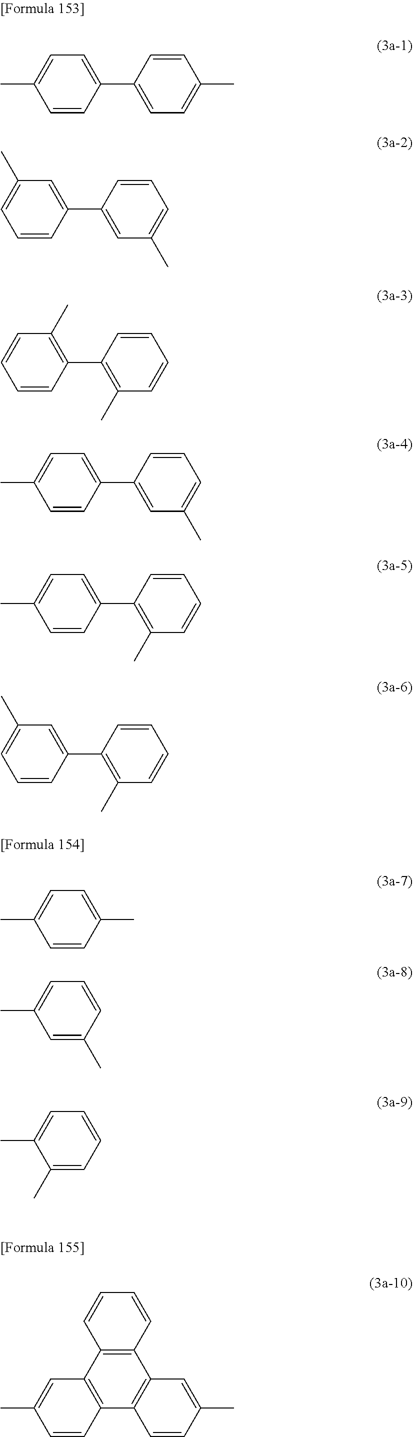

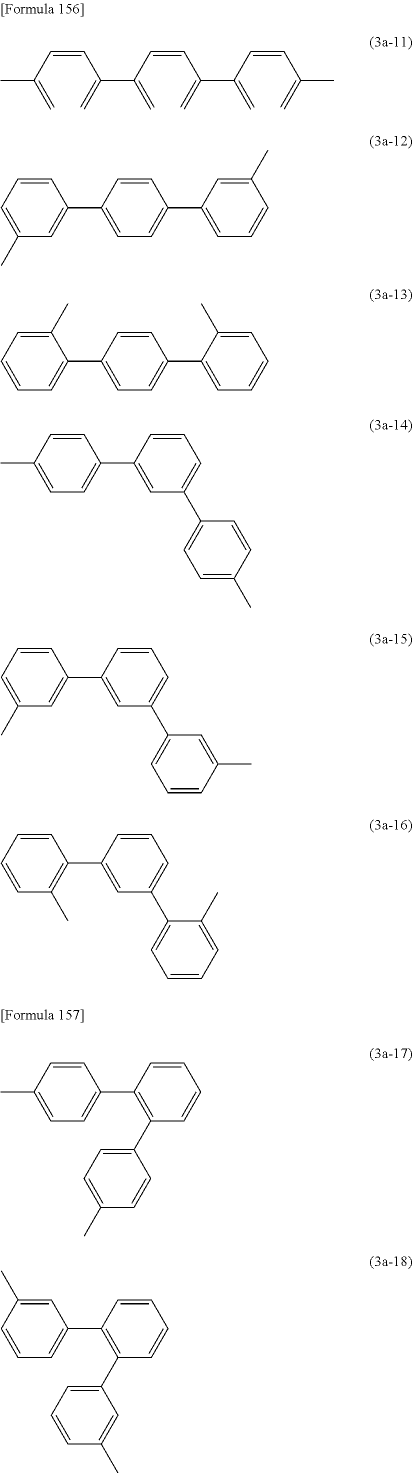

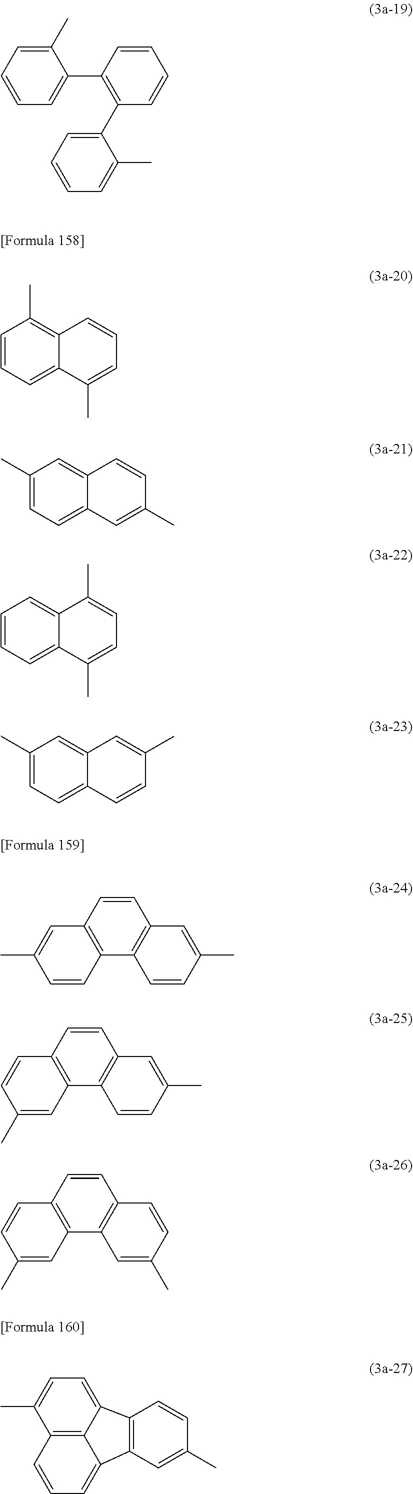

Ar₁ in a form of a monovalent aromatic hydrocarbon group and Ar₂ in a form of a monovalent aromatic hydrocarbon group are preferably each independently a group selected from the group consisting of the groups represented by formulae (3b-1) to (3b-3) below.

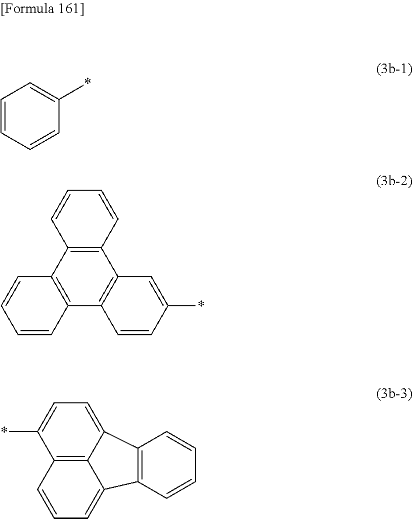 [SMILES: *c1ccccc1 ; *c1ccc2c3ccccc3c3ccccc3c2c1 ; *c1ccc2c3c(cccc13)-c1ccccc1-2]

Ar₁ in a form of a divalent aromatic hydrocarbon group and Ar₂ in a form of a divalent aromatic hydrocarbon group are preferably each independently a group selected from the group consisting of the groups represented by formulae (3b-4) to (3b-10) below.

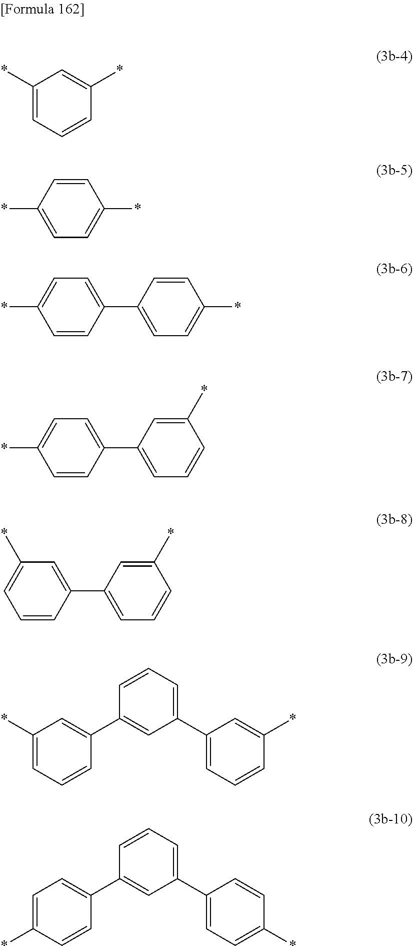

X₁ to X₈ in the formula (3B-1) are preferably each independently CR_{A}.

In the formula (3B-2), X₁ to X₄ are preferably each independently a carbon atom bonded to one of Ar₁ and Ar₂, or CR_{C}, one of X₁ to X₄ being the carbon atom bonded to one of Ar₁ and Ar₂ in the formula (3A), and X₅ to X₈ are preferably each independently CR_{D}.

In the formula (3B-1), it is preferable that a pair of adjacent ones of R_{A} are not mutually bonded.

R_{A} in the formula (3B-1) is preferably a hydrogen atom.

In the formula (3B-2), it is preferable that a pair of adjacent ones of R_{C} are not mutually bonded and a pair of adjacent ones of R_{D} are not mutually bonded.

R_{C} and R_{D} in the formula (3B-2) are each preferably a hydrogen atom.

R_{A} in the formula (3B-1), and R_{B}, R_{C} and R_{D} in the formula (3B-2) are preferably each independently a hydrogen atom, substituted or unsubstituted aryl group having 6 to 30 ring carbon atoms, substituted or unsubstituted alkyl group having 1 to 30 carbon atoms, substituted or unsubstituted alkyl halide group having 1 to 30 carbon atoms, carbazolyl group, substituted carbazolyl group, halogen atom, or cyano group, more preferably a hydrogen atom, substituted or unsubstituted aryl group having 6 to 30 ring carbon atoms, substituted or unsubstituted alkyl group having 1 to 30 carbon atoms, or substituted or unsubstituted alkyl halide group having 1 to 30 carbon atoms.

The third compound is preferably a compound represented by a formula (3C) below.

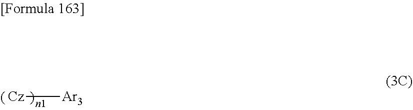

In the formula (3C), Cz represents the same as Cz in the formula (3).

Ar₃ represents the same as Ar₁ in the formula (3A), and n1 represents the same as n in the formula (3A).

n1 is preferably 2.

It is preferable that the third compound is a compound represented by the formula (3C),

n1 is 2, and

Ar₃ is at least one group selected from the group consisting of the groups represented by the formulae (3a-1) to (3a-26).

The groups represented by the formulae (3a-1) to (3a-26) each are substituted by a substituent E or are unsubstituted.

In the formulae (3A), (3B-1) and (3B-2), the substituent E meant by “substituted or unsubstituted” is preferably a substitutent selected from the group consisting of an unsubstituted aryl group having 6 to 30 ring carbon atoms, unsubstituted alkyl group having 1 to 30 carbon atoms, unsubstituted cycloalkyl group having 3 to 30 ring carbon atoms, cyano group, and halogen atom.

In the formulae (3A), (3B-1) and (3B-2), the substituent E meant by “substituted or unsubstituted” is more preferably a substitutent selected from the group consisting of an unsubstituted aryl group having 6 to 30 ring carbon atoms, and unsubstituted alkyl group having 1 to 30 carbon atoms.

In the formulae (3A), (3B-1) and (3B-2), the substituent E meant by “substituted or unsubstituted” is more preferably a substitutent selected from the group consisting of an unsubstituted aryl group having 6 to 18 ring carbon atoms, and unsubstituted alkyl group having 1 to 6 carbon atoms.

More preferable embodiment(s) of the third compound will be described below.

It is preferable in the third compound that: Cz is represented by the formula (3B-1);

n is 1 or 2;

R_{A} in the formula (3B-1) is each independently a hydrogen atom, a substituted or unsubstituted aryl group having 6 to 30 ring carbon atoms, a substituted or unsubstituted alkyl group having 1 to 30 carbon atoms, a carbazolyl group, a substituted carbazolyl group, a halogen atom, or a cyano group;

in the formula (3A), k is 0, and

Ar₁ is a monovalent or polyvalent residue derived from any one of benzene, biphenyl, terphenyl, naphthalene, anthracene, benzanthracene, phenanthrene, benzophenanthrene, phenalene, picene, pentacene, pyrene, chrysene, benzochrysene, fluoranthene, and triphenylene; and

in the formulae (3A) and (3B-1), the substituent E meant by “substituted or unsubstituted” is a substitutent selected from the group consisting of an unsubstituted aryl group having 6 to 30 ring carbon atoms, an unsubstituted alkyl group having 1 to 30 carbon atoms, an unsubstituted cycloalkyl group having 3 to 30 ring carbon atoms, a cyano group, and a halogen atom.

It is preferable that, in the formula (3), n is 1 or 2;

in the formula (3A), k is 0, and

Ar₁ and Ar₂ are each independently a monovalent or polyvalent residue derived from one of benzene, biphenyl, terphenyl, naphthalene, phenanthrene, fluoranthene, and triphenylene;

X₁ to X₈ in the formula (3B-1) are each independently CR_{A};

X₁ to X₄ in the formula (3B-2) are each independently a carbon atom bonded to Ar₁, or CR_{C}, one of X₁ to X₄ being the carbon atom bonded to Ar₁ in the formula (3A), X₅ to X₈ being each independently CR_{D};

R_{A} in the formula (3B-1) and R_{C} and R_{D} in the formula (3B-2) are each independently a hydrogen atom, a substituted or unsubstituted aryl group having 6 to 30 ring carbon atoms, or a substituted or unsubstituted alkyl group having 1 to 30 carbon atoms;

Ar₁ is at least one group selected from the group consisting of the groups represented by the formulae (3a-1) to (3a-26); and

in the formulae (3A), (3B-1), and (3B-2), the substituent E meant by “substituted or unsubstituted” is a substitutent selected from the group consisting of an unsubstituted aryl group having 6 to 30 ring carbon atoms, and an unsubstituted alkyl group having 1 to 30 carbon atoms.

It is more preferable that n in the formula (3) is 2;

k in the formula (3A) is 0;

in the formula (3B-1), X₁ to X₈ are each independently CR_{A} and a pair of adjacent ones of R_{A} are not mutually bonded;

in the formula (3B-2), X₁ to X₄ are each independently a carbon atom bonded to Ar₁, or CR_{C}, one of X₁ to X₄ being the carbon atom bonded to Ar₁ in the formula (3A), a pair of adjacent ones of R_{C} not being mutually bonded, X₅ to X₈ being each independently CR_{D}, a pair of adjacent ones of R_{D} being not mutually bonded;

R_{A} in the formula (3B-1) and R_{C} and R_{D} in the formula (3B-2) are each independently a hydrogen atom; and in the formulae (3A), (3B-1), and (3B-2), the substituent E meant by “substituted or unsubstituted” is a substitutent selected from the group consisting of an unsubstituted aryl group having 6 to 18 ring carbon atoms, an unsubstituted alkyl group having 1 to 6 carbon atoms, and an alkyl halide group having 1 to 30 carbon atoms.

It is preferable that n in the formula (3) is 2;

k in the formula (3A) is 0;

Ar₁ is at least one group selected from the group consisting of the groups represented by the formulae (3a-1) to (3a-26);

in the formula (3B-1), a pair of adjacent ones of R_{A} are not mutually bonded;

in the formula (3B-2), a pair of adjacent ones of R_{C} are not mutually bonded and a pair of adjacent ones of R_{D} are not mutually bonded;

R_{A} in the formula (3B-1) and R_{C} and R_{D} in the formula (3B-2) are each a hydrogen atom; and

in the formulae (3A), (3B-1), and (3B-2), the substituent E meant by “substituted or unsubstituted” is a substitutent selected from the group consisting of an unsubstituted aryl group having 6 to 18 ring carbon atoms, and an unsubstituted alkyl group having 1 to 6 carbon atoms.

It is preferable that n in the formula (3) is 2;

k in the formula (3A) is 0;

Ar₁ is at least one group selected from the group consisting of the groups represented by the formulae (3a-1) to (3a-26);

in the formula (3B-1), X₁ to X₈ are each independently CR_{A} and a pair of adjacent ones of R_{A} are not mutually bonded;

in the formula (3B-2), X₁ to X₄ are each independently a carbon atom bonded to Ar₁, or CR_{C}, one of X₁ to X₄ being the carbon atom bonded to Ar₁ in the formula (3A), a pair of adjacent ones of R_{C} not being mutually bonded, X₅ to X₈ being each independently CR_{D}, a pair of adjacent ones of R_{D} being not mutually bonded;

R_{A} in the formula (3B-1) and R_{C} and R_{D} in the formula (3B-2) are each a hydrogen atom; and

in the formulae (3A), (3B-1), and (3B-2), the substituent E meant by “substituted or unsubstituted” is a substitutent selected from the group consisting of an unsubstituted aryl group having 6 to 18 ring carbon atoms, and an unsubstituted alkyl group having 1 to 6 carbon atoms.

Cz in the formula (3) is preferably at least one group selected from the group consisting of the groups represented by formulae (3B11) to (3B22) below.

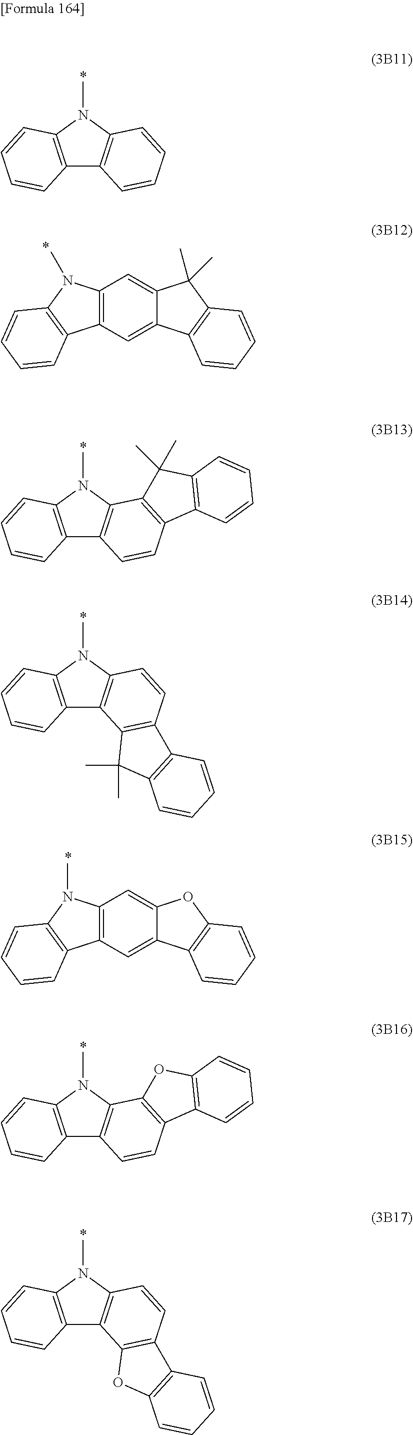

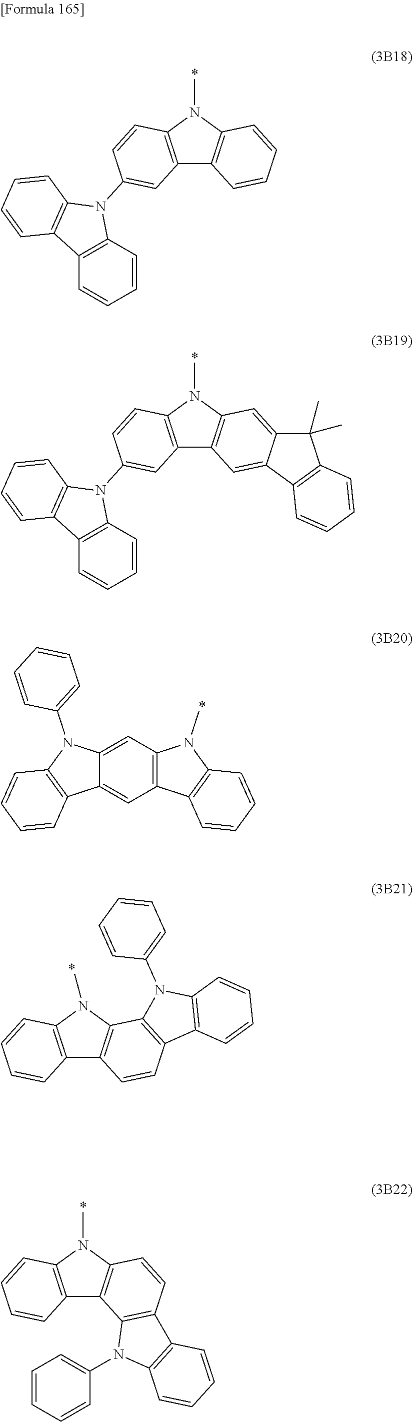 [SMILES: Cn1c2ccccc2c2cc(-n3c4ccccc4c4ccccc43)ccc21 ; Cn1c2ccc(-n3c4ccccc4c4ccccc43)cc2c2cc3c(cc21)C(C)(C)c1ccccc1-3 ; Cn1c2ccccc2c2cc3c4ccccc4n(-c4ccccc4)c3cc21 ; Cn1c2ccccc2c2ccc3c4ccccc4n(-c4ccccc4)c3c21 ; Cn1c2ccccc2c2c1ccc1c3ccccc3n(-c3ccccc3)c12]

In the groups represented by the formula (3B11) to (3B22), the carbon atom capable of being substituted by a substituent is substituted by a substituent X, which is the same as R_{A} as the substituent, or unsubstituted, a plurality of substituents X, when present, being mutually the same or different, * representing a bonding position.

Cz in the formula (3) is preferably a group selected from the group consisting of the groups represented by the formulae (3B11) to (3B17).

### Preparation Method of Third Compound

The third compound can be prepared by any known method.

Specific examples of the third compound in the present exemplary embodiment are shown below. It should be noted that the third compound according to the invention is not limited to these specific examples.

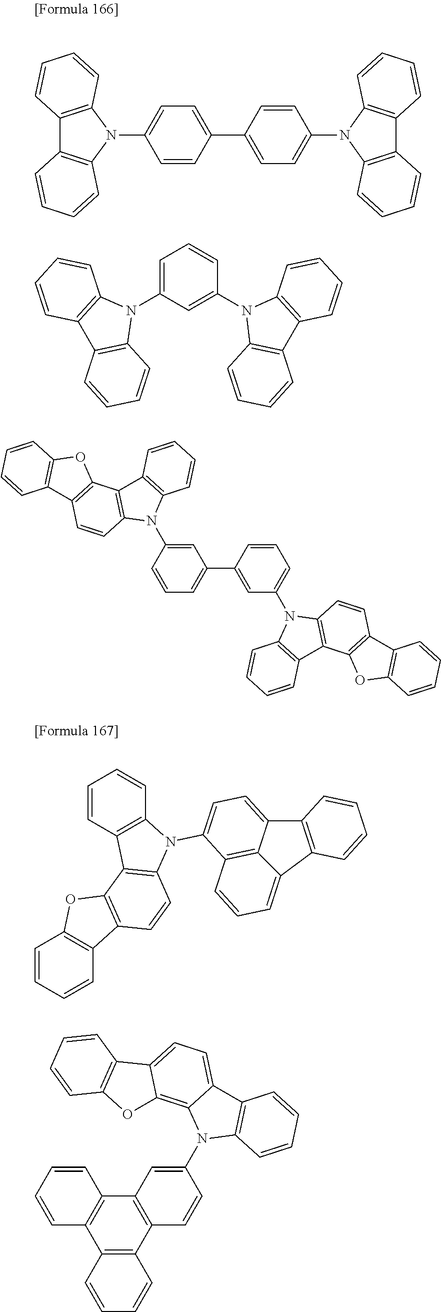

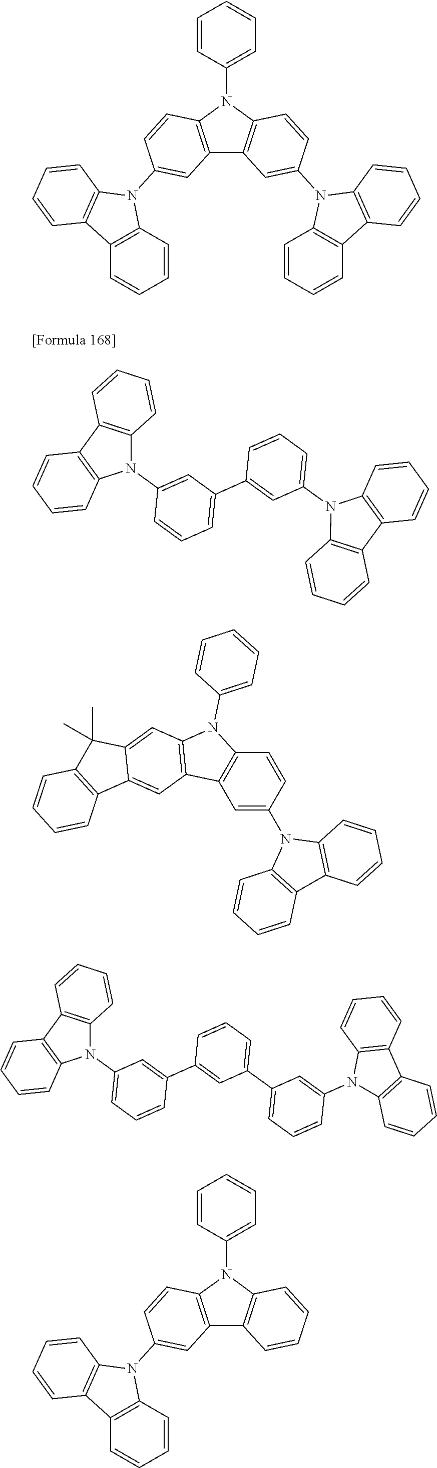 [SMILES: c1ccc(-n2c3ccc(-n4c5ccccc5c5ccccc54)cc3c3cc(-n4c5ccccc5c5ccccc54)ccc32)cc1 ; c1cc(-c2cccc(-n3c4ccccc4c4ccccc43)c2)cc(-n2c3ccccc3c3ccccc32)c1 ; CC1(C)c2ccccc2-c2cc3c4cc(-n5c6ccccc6c6ccccc65)ccc4n(-c4ccccc4)c3cc21 ; c1cc(-c2cccc(-n3c4ccccc4c4ccccc43)c2)cc(-c2cccc(-n3c4ccccc4c4ccccc43)c2)c1 ; c1ccc(-n2c3ccccc3c3cc(-n4c5ccccc5c5ccccc54)ccc32)cc1]

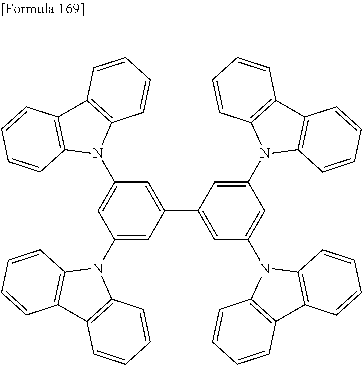 [SMILES: c1ccc2c(c1)c1ccccc1n2-c1cc(-c2cc(-n3c4ccccc4c4ccccc43)cc(-n3c4ccccc4c4ccccc43)c2)cc(-n2c3ccccc3c3ccccc32)c1]

### Relationship Between First Compound, Second Compound and Third Compound in Emitting Layer

In the present exemplary embodiment, the singlet energy S₁(Mat2) of the second compound and the singlet energy S₁(Mat3) of the third compound preferably satisfy a relationship of a numerical formula (Numerical Formula 2) below.

S₁(Mat3)>S₁(Mat2) (Numerical Formula 2).

The singlet energy S₁(Mat3) of the third compound is preferably larger than the singlet energy S₁(Mat1) of the first compound.

An energy gap T_{77K}(Mat3) at 77 [K] of the third compound is preferably larger than an energy gap T_{77K}(Mat1) at 77 [K] of the first compound.

An energy gap T_{77K}(Mat3) at 77 [K] of the third compound is preferably larger than an energy gap T_{77K}(Mat2) at 77 [K] of the second compound.

The singlet energy S₁(Mat1) of the first compound, the singlet energy S₁(Mat2) of the second compound and a singlet energy S₁(Mat3) of the third compound in the emitting layer preferably satisfy a relationship of a numerical formula (Numerical Formula 3) below.

S₁(Mat3)>S₁(Mat2)>S₁(Mat1) (Numerical Formula 3).

The first compound, the second compound, and the third compound in the emitting layer preferably satisfy the relationship of a numerical formula (Numerical Formula 4) below.

T_{77K}(Mat3)>T_{77K}(Mat2)>T_{77K}(Mat1) (Numerical Formula 4).

When the organic EL device of the present exemplary embodiment emits light, it is preferable that the fluorescent compound in the emitting layer mainly emits light.

### Film Thickness of Emitting Layer

A film thickness of the emitting layer of the organic EL device in the present exemplary embodiment is preferably in a range of 5 nm to 50 nm, more preferably in a range of 7 nm to 50 nm, further preferably in a range of 10 nm to 50 nm. At a film thickness of 5 nm or more, formation and chromaticity adjustment of the emitting layer can be facilitated. At a film thickness of 50 nm or less, the rise in the drive voltage is likely to be restrained.

### Content Ratio of Compounds in Emitting Layer

In the organic EL device of the present exemplary embodiment, a content ratio of the first compound in the emitting layer is preferably in a range from 0.01 mass % to 10 mass %, more preferably in a range from 0.01 mass % to 5 mass %, further preferably in a range from 0.01 mass % to 1 mass %.

The content ratio of the second compound preferably ranges from 10 mass % to 80 mass %, more preferably from 10 mass % to 60 mass %, further preferably from 20 mass % to 60 mass %.

The content ratio of the third compound preferably ranges from 10 mass % to 80 mass %.

An upper limit of the total of the respective content ratios of the first, second and third compounds in the emitting layer is 100 mass %. It should be noted that the emitting layer of the present exemplary embodiment may further contain a material other than the first, second and third compounds.

The emitting layer may contain a single type of the first compound or may contain two or more types of the first compound. The emitting layer may contain a single type of the second compound or may contain two or more types of the second compound. The emitting layer may contain a single type of the third compound or may contain two or more types of the third compound.

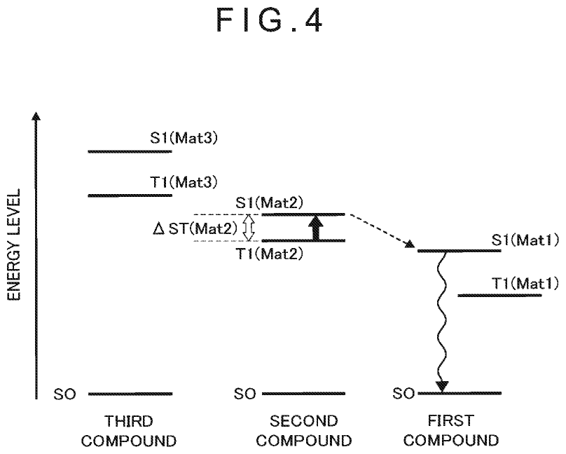

FIG. 4 shows an example of a relationship among energy levels of the first compound, the second compound and the third compound in the emitting layer. In FIG. 4, S0 represents a ground state. S1(Mat1) represents the lowest singlet state of the first compound. T1(Mat1) represents the lowest triplet state of the first compound. S1(Mat2) represents the lowest singlet state of the second compound. T1(Mat2) represents the lowest triplet state of the second compound. S1(Mat3) represents the lowest singlet state of the third compound. T1(Mat3) represents the lowest triplet state of the third compound. A dashed arrow directed from S1(Mat2) to S1(Mat1) in FIG. 4 represents Förster energy transfer from the lowest singlet state of the second compound to the lowest singlet state of the first compound.

As shown in FIG. 4, when a compound having a small ΔST(Mat2) is used as the second compound, inverse intersystem crossing from the lowest triplet state T1(Mat2) to the lowest singlet state S1(Mat2) can be caused by a heat energy. Subsequently, Förster energy transfer from the lowest singlet state S1(Mat2) of the second compound to the first compound occurs to generate the lowest singlet state S1(Mat1). As a result, fluorescence from the lowest singlet state S1(Mat1) of the first compound can be observed. It is inferred that the internal quantum efficiency can be theoretically raised up to 100% also by using delayed fluorescence by the TADF mechanism.

The organic EL device according to the present exemplary embodiment can achieve highly efficient emission.

The organic EL device according to the present exemplary embodiment is applicable to an electronic device such as a display device and a light-emitting device.

### Anode

Metal having a large work function (specifically, 4.0 eV or more), an alloy, an electrically conductive compound and a mixture thereof are preferably usable as the anode formed on the substrate. Specific examples of the material include indium oxide-tin oxide (ITO: Indium Tin Oxide), indium oxide-tin oxide containing silicon or silicon oxide, indium oxide-zinc oxide, indium oxide containing tungsten oxide and zinc oxide, and graphene. In addition, gold (Au), platinum (Pt), nickel (Ni), tungsten (W), chrome (Cr), molybdenum (Mo), iron (Fe), cobalt (Co), copper (Cu), palladium (Pd), titanium (Ti), and nitrides of a metal material (e.g., titanium nitride) are usable.

The material is typically formed into a film by a sputtering method. For instance, the indium oxide-zinc oxide can be formed by the sputtering method using a target in which zinc oxide in a range from 1 mass % to 10 mass % is added to indium oxide. Moreover, for instance, the indium oxide containing tungsten oxide and zinc oxide can be formed by the sputtering method using a target in which tungsten oxide in a range from 0.5 mass % to 5 mass % and zinc oxide in a range from 0.1 mass % to 1 mass % are added to indium oxide. In addition, the anode may be formed by a vacuum deposition method, a coating method, an inkjet method, a spin coating method or the like.

Among the EL layers formed on the anode, since the hole injecting layer adjacent to the anode is formed of a composite material into which holes are easily injectable irrespective of the work function of the anode, a material usable as an electrode material (e.g., metal, an alloy, an electroconductive compound, a mixture thereof, and the elements belonging to the group 1 or 2 of the periodic table) is also usable for the anode.

A material having a small work function such as elements belonging to Groups 1 and 2 in the periodic table of the elements, specifically, an alkali metal such as lithium (Li) and cesium (Cs), an alkaline earth metal such as magnesium (Mg), calcium (Ca) and strontium (Sr), alloys (e.g., MgAg and AlLi) including the alkali metal or the alkaline earth metal, a rare earth metal such as europium (Eu) and ytterbium (Yb), alloys including the rare earth metal are also usable for the anode. It should be noted that the vacuum deposition method and the sputtering method are usable for forming the anode using the alkali metal, alkaline earth metal and the alloy thereof. Further, when a silver paste is used for the anode, the coating method and the inkjet method are usable.

### Cathode

It is preferable to use metal, an alloy, an electroconductive compound, and a mixture thereof, which have a small work function (specifically, 3.8 eV or less) for the cathode. Examples of the material for the cathode include elements belonging to Groups 1 and 2 in the periodic table of the elements, specifically, the alkali metal such as lithium (Li) and cesium (Cs), the alkaline earth metal such as magnesium (Mg), calcium (Ca) and strontium (Sr), alloys (e.g., MgAg and AlLi) including the alkali metal or the alkaline earth metal, the rare earth metal such as europium (Eu) and ytterbium (Yb), and alloys including the rare earth metal.

It should be noted that the vacuum deposition method and the sputtering method are usable for forming the cathode using the alkali metal, alkaline earth metal and the alloy thereof. Further, when a silver paste is used for the cathode, the coating method and the inkjet method are usable.

By providing the electron injecting layer, various conductive materials such as Al, Ag, ITO, graphene, and indium oxide-tin oxide containing silicon or silicon oxide may be used for forming the cathode regardless of the work function. The conductive materials can be formed into a film using the sputtering method, inkjet method, spin coating method and the like.

### Hole Injecting Layer

The hole injecting layer is a layer containing a substance exhibiting a high hole injectability. Examples of the substance exhibiting a high hole injectability include molybdenum oxide, titanium oxide, vanadium oxide, rhenium oxide, ruthenium oxide, chrome oxide, zirconium oxide, hafnium oxide, tantalum oxide, silver oxide, tungsten oxide, and manganese oxide.

In addition, the examples of the highly hole-injectable substance include: an aromatic amine compound, which is a low-molecule organic compound, such that 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), 4,4′-bis[N-(4-diphenylaminophenyl)-N-phenylamino]biphenyl(abbreviation: DPAB), 4,4′-bis(N-{4-[N′-(3-methylphenyl)-N′-phenylamino]phenyl}-N-phenylamino)biphenyl (abbreviation: DNTPD), 1,3,5-tris[N-(4-diphenylaminophenyl)-N-phenylamino]benzene (abbreviation: DPA3B), 3-[N-(9-phenylcarbazole-3-yl)-N-phenylamino]-9-phenylcarbazole (abbreviation: PCzPCA1), 3,6-bis[N-(9-phenylcarbazole-3-yl)-N-phenylamino]-9-phenylcarbazole (abbreviation: PCzPCA2), and 3-[N-(1-naphthyl)-N-(9-phenylcarbazole-3-yl)amino]-9-phenylcarbazole (abbreviation: PCzPCN1); and dipyrazino[2,34:20,30-h]quinoxaline-2,3,6,7,10,11-hexacarbonitrile (HAT-CN).

In addition, a high polymer compound (e.g., oligomer, dendrimer and polymer) is usable as the substance exhibiting a high hole injectability. Examples of the high polymer compound include poly(N-vinylcarbazole) (abbreviation: PVK), poly(4-vinyltriphenylamine) (abbreviation: PVTPA), poly[N-(4-{N′-[4-(4-diphenylamino)phenyl]phenyl-′-phenylamino}phenyl)methacrylamido] (abbreviation: PTPDMA), and poly[N, N′-bis(4-butylphenyl)-N,N′-bis(phenyl)benzidine] (abbreviation: Poly-TPD). Moreover, an acid-added high polymer compound such as poly(3,4-ethylenedioxythiophene)/poly(styrene sulfonic acid) (PEDOT/PSS) and polyaniline/poly (styrene sulfonic acid)(PAni/PSS) are also usable.

### Hole Transporting Layer

The hole transporting layer is a layer containing a highly hole-transporting substance. An aromatic amine compound, carbazole derivative, anthracene derivative and the like are usable for the hole transporting layer. Specific examples of a material for the hole transporting layer include 4,4′-bis[N-(1-naphthyl)-N-phenylamino]biphenyl (abbreviation: NPB), N,N′-bis(3-methylphenyl)-N,N′-diphenyl-[1,1′-biphenyl]-4,4′-diamine (abbreviation: TPD), 4-phenyl-4′-(9-phenylfluorene-9-yl)triphenylamine (abbreviation: BAFLP), 4,4′-bis[N-(9,9-dimethylfluorene-2-yl)-N-phenylamino]biphenyl (abbreviation: DFLDPBi), 4,4′,4″-tris(N, N-diphenylamino)triphenylamine (abbreviation: TDATA), 4,4′,4″-tris[N-(3-methylphenyl)-N-phenylamino]triphenylamine (abbreviation: MTDATA), and 4,4′-bis[N-(spiro-9,9′-bifluorene-2-yl)-N-phenylamino]biphenyl (abbreviation: BSPB). The above-described substances mostly have a hole mobility of 10⁻⁶ cm²/(V·s) or more.

For the hole transporting layer, a carbazole derivative such as CBP, 9-[4-(N-carbazolyl)]phenyl-10-phenylanthracene (CzPA), and 9-phenyl-3-[4-(10-phenyl-9-anthryl)phenyl]-9H-carbazole (PCzPA) and an anthracene derivative such as t-BuDNA, DNA, and DPAnth may be used. A high polymer compound such as poly(N-vinylcarbazole) (abbreviation: PVK) and poly(4-vinyltriphenylamine) (abbreviation: PVTPA) is also usable.

However, in addition to the above substances, any substance exhibiting a higher hole transportability than an electron transportability may be used. It should be noted that the layer containing the substance exhibiting a high hole transportability may be not only a single layer but also a laminate of two or more layers formed of the above substance.

When the hole transporting layer includes two or more layers, one of the layers with a larger energy gap is preferably provided closer to the emitting layer. An example of the material with a larger energy gap is HT-2 used in later-described Examples.

### Electron Transporting Layer

The electron transporting layer is a layer containing a highly electron-transporting substance. For the electron transporting layer, 1) a metal complex such as an aluminum complex, beryllium complex, and zinc complex, 2) a hetero aromatic compound such as imidazole derivative, benzimidazole derivative, azine derivative, carbazole derivative, and phenanthroline derivative, and 3) a high polymer compound are usable. Specifically, as a low-molecule organic compound, a metal complex such as Alq, tris(4-methyl-8-quinolinato)aluminum (abbreviation: Almq₃), bis(10-hydroxybenzo[h]quinolinato)beryllium (abbreviation: BeBq₂), BAlq, Znq, ZnPBO and ZnBTZ is usable. In addition to the metal complex, a heteroaromatic compound such as 2-(4-biphenylyl)-5-(4-tert-butylphenyl)-1,3,4-oxadiazole (abbreviation: PBD), 1,3-bis[5-(ptert-butylphenyl)-1,3,4-oxadiazole-2-yl]benzene (abbreviation: OXD-7), 3-(4-tert-butylphenyl)-4-phenyl-5-(4-biphenylyl)-1,2,4-triazole (abbreviation: TAZ), 3-(4-tert-butylphenyl)-4-(4-ethylphenyl)-5-(4-biphenylyl)-1,2,4-triazole (abbreviation: p-EtTAZ), bathophenanthroline (abbreviation: BPhen), bathocuproine (abbreviation: BCP), and 4,4′-bis(5-methylbenzoxazole-2-yl)stilbene (abbreviation: BzOs) is usable. In the present exemplary embodiment, a benzimidazole compound is preferably usable. The above-described substances mostly have an electron mobility of 10⁻⁶ cm²/(V·s) or more. It should be noted that any substance other than the above substance may be used for the electron transporting layer as long as the substance exhibits a higher electron transportability than the hole transportability. The electron transporting layer may be provided in the form of a single layer or a laminate of two or more layers of the above substance(s).

Moreover, a high polymer compound is usable for the electron transporting layer. For instance, poly[(9,9-dihexylfluorene-2,7-diyl)-co-(pyridine-3,5-diyl)] (abbreviation: PF-Py), poly[(9,9-dioctylfluorene-2,7-diyl)-co-(2,2′-bipyridine-6,6′-diyl)] (abbreviation: PF-BPy) and the like are usable.

### Electron Injecting Layer

The electron injecting layer is a layer containing a highly electron-injectable substance. Examples of a material for the electron injecting layer include an alkali metal, alkaline earth metal and a compound thereof, examples of which include lithium (Li), cesium (Cs), calcium (Ca), lithium fluoride (LiF), cesium fluoride (CsF), calcium fluoride (CaF₂), and lithium oxide (LiOx). In addition, the alkali metal, alkaline earth metal or the compound thereof may be added to the substance exhibiting the electron transportability in use. Specifically, for instance, magnesium (Mg) added to Alq may be used. In this case, the electrons can be more efficiently injected form the anode.

Alternatively, the electron injecting layer may be provided by a composite material in a form of a mixture of the organic compound and the electron donor. Such a composite material exhibits excellent electron injectability and electron transportability since electrons are generated in the organic compound by the electron donor. In this case, the organic compound is preferably a material excellent in transporting the generated electrons. Specifically, the above examples (e.g., the metal complex and the hetero aromatic compound) of the substance forming the electron transporting layer are usable. As the electron donor, any substance exhibiting electron donating property to the organic compound is usable. Specifically, the electron donor is preferably alkali metal, alkaline earth metal and rare earth metal such as lithium, cesium, magnesium, calcium, erbium and ytterbium. The electron donor is also preferably alkali metal oxide and alkaline earth metal oxide such as lithium oxide, calcium oxide, and barium oxide. Moreover, a Lewis base such as magnesium oxide is usable. Further, the organic compound such as tetrathiafulvalene (abbreviation: TTF) is usable.

### Layer Formation Method

A method for forming each layer of the organic EL device in the present exemplary embodiment is subject to no limitation except for the above particular description. However, known methods of dry film-forming such as vacuum deposition, sputtering, plasma or ion plating and wet film-forming such as spin coating, dipping, flow coating or ink-jet are applicable.

### Film Thickness

A thickness of each of the organic layers in the organic EL device according to the present exemplary embodiment is not limited except for the above particular description. In general, the thickness preferably ranges from several nanometers to 1 μm in order to avoid defects such as a pin hole and to prevent efficiency from being deteriorated since a high voltage needs to be applied.

### Electronic Device

An electronic device of the present exemplary embodiment is provided with the organic EL device according to the present exemplary embodiment. Examples of the electronic device include a display device and a light-emitting unit. Examples of the display device include a display component (e.g., an organic EL panel module), TV, mobile phone, tablet and personal computer. Examples of the light-emitting unit include an illuminator and a vehicle light.

### Modification of Embodiment(s)

It should be noted that the invention is not limited to the above exemplary embodiment but may include any modification and improvement as long as such modification and improvement are compatible with the invention.

For instance, the emitting layer is not limited to a single layer, but may be provided by laminating a plurality of emitting layers. When the organic EL device has the plurality of emitting layers, it is only required that at least one of the emitting layers satisfies the conditions described in the above exemplary embodiment. For instance, in some embodiments, the rest of the emitting layers is a fluorescent emitting layer or a phosphorescent emitting layer with use of emission caused by electron transfer from the triplet excited state directly to the ground state.

When the organic EL device includes the plurality of emitting layers, in some embodiments, the plurality of emitting layers are adjacent to each other, or provide a so-called tandem-type organic EL device in which a plurality of emitting units are layered through an intermediate layer.

Further, for instance, a blocking layer is provided adjacent to the emitting layer on at least one side thereof near the anode or the cathode, in some embodiments. The blocking layer is preferably adjacent to the emitting layer to block at least one of holes, electrons, or excitons.

When the blocking layer is provided in contact with the cathode-side of the emitting layer, the blocking layer permits transport of electrons, but blocks holes from reaching a layer provided near the cathode (e.g., the electron transporting layer) beyond the blocking layer. When the organic EL device includes the electron transporting layer, the organic EL device preferably includes the blocking layer between the emitting layer and the electron transporting layer.

When the blocking layer is provided in contact with the anode-side of the emitting layer, the blocking layer permits transport of holes, but blocks electrons from reaching a layer provided near the anode (e.g., the hole transporting layer) beyond the blocking layer. When the organic EL device includes the hole transporting layer, the organic EL device preferably includes the blocking layer between the emitting layer and the hole transporting layer.

Moreover, the blocking layer may be adjacent to the emitting layer so that excited energy does not leak out from the emitting layer toward neighboring layer(s). The blocking layer blocks excitons generated in the emitting layer from transferring to a layer(s) (e.g., the electron transporting layer and the hole transporting layer) closer to the electrode(s) beyond the blocking layer.

The emitting layer and the blocking layer are preferably bonded with each other.

In addition, the specific structure and shapes for practicing the invention may be altered to other structures and shapes as long as such other structures and shapes are compatible with the invention.

Herein, numerical ranges represented by “x to y” represents a range whose lower limit is the value (x) recited before “to” and whose upper limit is the value (y) recited after “to.”

Herein, the phrase “Rx and Ry are mutually bonded to form a ring” means, for instance, that Rx and Ry include a carbon atom, a nitrogen atom, an oxygen atom, a sulfur atom or a silicon atom, the atom(s) contained in Rx (a carbon atom, a nitrogen atom, an oxygen atom, a sulfur atom or a silicon atom) and the atom(s) contained in Ry (a carbon atom, a nitrogen atom, an oxygen atom, a sulfur atom or a silicon atom) are bonded via a single bond(s), a double bond(s), a triple bond, and/or a divalent linking group(s) to form a ring having 5 or more ring atoms (specifically, a heterocycle or an aromatic hydrocarbon ring). x represents a number, a character or a combination of a number and a character. y represents a number, a character or a combination of a number and a character.

The divalent linking group is not limited. Examples of the divalent linking group include —O—, —CO—, —CO₂—, —S—, —SO—, —SO₂—, —NH—, —NRa—, and a group provided by a combination of two or more of these linking group.

Specific examples of the heterocycle include a cyclic structure (heterocycle) provided by removing a bond from the “heteroaryl group having ring 5 to 30 ring atoms” exemplified in later-described “Description of Substituents in the Formula.” The heterocyclic ring may have a substituent.

Specific examples of the aromatic hydrocarbon ring include a cyclic structure (aromatic hydrocarbon ring) provided by removing a bond from the “aryl group having ring 6 to 30 ring carbon atoms” exemplified in later-described “Description of Substituents in the Formula.” The aromatic hydrocarbon ring may have a substituent.

Examples of Ra include a substituted or unsubstituted alkyl group having 1 to 30 carbon atoms, a substituted or unsubstituted aryl group having 6 to 30 ring carbon atoms, and a substituted or unsubstituted heteroaryl group having 5 to 30 ring atoms.

For instance, when “Rx and Ry are mutually bonded to form a ring,” an atom(s) contained in Rx₁ and an atom(s) contained in Ry₁ in a molecular structure represented by a formula (E1) below form a ring (cyclic structure) E represented by a formula (E2); an atom(s) contained in Rx₁ and an atom(s) contained in Ry₁ in a molecular structure represented by a formula (F1) below form a ring (cyclic structure) F represented by a formula (F2); an atom(s) contained in Rx₁ and an atom(s) contained in Ry₁ in a molecular structure represented by a formula (G1) below form a ring (cyclic structure) G represented by a formula (G2); an atom(s) contained in Rx₁ and an atom(s) contained in Ry₁ in a molecular structure represented by a formula (H1) below form a ring (cyclic structure) H represented by a formula (H2); or an atom(s) contained in Rx₁ and an atom(s) contained in Ry₁ in a molecular structure represented by a formula (11) below form a ring (cyclic structure) I represented by a formula (12).

In the formulae (E1) to (I1), * each independently represent a bonding position to another atom in a molecule. The two * in the formulae (E1), (F1), (G1), (H1) and (I1) correspond to two * in the formula (E2), (F2), (G2), (H2) and (I2), respectively.

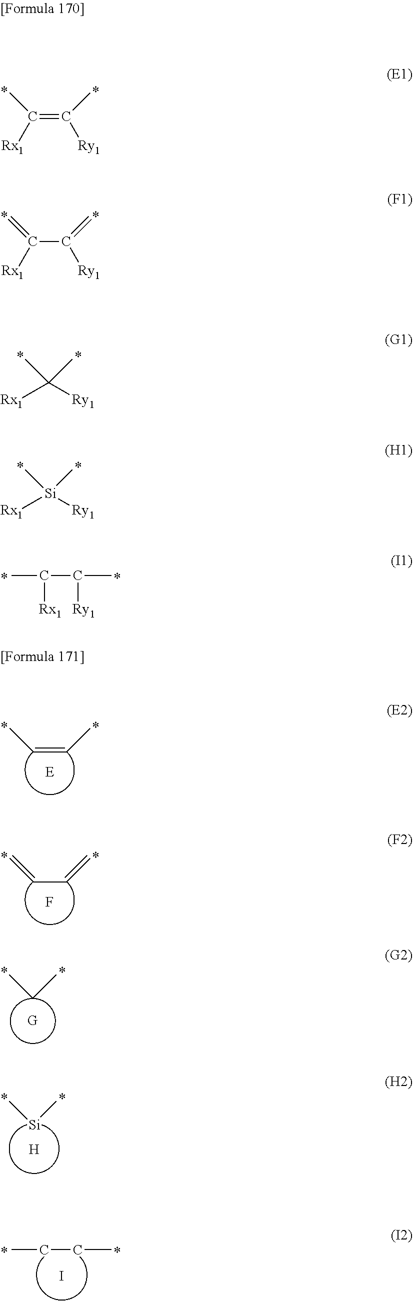

In the molecular structures represented by the formulae (E2) to (I2), E to I each represent a cyclic structure (the ring having 5 or more ring atoms). In the formulae (E2) to (I2), * each independently represent a bonding position to another atom in a molecule. The two * in the formula (E2) correspond to two * in the formula (E1). Similarly, two * in each of the formulae (F2) to (I2) correspond one-to-one to two * in in each of the formulae (F1) to (I1).

For instance, in the formula (E1), Rx₁ and Ry₁ are mutually bonded to form the ring E in the formula (E2) and the ring E is an unsubstituted benzene ring, the molecular structure represented by the formula (E1) is a molecular structure represented by a formula (E3) below. Herein, two * in the formula (E3) each independently correspond to two * in the formula (E2) and the formula (E1).

For instance, in the formula (E1), Rx₁ and Ry₁ are mutually bonded to form the ring E in the formula (E2) and the ring E is an unsubstituted pyrrole ring, the molecular structure represented by the formula (E1) is a molecular structure represented by a formula (E4) below. Herein, two * in the formula (E4) each independently correspond to two * in the formula (E2) and the formula (E1). In the formulae (E3) and (E4), * each independently represent a bonding position to another atom in a molecule.

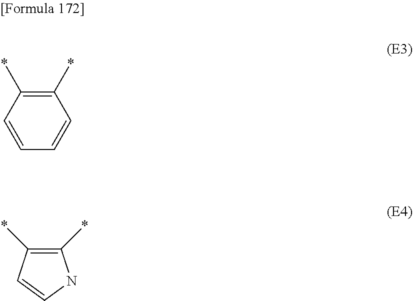 [SMILES: *c1ccccc1* ; *c1cc[nH]c1*]

Herein, the ring carbon atoms refer to the number of carbon atoms among atoms forming a ring of a compound (e.g., a monocyclic compound, fused-ring compound, crosslinking compound, carbon ring compound, and heterocyclic compound) in which the atoms are bonded to each other to form the ring. When the ring is substituted by a substituent(s), carbon atom(s) contained in the substituent(s) is not counted in the ring carbon atoms. Unless specifically described, the same applies to the “ring carbon atoms” described later. For instance, a benzene ring has 6 ring carbon atoms, a naphthalene ring has 10 ring carbon atoms, a pyridinyl group has 5 ring carbon atoms, and a furanyl group has 4 ring carbon atoms. When a benzene ring and/or a naphthalene ring is substituted by a substituent (e.g., an alkyl group), the number of carbon atoms of the alkyl group is not counted in the number of the ring carbon atoms. When a fluorene ring is substituted by a substituent (e.g., a fluorene ring) (i.e., a spirofluorene ring is included), the number of carbon atoms of the fluorene ring as the substituent is not counted in the number of the ring carbon atoms of the fluorene ring.

Herein, the ring atoms refer to the number of atoms forming a ring of a compound (e.g., a monocyclic compound, fused-ring compound, crosslinking compound, carbon ring compound, and heterocyclic compound) in which the atoms are bonded to each other to form the ring (e.g., monocyclic ring, fused ring, ring assembly). Atom(s) not forming a ring and atom(s) included in a substituent when the ring is substituted by the substituent are not counted in the number of the ring atoms. Unless specifically described, the same applies to the “ring atoms” described later. For instance, a pyridine ring has six ring atoms, a quinazoline ring has ten ring atoms, and a furan ring has five ring atoms. A hydrogen atom(s) and/or an atom(s) of a substituent which are bonded to carbon atoms of a pyridine ring and/or quinazoline ring are not counted in the ring atoms. When a fluorene ring is substituted by a substituent (e.g., a fluorene ring) (i.e., a spirofluorene ring is included), the number of atoms of the fluorene ring as the substituent is not counted in the number of the ring atoms of the fluorene ring.

### Description of Each Substituent in Formula Herein

Examples of the aryl group having 6 to 30 ring carbon atoms (occasionally referred to as an aromatic hydrocarbon group) herein are a phenyl group, biphenyl group, terphenyl group, naphthyl group, anthryl group, phenanthryl group, fluorenyl group, pyrenyl group, chrysenyl group, fluoranthenyl group, benz[a]anthryl group, benzo[c]phenanthryl group, triphenylenyl group, benzo[k]fluoranthenyl group, benzo[g]chrysenyl group, benzo[b]triphenylenyl group, picenyl group, and perylenyl group.

Herein, the aryl group preferably has 6 to 20 ring carbon atoms, more preferably 6 to 14 ring carbon atoms, further preferably 6 to 12 ring carbon atoms. Among the aryl group, a phenyl group, biphenyl group, naphthyl group, phenanthryl group, terphenyl group and fluorenyl group are particularly preferable. A carbon atom in a position 9 of each of 1-fluorenyl group, 2-fluorenyl group, 3-fluorenyl group and 4-fluorenyl group is preferably substituted by a substituted or unsubstituted alkyl group having 1 to 30 carbon atoms or a substituted or unsubstituted aryl group having 6 to 18 ring carbon atoms described later herein.

The heteroaryl group (occasionally, referred to as heterocyclic group, heteroaromatic ring group or aromatic heterocyclic group) having 5 to 30 ring atoms herein preferably contains at least one atom selected from the group consisting of nitrogen, sulfur, oxygen, silicon, selenium atom and germanium atom, and more preferably contains at least one atom selected from the group consisting of nitrogen, sulfur and oxygen.

Examples of the heterocyclic group having 5 to 30 ring atoms herein are a pyridyl group, pyrimidinyl group, pyrazinyl group, pyridazynyl group, triazinyl group, quinolyl group, isoquinolinyl group, naphthyridinyl group, phthalazinyl group, quinoxalinyl group, quinazolinyl group, phenanthirdinyl group, acridinyl group, phenanthrolinyl group, pyrrolyl group, imidazolyl group, pyrazolyl group, triazolyl group, tetrazolyl group, indolyl group, benzimidazolyl group, indazolyl group, imidazopyridinyl group, benzotriazolyl group, carbazolyl group, furyl group, thienyl group, oxazolyl group, thiazolyl group, isoxazolyl group, isothiazolyl group, oxadiazolyl group, thiadiazolyl group, benzofuranyl group, benzothienyl group, benzoxazolyl group, benzothiazolyl group, benzisoxazolyl group, benzisothiazolyl group, benzoxadiazolyl group, benzothiadiazolyl group, dibenzofuranyl group, dibenzothienyl group, piperidinyl group, pyrrolidinyl group, piperazinyl group, morpholyl group, phenazinyl group, phenothiazinyl group, and phenoxazinyl group.

Herein, the heterocyclic group preferably has 5 to 20 ring atoms, more preferably 5 to 14 ring atoms. Among the above heterocyclic group, a 1-dibenzofuranyl group, 2-dibenzofuranyl group, 3-dibenzofuranyl group, 4-dibenzofuranyl group, 1-dibenzothienyl group, 2-dibenzothienyl group, 3-dibenzothienyl group, 4-dibenzothienyl group, 1-carbazolyl group, 2-carbazolyl group, 3-carbazolyl group, 4-carbazolyl group, and 9-carbazolyl group are further preferable. A nitrogen atom in position 9 of 1-carbazolyl group, 2-carbazolyl group, 3-carbazolyl group and 4-carbazolyl group is preferably substituted by the substituted or unsubstituted aryl group having 6 to 30 ring carbon atoms or the substituted or unsubstituted heterocyclic group having 5 to 30 ring atoms described herein.

Herein, the heterocyclic group may be a group derived from any one of moieties represented by formulae (XY-1) to (XY-18) below.

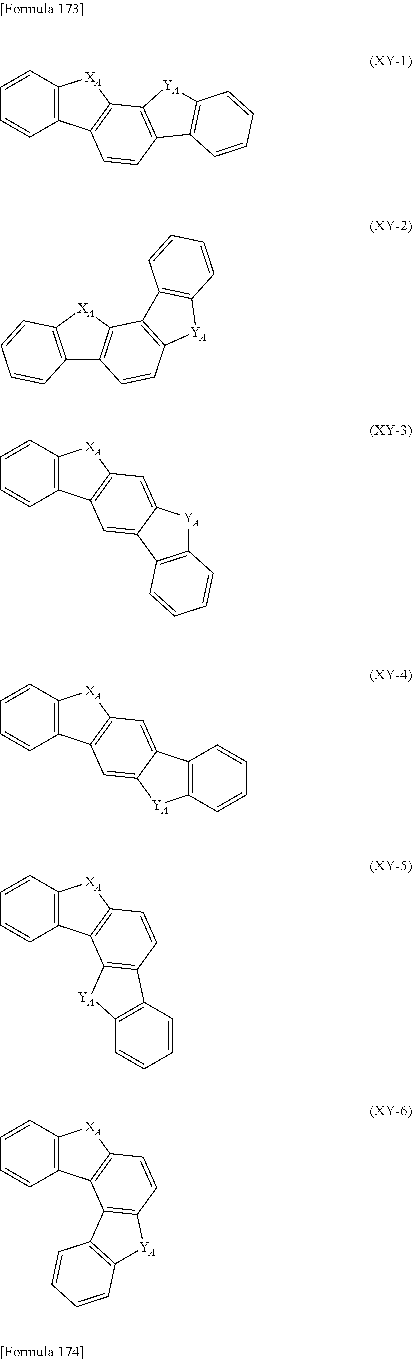

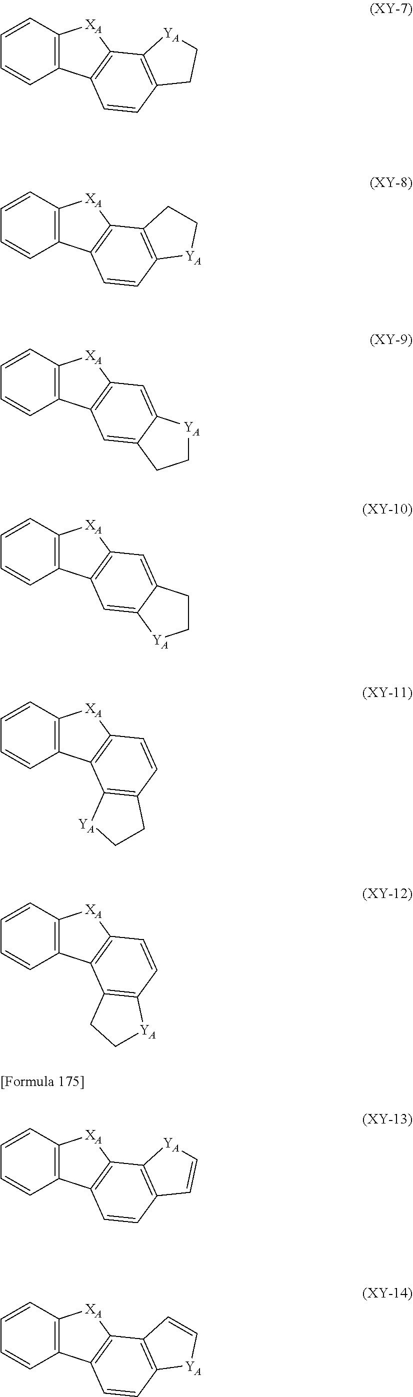

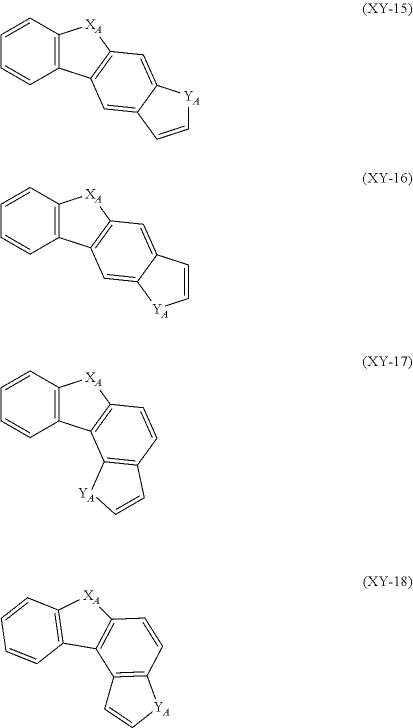

In the formulae (XY-1) to (XY-18), X_{A} and Y_{A} each independently represent a hetero atom, and preferably represent an oxygen atom, sulfur atom, selenium atom, silicon atom or germanium atom. Each of the moieties represented by the respective formulae (XY-1) to (XY-18) has a bond at any position to provide a heterocyclic group. The heterocyclic group may be substituted.

Examples of a substituted or unsubstituted carbazolyl group herein may contain groups represented by formulae (XY-19) to (XY-22) in which a further group is fused to a carbazole ring. Such a group may have a substituent. Moreover, the position of the bond may be changed as needed

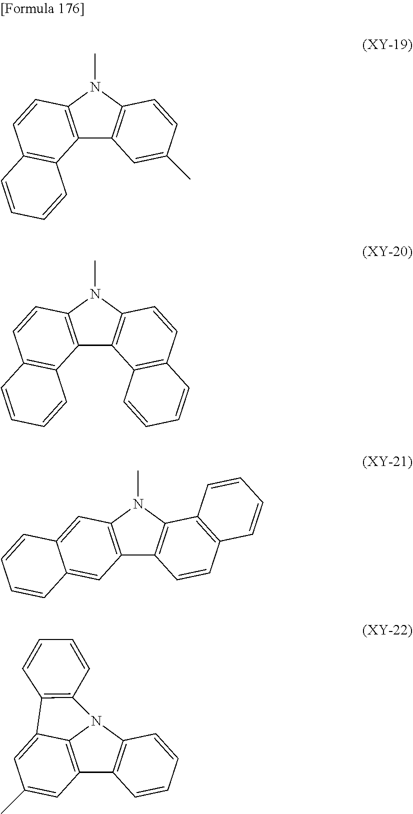

The alkyl group having 1 to 30 carbon atoms herein may be linear, branched or cyclic. Also, the alkyl group may be an alkyl halide group.

Examples of the linear or branched alkyl group include: a methyl group, ethyl group, n-propyl group, isopropyl group, n-butyl group, s-butyl group, isobutyl group, t-butyl group, n-pentyl group, n-hexyl group, n-heptyl group, n-octyl group, n-nonyl group, n-decyl group, n-undecyl group, n-dodecyl group, n-tridecyl group, n-tetradecyl group, n-pentadecyl group, n-hexadecyl group, n-heptadecyl group, n-octadecyl group, neopentyl group, amyl group, isoamyl group, 1-methylpentyl group, 2-methylpentyl group, 1-pentylhexyl group, 1-butylpentyl group, 1-heptyloctyl group, and 3-methylpentyl group.

Herein, the linear or branched alkyl group preferably has 1 to 10 carbon atoms, more preferably 1 to 6 carbon atoms. Among the linear or branched alkyl group, a methyl group, ethyl group, propyl group, isopropyl group, n-butyl group, s-butyl group, isobutyl group, t-butyl group, n-pentyl group, n-hexyl group, amyl group, isoamyl group and neopentyl group are preferable.

Herein, examples of the cyclic alkyl group include a cycloalkyl group having 3 to 30 ring carbon atoms.

Examples of the cycloalkyl group having 3 to 30 ring carbon atoms herein are a cyclopropyl group, cyclobutyl group, cyclopentyl group, cyclohexyl group, 4-metylcyclohexyl group, adamantyl group and norbornyl group. The cycloalkyl group preferably has 3 to 10 ring carbon atoms, more preferably 5 to 8 ring carbon atoms. Among the cycloalkyl group, a cyclopentyl group and a cyclohexyl group are more preferable.

Herein, the alkyl halide group provided by substituting the alkyl group with a halogen atom is exemplified by an alkyl halide group provided by substituting the alkyl group having 1 to 30 carbon atoms with at least one halogen atom, preferably at least one fluorine atom.

Herein, examples of the alkyl halide group having 1 to 30 carbon atoms include a fluoromethyl group, difluoromethyl group, trifluoromethyl group, fluoroethyl group, trifluoromethylmethyl group, trifluoroethyl group, and pentafluoroethyl group.

Herein, examples of a substituted silyl group include an alkylsilyl group having 3 to 30 carbon atoms and an arylsilyl group having 6 to 30 ring carbon atoms.

Herein, the alkylsilyl group having 3 to 30 carbon atoms is exemplified by a trialkylsilyl group having the above examples of the alkyl group having 1 to 30 carbon atoms. Specific examples of the alkylsilyl group are a trimethylsilyl group, triethylsilyl group, tri-n-butylsilyl group, tri-n-octylsilyl group, triisobutylsilyl group, dimethylethylsilyl group, dimethylisopropylsilyl group, dimethyl-n-propylsilyl group, dimethyl-n-butylsilyl group, dimethyl-t-butylsilyl group, diethylisopropylsilyl group, vinyl dimethylsilyl group, propyldimethylsilyl group, and triisopropylsilyl group. Three alkyl groups in the trialkylsilyl group may be mutually the same or different.

Herein, examples of the arylsilyl group having 6 to 30 ring carbon atoms include a dialkylarylsilyl group, alkyldiarylsilyl group and triarylsilyl group.

The dialkylarylsilyl group is exemplified by a dialkylarylsilyl group including two of the alkyl group listed as the examples of the alkyl group having 1 to 30 carbon atoms and one of the aryl group listed as the examples of the aryl group having 6 to 30 ring carbon atoms. The dialkylarylsilyl group preferably has 8 to 30 carbon atoms.

The alkyldiarylsilyl group is exemplified by an alkyldiarylsilyl group including one of the alkyl group listed as the examples of the alkyl group having 1 to 30 carbon atoms and two of the aryl group listed as the examples of the aryl group having 6 to 30 ring carbon atoms. The alkyldiarylsilyl group preferably has 13 to 30 carbon atoms.

The triarylsilyl group is exemplified by a triarylsilyl group including three of the aryl group listed as the examples of the aryl group having 6 to 30 ring carbon atoms. The triarylsilyl group preferably has 18 to 30 carbon atoms.

Herein, alkylsulfonyl group is represented by —SO₂R_{w}. R_{w} in —SO₂R_{w} represents a substituted or unsubstituted alkyl group.

Examples of the substituted or unsubstituted alkylsulfonyl having 1 to 30 carbon atoms herein include a group represented by the above —SO₂R_{w}, where R_{w} is substituted or unsubstituted alkyl group having 1 to 30 carbon atoms.

Herein, an aryl group in an aralkyl group (occasionally referred to as an arylalkyl group) is an aromatic hydrocarbon group or a heterocyclic group.

The aralkyl group having 7 to 30 carbon atoms herein is preferably a group having an aryl group having 6 to 30 ring carbon atoms and is represented by —Z₃-Z₄. Z₃ is exemplified by an alkylene group corresponding to the above alkyl group having 1 to 30 carbon atoms. Z₄ is exemplified by the above aryl group having 6 to 30 ring carbon atoms. In this aralkyl group, an aryl moiety has 6 to 30 carbon atoms, preferably 6 to 20 carbon atoms, more preferably 6 to 12 carbon atoms and an alkyl moiety has 1 to 30 carbon atoms, preferably 1 to 20 carbon atoms, more preferably 1 to 10 carbon atoms, further preferably 1 to 6 carbon atoms. Examples of the aralkyl group are a benzyl group, 2-phenylpropane-2-yl group, 1-phenylethyl group, 2-phenylethyl group, 1-phenylisopropyl group, 2-phenylisopropyl group, phenyl-t-butyl group, α-naphthylmethyl group, 1-α-naphthylethyl group, 2-α-naphthylethyl group, 1-α-naphthylisopropyl group, 2-α-naphthylisopropyl group, β-naphthylmethyl group, 1-β-naphthylethyl group, 2-β-naphthylethyl group, 1-β-naphthylisopropyl group, and 2-β-naphthylisopropyl group.

The alkoxy group having 1 to 30 carbon atoms herein is represented by —OZ₁. Z₁ is exemplified by the above alkyl group having 1 to 30 carbon atoms. Examples of the alkoxy group include a methoxy group, ethoxy group, propoxy group, butoxy group, pentyloxy group and hexyloxy group. The alkoxy group preferably has 1 to 20 carbon atoms.

A halogenated alkoxy group provided by substituting an alkoxy group with a halogen atom is exemplified by one provided by substituting an alkoxy group having 1 to 30 carbon atoms with one or more fluorine atoms.

Herein, an aryl group in an aryloxy group (sometime referred to as an arylalkoxy group) also includes a heteroaryl group.

The arylalkoxy group having 6 to 30 ring carbon atoms herein is represented by —OZ₂. Z₂ is exemplified by the above aryl group having 6 to 30 ring carbon atoms. The arylalkoxy group preferably has 6 to 20 ring carbon atoms. The arylalkoxy group is exemplified by a phenoxy group.

Herein, the substituted amino group is represented by —NHR_{V} or —N(R_{V})₂. R_{V} is exemplified by the above alkyl group having 1 to 30 carbon atoms or aryl group having 6 to 30 ring carbon atoms.

Herein, the alkenyl group having 2 to 30 carbon atoms is linear or branched. Examples of the alkenyl group include a vinyl group, propenyl group, butenyl group, oleyl group, eicosapentaenyl group, docosahexaenyl group, styryl group, 2,2-diphenylvinyl group, 1,2,2-triphenylvinyl group, and 2-phenyl-2-propenyl group.

The alkynyl group having 2 to 30 carbon atoms herein may be linear or branched. Examples of the alkynyl group having 2 to 30 carbon atoms are an ethynyl group, a propynyl group and a 2-phenylethynyl group.

Herein, the alkylthio group having 1 to 30 ring carbon atoms and the arylthio group having 6 to 30 ring carbon atoms is represented by —SR_{V}. R_{V} is exemplified by the above alkyl group having 1 to 30 carbon atoms or aryl group having 6 to 30 ring carbon atoms. The alkylthio group preferably has 1 to 20 carbon atoms. The arylthio group preferably has 6 to 20 ring carbon atoms.

Herein, examples of the halogen atom include a fluorine atom, a chlorine atom, a bromine atom and an iodine atom, among which a fluorine atom is preferable.

Herein, examples of a substituted phosphino group include a phenyl phosphanyl group.

Herein, the arylcarbonyl group having 6 to 30 ring carbon atoms is represented by —COY′. Y′ is exemplified by the above “aryl group having 6 to 30 ring carbon atoms.” Herein, examples of the arylcarbonyl group having 6 to 30 ring carbon atoms include a phenyl carbonyl group, diphenyl carbonyl group, naphthyl carbonyl group, and triphenyl carbonyl group.

The acyl group having 2 to 31 carbon atoms herein is represented by —COR′. R′ is exemplified by the above alkyl group having 1 to 30 carbon atoms. Herein, the acyl group having 2 to 31 carbon atoms is exemplified by an acetyl group and a propionyl group.

A substituted phosphoryl group herein is represented by a formula (P).

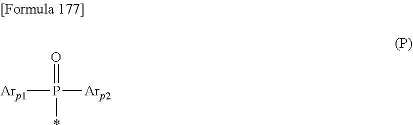

Examples of the ester group herein include a group represented by —C(═O)OR^{E}. R^{E} is exemplified by a substituted or unsubstituted aryl group having 6 to 18 (preferably 6 to 10) ring carbon atoms.

A siloxanyl group herein is a silicon compound group via an ether bond, examples of which include a trimethylsiloxanyl group.

In the formula (P), examples of Ar_{P1} and Ar_{P2} include a substituent selected from the group consisting of an alkyl group having 1 to 30 (preferably 1 to 10, more preferably 1 to 6) carbon atoms, and an aryl group having 6 to 30 (preferably 6 to 20, more preferably 6 to 14) ring carbon atoms. The alkyl group having 1 to 30 carbon atoms is exemplified by the above alkyl groups having 1 to 30 carbon atoms. The aryl group having 6 to 30 ring carbon atoms is exemplified by the above aryl groups having 6 to 30 ring carbon atoms.

Herein, “carbon atoms forming a ring (ring carbon atoms)” mean carbon atoms forming a saturated ring, unsaturated ring, or aromatic ring. “Atoms forming a ring (ring atoms)” mean carbon atoms and hetero atoms forming a ring including a saturated ring, unsaturated ring, or aromatic ring.

Herein, a hydrogen atom includes isotope having different numbers of neutrons, specifically, protium, deuterium and tritium.

Herein, the substituent meant by “substituted or unsubstituted” is at least one group selected from the group consisting of an aryl group having 6 to 30 ring carbon atoms, heteroaryl group having 5 to 30 ring atoms, linear alkyl group having 1 to 30 carbon atoms, branched alkyl group having 3 to 30 carbon atoms, cycloalkyl group having 3 to 30 ring carbon atoms, alkyl halide group having 1 to 30 carbon atoms, substituted or unsubstituted silyl group (e.g. alkylsilyl group having 3 to 30 carbon atoms and arylsilyl group having 6 to 30 ring carbon atoms), alkoxy group having 1 to 30 carbon atoms, aryloxy group having 6 to 30 carbon atoms, substituted or unsubstituted amino group, alkylthio group having 1 to 30 carbon atoms, arylthio group having 6 to 30 ring carbon atoms, aralkyl group having 7 to 30 carbon atoms, alkenyl group having 2 to 30 carbon atoms, halogen atom, alkynyl group having 2 to 30 carbon atoms, cyano group, hydroxy group, nitro group, carboxy group, and substituted phosphoryl group.

It should be noted that the substituent meant by “substituted or unsubstituted” in the third compound does not include a heteroaryl group having 5 to 30 ring atoms and substituted or unsubstituted amino group.

Herein, the substituent meant by “substituted or unsubstituted” is also exemplified by a diaryl boron group (Ar_{B1}Ar_{B2}B—). Examples of Ar_{B1} and Ar_{B2} include the above “aryl group having 6 to 30 ring carbon atoms.”

Examples of the substituent meant by “substituted or unsubstituted” and preferable ones of the examples thereof are the same as the examples of the substituent and the preferable ones of the examples thereof in “Description of Each Substituent.”

The substituent E meant by “substituted or unsubstituted” in the third compound is preferably at least one group selected from the group consisting of an aryl group having 6 to 20 (preferably 6 to 12) ring carbon atoms, a linear alkyl group having 1 to 10 (preferably 1 to 6) carbon atoms, a branched alkyl group having 3 to 10 (preferably 3 to 6) carbon atoms, an alkyl halide group having 1 to 10 (preferably 1 to 6) carbon atoms, a cycloalkyl group having 3 to 20 (preferably 3 to 12) ring carbon atoms, a silyl group, an alkoxy group having 1 to 10 (preferably 1 to 6) carbon atoms, an alkoxy halide group having 1 to 10 (preferably 1 to 6) carbon atoms, an aryloxy group having 6 to 20 (preferably 6 to 12) ring carbon atoms, an alkylthio group having 1 to 10 (preferably 1 to 6) carbon atoms, an arylthio group having 6 to 20 (preferably 6 to 12) ring carbon atoms, an aralkyl group having 7 to 20 (preferably 7 to 12) ring carbon atoms, an alkenyl group having 2 to 10 (preferably 2 to 6) carbon atoms, a halogen atom, an alkynyl group having 2 to 10 (preferably 2 to 6) carbon atoms, a cyano group, a hydroxy group, a nitro group, and a carboxy group, more preferably the specific preferable ones of substituents in the description of the substituents.

When the substituent E meant by “substituted or unsubstituted” in the third compound is further substituted by the substituent F, the substituent F is preferably at least one group selected from the group consisting of an aryl group having 6 to 30 ring carbon atoms, an alkyl group having 1 to 10 (preferably 1 to 6) carbon atoms, an alkyl halide group having 1 to 10 (preferably 1 to 6) carbon atoms, a cycloalkyl group having 3 to 20 (preferably 3 to 12) ring carbon atoms, a silyl group, an alkoxy group having 1 to 10 carbon atoms, an alkoxy halide group having 1 to 10 (preferably 1 to 6) carbon atoms, an aryloxy group having 6 to 20 carbon atoms (preferably 6 to 12 ring carbon atoms), an alkylthio group having 1 to 10 (preferably 1 to 6) carbon atoms, an arylthio group having 6 to 20 (preferably 6 to 12) ring carbon atoms, an aralkyl group having 7 to 20 carbon atoms (preferably 7 to 12 ring carbon atoms), an alkenyl group having 2 to 10 (preferably 2 to 6) carbon atoms, a halogen atom, an alkynyl group having 2 to 10 (preferably 2 to 6) carbon atoms, a cyano group, a hydroxy group, a nitro group, and a carboxy group. In addition, adjacent two or more of the substituents may be bonded to each other to form a ring. It should be noted that the substituent F is not further substituted.

The substituent meant by “substituted or unsubstituted” for the first and second compounds is preferably at least one group selected from the group consisting of an aryl group having 6 to 30 (preferably 6 to 12) ring carbon atoms, heteroaryl group having 5 to 30 (preferably 5 to 12) ring atoms, linear alkyl group having 1 to 30 (preferably 1 to 6) carbon atoms, branched alkyl group having 3 to 30 (preferably 3 to 6) carbon atoms, alkyl halide group having 1 to 30 (preferably 1 to 6) carbon atoms, and cycloalkyl group having 3 to 30 (preferably 3 to 12) ring carbon atoms, further preferably the specific preferable examples described in the description of the substituents.

The substituent meant by “substituted or unsubstituted” for the first and second compounds may be further substituted by at least one group selected from the group consisting of an aryl group having 6 to 30 ring carbon atoms, heteroaryl group having 5 to 30 ring atoms, linear alkyl group having 1 to 30 carbon atoms, branched alkyl group having 3 to 30 carbon atoms, cycloalkyl group having 3 to 30 ring carbon atoms, alkyl halide group having 1 to 30 carbon atoms, alkylsilyl group having 3 to 30 carbon atoms, arylsilyl group having 6 to 30 ring carbon atoms, alkoxy group having 1 to 30 carbon atoms, aryloxy group having 6 to 30 carbon atoms, substituted amino group, alkylthio group having 1 to 30 carbon atoms, arylthio group having 6 to 30 ring carbon atoms, aralkyl group having 7 to 30 carbon atoms, alkenyl group having 2 to 30 carbon atoms, alkynyl group having 2 to 30 carbon atoms, halogen atom, cyano group, hydroxy group, nitro group, and carboxy group. In addition, adjacent two or more of the substituents may be bonded to each other to form a ring.

The substituent to be the substituent for the substituent meant by “substituted or unsubstituted” for the first and second compounds is preferably at least one group selected from the group consisting of an aryl group having 6 to 30 ring carbon atoms, a heteroaryl group having 5 to 30 ring atoms, a linear alkyl group having 1 to 30 carbon atoms, a branched alkyl group having 3 to 30 carbon atoms, a halogen atom, and a cyano group, further preferably the specific preferable examples described in the description of the substituents.

The substituent to be the substituent for the substituent meant by “substituted or unsubstituted” in the first and second compounds may be an acyl group having 2 to 31 carbon atoms.

“Unsubstituted” in “substituted or unsubstituted” means that a group is not substituted by the above-described substituents but bonded with a hydrogen atom.

Herein, “XX to YY carbon atoms” in the description of “substituted or unsubstituted ZZ group having XX to YY carbon atoms” represent carbon atoms of an unsubstituted ZZ group and do not include carbon atoms of a substituent(s) of the substituted ZZ group.

Herein, “XX to YY atoms” in the description of “substituted or unsubstituted ZZ group having XX to YY atoms” represent atoms of an unsubstituted ZZ group and does not include atoms of a substituent(s) of the substituted ZZ group.

The same description as the above applies to “substituted or unsubstituted” in compounds or moieties thereof described herein.

Herein, when the substituents are bonded to each other to form a ring, the ring is structured to be a saturated ring, an unsaturated ring, an aromatic hydrocarbon ring or a hetero ring.

Herein, examples of the aromatic hydrocarbon group and the heterocyclic group in the linking group include a divalent or multivalent group obtained by eliminating one or more atoms from the above monovalent groups.

### EXAMPLES

Example(s) of the invention will be described below. However, the invention is not limited to Example(s).

### Compounds

Compounds used for preparing an organic EL device will be shown below.

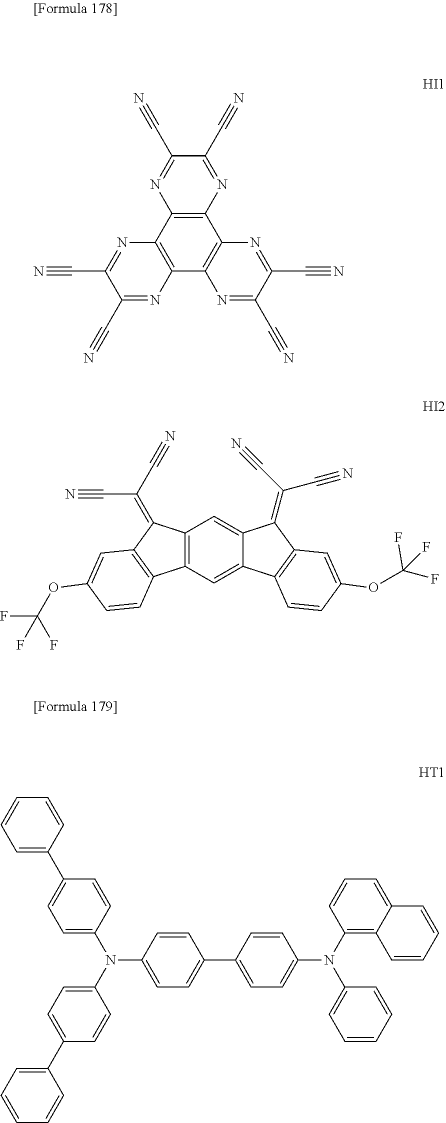 [SMILES: N#Cc1nc2c3nc(C#N)c(C#N)nc3c3nc(C#N)c(C#N)nc3c2nc1C#N ; N#CC(C#N)=C1c2cc(OC(F)(F)F)ccc2-c2cc3c(cc21)C(=C(C#N)C#N)c1cc(OC(F)(F)F)ccc1-3 ; c1ccc(-c2ccc(N(c3ccc(-c4ccccc4)cc3)c3ccc(-c4ccc(N(c5ccccc5)c5cccc6ccccc56)cc4)cc3)cc2)cc1]

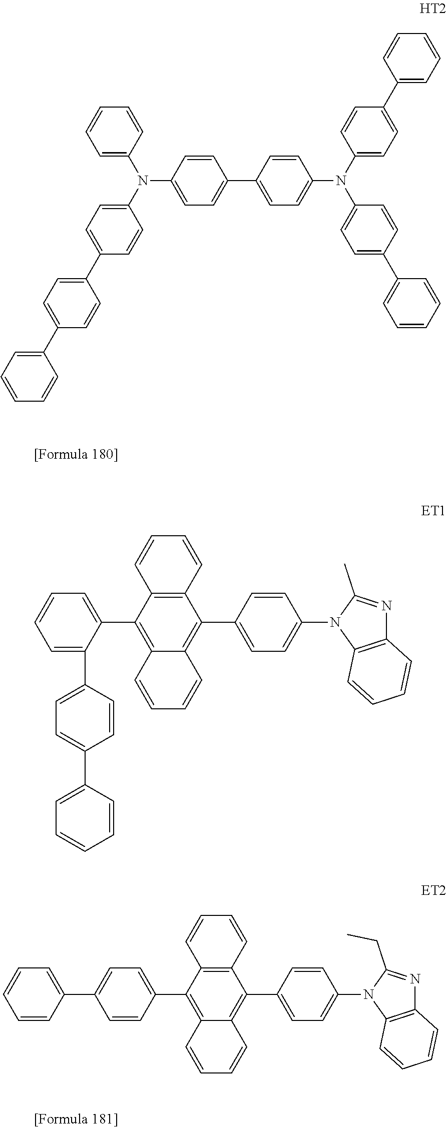 [SMILES: c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; Cc1nc2ccccc2n1-c1ccc(-c2c3ccccc3c(-c3ccccc3-c3ccc(-c4ccccc4)cc3)c3ccccc23)cc1 ; CCc1nc2ccccc2n1-c1ccc(-c2c3ccccc3c(-c3ccc(-c4ccccc4)cc3)c3ccccc23)cc1]

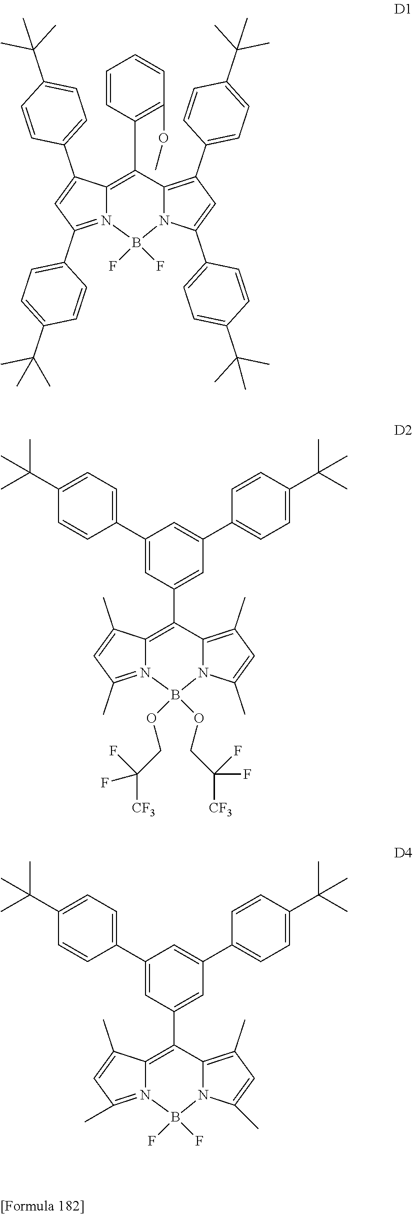

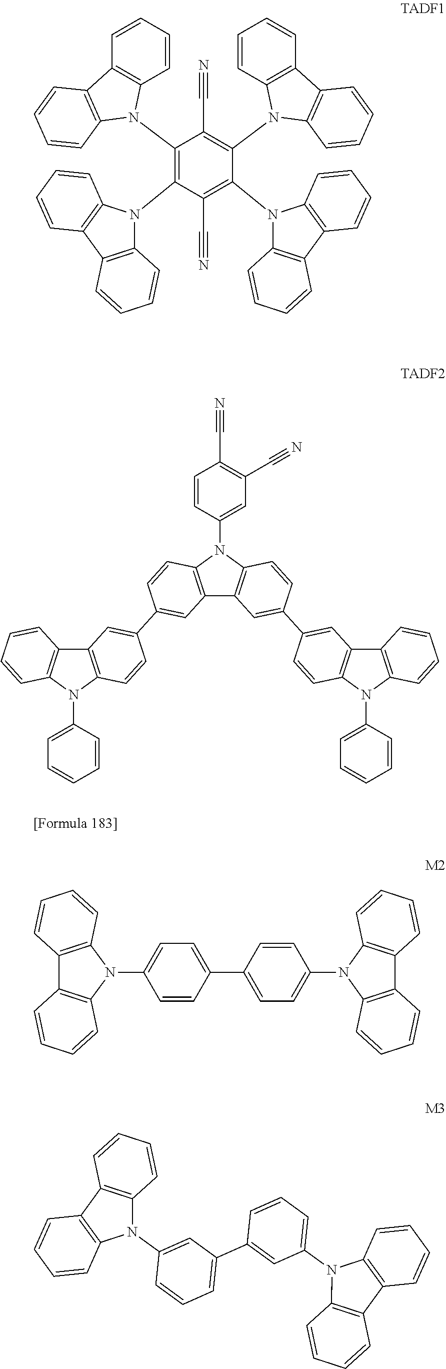 [SMILES: N#Cc1c(-n2c3ccccc3c3ccccc32)c(-n2c3ccccc3c3ccccc32)c(C#N)c(-n2c3ccccc3c3ccccc32)c1-n1c2ccccc2c2ccccc21 ; N#Cc1ccc(-n2c3ccc(-c4ccc5c(c4)c4ccccc4n5-c4ccccc4)cc3c3cc(-c4ccc5c(c4)c4ccccc4n5-c4ccccc4)ccc32)cc1C#N ; c1ccc2c(c1)c1ccccc1n2-c1ccc(-c2ccc(-n3c4ccccc4c4ccccc43)cc2)cc1 ; c1cc(-c2cccc(-n3c4ccccc4c4ccccc43)c2)cc(-n2c3ccccc3c3ccccc32)c1]

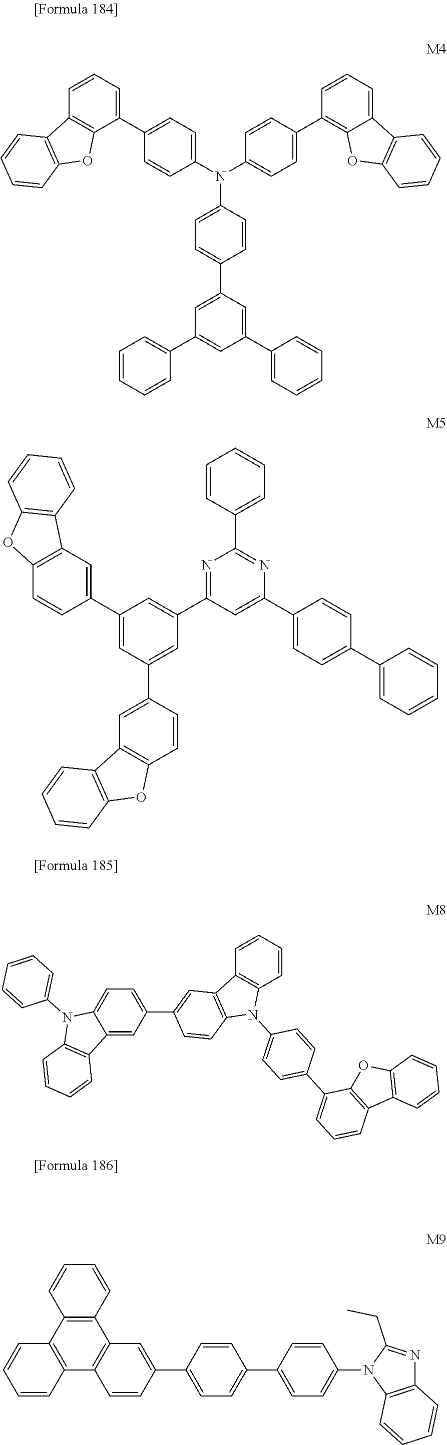 [SMILES: c1ccc(-c2cc(-c3ccccc3)cc(-c3ccc(N(c4ccc(-c5cccc6c5oc5ccccc56)cc4)c4ccc(-c5cccc6c5oc5ccccc56)cc4)cc3)c2)cc1 ; c1ccc(-c2ccc(-c3cc(-c4cc(-c5ccc6oc7ccccc7c6c5)cc(-c5ccc6oc7ccccc7c6c5)c4)nc(-c4ccccc4)n3)cc2)cc1 ; c1ccc(-n2c3ccccc3c3cc(-c4ccc5c(c4)c4ccccc4n5-c4ccc(-c5cccc6c5oc5ccccc56)cc4)ccc32)cc1 ; CCc1nc2ccccc2n1-c1ccc(-c2ccc(-c3ccc4c5ccccc5c5ccccc5c4c3)cc2)cc1]

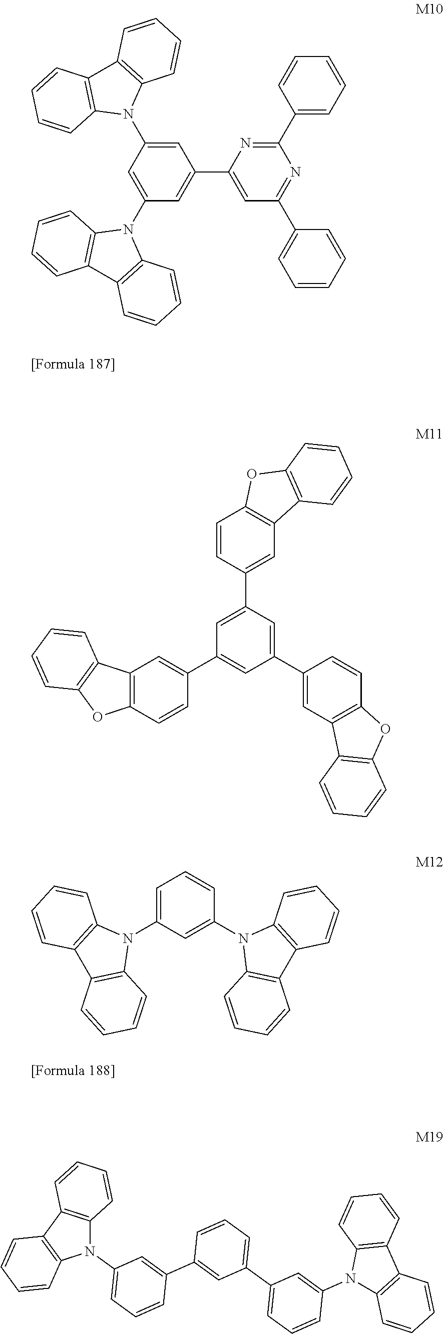 [SMILES: c1ccc(-c2cc(-c3cc(-n4c5ccccc5c5ccccc54)cc(-n4c5ccccc5c5ccccc54)c3)nc(-c3ccccc3)n2)cc1 ; c1ccc2c(c1)oc1ccc(-c3cc(-c4ccc5oc6ccccc6c5c4)cc(-c4ccc5oc6ccccc6c5c4)c3)cc12 ; c1cc(-n2c3ccccc3c3ccccc32)cc(-n2c3ccccc3c3ccccc32)c1 ; c1cc(-c2cccc(-n3c4ccccc4c4ccccc43)c2)cc(-c2cccc(-n3c4ccccc4c4ccccc43)c2)c1]

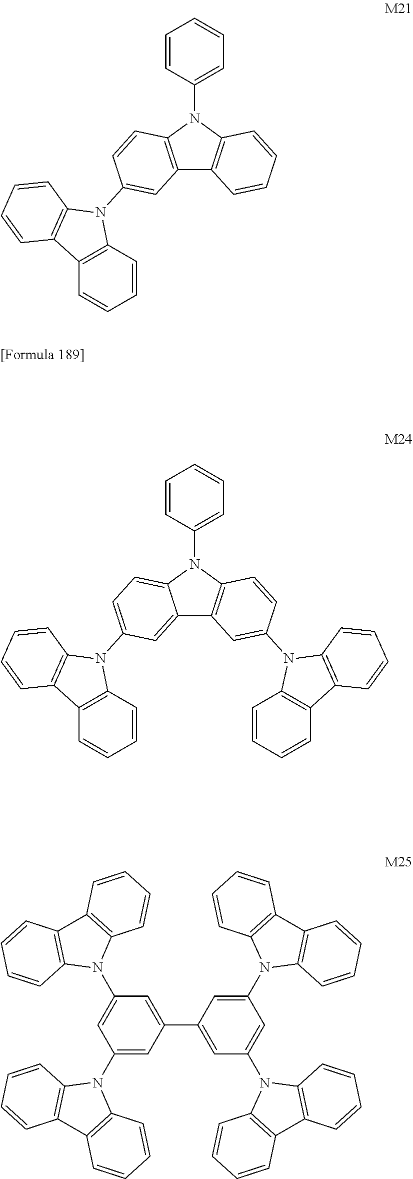 [SMILES: c1ccc(-n2c3ccccc3c3cc(-n4c5ccccc5c5ccccc54)ccc32)cc1 ; c1ccc(-n2c3ccc(-n4c5ccccc5c5ccccc54)cc3c3cc(-n4c5ccccc5c5ccccc54)ccc32)cc1 ; c1ccc2c(c1)c1ccccc1n2-c1cc(-c2cc(-n3c4ccccc4c4ccccc43)cc(-n3c4ccccc4c4ccccc43)c2)cc(-n2c3ccccc3c3ccccc32)c1]

### Delayed Fluorescence

### Delayed Fluorescence Properties of Compound TADF1

Delayed fluorescence properties were checked by measuring transient photoluminescence (PL) using a device shown in FIG. 2. A sample was prepared by co-depositing the compounds TADF1 and TH-2 on a quartz substrate at a ratio of the compound TADF1 of 12 mass % to form a 100-nm-thick thin film. Prompt emission was observed immediately when the excited state was achieved by exciting the compound TADF1 with a pulse beam (i.e., a beam emitted from a pulse laser) having a wavelength to be absorbed by the compound TADF1, and Delay emission was observed not immediately when the excited state was achieved but after the excited state was achieved. The delayed fluorescence in Examples means that an amount of Delay Emission is 5% or more with respect to an amount of Prompt Emission. Specifically, provided that the amount of Prompt emission is denoted by X_{P} and the amount of Delay emission is denoted by X_{D}, a value of X_{D}/X_{P} is 0.05 or more.

It was found that the amount of Delay Emission was 5% or more with respect to the amount of Prompt Emission in the compound TADF1. Specifically, it was found that the value of X_{D}/X_{P} was 0.05 or more in the compound TADF1.

The amount of Prompt Emission and the amount of Delay Emission can be obtained according to the same method as a method described in “Nature 492, 234-238, 2012.” A device used for calculating the amounts of Prompt Emission and Delay Emission is not limited to the device of FIG. 2 and a device described in the above document.

### Delayed Fluorescence Properties of Compound TADF2

Delayed fluorescence properties of the compound TADF2 were checked in the same manner as in the above except for the use of the compound TADF2 instead of the use of the compound TADF1.

As a result, it was found that the amount of Delay Emission was 5% or more with respect to the amount of Prompt Emission in the compound TADF2. Specifically, it was found that the value of X_{D}/X_{P} was 0.05 or more in the compound TADF2.

### Singlet Energy S₁

Singlet energies S₁ of the compounds TADF1, TADF2, D1, D2, D4, M2, M3, M9, M11, M12, and M21 were measured according to the above-described solution method.

The singlet energy S₁ of the compound TADF1 was 2.37 eV.

The singlet energy S₁ of the compound TADF2 was 2.80 eV.

The singlet energy S₁ of the compound D1 was 2.02 eV.

The singlet energy S₁ of the compound D2 was 2.00 eV.

The singlet energy S₁ of the compound D4 was 2.39 eV.

The singlet energy S₁ of the compound M2 was 3.52 eV.

The singlet energy S₁ of the compound M3 was 3.56 eV.

The singlet energy S₁ of the compound M9 was 3.51 eV.

The singlet energy S₁ of the compound M11 was 3.75 eV.

The singlet energy S₁ of the compound M12 was 3.40 eV.

The singlet energy S₁ of the compound M21 was 3.44 eV.

### Energy Gap T_{77K} at 77[K]

The energy gap T_{77K} of the compound TADF2 was measured by the above-described measurement method of the energy gap T_{77K}.

T_{77K} of the compound TADF2 was 2.71 eV. Accordingly, ΔST of the compound TADF2 was 0.09 eV.

### Main Peak Wavelength of Compounds

A 5 μmol/L toluene solution of a measurement target compound was prepared and put in a quartz cell. A fluorescence spectrum (ordinate axis: fluorescence intensity, abscissa axis: wavelength) of the thus-obtained sample was measured at a normal temperature (300K). In Examples, the fluorescence spectrum was measured using a spectrophotometer (F-7000 manufactured by Hitachi, Ltd.). It should be noted that the fluorescence spectrum measuring device may be different from the above device. A peak wavelength of the fluorescence spectrum exhibiting the maximum luminous intensity was defined as a main peak wavelength.

The main peak wavelength of the compound D1 was 609 nm.

The main peak wavelength of the compound D2 was 613 nm.

The main peak wavelength of the compound D4 was 516 nm.

### Preparation of Red-Emitting Organic EL Device A

A red-emitting organic EL device (sometimes referred to as a red-emitting device hereinafter) was prepared and evaluated as follows.

### Example 1-1

A glass substrate (size: 25 mm×75 mm×1.1 mm thick, manufactured by Geomatec Co., Ltd.) having an ITO transparent electrode (anode) was ultrasonic-cleaned in isopropyl alcohol for five minutes, and then UV/ozone-cleaned for 1 minute. A film of ITO was 130 nm thick.

After the glass substrate having the transparent electrode line was cleaned, the glass substrate was mounted on a substrate holder of a vacuum deposition apparatus. Initially, the compound HI1 was vapor-deposited on a surface of the glass substrate where the transparent electrode line was provided in a manner to cover the transparent electrode, thereby forming a 5-nm-thick hole injecting layer.

Next, the compound HT1 was vapor-deposited on the hole injecting layer to form a 55-nm-thick hole transporting layer on the HI1 film.

Then, the compound M4 was vapor-deposited on the hole transporting layer to form a 10-nm-thick electron blocking layer.

Subsequently, the compound D1 (first compound), the compound TADF1 (second compound) and the compound M3 (third compound) were co-deposited on the electron blocking layer to form a 25-nm-thick emitting layer (first organic layer). A concentration of the compound TADF1 was 10 mass %, a concentration of the compound D1 was 0.5 mass %, and a concentration of the compound M3 was 89.5 mass % in the emitting layer.

Next, the compound M5 was vapor-deposited on the emitting layer to form a 10-nm-thick first electron transporting layer.

The compound ET1 was then vapor-deposited on the first electron transporting layer to form a 30-nm-thick second electron transporting layer.

Next, lithium fluoride (LiF) was vapor-deposited on the second electron transporting layer to form a 1-nm-thick electron injecting electrode (cathode).

Metal aluminum (Al) was then vapor-deposited on the electron injecting electrode to form an 80-nm-thick metal Al cathode.

A device arrangement of the red-emitting device in Example 1-1 is schematically shown as follows.

ITO(130)/HI1(5)/HT1(55)/M4(10)/M3:TADF1:D1(25, 89.5%:10%:0.5%)/M5(1 0)/ET1(30)/LiF(1)/Al(80)

Numerals in parentheses each represent a film thickness (unit: nm). The numerals represented by percentage in the same parentheses each indicate a ratio (mass %) of the third compound, the second compound and the first compound in the emitting layer. The above is also applicable in the description below.

### Example 1-2

A red-emitting device of Example 1-2 was prepared in the same manner as in Example 1-1 except that the compound M2 was used in place of the compound M3 in the emitting layer of Example 1-1.

A device arrangement of the red-emitting device in Example 1-2 is schematically shown as follows.

ITO(130)/HI1(5)/HT1(55)/M4(10)/M2:TADF1:D1(25, 89.5%:10%:0.5%)/M5(1 0)/ET1(30)/LiF(1)/Al(80)

### Example 1-3

A red-emitting device of Example 1-3 was prepared in the same manner as in Example 1-1 except that the compound M19 was used in place of the compound M3 in the emitting layer of Example 1-1.

A device arrangement of the red-emitting device in Example 1-3 is schematically shown as follows.

ITO(130)/HI1(5)/HT1(55)/M4(10)/M19:TADF1:D1(25,89.5%:10%:0.5%)/M5(10)/ET1(30)/LiF(1)/Al(80)

### Example 1-4

A red-emitting device of Example 1-4 was prepared in the same manner as in Example 1-1 except that the compound M12 was used in place of the compound M3 in the emitting layer of Example 1-1.

A device arrangement of the red-emitting device in Example 1-4 is schematically shown as follows.

ITO(130)/HI1(5)/HT1(55)/M4(10)/M12:TADF1:D1(25,89.5%:10%:0.5%)/M5(10)/ET1(30)/LiF(1)/Al(80)

### Example 1-5

A red-emitting device of Example 1-5 was prepared in the same manner as in Example 1-1 except that the concentrations of the compound M3 and TADF1, which were used in the emitting layer of Example 1-1, were 74.5 mass % and 25%, respectively.

A device arrangement of the red-emitting device in Example 1-5 is schematically shown as follows.

ITO(130)/HI1(5)/HT1(55)/M4(10)/M3:TADF1:D1(25,74.5%:25%:0.5%)/M5(1 0)/ET1(30)/LiF(1)/Al(80)

### Example 1-6

A red-emitting device of Example 1-6 was prepared in the same manner as in Example 1-5 except that the compound M2 was used in place of the compound M3 in the emitting layer of Example 1-5.

A device arrangement of the red-emitting device in Example 1-6 is schematically shown as follows.

ITO(130)/HI1(5)/HT1(55)/M4(10)/M2:TADF1:D1(25,74.5%:25%:0.5%)/M5(1 0)/ET1(30)/LiF(1)/Al(80)

### Example 1-7

A red-emitting device of Example 1-7 was prepared in the same manner as in Example 1-5 except that the compound M19 was used in place of the compound M3 in the emitting layer of Example 1-5.

A device arrangement of the red-emitting device in Example 1-7 is schematically shown as follows.

ITO(130)/HI1(5)/HT1(55)/M4(10)/M19:TADF1:D1(25,74.5%:25%:0.5%)/M5(10)/ET1(30)/LiF(1)/Al(80)

### Example 1-8

A red-emitting device of Example 1-8 was prepared in the same manner as in Example 1-5 except that the compound M12 was used in place of the compound M3 in the emitting layer of Example 1-5.

A device arrangement of the red-emitting device in Example 1-8 is schematically shown as follows.

ITO(130)/HI1(5)/HT1(55)/M4(10)/M12:TADF1:D1(25,74.5%:25%:0.5%)/M5(10)/ET1(30)/LiF(1)/Al(80)

### Comparative 1-1

A red-emitting device of Comparative 1-1 was prepared in the same manner as in Example 1-1 except that the compound M11 was used in place of the compound M3 in the emitting layer of Example 1-1.

A device arrangement of the red-emitting device in Comparative 1-1 is schematically shown as follows.

ITO(130)/HI1(5)/HT1(55)/M4(10)/M11:TADF1:D1(25,89.5%:10%:0.5%)/M5(10)/ET1(30)/LiF(1)/Al(80)

### Comparative 1-2

A red-emitting device of Comparative 1-2 was prepared in the same manner as in Example 1-1 except that the compound M9 was used in place of the compound M3 in the emitting layer of Example 1-1.

A device arrangement of the red-emitting device in Comparative 1-2 is schematically shown as follows.

ITO(130)/HI1(5)/HT1(55)/M4(10)/M9:TADF1:D1(25,89.5%:10%:0.5%)/M5(1 0)/ET1(30)/LiF(1)/Al(80)

### Comparative 1-3

A red-emitting device of Comparative 1-3 was prepared in the same manner as in Example 1-5 except that the compound M11 was used in place of the compound M3 in the emitting layer of Example 1-5.

A device arrangement of the red-emitting device in Comparative 1-3 is schematically shown as follows.

ITO(130)/HI1(5)/HT1(55)/M4(10)/M11:TADF1:D1(25,74.5%:25%:0.5%)/M5(10)/ET1(30)/LiF(1)/Al(80)

### Comparative 1-4

A red-emitting device of Comparative 1-4 was prepared in the same manner as in Example 1-5 except that the compound M9 was used in place of the compound M3 in the emitting layer of Example 1-5.

A device arrangement of the red-emitting device in Comparative 1-4 is schematically shown as follows.

ITO(130)/HI1(5)/HT1(55)/M4(10)/M9:TADF1:D1(25,74.5%:25%:0.5%)/M5(1 0)/ET1(30)/LiF(1)/Al(80)

### Evaluation of Red-Emitting Device

The red-emitting devices prepared in Examples 1-1 to 1-8 and Comparatives 1-1 to 1-4 were evaluated as follows. The evaluation results are shown in Tables 14 and 15. In Tables 14 and 15, % means “mass %”, @0.1 means 0.1 mA/cm², and @10 means 10 mA/cm².

### External Quantum Efficiency EQE

Voltage was applied on each of the organic EL devices such that a current density was 0.1 mA/cm², where spectral radiance spectra were measured by a spectroradiometer CS-2000 (manufactured by Konica Minolta, Inc.).

The external quantum efficiency EQE (unit: %) was calculated based on the obtained spectral-radiance spectra, assuming that the spectra were provided under a Lambertian radiation.

Further, voltage was applied on the device such that the current density became 10 mA/cm² and the external quantum efficiency EQE (unit: %) was calculated in the same manner as the above.

### Values of Chromaticities CIEx, CIEy, and Main Peak Wavelength λₚ

Voltage was applied on each of the organic EL devices such that a current density was 10 mA/cm², where spectral radiance spectra were measured by a spectroradiometer CS-2000 (manufactured by Konica Minolta, Inc.). The chromaticities CIEx and CIEy and main peak wavelength λₚ (unit: nm) were calculated based on the obtained spectral-radiance spectra.

TABLE 14

| | First organic layer | | | Evaluation | | | | |
|---|---|---|---|---|---|---|---|---|
| | 1st | 2nd | 3rd | EQE | EQE | | | λ p |
| | com- | com- | com- | (%) | (%) | CIEx | CIE y | (nm) |
| | pound | pound | pound | @ 0.1 | @ 10 | @ 10 | @ 10 | @ 10 |
| Ex: | D1 | TADF1 | Mat 3 | 20.1 | 16.0 | 0.610 | 0.387 | 616 |
| 1-1 | (0.5%) | (10%) | (89.5%) | | | | | |
| Ex | D1 | TADF1 | Mat 2 | 21.0 | 17.6 | 0.622 | 0.377 | 618 |
| 1-2 | (0.5%) | (10%) | (89.5%) | | | | | |
| Ex | D1 | TADF1 | Mat 19 | 18.5 | 15.5 | 0.631 | 0.367 | 618 |
| 1-3 | (0.5%) | (10%) | (89.5%) | | | | | |
| Ex | D1 | TADF1 | Mat 12 | 18.5 | 15.0 | 0.635 | 0.364 | 617 |
| 1-4 | (0.5%) | (10%) | (89.5%) | | | | | |
| Comp. | D1 | TADF1 | Mat11 | 12.0 | 10.7 | 0.626 | 0.367 | 617 |
| 1-1 | (0.5%) | (10%) | (89.5%) | | | | | |
| Comp. | D1 | TADF1 | Mat9 | 15.6 | 13.4 | 0.626 | 0.369 | 617 |
| 1-2 | (0.5%) | (10%) | (89.5%) | | | | | |

The red-emitting devices according to Examples 1-1 to 1-4, whose first organic layer contained the first compound represented by the formula (1), the delayed fluorescent second compound, and the third compound represented by the formula (3), exhibited higher value of external quantum efficiency than those of devices of Comparatives 1-1 to 1-2 whose first organic layer contained the compound not satisfying the formula (3) in place of the third compound, at the current density of both of 0.1 mA/cm² and 10 mA/cm².

It is thus found that the red-emitting devices of the Examples highly efficiently emitted light.

TABLE 15

| | First organic layer | | | Evaluation | | | | |
|---|---|---|---|---|---|---|---|---|
| | 1st | 2nd | 3rd | EQE | EQE | | | λ p |
| | com- | com- | com- | (%) | (%) | CIEx | CIE y | (nm) |
| | pound | pound | pound | @ 0.1 | @ 10 | @ 10 | @ 10 | @ 10 |
| Ex. | D1 | TADF1 | M3 | 15.8 | 15.4 | 0.639 | 0.360 | 617 |
| 1-5 | (0.5%) | (25%) | (74.5%) | | | | | |
| Ex. | D1 | TADF1 | M2 | 16.4 | 17.3 | 0.642 | 0.357 | 619 |
| 1-6 | (0.5%) | (25%) | (74.5%) | | | | | |
| Ex. | D1 | TADF1 | M19 | 13.6 | 14.1 | 0.638 | 0.361 | 617 |
| 1-7 | (0.5%) | (25%) | (74.5%) | | | | | |
| Ex. | D1 | TADF1 | M1 | 14.9 | 14.7 | 0.642 | 0.358 | 617 |
| 1-8 | (0.5%) | (25%) | (74.5%) | | | | | |
| Comp. | D1 | TADF1 | M11 | 19.2 | 8.8 | 0.639 | 0.359 | 618 |
| 1-3 | (0.5%) | (25%) | (74.5%) | | | | | |
| Comp. | D1 | TADF1 | M9 | 10.8 | 8.8 | 0.642 | 0.356 | 618 |
| 1-4 | (0.5%) | (25%) | (74.5%) | | | | | |

The red-emitting devices according to Examples 1-5 to 1-8, whose first organic layer contained the first compound represented by the formula (1), the delayed fluorescent second compound, and the third compound represented by the formula (3), exhibited higher value of external quantum efficiency than those of devices of Comparatives 1-3 to 1-4 whose first organic layer contained the compound not satisfying the formula (3) in place of the third compound, at the current density of both of 0.1 mA/cm² and 10 mA/cm².

It is thus found that the red-emitting devices of the Examples highly efficiently emitted light.

### Preparation of Green-Emitting Organic EL Device

A green-emitting organic EL device (sometimes referred to as green-emitting device hereinafter) was prepared and evaluated as follows.

### Example 2-1

A glass substrate (size: 25 mm×75 mm×1.1 mm thick, manufactured by Geomatec Co., Ltd.) having an ITO transparent electrode (anode) was ultrasonic-cleaned in isopropyl alcohol for five minutes, and then UV/ozone-cleaned for 1 minute. A film of ITO was 130 nm thick.

After the glass substrate having the transparent electrode line was cleaned, the glass substrate was mounted on a substrate holder of a vacuum deposition apparatus. Initially, the compound H12 was vapor-deposited on a surface of the glass substrate where the transparent electrode line was provided in a manner to cover the transparent electrode, thereby forming a 5-nm-thick hole injecting layer.

Next, the compound HT2 was vapor-deposited on the hole injecting layer to form a 110-nm-thick hole transporting layer on the H12 film.

Next, the compound M8 was vapor-deposited on the hole transporting layer to form a 15-nm-thick electron blocking layer.

Subsequently, the compound D4 (first compound), the compound TADF2 (second compound) and the compound M3 (third compound) were co-deposited on the electron blocking layer to form a 25-nm-thick emitting layer (first organic layer). A concentration of the compound TADF2 was 25 mass %, a concentration of the compound D4 was 0.2 mass %, and a concentration of the compound M3 was 74.8 mass % in the emitting layer.

Next, the compound M10 was vapor-deposited on the emitting layer to form a 5-nm-thick first electron transporting layer.

The compound ET2 was then vapor-deposited on the first electron transporting layer to form a 50-nm-thick second electron transporting layer.

Next, lithium fluoride (LiF) was vapor-deposited on the second electron transporting layer to form a 1-nm-thick electron injecting electrode (cathode).

Metal aluminum (Al) was then vapor-deposited on the electron injecting electrode to form an 80-nm-thick metal Al cathode.

A device arrangement of the green-emitting device in Example 2-1 is schematically shown as follows.

ITO(130)/H12(5)/HT2(110)/M8(15)/M3:TADF2:D4(25,74.8%:25%:0.2%)/M10(5)/ET2(50)/LiF(1)/Al(80)

Numerals in parentheses each represent a film thickness (unit: nm). The numerals represented by percentage in the same parentheses each indicate a ratio (mass %) of the third compound, the second compound and the first compound in the emitting layer. The above is also applicable in the description below.

### Example 2-2

A green-emitting device of Example 2-2 was prepared in the same manner as in Example 2-1 except that the compound M21 was used in place of the compound M3 in the emitting layer of Example 2-1.

A device arrangement of the green-emitting device in Example 2-2 is schematically shown as follows.

ITO(130)/H12(5)/HT2(110)/M8(15)/M21:TAD F2:D4(25,74.8%:25%:0.2%)/M 10(5)/ET2(50)/LiF(1)/Al(80)

### Example 2-3

A green-emitting device of Example 2-3 was prepared in the same manner as in Example 2-1 except that the compound D2 was used in place of the compound D4 in the emitting layer of Example 2-1.

A device arrangement of the green-emitting device in Example 2-3 is schematically shown as follows.

ITO(130)/H12(5)/HT2(110)/M8(15)/M3:TADF2:D2(25,74.8%:25%:0.2%)/M10(5)/ET2(50)/LiF(1)/Al(80)

### Example 2-X

A green-emitting device of Example 2-X was prepared in the same manner as in Example 2-1 except that the compound M12 was used in place of the compound M3 in the emitting layer of Example 2-1.

A device arrangement of the green-emitting device in Example 2-X is schematically shown as follows.

ITO(130)/H12(5)/HT2(110)/M8(15)/M12:TADF2:D4(25,74.8%:25%:0.2%)/M 10(5)/ET2(50)/LiF(1)/Al(80)

### Example 2-Y

A green-emitting device of Example 2-Y was prepared in the same manner as in Example 2-1 except that the compound M24 was used in place of the compound M3 in the emitting layer of Example 2-1.

A device arrangement of the green-emitting device in Example 2-Y is schematically shown as follows.

ITO(130)/H12(5)/HT2(110)/M8(15)/M24:TADF2:D4(25,74.8%:25%:0.2%)/M 10(5)/ET2(50)/LiF(1)/Al(80)

### Example 2-Z

A green-emitting device of Example 2-Z was prepared in the same manner as in Example 2-1 except that the compound M25 was used in place of the compound M3 in the emitting layer of Example 2-1.

A device arrangement of the green-emitting device in Example 2-Z is schematically shown as follows.

ITO(130)/H12(5)/HT2(110)/M8(15)/M25:TADF2:D4(25,74.8%:25%:0.2%)/M 10(5)/ET2(50)/LiF(1)/Al(80)

A green-emitting device of Example 2-Q was prepared in the same manner as in Example 2-1 except that the compound M2 was used in place of the compound M3 in the emitting layer of Example 2-1.

A device arrangement of the green-emitting device in Example 2-Q is schematically shown as follows.

ITO(130)/H12(5)/HT2(110)/M8(15)/M2:TADF2:D4(25,74.8%:25%:0.2%)/M10(5)/ET2(50)/LiF(1)/Al(80)

### Example 2-4

A green-emitting device of Example 2-4 was prepared in the same manner as in Example 2-1 except that the concentrations of the compound M3 and TADF2, which were used in the emitting layer of Example 2-1, were 49.8 mass % and 50 mass %, respectively.

A device arrangement of the green-emitting device in Example 2-4 is schematically shown as follows.

ITO(130)/H12(5)/HT2(110)/M8(15)/M3:TADF2:D4(25,49.8%:50%:0.2%)/M10(5)/ET2(50)/LiF(1)/Al(80)

### Example 2-5

A green-emitting device of Example 2-5 was prepared in the same manner as in Example 2-4 except that the compound M21 was used in place of the compound M3 in the emitting layer of Example 2-4.

A device arrangement of the green-emitting device in Example 2-5 is schematically shown as follows.

ITO(130)/H12(5)/HT2(110)/M8(15)/M21:TADF2:D4(25,49.8%:50%:0.2%)/M 10(5)/ET2(50)/LiF(1)/Al(80)

### Example 2-6

A green-emitting device of Example 2-6 was prepared in the same manner as in Example 2-4 except that the compound D2 was used in place of the compound D4 in the emitting layer of Example 2-4.

A device arrangement of the green-emitting device in Example 2-6 is schematically shown as follows.

ITO(130)/H12(5)/HT2(110)/M8(15)/M3:TADF2:D2(25,49.8%:50%:0.2%)/M10(5)/ET2(50)/LiF(1)/Al(80)

### Example 2-A

A green-emitting device of Example 2-A was prepared in the same manner as in Example 2-4 except that the compound M12 was used in place of the compound M3 in the emitting layer of Example 2-4.

A device arrangement of the green-emitting device in Example 2-A is schematically shown as follows.

ITO(130)/H12(5)/HT2(110)/M8(15)/M12:TADF2:D4(25,49.8%: 50%: 0.2%)/M 10(5)/ET2(50)/LiF(1)/Al(80)

### Example 2-B

A green-emitting device of Example 2-B was prepared in the same manner as in Example 2-4 except that the compound M24 was used in place of the compound M3 in the emitting layer of Example 2-4.

A device arrangement of the green-emitting device in Example 2-B is schematically shown as follows.

ITO(130)/H12(5)/HT2(110)/M8(15)/M24:TADF2:D4(25,49.8%:50%:0.2%)/M 10(5)/ET2(50)/LiF(1)/Al(80)

### Example 2-C

A green-emitting device of Example 2-C was prepared in the same manner as in Example 2-4 except that the compound M25 was used in place of the compound M3 in the emitting layer of Example 2-4.

A device arrangement of the green-emitting device in Example 2-C is schematically shown as follows.

ITO(130)/H12(5)/HT2(110)/M8(15)/M25:TADF2:D4(25,49.8%:50%:0.2%)/M 10(5)/ET2(50)/LiF(1)/Al(80)

### Comparative 2-1

A green-emitting device of Comparative 2-1 was prepared in the same manner as in Example 2-1 except that the compound M11 was used in place of the compound M3 in the emitting layer of Example 2-1.

A device arrangement of the green-emitting device in Comparative 2-1 is schematically shown as follows.

ITO(130)/H12(5)/HT2(110)/M8(15)/M11:TADF2:D4(25,74.8%:25%:0.2%)/M 10(5)/ET2(50)/LiF(1)/Al(80)

### Comparative 2-2

A green-emitting device of Comparative 2-2 was prepared in the same manner as in Example 2-4 except that the compound M11 was used in place of the compound M3 in the emitting layer of Example 2-4.

A device arrangement of the green-emitting device in Comparative 2-2 is schematically shown as follows.

ITO(130)/H12(5)/HT2(110)/M8(15)/M11:TADF2:D4(25,49.8%:50%:0.2%)/M 10(5)/ET2(50)/LiF(1)/Al(80)

### Evaluation of Green-Emitting Device

The green-emitting devices prepared in Examples 2-1 to 2-6, Examples 2-X to 2-Z, Example 2-Q, Examples 2-A to 2-C, and Comparatives 2-1 to 2-2 were evaluated as follows. The evaluation results are shown in Tables 16 and 17. In Tables 16 and 17, % means “mass %”, 0.1 means @0.1 mA/cm², and @10 means 10 mA/cm².

### External Quantum Efficiency EQE

Voltage was applied on each of the organic EL devices such that a current density was 0.1 mA/cm², where spectral radiance spectra were measured by a spectroradiometer CS-2000 (manufactured by Konica Minolta, Inc.).

The external quantum efficiency EQE (unit: %) was calculated based on the obtained spectral-radiance spectra, assuming that the spectra were provided under a Lambertian radiation.

### Values of Chromaticities CIEx, CIEy, and Main Peak Wavelength λₚ

Voltage was applied on each of the organic EL devices such that a current density was 10 mA/cm², where spectral radiance spectra were measured by a spectroradiometer CS-2000 (manufactured by Konica Minolta, Inc.). The chromaticities CIEx and CIEy and main peak wavelength λₚ (unit: nm) were calculated based on the obtained spectral-radiance spectra.

TABLE 16

| | First organic layer | | | Evaluation | | | |
|---|---|---|---|---|---|---|---|
| | 1st | 2nd | 3rd | EQE | | | λ p |
| | com- | com- | com- | (%) | CIEx | CIE y | (nm) |
| | pound | pound | pound | @ 0.1 | @ 10 | @ 10 | @ 10 |
| Ex. | D4 | TADF2 | M3 | 15.7 | 0.284 | 0.583 | 519 |
| 2-1 | (0.2%) | (25%) | (74.8%) | | | | |
| Ex. | D4 | TADF2 | M21 | 15.6 | 0.284 | 0.625 | 520 |
| 2-2 | (0.2%) | (25%) | (74.8%) | | | | |
| Ex. | D2 | TADF2 | M3 | 14.0 | 0.300 | 0.604 | 518 |
| 2-3 | (0.2%) | (25%) | (74.8%) | | | | |
| Ex. | D4 | TADF2 | M3 | 15.1 | 0.259 | 0.617 | 519 |
| 2-X | (0.2%) | (25%) | (74.8%) | | | | |
| Ex. | D4 | TADF2 | M24 | 14.9 | 0.302 | 0.619 | 521 |
| 2-Y | (0.2%) | (25%) | (74.8%) | | | | |
| Ex. | D4 | TADF2 | M25 | 14.9 | 0.297 | 0.621 | 522 |
| 2-Z | (0.2%) | (25%) | (74.8%) | | | | |
| Ex. | D4 | TADF2 | M2 | 13.6 | 0.278 | 0.621 | 520 |
| 2-Q | (0.2%) | (25%) | (74.8%) | | | | |
| Comp. | D4 | TADF2 | M11 | 11.4 | 0.317 | 0.618 | 523 |
| 2-1 | (0.2%) | (25%) | (74.8%) | | | | |

The green-emitting devices according to Examples 2-1 to 2-3, Examples 2-X to 2-Z, and Example 2-Q, whose first organic layer contained the first compound represented by the formula (1), the delayed fluorescent second compound, and the third compound represented by the formula (3), exhibited higher value of external quantum efficiency than those of devices of Comparative 2-1 whose first organic layer contained the compound not satisfying the formula (3) in place of the third compound.

It is thus found that the green-emitting devices of the Examples highly efficiently emitted light.

TABLE 17

| | First organic layer | | | Evaluation | | | |
|---|---|---|---|---|---|---|---|
| | 1st | 2nd | 3rd | | | | λ p |
| | com- | com- | com- | DEM | CIEx | CIE y | (nm) |
| | pound | pound | pound | @ 0.1 | @ 10 | @ 10 | @ 10 |
| Ex. | D4 | TADF2 | M3 | 13.1 | 0.325 | 0.613 | 521 |
| 2-4 | (0.2%) | (50%) | (49.8%) | | | | |
| Ex. | D4 | TADF2 | M21 | 12.3 | 0.314 | 0.626 | 521 |
| 2-5 | (0.2%) | (50%) | (49.80) | | | | |
| Ex. | D2 | TADF2 | M3 | 11.2 | 0.341 | 0.604 | 521 |
| 2-6 | (0.2%) | (5 0%) | (49.8%) | | | | |
| Ex. | D4 | TADF2 | M12 | 12.8 | 0.316 | 0.623 | 521 |
| 2-A | (0.2%) | (50%) | (49.8%) | | | | |
| Ex. | D4 | TADF2 | M24 | 11.3 | 0.344 | 0.610 | 524 |
| 2-B | (0.2%) | (50%) | (49.8%) | | | | |
| Ex. | D4 | TADF2 | M25 | 11.3 | 0.397 | 0.621 | 593 |
| 2-C | (0.2%) | (50%) | (49.8 %) | | | | |
| Comp. | D4 | TADF2 | M11 | 10.0 | 0.347 | 0.608 | 525 |
| 2-2 | (0.2%) | (50%) | (49.8%) | | | | |

The green-emitting devices according to Examples 2-4 to 2-6 and Examples 2-A to 2-C, whose first organic layer contained the first compound represented by the formula (1), the delayed fluorescent second compound, and the third compound represented by the formula (3), exhibited higher value of external quantum efficiency than those of devices of Comparative 2-2 whose first organic layer contained the compound not satisfying the formula (3) in place of the third compound.

It is thus found that the green-emitting devices of the Examples highly efficiently emitted light.

### EXPLANATION OF CODE(S)

1 . . . organic EL device, 2 . . . substrate, 3 . . . anode, 4 . . . cathode, 5 . . . emitting layer (an example of first organic layer), 6 . . . hole injecting layer, 7 . . . hole transporting layer, 8 . . . electron transporting layer, 9 . . . electron injecting layer, 10 . . . organic layer

1. An organic electroluminescence device, comprising:
an anode;
a cathode; and
a first organic layer interposed between the anode and the cathode, wherein
the first organic layer comprises a first compound, a second compound, and a third compound,
the first compound is a compound represented by a formula (1): 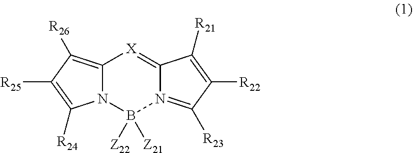
where, in the formula (1): X is a nitrogen atom, or a carbon atom bonded to Y;
Y is a hydrogen atom or a substituent;
R₂₁ to R₂₆ are each independently a hydrogen atom or a substituent, or at least one of a pair of R₂₁ and R₂₂, a pair of R₂₂ and R₂₃, a pair of R₂₄ and R₂₅, or a pair of R₂₅ and R₂₆ are mutually bonded to form a ring;
Y and R₂₁ to R₂₆ as the substituents are each independently selected from the group consisting of a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted aryl group having 6 to 30 ring carbon atoms, a substituted or unsubstituted alkoxy group having 1 to 30 carbon atoms, a substituted or unsubstituted alkoxy halide group having 1 to 30 carbon atoms, a substituted or unsubstituted alkylthio group having 6 to 30 carbon atoms, a substituted or unsubstituted aryloxy group having 6 to 30 ring carbon atoms, a substituted or unsubstituted arylthio group having 6 to 30 ring carbon atoms, a substituted or unsubstituted alkenyl group having 2 to 30 carbon atoms, a substituted or unsubstituted aralkyl group having 7 to 30 carbon atoms, a substituted or unsubstituted heteroaryl group having 5 to 30 ring atoms, a halogen atom, a carboxy group, a substituted or unsubstituted ester group, a substituted or unsubstituted carbamoyl group, a substituted or unsubstituted amino group, a nitro group, a cyano group, a substituted or unsubstituted silyl group, and a substituted or unsubstituted siloxanyl group;
Z₂₁ and Z₂₂ are each independently a substituent, or are mutually bonded to form a ring; and
Z₂₁ and Z₂₂ as the substituent are each independently selected from the group consisting of a halogen atom, a substituted or unsubstituted alkyl group having 1 to 30 carbon atoms, a substituted or unsubstituted alkyl halide group having 1 to 30 carbon atoms, a substituted or unsubstituted aryl group having 6 to 30 ring carbon atoms, a substituted or unsubstituted alkoxy group having 1 to 30 carbon atoms, a substituted or unsubstituted alkoxy halide group having 1 to 30 carbon atoms, and a substituted or unsubstituted aryloxy group having 6 to 30 ring carbon atoms,
the second compound is a delayed fluorescent compound, and
the third compound is a compound represented by a formula (3): 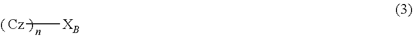
where, in the formula (3): n is 1, 2, 3 or 4;
when n is 2, 3, or 4, a plurality of Cz are mutually the same or different;
X_{B} is a group represented by a formula (3A):
Ar₁—(Ar₂)ₖ (3A)
where, in the formula (3A): Ar₁ and Ar₂ are each independently a substituted or unsubstituted monovalent or polyvalent aromatic hydrocarbon group having 6 to 18 ring carbon atoms;
k is 0, 1, or 2;
when k is 2, a plurality of Ar₂ are mutually the same or different;
Ar₁ is optionally substituted by a substituent D1, the substituent D1 being each independently selected from the group consisting of a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted aryl group having 6 to 30 ring carbon atoms, a substituted or unsubstituted aralkyl group having 7 to 30 carbon atoms, a substituted or unsubstituted alkoxy group having 1 to 30 carbon atoms, a substituted or unsubstituted alkoxy halide group having 1 to 30 carbon atoms, a substituted or unsubstituted aryloxy group having 6 to 30 ring carbon atoms, a substituted or unsubstituted silyl group, a halogen atom, a cyano group, and a nitro group;
Ar₂ is optionally substituted by a substituent D2, the substituent D2 representing the same as the substituent D1;
when n is 1, Cz is bonded to Ar₁ or Ar₂; and
when n is 2, 3 or 4, a plurality of Cz are each independently bonded to Ar₁ or Ar₂, and
Cz is a group represented by a formula (3B-1) or a formula (3B-2): 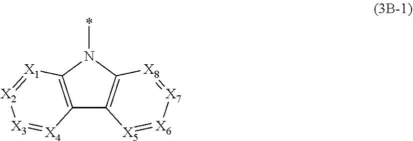
where, in the formula (3B-1): X₁ to X₈ each independently represent a nitrogen atom or CR_{A}, R_{A} being a hydrogen atom or a substituent, at least one pair of adjacent ones of R_{A} being optionally mutually bonded to form a ring;
R_{A} as the substituent is each independently selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted silyl group, a substituted or unsubstituted alkoxy group having 1 to 30 carbon atoms, a substituted or unsubstituted alkoxy halide group having 1 to 30 carbon atoms, a substituted or unsubstituted aryloxy group having 6 to 30 carbon atoms, a substituted or unsubstituted alkylthio group having 1 to 30 carbon atoms, a substituted or unsubstituted arylthio group having 6 to 30 ring carbon atoms, a substituted or unsubstituted aralkyl group having 7 to 30 carbon atoms, a substituted or unsubstituted alkenyl group having 2 to 30 carbon atoms, a halogen atom, a substituted or unsubstituted alkynyl group having 2 to 30 carbon atoms, a cyano group, a hydroxy group; a nitro group, and a carboxy group;
the substituted or unsubstituted heteroaryl group having 5 to 30 ring atoms for R_{A} is each independently a carbazolyl group or an azacarbazolyl group;
a plurality of R_{A} are mutually the same or different; and
the nitrogen atom in the formula (3B-1) is bonded to one of Ar₁ or Ar₂ in the formula (3A), 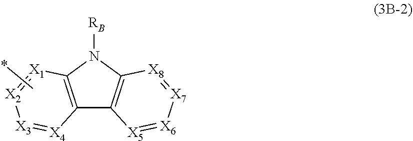
where, in the formula (3B-2): X₁ to X₄ are each a carbon atom bonded to one of Ar₁ and Ar₂ in the formula (3A), a nitrogen atom, or CR_{C}, R_{C} being a hydrogen atom or a substituent, at least least one pair of adjacent ones of R_{C} being optionally mutually bonded to form a ring, at least one of X₁ to X₄ being a carbon atom bonded to one of Ar₁ and Ar₂ in the formula (3A);
X₅ to X₈ are each a nitrogen atom or CR_{D}, R_{D} being a hydrogen atom or a substituent, at least one pair of adjacent ones of R_{D} being optionally mutually bonded to form a ring;
R_{B}, R_{C} and R_{D} each independently represent the same as R_{A} in the formula (3B-1), a plurality of R_{C} being mutually the same or different, a plurality of R_{D} being mutually the same or different;
the substituent E in the third compound meant by “substituted or unsubstituted” is a substitutent selected from the group consisting of a substituted or unsubstituted aryl group having 6 to 30 ring carbon atoms, a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted silyl group, a substituted or unsubstituted alkoxy group having 1 to 30 carbon atoms, a substituted or unsubstituted alkoxy halide group having 1 to 30 carbon atoms, a substituted or unsubstituted aryloxy group having 6 to 30 carbon atoms, a substituted or unsubstituted alkylthio group having 1 to 30 carbon atoms, a substituted or unsubstituted arylthio group having 6 to 30 ring carbon atoms, a substituted or unsubstituted aralkyl group having 7 to 30 carbon atoms, a substituted or unsubstituted alkenyl group having 2 to 30 carbon atoms, a halogen atom, a substituted or unsubstituted alkynyl group having 2 to 30 carbon atoms, a cyano group, a hydroxy group; a nitro group, and a carboxy group;
the substituent E optionally comprises a substituent F, the substituent F being selected from the group consisting of an unsubstituted aryl group having 6 to 30 ring carbon atoms, an unsubstituted alkyl group having 1 to 30 carbon atoms, an unsubstituted alkyl halide group having 1 to 30 carbon atoms, an unsubstituted cycloalkyl group having 3 to 30 ring carbon atoms, an unsubstituted silyl group, an unsubstituted alkoxy group having 1 to 30 carbon atoms, an unsubstituted halogenated alkoxy group having 1 to 30 carbon atoms, an unsubstituted aryloxy group having 6 to 30 carbon atoms, an unsubstituted alkylthio group having 1 to 30 carbon atoms, an unsubstituted arylthio group having 6 to 30 ring carbon atoms, an unsubstituted aralkyl group having 7 to 30 carbon atoms, an unsubstituted alkenyl group having 2 to 30 carbon atoms, a halogen atom, an unsubstituted alkynyl group having 2 to 30 carbon atoms, a cyano group, a hydroxy group; a nitro group, and a carboxy group; and
the substituent F is not further substituted.

2. The organic electroluminescence device according to claim 1, wherein
X₁ to X₈ in the formula (3B-1) are each independently CR_{A}; and
in the formula (3B-2), X₁ to X₄ are each independently a carbon atom bonded to one of Ar₁ and Ar₂, or CR_{C}, one of X₁ to X₄ being the carbon atom bonded to one of Ar₁ or Ar₂ in the formula (3A), X₅ to X₈ being each independently CR_{D}.

3. The organic electroluminescence device according to claim 1, wherein
in the formula (3B-1), a pair of adjacent ones of R_{A} are not mutually bonded; and
in the formula (3B-2), a pair of adjacent ones of R_{C} are not mutually bonded and a pair of adjacent ones of R_{D} are not mutually bonded.

4. The organic electroluminescence device according to claim 1, wherein
R_{A} in the formula (3B-1) and R_{B}, R_{C} and R_{D} in the formula (3B-2) are each independently a hydrogen atom, a substituted or unsubstituted aryl group having 6 to 30 ring carbon atoms, a substituted or unsubstituted alkyl group having 1 to 30 carbon atoms, a substituted or unsubstituted alkyl halide group having 1 to 30 carbon atoms, a carbazolyl group, a substituted carbazolyl group, a halogen atom, or a cyano group.

5. The organic electroluminescence device according to claim 1, wherein
R_{A} in the formula (3B-1) and R_{B}, R_{C} and R_{D} in the formula (3B-2) are each independently a hydrogen atom, a substituted or unsubstituted aryl group having 6 to 30 ring carbon atoms, a substituted or unsubstituted alkyl group having 1 to 30 carbon atoms, or a substituted or unsubstituted alkyl halide group having 1 to 30 carbon atoms.

6. The organic electroluminescence device according to claim 1, wherein
R_{A} in the formula (3B-1) and R_{C} and R_{D} in the formula (3B-2) are each a hydrogen atom.

7. The organic electroluminescence device according to claim 1, wherein
Cz in the formula (3) is a group represented by the formula (3B-1).

8. The organic electroluminescence device according to claim 1, wherein
n in the formula (3) is 1 or 2.

9. The organic electroluminescence device according to claim 1, wherein
Ar₁ and Ar₂ in the formula (3A) are each independently a monovalent or polyvalent residue derived from any one of benzene, biphenyl, terphenyl, naphthalene, anthracene, benzanthracene, phenanthrene, benzophenanthrene, phenalene, picene, pentacene, pyrene, chrysene, benzochrysene, fluoranthene, and triphenylene.

10. The organic electroluminescence device according claim 1, wherein
Ar₁ and Ar₂ in the formula (3A) are each independently a monovalent or polyvalent residue derived from any one of benzene, biphenyl, terphenyl, naphthalene, phenanthrene, fluoranthene, and triphenylene.

11. The organic electroluminescence device according to claim 1, wherein
Cz in the formula (3) is a group selected from the group consisting of groups represented by formulae (3B11) to (3B22): 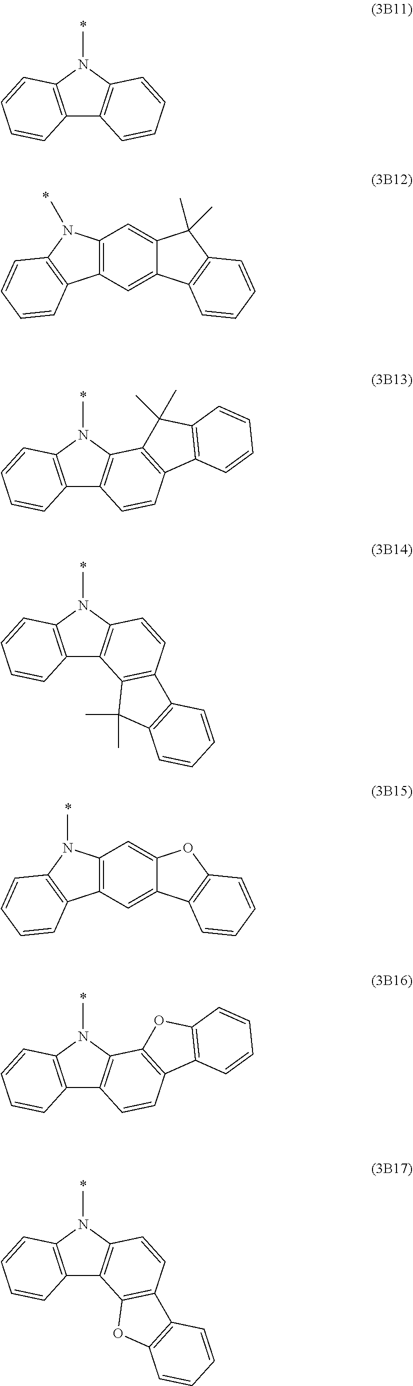 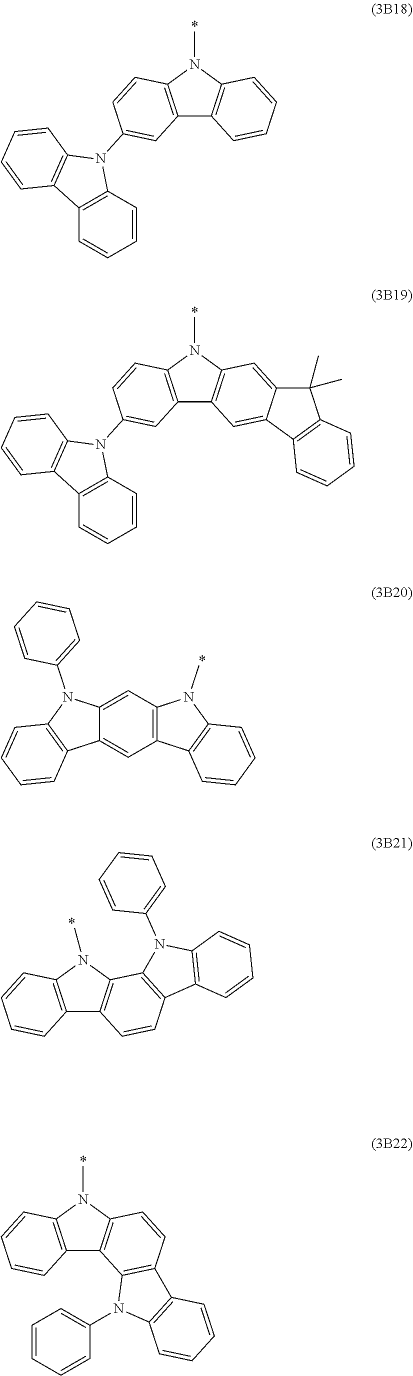 [SMILES: Cn1c2ccccc2c2cc(-n3c4ccccc4c4ccccc43)ccc21 ; Cn1c2ccc(-n3c4ccccc4c4ccccc43)cc2c2cc3c(cc21)C(C)(C)c1ccccc1-3 ; Cn1c2ccccc2c2cc3c4ccccc4n(-c4ccccc4)c3cc21 ; Cn1c2ccccc2c2ccc3c4ccccc4n(-c4ccccc4)c3c21 ; Cn1c2ccccc2c2c1ccc1c3ccccc3n(-c3ccccc3)c12]
where, in the groups represented by the formula (3B11) to (3B22), the carbon atom capable of being substituted by a substituent is substituted by a substituent X, which is the same as R_{A} as the substituent, or unsubstituted, the substituent X optionally comprising a plurality of substituents X that are mutually the same or different.

12. The organic electroluminescence device according to claim 11, wherein
Cz in the formula (3) is a group selected from the group consisting of groups represented by the formulae (3B11) to (3B17).

13. The organic electroluminescence device according to claim 1, wherein
the third compound is a compound represented by a formula (3C): 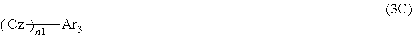
where, in the formula (3C): Cz represents the same as Cz in the formula (3);
Ar₃ represents the same as Ar₁ in the formula (3A); and
n1 represents the same as n in the formula (3A).

14. The organic electroluminescence device according to claim 13, wherein
n1 is 2.

15. The organic electroluminescence device according to claim 13, wherein
n1 is 2, and
Ar₃ in the formula (3C) is at least one group selected from the group consisting of groups represented by formulae (3a-1) to (3a-27): 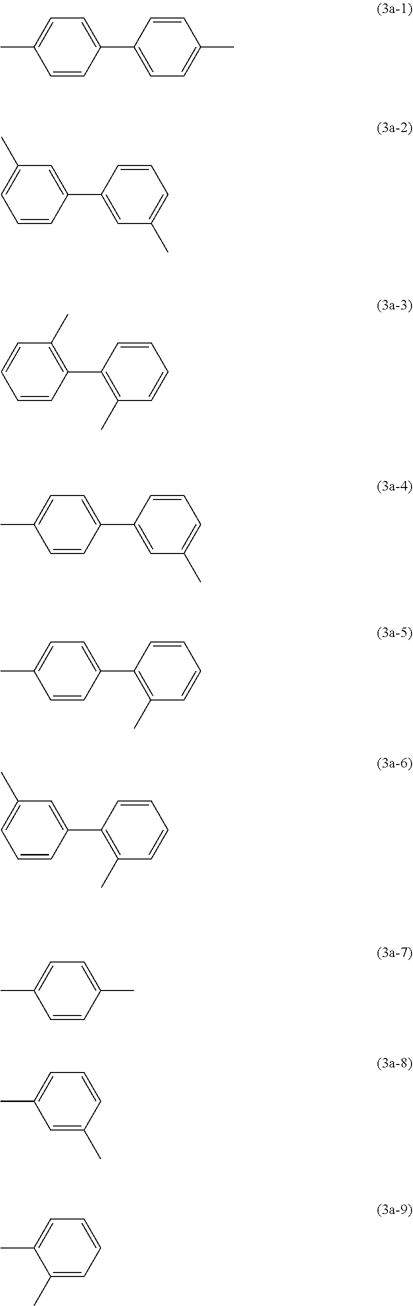 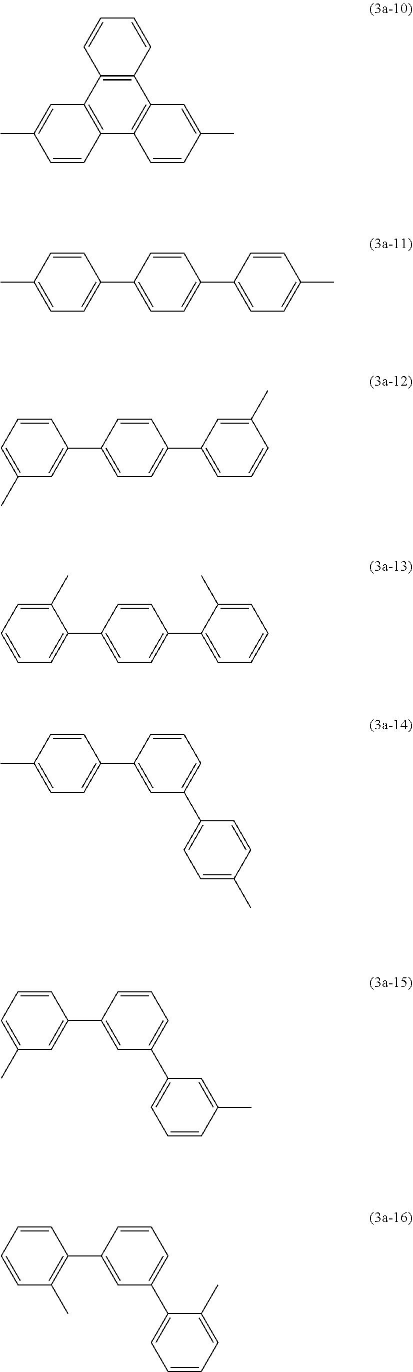 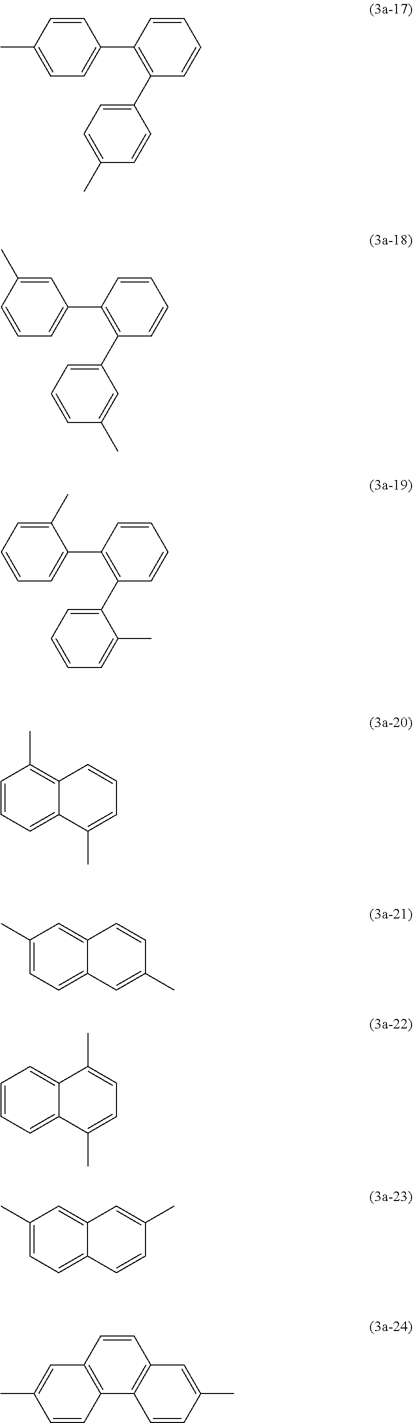 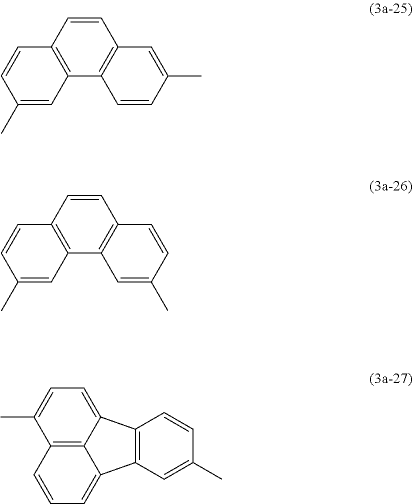
where the groups represented by the formulae (3a-1) to (3a-27) comprise a substituent E or are unsubstituted.

16. The organic electroluminescence device according to claim 1, wherein
in the formulae (3A), (3B-1), and (3B-2), the substituent E meant by “substituted or unsubstituted” is a substitutent selected from the group consisting of an unsubstituted aryl group having 6 to 30 ring carbon atoms, an unsubstituted alkyl group having 1 to 30 carbon atoms, an unsubstituted cycloalkyl group having 3 to 30 ring carbon atoms, a cyano group, and a halogen atom.

17. The organic electroluminescence device according to claim 1, wherein
in the formulae (3A), (3B-1), and (3B-2), the substituent E meant by “substituted or unsubstituted” is a substitutent selected from the group consisting of an unsubstituted aryl group having 6 to 30 ring carbon atoms, and an unsubstituted alkyl group having 1 to 30 carbon atoms.

18. The organic electroluminescence device according to claim 1, wherein
in the formulae (3A), (3B-1), and (3B-2), the substituent E meant by “substituted or unsubstituted” is a substitutent selected from the group consisting of an unsubstituted aryl group having 6 to 18 ring carbon atoms, and an unsubstituted alkyl group having 1 to 6 carbon atoms.

19. The organic electroluminescence device according to claim 1, wherein:
Cz is a group represented by the formula (3B-1);
n is 1 or 2;
R_{A} in the formula (3B-1) is each independently a hydrogen atom, a substituted or unsubstituted aryl group having 6 to 30 ring carbon atoms, a substituted or unsubstituted alkyl group having 1 to 30 carbon atoms, a carbazolyl group, a substituted carbazolyl group, a halogen atom, or a cyano group;
in the formula (3A), k is 0, and
Ar₁ is a monovalent or polyvalent residue derived from any one of benzene, biphenyl, terphenyl, naphthalene, anthracene, benzanthracene, phenanthrene, benzophenanthrene, phenalene, picene, pentacene, pyrene, chrysene, benzochrysene, fluoranthene, and triphenylene; and
in the formulae (3A) and (3B-1), a substituent E meant by “substituted or unsubstituted” is a substitutent selected from the group consisting of an unsubstituted aryl group having 6 to 30 ring carbon atoms, an unsubstituted alkyl group having 1 to 30 carbon atoms, an unsubstituted cycloalkyl group having 3 to 30 ring carbon atoms, a cyano group, and a halogen atom.

20. The organic electroluminescence device according to claim 1, wherein:
in the formula (3), n is 1 or 2;
in the formula (3A), k is 0, and
Ar₁ is a monovalent or polyvalent residue derived from one of benzene, biphenyl, terphenyl, naphthalene, phenanthrene, fluoranthene, and triphenylene;
X₁ to X₈ in the formula (3B-1) are each independently CR_{A};
X₁ to X₄ in the formula (3B-2) are each independently a carbon atom bonded to Ar₁, or CR_{C}, one of X₁ to X₄ being the carbon atom bonded to Ar₁ in the formula (3A), X₅ to X₈ being each independently CR_{D};
R_{A} in the formula (3B-1) and R_{C} and R_{D} in the formula (3B-2) are each independently a hydrogen atom, a substituted or unsubstituted aryl group having 6 to 30 ring carbon atoms, or a substituted or unsubstituted alkyl group having 1 to 30 carbon atoms;
Ar₁ is at least one group selected from the group consisting of groups represented by formulae (3a-1) to (3a-27): 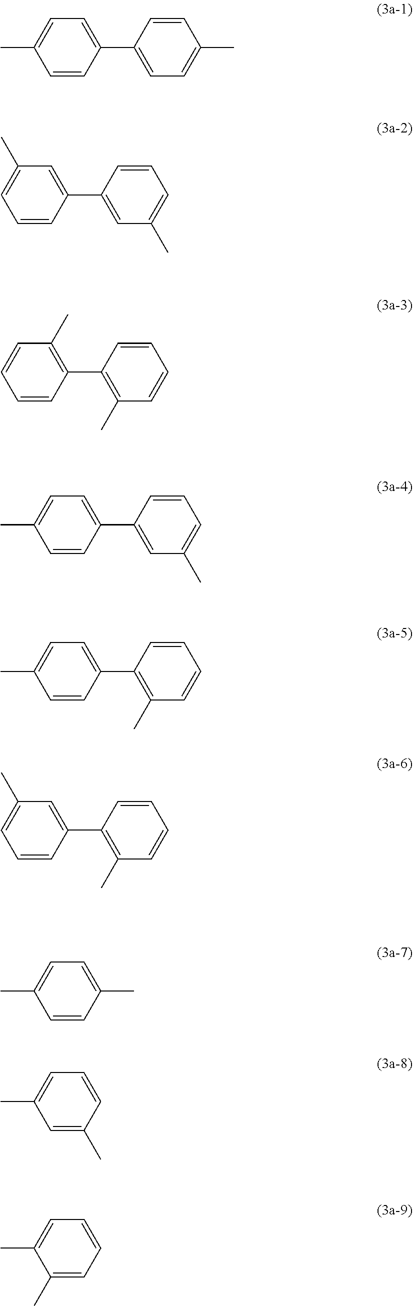 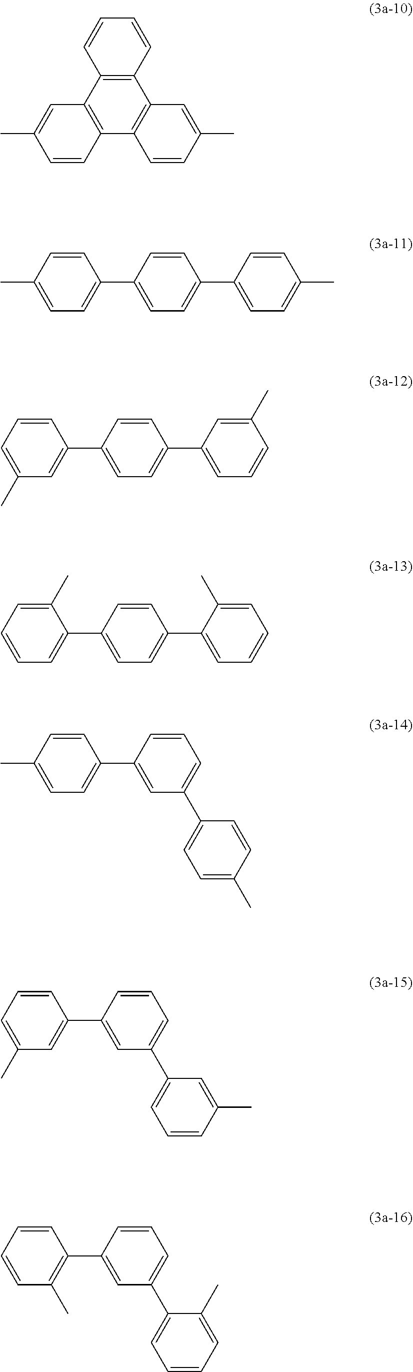 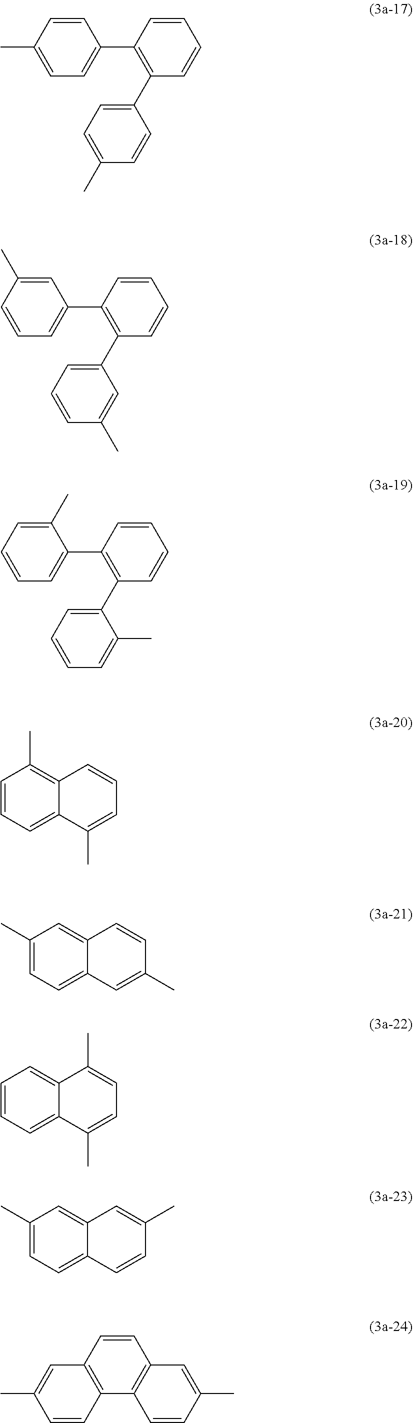 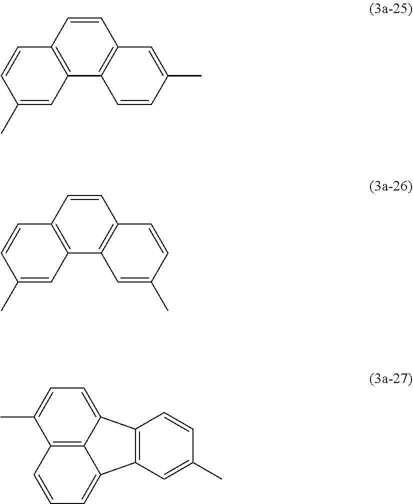
where the groups represented by the formulae (3a-1) to (3a-27) have the substituent E or are unsubstituted; and
in the formulae (3A), (3B-1), and (3B-2), a substituent E meant by “substituted or unsubstituted” is a substitutent selected from the group consisting of an unsubstituted aryl group having 6 to 30 ring carbon atoms, and an unsubstituted alkyl group having 1 to 30 carbon atoms.

21. The organic electroluminescence device according to claim 1,
wherein:
n in the formula (3) is 2;
k in the formula (3A) is 0;
in the formula (3B-1), a pair of adjacent ones of R_{A} are not mutually bonded;
in the formula (3B-2), a pair of adjacent ones of R_{C} are not mutually bonded and a pair of adjacent ones of R_{D} are not mutually bonded;
R_{A} in the formula (3B-1) and R_{C} and R_{D} in the formula (3B-2) are each a hydrogen atom; and in the formulae (3A), (3B-1), and (3B-2), a substituent E meant by “substituted or unsubstituted” is a substitutent selected from the group consisting of an unsubstituted aryl group having 6 to 18 ring carbon atoms, and an unsubstituted alkyl group having 1 to 6 carbon atoms.

22. The organic electroluminescence device according to claim 1, wherein
n in the formula (3) is 2;
k in the formula (3A) is 0;
Ar₁ is at least one group selected from the group consisting of groups represented by formulae (3a-1) to (3a-27); 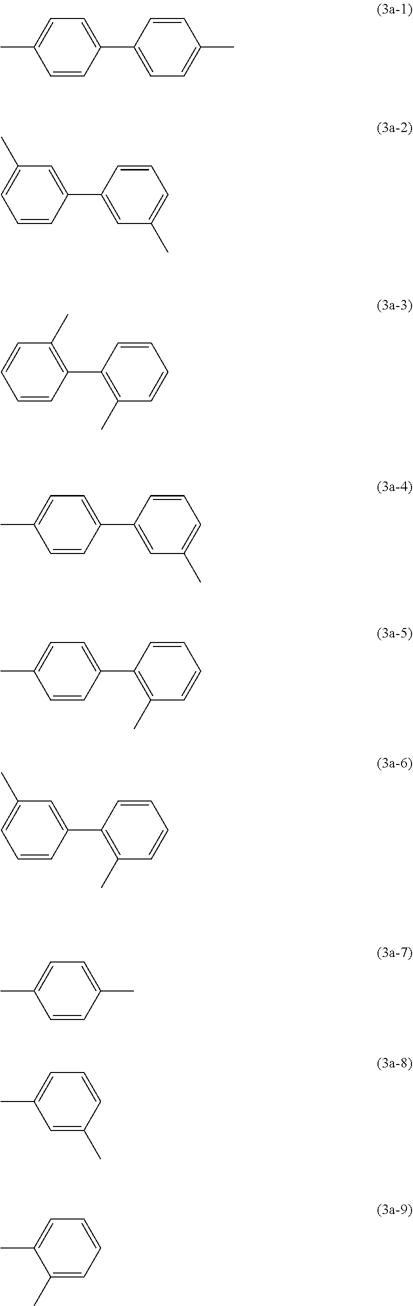 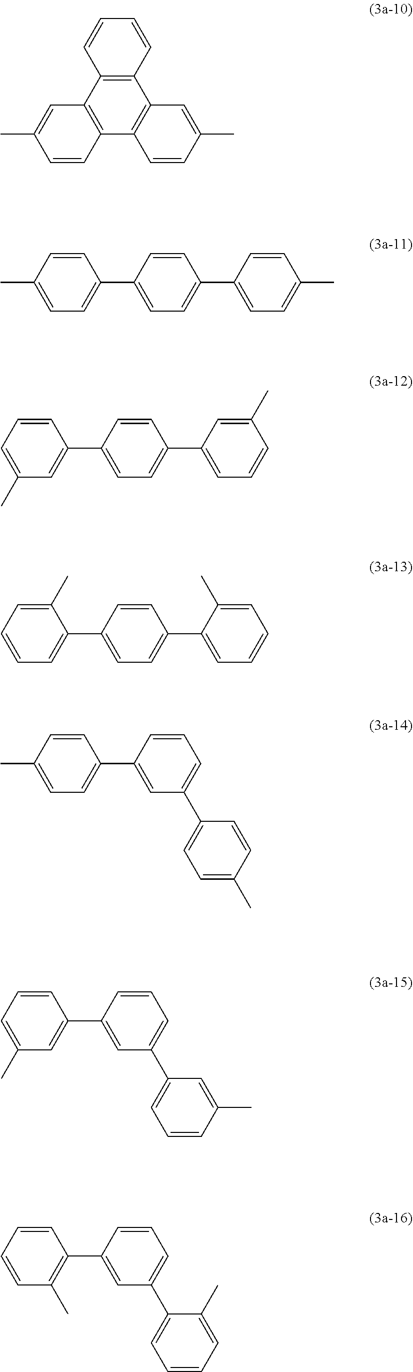 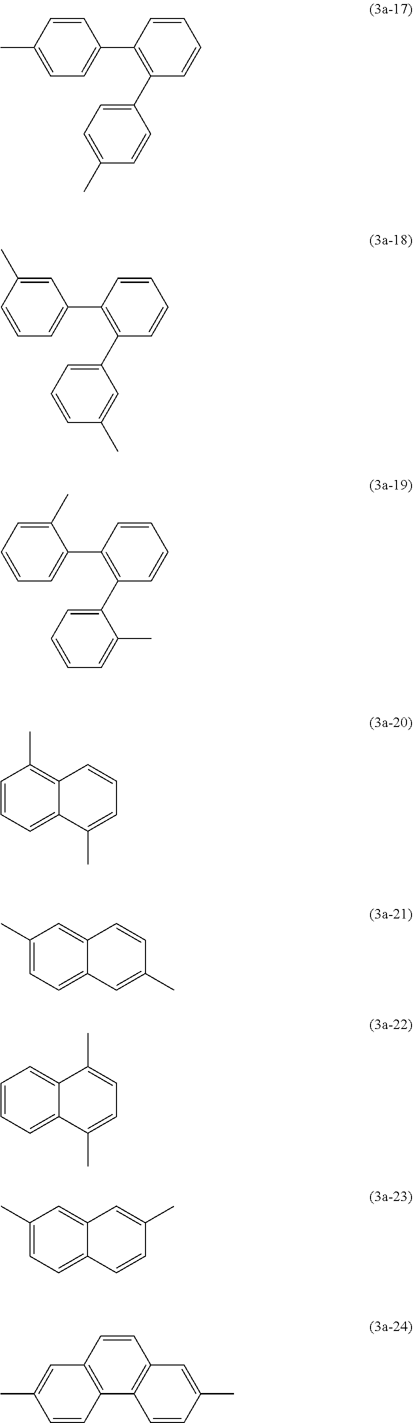 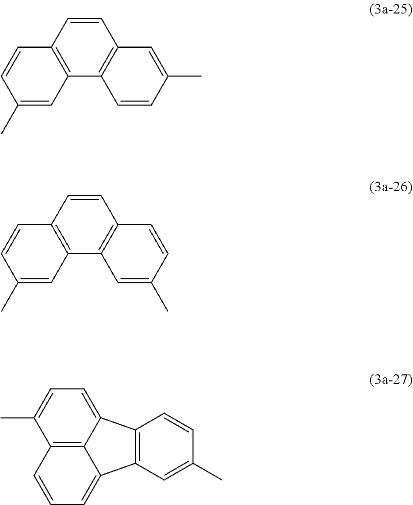
where the groups represented by the formulae (3a-1) to (3a-27) have the substituent E or are unsubstituted, and
in the formula (3B-1), a pair of adjacent ones of R_{A} are not mutually bonded,
in the formula (3B-2), a pair of adjacent ones of R_{C} are not mutually bonded and a pair of adjacent ones of R_{D} are not mutually bonded;
R_{A} in the formula (3B-1) and R_{C} and R_{D} in the formula (3B-2) are each a hydrogen atom; and
in the formulae (3A), (3B-1), and (3B-2), a substituent E meant by “substituted or unsubstituted” is a substitutent selected from the group consisting of an unsubstituted aryl group having 6 to 18 ring carbon atoms, and an unsubstituted alkyl group having 1 to 6 carbon atoms.

23. The organic electroluminescence device according to claim 1, wherein:
Ar₁ in a form of a monovalent aromatic hydrocarbon group and Ar₂ in a form of a monovalent aromatic hydrocarbon group are each independently a group selected from the group consisting of groups represented by formulae (3b-1) to (3b-3): 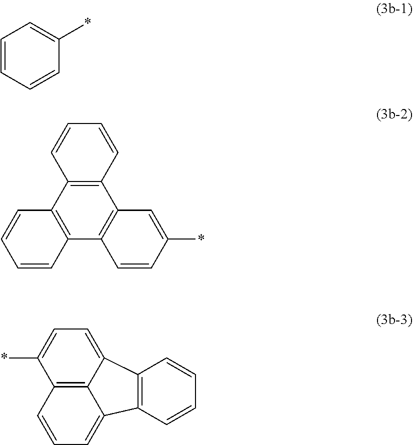 [SMILES: *c1ccccc1 ; *c1ccc2c3ccccc3c3ccccc3c2c1 ; *c1ccc2c3c(cccc13)-c1ccccc1-2]
and
Ar₁ in a form of a divalent aromatic hydrocarbon group and Ar₂ in a form of a divalent aromatic hydrocarbon group are each independently a group selected from the group consisting of groups represented by formulae (3b-4) to (3b-10):

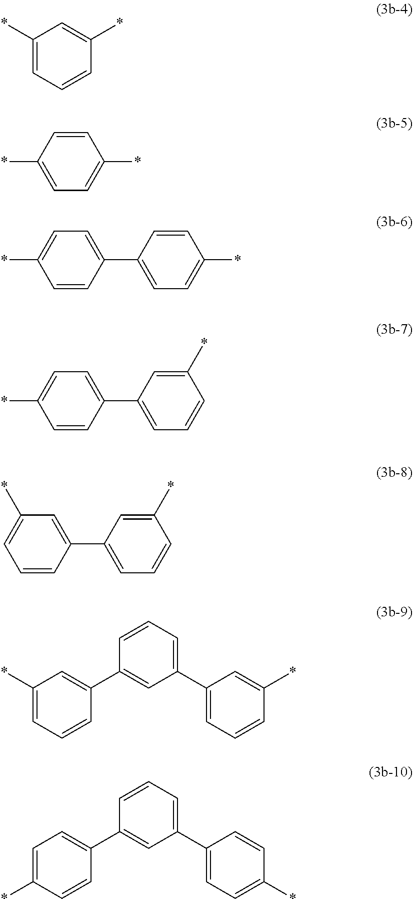

24. The organic electroluminescence device according to claim 1, wherein
both of Z₂₁ and Z₂₂ are fluorine atoms or are fluorine-atom-substituted alkoxy groups having 1 to 30 carbon atoms.

25. The organic electroluminescence device according claim 1, wherein
both of Z₂₁ and Z₂₂ are fluorine atoms.

26. The organic electroluminescence device according to claim 1, wherein
the first compound is a compound represented by a formula (10): 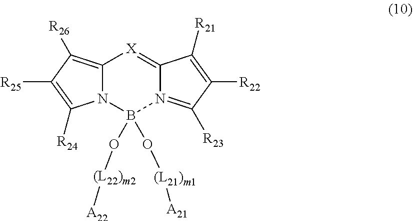
where, in the formula (10), X represents the same as X in the formula (1), Y when X is a carbon atom bonded to Y represents the same as Y in the formula (1);
R₂₁ to R₂₆ each independently represent the same as R₂₁ to R₂₆ in the formula (1);
L₂₁ and L₂₂ each independently represent a substituted or unsubstituted alkylene group having 1 to 6 carbon atoms or a substituted or unsubstituted arylene group having 6 to 12 ring carbon atoms;
A₂₁ and A₂₂ are each independently a substituted or unsubstituted alkyl group having 1 to 6 carbon atoms, a substituted or unsubstituted alkyl halide group having 1 to 6 carbon atoms, or a substituted or unsubstituted aryl group having 6 to 12 ring carbon atoms;
m1 and m2 are each an integer in a range from 0 to 7; and
when m1 is an integer in a range from 2 to 7, a plurality of L₂₁ are mutually the same or different, when m2 is an integer in a range from 2 to 7, a plurality of L₂₂ are mutually the same or different, when m1 is 0, A₂₁ is directly bonded to an oxygen atom, and when m2 is 0, A₂₂ is directly bonded to an oxygen atom.

27. The organic electroluminescence device according to claim 26, wherein
the first compound is a compound represented by a formula (10a): 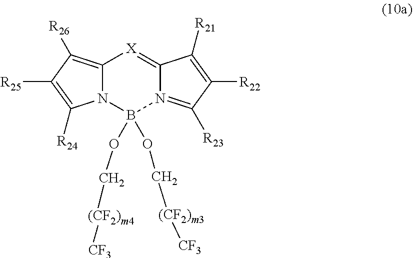
where, in the formula (10a): X represents the same as X in the formula (1), Y when X is a carbon atom bonded to Y represents the same as Y in the formula (1);
R₂₁ to R₂₆ each independently represent the same as R₂₁ to R₂₆ in the formula (1);
m3 is in a range from 0 to 4;
m4 is in a range from 0 to 4; and
m3 and m4 are mutually the same or different.

28. The organic electroluminescence device according to claim 1, wherein
X is a carbon atom bonded to Y; Y is a hydrogen atom or a substituent;
Y as a substituent is a substituted or unsubstituted aryl group having 6 to 30 ring carbon atoms; and when Y as a substituent is an aryl group having 6 to 30 ring carbon atoms having a substituent, the substituent is a substituted or unsubstituted alkyl group having 1 to 30 carbon atoms, a substituted or unsubstituted alkyl halide group having 1 to 30 carbon atoms, a substituted or unsubstituted alkoxy group having 1 to 30 carbon atoms, a substituted or unsubstituted alkoxy halide group having 1 to 30 carbon atoms, or an aryl group having 6 to 30 ring carbon atoms substituted by an alkyl group having 1 to 30 carbon atoms.

29. The organic electroluminescence device according to claim 1, wherein
R₂₁, R₂₃, R₂₄ and R₂₆ are each independently a substituted or unsubstituted alkyl group having 1 to 30 carbon atoms, a substituted or unsubstituted alkyl halide group having 1 to 30 carbon atoms, or an aryl group having 6 to 30 ring carbon atoms substituted by an alkyl group having 1 to 30 carbon atoms, and
R₂₂ and R₂₅ are hydrogen atoms.

30. The organic electroluminescence device according to claim 1, wherein
R₂₁, R₂₃, R₂₄ and R₂₆ are each independently a substituted or unsubstituted alkyl group having 1 to 6 carbon atoms, a substituted or unsubstituted alkyl halide group having 1 to 6 carbon atoms, or an aryl group having 6 to 12 ring carbon atoms substituted by an alkyl group having 1 to 6 carbon atoms, and
R₂₂ and R₂₅ are hydrogen atoms.

31. The organic electroluminescence device according to claim 1, wherein
the substituent in the first compound meant by “substituted or unsubstituted” is a substitutent selected from the group consisting of a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted aryl group having 6 to 30 ring carbon atoms, a substituted or unsubstituted heteroaryl group having 5 to 30 ring atoms, a substituted or unsubstituted alkoxy group having 1 to 30 carbon atoms, a substituted or unsubstituted alkoxy halide group having 1 to 30 carbon atoms, a substituted or unsubstituted alkylthio group having 1 to 30 carbon atoms, a substituted or unsubstituted aryloxy group having 6 to 30 ring carbon atoms, a substituted or unsubstituted arylthio group having 6 to 30 ring carbon atoms, a substituted or unsubstituted alkenyl group having 2 to 30 carbon atoms, a substituted or unsubstituted alkynyl group having 2 to 30 carbon atoms, a substituted or unsubstituted aralkyl group having 7 to 30 carbon atoms, a halogen atom, a carboxy group, a substituted or unsubstituted amino group, a nitro group, a cyano group, a substituted or unsubstituted silyl group, a substituted phosphoryl group, and a hydroxy group.

32. The organic electroluminescence device according to claim 1, wherein
the substituent in the first compound meant by “substituted or unsubstituted” is a substitutent selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted alkyl halide group having 1 to 30 carbon atoms, and a substituted or unsubstituted cycloalkyl group having 3 to 30 ring carbon atoms.

33. The organic electroluminescence device according to claim 1, wherein
the substituent in the first compound meant by “substituted or unsubstituted” is a substitutent selected from the group consisting of a substituted or unsubstituted aryl group having 6 to 12 ring carbon atoms, a substituted or unsubstituted heteroaryl group having 5 to 12 ring atoms, a substituted or unsubstituted alkyl group having 1 to 6 carbon atoms, a substituted or unsubstituted alkyl halide group having 1 to 6 carbon atoms, and a substituted or unsubstituted cycloalkyl group having 3 to 12 ring carbon atoms.

34. The organic electroluminescence device according to claim 1, wherein
a singlet energy S₁(Mat1) of the first compound and a singlet energy S₁(Mat2) of the second compound satisfy a relationship of Numerical Formula 1:
S₁(Mat2)>S₁(Mat1) (Numerical Formula 1).

35. The organic electroluminescence device according to claim 1, wherein
a singlet energy S₁(Mat2) of the second compound and a singlet energy S₁(Mat3) of the third compound satisfy a relationship of Numerical Formula 2:
S₁(Mat3)>S₁(Mat2) (Numerical Formula 2)

36. An electronic device comprising the organic electroluminescence device according to claim 1.
